(19) United States
(12) Patent Application Publication
(10) Pub. No.: US 2019/0023791 A1
(43) Pub. Date: Jan. 24, 2019

(54) Combination Treatments and Uses and Methods Thereof

(71) Applicants: GLAXOSMITHKLINE INTELLECTUAL PROPERTY DEVELOPMENT LIMITED, Brentford, Middlesex (GB); MERCK SHARP & DOHME CORP., Rahway, NJ (US)

(72) Inventors: Axel HOOS, Collegeville, PA (US); David KAUFMAN, North Wales, PA (US); Elaine PINHEIRO, Boston, MA (US); Herbert STRUEMPER, Durham, NC (US); Niranjan YANAMANDRA, Collegeville, PA (US)

(73) Assignees: GLAXOSMITHKLINE INTELLECTUAL PROPERTY DEVELOPMENT LIMITED, Brentford, Middlesex (GB); MERCK SHARP & DOHME CORP., Rahway, NJ (US)

(21) Appl. No.: 15/749,141

(22) PCT Filed: Aug. 3, 2016
(86) PCT No.: PCT/IB2016/054692

§ 371 Date: Jan. 31, 2018

Related U.S. Application Data
(60) Provisional application No. 62/204,555, filed on Aug. 13, 2015.
(60) Provisional application No. 62/200,779, filed on Aug. 4, 2015.

(51) Int. Cl. C07K 16/28; A61P 35/00; A61K 9/00

(52) U.S. Cl. CPC C07K 16/2818; A61P 35/00; C07K 2317/24; C07K 16/2878; C07K 2317/565; A61K 9/0019

(57) ABSTRACT

The present invention provides methods of treating cancer in a human in need thereof comprising administering to the human:
a therapeutically effective amount of a monoclonal antibody that binds to human OX40 comprising: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9; and
a therapeutically effective amount of a monoclonal antibody that binds to human PD-1 comprising: (a) a heavy chain variable region CDR1 comprising the amino acid sequence of SEQ ID NO:54; (b) a heavy chain variable region CDR2 comprising the amino acid sequence of SEQ ID NO:55; (c) a heavy chain variable region CDR3 comprising the amino acid sequence of SEQ ID NO:56; (d) a light chain variable region CDR1 comprising the amino acid sequence of SEQ ID NO:57; (e) a light chain variable region CDR2 comprising the amino acid sequence of SEQ ID NO:58; and (f) a light chain variable region CDR3 comprising the amino acid sequence of SEQ ID NO:59.

### CROSS REFERENCE TO RELATED APPLICATIONS

This application claims priority to U.S. Application Ser. No. 62/200,779, filed on Aug. 4, 2015 and U.S. Application Ser. No. 62/204,555, filed on Aug. 13, 2015. The disclosure of the prior applications are considered part of (and are incorporated by reference in) the disclosure of this application.

### SEQUENCE LISTING

The instant application contains a Sequence Listing which has been submitted electronically in ASCII format and is hereby incorporated by reference in its entirety. Said ASCII copy, created on Jul. 29, 2016, is named PU65945PCT_SL.txt and is 110,170 bytes in size.

### FIELD OF THE INVENTION

The present invention relates to a method of treating cancer in a mammal and to combinations useful in such treatment. In particular the present invention relates to combinations of anti-OX40 antigen binding proteins (ABPs), including monoclonal antibodies to human OX40 and one or more anti-PD-1 ABPs, including monoclonal antibodies to human PD-1.

### BACKGROUND OF THE INVENTION

Effective treatment of hyperproliferative disorders including cancer is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes include abnormalities in signal transduction pathways and response to factors which differ from those found in normal cells.

Immunotherapies are one approach to treat hyperproliferative disorders. A major hurdle that scientists and clinicians have encountered in the development of various types of cancer immunotherapies has been to break tolerance to self antigen (cancer) in order to mount a robust anti-tumor response leading to tumor regression. Unlike traditional development of small and large molecule agents that target the tumor, cancer immunotherapies target cells of the immune system that have the potential to generate a memory pool of effector cells to induce more durable effects and minimize recurrences.

OX40 is a costimulatory molecule involved in multiple processes of the immune system. Antigen binding proteins and antibodies that bind OX-40 receptor and modulate OX40 signaling are known in the art and are disclosed as immunotherapy, for example for cancer.

Binding of the PD-1 ligands, PD-L1 and PD-L2, to the PD-1 receptor found on T cells, inhibits T cell proliferation and cytokine production. Upregulation of PD-1 ligands occurs in some tumors and signaling through this pathway can contribute to inhibition of active T-cell immune surveillance of tumors. Antigen binding proteins and antibodies that bind to the PD-1 receptor and block its interaction with PD-L1 and PD-L2 may release PD-1 pathway-mediated inhibition of the immune response, including the anti-tumor immune response.

Enhancing anti-tumor T cell function and inducing T cell proliferation is a powerful and new approach for cancer treatment. Three immune-oncology antibodies (e.g., immuno-modulators) are presently marketed. Anti-CTLA-4 (YERVOY®/ipilimumab) is thought to augment immune responses at the point of T cell priming and anti-PD-1 antibodies (OPDIVO®/nivolumab and KEYTRUDA®/pembrolizumab) are thought to act in the local tumor microenvironment, by relieving an inhibitory checkpoint in tumor specific T cells that have already been primed and activated.

Though there have been many recent advances in the treatment of cancer, there remains a need for more effective and/or enhanced treatment of an individual suffering the effects of cancer. The combinations and methods herein that relate to combining therapeutic approaches for enhancing anti-tumor immunity address this need.

### SUMMARY OF THE INVENTION

Provided herein are methods of treating cancer in a human in need thereof comprising administering a therapeutically effective amount of a monoclonal antibody that binds to human OX40 comprising (a) a heavy chain variable region CDR1 comprising the amino acid sequence of SEQ ID NO: 1; (b) a heavy chain variable region CDR2 comprising the amino acid sequence of SEQ ID NO: 2; (c) a heavy chain variable region CDR3 comprising the amino acid sequence of SEQ ID NO. 3; (d) a light chain variable region CDR1 comprising the amino acid sequence of SEQ ID NO. 7; (e) a light chain variable region CDR2 comprising the amino acid sequence of SEQ ID NO. 8; and (f) a light chain variable region CDR3 comprising the amino acid sequence of SEQ ID NO. 9, and pembrolizumab, or an antibody having 90%, 91%, 92%, 93%, 94%, 95%, 96%, 97%, 98%, 99% or 100% sequence identity thereto. Also provided herein are pharmaceutical compositions and kits comprising a monoclonal antibody that binds human OX40 and pembrolizumab.

Further provided herein are methods of treating cancer in a human in need thereof comprising administering a therapeutically effective amount of a monoclonal antibody that binds to human OX40 comprising a VH (variable heavy chain) region comprising an amino acid sequence with at least 90%, 91%, 92%, 93%, 94%, 95%, 96%, 97%, 98%, 99% or 100% sequence identity to the amino acid sequence as set forth in SEQ ID NO: 4 and a VL (variable light chain) region comprising and amino acid sequence with at least 90%, 91%, 92%, 93%, 94%, 95%, 96%, 97%, 98%, 99% or 100% sequence identity to the amino acid sequence as set forth in SEQ ID NO:10, and pembrolizumab, or an antibody having 90%, 91%, 92%, 93%, 94%, 95%, 96%, 97%, 98%, 99% or 100% sequence identity thereto. Also provided herein are pharmaceutical compositions and kits comprising a monoclonal antibody that binds human OX40 and pembrolizumab.

Also provided herein are methods of treating cancer in a human in need thereof comprising administering a therapeutically effective amount of a monoclonal antibody that binds to human OX40 comprising a VH (variable heavy chain) region comprising an amino acid sequence with at least 90%, 91%, 92%, 93%, 94%, 95%, 96%, 97%, 98%, 99% or 100% sequence identity to the amino acid sequence as set forth in SEQ ID NO: 5 and a VL (variable light chain) region comprising and amino acid sequence with at least 90%, 91%, 92%, 93%, 94%, 95%, 96%, 97%, 98%, 99% or 100% sequence identity to the amino acid sequence as set forth in SEQ ID NO:11, and pembrolizumab, or an antibody having 90%, 91%, 92%, 93%, 94%, 95%, 96%, 97%, 98%, 99% or 100% sequence identity thereto. Also provided herein are pharmaceutical compositions and kits comprising a monoclonal antibody that binds human OX40 and pembrolizumab.

Also provided herein are methods of reducing tumor size in a human having cancer comprising administering a therapeutically effective amount of ANTIBODY 106-222 and a therapeutically effective amount of pembrolizumab to said human.

In some aspects, the disclosure provides a method of treating cancer in a human in need thereof comprising administering to the human:
a therapeutically effective amount of a monoclonal antibody that binds to human OX40 comprising: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9; and
a therapeutically effective amount of a monoclonal antibody that binds to human PD-1 comprising: (a) a heavy chain variable region CDR1 comprising the amino acid sequence of SEQ ID NO:54; (b) a heavy chain variable region CDR2 comprising the amino acid sequence of SEQ ID NO:55; (c) a heavy chain variable region CDR3 comprising the amino acid sequence of SEQ ID NO:56; (d) a light chain variable region CDR1 comprising the amino acid sequence of SEQ ID NO:57; (e) a light chain variable region CDR2 comprising the amino acid sequence of SEQ ID NO:58; and (f) a light chain variable region CDR3 comprising the amino acid sequence of SEQ ID NO:59.

In some embodiments, the monoclonal antibody that binds to human OX40 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:5 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:11.

In some embodiments, the monoclonal antibody that binds to human PD-1 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:52 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:53.

In some embodiments, the monoclonal antibody that binds to human OX40 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:5 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:11, and the monoclonal antibody that binds to human PD-1 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:52 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:53.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the monoclonal antibody that binds to human PD-1 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:50 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:51.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49, and the monoclonal antibody that binds to human PD-1 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO: 50 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:51.

In some embodiments, the monoclonal antibody that binds to human OX40 comprises a heavy chain comprising the amino acid sequence set forth in SEQ ID NO:48 and a light chain comprising the amino acid sequence set forth in SEQ ID NO:49, and the monoclonal antibody that binds to human PD-1 comprises a heavy chain comprising the amino acid sequence set forth in SEQ ID NO: 50 and a light chain comprising the amino acid sequence set forth in SEQ ID NO:51.

In some embodiments, the cancer is a solid tumor. In some embodiments, the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

In some embodiments, the monoclonal antibody that binds to OX40 and the monoclonal antibody that binds to human PD-1 are administered at the same time.

In some embodiments, the monoclonal antibody that binds to human OX40 and the monoclonal antibody that binds to human PD-1 are administered sequentially, in any order.

In some embodiments, the monoclonal antibody that binds to OX40 and/or the monoclonal antibody that binds to human PD-1 are administered intravenously.

In some embodiments, the monoclonal antibody that binds to OX40 and/or the monoclonal antibody that binds to human PD-1 are administered intratumorally.

In some embodiments, the monoclonal antibody that binds to OX40 is administered at a dose of about 0.1 mg/kg to about 10 mg/kg.

In some embodiments, the monoclonal antibody that binds to OX40 is administered at a frequency selected from the group consisting of: once daily, once weekly, once every two weeks (Q2W), and once every three weeks (Q3W).

In some embodiments, the monoclonal antibody that binds to human PD-1 is administered at a dose of about 200 mg.

In some embodiments, the monoclonal antibody that binds to human PD-1 is administered Q3W.

In some aspects, the disclosure provides a method of reducing tumor size in a human having cancer comprising administering a therapeutically effective amount of a monoclonal antibody that binds to human OX40 that comprises a heavy chain comprising the amino acid sequence set forth in SEQ ID NO:48 and a light chain comprising the amino acid sequence set forth in SEQ ID NO:49, and a therapeutically effective amount of a monoclonal antibody that binds to human PD-1 that comprises a heavy chain comprising the amino acid sequence set forth in SEQ ID NO: 50 and a light chain comprising the amino acid sequence set forth in SEQ ID NO:51. to said human.

In some embodiments, the human demonstrates complete response or partial response according to RECIST version 1.1.

In some embodiments, the monoclonal antibody that binds to human PD-1 is intravenously administered to the human starting at least 1 hour and no more than 2 hours following the end of intravenous administration of the monoclonal antibody that binds to human OX40.

In some aspects, the disclosure provides a pharmaceutical composition or kit comprising
a therapeutically effective amount of a monoclonal antibody that binds to human OX40 comprising: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9; and
a therapeutically effective amount of a monoclonal antibody that binds to human PD-1 comprising: (a) a heavy chain variable region CDR1 comprising the amino acid sequence of SEQ ID NO:54; (b) a heavy chain variable region CDR2 comprising the amino acid sequence of SEQ ID NO:55; (c) a heavy chain variable region CDR3 comprising the amino acid sequence of SEQ ID NO:56; (d) a light chain variable region CDR1 comprising the amino acid sequence of SEQ ID NO:57; (e) a light chain variable region CDR2 comprising the amino acid sequence of SEQ ID NO:58; and (f) a light chain variable region CDR3 comprising the amino acid sequence of SEQ ID NO:59.

In some embodiments, the monoclonal antibody that binds to human OX40 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:5 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:11.

In some embodiments, the monoclonal antibody that binds to human PD-1 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:52 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:53.

In some embodiments, the monoclonal antibody that binds to human OX40 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:5 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:11, and the monoclonal antibody that binds to human PD-1 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:52 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:53.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the monoclonal antibody that binds to human PD-1 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:50 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:51.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49, and the monoclonal antibody that binds to human PD-1 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO: 50 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:51.

In some embodiments, the monoclonal antibody that binds to human OX40 comprises a heavy chain comprising the amino acid sequence set forth in SEQ ID NO:48 and a light chain comprising the amino acid sequence set forth in SEQ ID NO:49, and the monoclonal antibody that binds to human PD-1 comprises a heavy chain comprising the amino acid sequence set forth in SEQ ID NO: 50 and a light chain comprising the amino acid sequence set forth in SEQ ID NO:51.

In some aspects, the disclosure provides a combination kit comprising a pharmaceutical composition or kit described herein together with one or more pharmaceutically acceptable carriers.

In some aspects, the disclosure provides use of the pharmaceutical composition or kit described herein in the manufacture of a medicament for the treatment of cancer.

In some aspects, the disclosure provides a kit for use in the treatment of cancer comprising:
a therapeutically effective amount of a monoclonal antibody that binds to human OX40 comprising: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9; and
a therapeutically effective amount of a monoclonal antibody that binds to human PD-1 comprising: (a) a heavy chain variable region CDR1 comprising the amino acid sequence of SEQ ID NO:54; (b) a heavy chain variable region CDR2 comprising the amino acid sequence of SEQ ID NO:55; (c) a heavy chain variable region CDR3 comprising the amino acid sequence of SEQ ID NO:56; (d) a light chain variable region CDR1 comprising the amino acid sequence of SEQ ID NO:57; (e) a light chain variable region CDR2 comprising the amino acid sequence of SEQ ID NO:58; and (f) a light chain variable region CDR3 comprising the amino acid sequence of SEQ ID NO:59; and
instructions for use in the treatment of cancer.

In some embodiments, the monoclonal antibody that binds to human OX40 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:5 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:11.

In some embodiments, the monoclonal antibody that binds to human PD-1 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:52 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:53.

In some embodiments, the monoclonal antibody that binds to human OX40 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:5 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:11, and the monoclonal antibody that binds to human PD-1 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:52 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:53.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the monoclonal antibody that binds to human PD-1 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:50 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:51.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49, and the monoclonal antibody that binds to human PD-1 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO: 50 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:51.

In some embodiments, the monoclonal antibody that binds to human OX40 comprises a heavy chain comprising the amino acid sequence set forth in SEQ ID NO:48 and a light chain comprising the amino acid sequence set forth in SEQ ID NO:49, and the monoclonal antibody that binds to human PD-1 comprises a heavy chain comprising the amino acid sequence set forth in SEQ ID NO: 50 and a light chain comprising the amino acid sequence set forth in SEQ ID NO:51.

In some embodiments, the monoclonal antibody that binds to human OX40 and the monoclonal antibody that binds to human PD-1 are each individually formulated with one or more pharmaceutically acceptable carriers.

In some embodiments, the cancer is a solid tumor. In some embodiments, the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

In some aspects, the disclosure provides
a therapeutically effective amount of a monoclonal antibody that binds to human OX40 comprising: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9; and
a therapeutically effective amount of a monoclonal antibody that binds to human PD-1 comprising: (a) a heavy chain variable region CDR1 comprising the amino acid sequence of SEQ ID NO:54; (b) a heavy chain variable region CDR2 comprising the amino acid sequence of SEQ ID NO:55; (c) a heavy chain variable region CDR3 comprising the amino acid sequence of SEQ ID NO:56; (d) a light chain variable region CDR1 comprising the amino acid sequence of SEQ ID NO:57; (e) a light chain variable region CDR2 comprising the amino acid sequence of SEQ ID NO:58; and (f) a light chain variable region CDR3 comprising the amino acid sequence of SEQ ID NO:59, for use (e.g., simultaneous or sequential use) in treating cancer in a human in need thereof.

In some embodiments, the monoclonal antibody that binds to human OX40 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:5 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:11.

In some embodiments, the monoclonal antibody that binds to human PD-1 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:52 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:53.

In some embodiments, the monoclonal antibody that binds to human OX40 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:5 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:11, and the monoclonal antibody that binds to human PD-1 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:52 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:53.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the monoclonal antibody that binds to human PD-1 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:50 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:51.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49, and the monoclonal antibody that binds to human PD-1 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO: 50 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:51.

In some embodiments, the monoclonal antibody that binds to human OX40 comprises a heavy chain comprising the amino acid sequence set forth in SEQ ID NO:48 and a light chain comprising the amino acid sequence set forth in SEQ ID NO:49, and the monoclonal antibody that binds to human PD-1 comprises a heavy chain comprising the amino acid sequence set forth in SEQ ID NO: 50 and a light chain comprising the amino acid sequence set forth in SEQ ID NO:51.

In some embodiments, the cancer is a solid tumor. In some embodiments, the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

In some embodiments, the monoclonal antibody that binds to OX40 and the monoclonal antibody that binds to human PD-1 are to be administered at the same time.

In some embodiments, the monoclonal antibody that binds to human OX40 and the monoclonal antibody that binds to human PD-1 are to be administered sequentially, in any order.

In some embodiments, the monoclonal antibody that binds to OX40 and/or the monoclonal antibody that binds to human PD-1 are to be administered intravenously.

In some embodiments, the monoclonal antibody that binds to OX40 and/or the monoclonal antibody that binds to human PD-1 are to be administered intratumorally.

In some embodiments, the monoclonal antibody that binds to OX40 is to be administered at a dose of about 0.1 mg/kg to about 10 mg/kg.

In some embodiments, the monoclonal antibody that binds to OX40 is to be administered at a frequency selected from the group consisting of: once daily, once weekly, once every two weeks (Q2W), and once every three weeks (Q3W).

In some embodiments, the monoclonal antibody that binds to human PD-1 is to be administered at a dose of about 200 mg.

In some embodiments, the monoclonal antibody that binds to human PD-1 is to be administered Q3W.

In some aspects, the disclosure provides a therapeutically effective amount of a monoclonal antibody that binds to human OX40 that comprises a heavy chain comprising the amino acid sequence set forth in SEQ ID NO:48 and a light chain comprising the amino acid sequence set forth in SEQ ID NO:49, and a therapeutically effective amount of a monoclonal antibody that binds to human PD-1 that comprises a heavy chain comprising the amino acid sequence set forth in SEQ ID NO: 50 and a light chain comprising the amino acid sequence set forth in SEQ ID NO:51 for use (e.g., simultaneous or sequential use) in reducing tumor size in a human having cancer.

In some embodiments, the human demonstrates complete response or partial response according to RECIST version 1.1.

In some embodiments, the monoclonal antibody that binds to human PD-1 is to be intravenously administered to the human starting at least 1 hour and no more than 2 hours following the end of intravenous administration of the monoclonal antibody that binds to human OX40.

In some aspects, the disclosure provides use (e.g., simultaneous or sequential use) of
a therapeutically effective amount of a monoclonal antibody that binds to human OX40 comprising: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9; and
a therapeutically effective amount of a monoclonal antibody that binds to human PD-1 comprising: (a) a heavy chain variable region CDR1 comprising the amino acid sequence of SEQ ID NO:54; (b) a heavy chain variable region CDR2 comprising the amino acid sequence of SEQ ID NO:55; (c) a heavy chain variable region CDR3 comprising the amino acid sequence of SEQ ID NO:56; (d) a light chain variable region CDR1 comprising the amino acid sequence of SEQ ID NO:57; (e) a light chain variable region CDR2 comprising the amino acid sequence of SEQ ID NO:58; and (f) a light chain variable region CDR3 comprising the amino acid sequence of SEQ ID NO:59 for the preparation of a medicament for treating cancer in a human in need thereof.

In some embodiments, the monoclonal antibody that binds to human OX40 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:5 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:11.

In some embodiments, the monoclonal antibody that binds to human PD-1 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:52 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:53.

In some embodiments, the monoclonal antibody that binds to human OX40 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:5 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:11, and the monoclonal antibody that binds to human PD-1 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:52 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:53.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

In some embodiments, the monoclonal antibody that binds to human PD-1 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:50 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:51.

In some embodiments, the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49, and the monoclonal antibody that binds to human PD-1 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO: 50 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:51.

In some embodiments, the monoclonal antibody that binds to human OX40 comprises a heavy chain comprising the amino acid sequence set forth in SEQ ID NO:48 and a light chain comprising the amino acid sequence set forth in SEQ ID NO:49, and the monoclonal antibody that binds to human PD-1 comprises a heavy chain comprising the amino acid sequence set forth in SEQ ID NO: 50 and a light chain comprising the amino acid sequence set forth in SEQ ID NO:51.

In some embodiments, the cancer is a solid tumor. In some embodiments, the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

In some embodiments, the monoclonal antibody that binds to OX40 and the monoclonal antibody that binds to human PD-1 are administered at the same time.

In some embodiments, the monoclonal antibody that binds to human OX40 and the monoclonal antibody that binds to human PD-1 are administered sequentially, in any order.

In some embodiments, the monoclonal antibody that binds to OX40 and/or the monoclonal antibody that binds to human PD-1 are administered intravenously.

In some embodiments, the monoclonal antibody that binds to OX40 and/or the monoclonal antibody that binds to human PD-1 are administered intratumorally.

In some embodiments, the monoclonal antibody that binds to OX40 is administered at a dose of about 0.1 mg/kg to about 10 mg/kg.

In some embodiments, the monoclonal antibody that binds to OX40 is administered at a frequency selected from the group consisting of: once daily, once weekly, once every two weeks (Q2W), and once every three weeks (Q3W).

In some embodiments, the monoclonal antibody that binds to human PD-1 is administered at a dose of about 200 mg.

In some embodiments, the monoclonal antibody that binds to human PD-1 is administered Q3W.

In some aspects, the disclosure provides use (e.g., simultaneous or sequential use) of a therapeutically effective amount of a monoclonal antibody that binds to human OX40 that comprises a heavy chain comprising the amino acid sequence set forth in SEQ ID NO:48 and a light chain comprising the amino acid sequence set forth in SEQ ID NO:49, and a therapeutically effective amount of a monoclonal antibody that binds to human PD-1 that comprises a heavy chain comprising the amino acid sequence set forth in SEQ ID NO: 50 and a light chain comprising the amino acid sequence set forth in SEQ ID NO:51 for the preparation of a medicament for reducing tumor size in a human having cancer.

In some embodiments, the human demonstrates complete response or partial response according to RECIST version 1.1.

In some embodiments, the monoclonal antibody that binds to human PD-1 is intravenously administered to the human starting at least 1 hour and no more than 2 hours following the end of intravenous administration of the monoclonal antibody that binds to human OX40.

### BRIEF DESCRIPTION OF THE DRAWINGS

FIGS. 1-12 show sequences of the anti-OX40 ABPs of a combination of the invention, or a method or use thereof, e.g. CDRs and VH and VL sequences.

FIGS. 13 a and b is a pair of line graphs showing OX86 monotherapy results in a statistically significant increase in survival in nonclinical mouse model; (a) all dose levels tested and (b) the 5 ug (microgram) dose.

FIG. 14 is a line graph showing OX86 and anti-PD1 in CT26 syngeneic mouse tumor model: combination therapy vs. monotherapy.

FIG. 15 is a schematic showing the study design.

FIG. 16 is a series of four panels showing the anti-tumor effect of concurrent administration of a PD-1 antagonist and an OX40 agonist. Combination treatment is superior to monotherapy with either agent alone in MC38 tumor-bearing mice. CR (complete responses); PR (partial responses). Experimental details are described in Example 2.

### DETAILED DESCRIPTION OF THE INVENTION

Compositions and Combinations

Improved function of the immune system is a goal of immunotherapy for cancer. While not being bound by theory, it is thought that for the immune system to be activated and effectively cause regression or eliminate tumors, there must be efficient cross talk among the various compartments of the immune system as well at the at the tumor bed. The tumoricidal effect is dependent on one or more steps, e.g. the uptake of antigen by immature dendritic cells and presentation of processed antigen via MHC I and II by mature dendritic cells to naïve CD8 (cytotoxic) and CD4 (helper) lymphocytes, respectively, in the draining lymph nodes. Naive T cells express molecules such as CTLA-4 and CD28 that engage with co-stimulatory molecules of the B7 family on antigen presenting cells (APCs) such as dendritic cells. In order to keep T cells in check during immune surveillance, B7 on APCs preferentially binds to CTLA-4, an inhibitory molecule on T lymphocytes. However, upon engagement of the T cell receptor (TCR) with MHC Class I or II receptors via cognate peptide presentation on APCs, the co-stimulatory molecule disengages from CTLA-4 and instead binds to the lower affinity stimulatory molecule CD28, causing T cell activation and proliferation. This expanded population of primed T lymphocytes retains memory of the antigen that was presented to them as they traffic to distant tumor sites. Upon encountering a tumor cell bearing the cognate antigen, they eliminate the tumor via cytolytic mediators such as granzyme B and perforins. This apparently simplistic sequence of events is highly dependent on several cytokines, co-stimulatory molecules and check point modulators to activate and differentiate these primed T lymphocytes to a memory pool of cells that can eliminate the tumor.

Thus, an emerging immunotherapeutic strategy is to target T cell co-stimulatory molecules, e.g. OX40. OX40 (e.g. human OX40 (hOX40) or hOX40R) is a tumor necrosis factor receptor family member that is expressed, among other cells, on activated CD4 and CD8 T cells. One of its functions is in the differentiation and long-term survival of these cells. The ligand for OX40 (OX40L) is expressed by activated antigen-presenting cells. Not wishing to be bound by theory, the anti-OX40 ABPs of a combination of the invention, or a method or use thereof, modulate OX40 and promote growth and/or differentiation of T cells and increase long-term memory T-cell populations, e.g. in overlapping mechanisms as those of OX40L, by “engaging” OX40. Thus, in one embodiment of the ABPs of a combination of the invention, or a method or use thereof, bind and engage OX40. In another embodiment, the anti-OX40 ABPs of a combination of the invention, or a method or use thereof, modulate OX40. In a further embodiment, the ABPs of a combination of the invention, or a method or use thereof, modulate OX40 by mimicking OX40L. In another embodiment, the anti-OX40 ABPs of a combination of the invention, or a method or use thereof, are agonist antibodies. In another embodiment, the anti-OX40 ABPs of a combination of the invention, or a method or use thereof, modulate OX40 and cause proliferation of T cells. In a further embodiment, the anti-OX40 ABPs of a combination of the invention, or a method or use thereof, modulate OX40 and improve, augment, enhance, or increase proliferation of CD4 T cells. In another embodiment, the anti-OX40 ABPs of a combination of the invention, or a method or use thereof, improve, augment, enhance, or increase proliferation of CD8 T cells. In further embodiment, the anti-OX40 ABPs of a combination of the invention, or a method or use thereof, improve, augment, enhance, or increase proliferation of both CD4 and CD8 T cells. In another embodiment, the anti-OX40 ABPs of a combination of the invention, or a method or use thereof, enhance T cell function, e.g. of CD4 or CD8 T cells, or both CD4 and CD8 T cells. In a further embodiment, the anti-OX40 ABPs of a combination of the invention, or a method or use thereof, enhance effector T cell function. In another embodiment, the anti-OX40 ABPs of a combination of the invention, or a method or use thereof, improve, augment, enhance, or increase long-term survival of CD8 T cells. In further embodiments, any of the preceding effects occur in a tumor microenvironment.

Not being bound by theory, of equal importance is the blockade of a potentially robust immunosuppressive response at the tumor site by mediators produced both by T regulatory cells (Tregs) as well as the tumor itself (e.g. Transforming Growth Factor (TGF-B) and interleukin-10 (IL-10)). Not wishing to be bound by theory, a key immune pathogenesis of cancer can be the involvement of Tregs that are found in tumor beds and sites of inflammation. In general, Treg cells occur naturally in circulation and help the immune system to return to a quiet, although vigilant state, after encountering and eliminating external pathogens. They help to maintain tolerance to self antigens and are naturally suppressive in function. They are phenotypically characterized as CD4+, CD25+, FOXP3+ cells. Not wishing to be bound by theory, but in order to break tolerance to effectively treat certain cancers, one mode of therapy is to eliminate Tregs preferentially at tumor sites. Targeting and eliminating Tregs leading to an antitumor response has been more successful in tumors that are immunogenic compared to those that are poorly immunogenic. Many tumors secrete cytokines, e.g. TGF-B that may hamper the immune response by causing precursor CD4+25+ cells to acquire the FOXP3+ phenotype and function as Tregs.

“Modulate” as used herein, for example with regard to a receptor or other target means to change any natural or existing function of the receptor, for example it means affecting binding of natural or artificial ligands to the receptor or target; it includes initiating any partial or full conformational changes or signaling through the receptor or target, and also includes preventing partial or full binding of the receptor or target with its natural or artificial ligands. Also included in the case of membrane bound receptors or targets are any changes in the way the receptor or target interacts with other proteins or molecules in the membrane or change in any localization (or co-localization with other molecules) within membrane compartments as compared to its natural or unchanged state. Modulators are therefore compounds or ligands or molecules that modulate a target or receptor. Modulate includes agonizing, e.g. signaling, as well as antagonizing, or blocking signaling or interactions with a ligand or compound or molecule that happen in the unchanged or unmodulated state. Thus, modulators may be agonists or antagonists. Further, one of skill in the art will recognize that not all modulators will be have absolute selectivity for one target or receptor, but are still considered a modulator for that target or receptor; for example, a modulator may also engage multiple targets.

As used herein the term “agonist” refers to an antigen binding protein including but not limited to an antibody, which upon contact with a co-signaling receptor causes one or more of the following (1) stimulates or activates the receptor, (2) enhances, increases or promotes, induces or prolongs an activity, function or presence of the receptor (3) mimics one or more functions of a natural ligand or molecule that interacts with a target or receptor and includes initiating one or more signaling events through the receptor, mimicking one or more functions of a natural ligand, or initiating one or more partial or full conformational changes that are seen in known functioning or signaling through the receptor and/or (4) enhances, increases, promotes or induces the expression of the receptor. Agonist activity can be measured in vitro by various assays know in the art such as, but not limited to, measurement of cell signaling, cell proliferation, immune cell activation markers, cytokine production. Agonist activity can also be measured in vivo by various assays that measure surrogate end points such as, but not limited to the measurement of T cell proliferation or cytokine production.

As used herein the term “antagonist” refers to an antigen binding protein including but not limited to an antibody, which upon contact with a co-signaling receptor causes one or more of the following (1) attenuates, blocks or inactivates the receptor and/or blocks activation of a receptor by its natural ligand, (2) reduces, decreases or shortens the activity, function or presence of the receptor and/or (3) reduces, decrease, abrogates the expression of the receptor. Antagonist activity can be measured in vitro by various assays know in the art such as, but not limited to, measurement of an increase or decrease in cell signaling, cell proliferation, immune cell activation markers, cytokine production. Antagonist activity can also be measured in vivo by various assays that measure surrogate end points such as, but not limited to the measurement of T cell proliferation or cytokine production.

Thus, in one embodiment, an agonist anti-OX40 ABP inhibits the suppressive effect of Treg cells on other T cells, e.g. within the tumor environment.

Accumulating evidence suggests that the ratio of Tregs to T effector cells in the tumor correlates with anti tumor response. Therefore, in one embodiment, the OX40 ABPs (anti-OX40 ABPs) of a combination of the invention, or a method or use thereof, modulate OX40 to augment T effector number and function and inhibit Treg function.

Enhancing, augmenting, improving, increasing, and otherwise changing the anti-tumor effect of OX40 is an object of a combination of the invention, or a method or use thereof. Described herein are combinations of an anti-OX40 ABP of a combination of the invention, or a method or use thereof, and another compound, such as a PD-1 modulator (e.g. anti-PD-1 ABP) described herein.

Thus, as used herein the term “combination of the invention” refers to a combination comprising an anti-OX40 ABP, suitably an agonist anti-OX40 ABP, and an anti-PD-1 ABP, suitably an antagonist anti-PD-1 ABP, each of which may be administered separately or simultaneously as described herein.

As used herein, the terms “cancer,” “neoplasm,” and “tumor,” are used interchangeably and in either the singular or plural form, refer to cells that have undergone a malignant transformation or undergone cellular changes that result in aberrant or unregulated growth or hyperproliferation Such changes or malignant transformations usually make such cells pathological to the host organism, thus precancers or precancerous cells that are or could become pathological and require or could benefit from intervention are also intended to be included. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. In other words, the terms herein include cells, neoplasms, cancers, and tumors of any stage, including what a clinician refers to as precancer, tumors, in situ growths, as well as late stage metastatic growths, Tumors may be hematopoietic tumor, for example, tumors of blood cells or the like, meaning liquid tumors. Specific examples of clinical conditions based on such a tumor include leukemia such as chronic myelocytic leukemia or acute myelocytic leukemia; myeloma such as multiple myeloma; lymphoma and the like.

As used herein the term “agent” is understood to mean a substance that produces a desired effect in a tissue, system, animal, mammal, human, or other subject. Accordingly, the term “anti-neoplastic agent” is understood to mean a substance producing an anti-neoplastic effect in a tissue, system, animal, mammal, human, or other subject. It is also to be understood that an “agent” may be a single compound or a combination or composition of two or more compounds.

By the term “treating” and derivatives thereof as used herein, is meant therapeutic therapy. In reference to a particular condition, treating means: (1) to ameliorate the condition or one or more of the biological manifestations of the condition; (2) to interfere with (a) one or more points in the biological cascade that leads to or is responsible for the condition or (b) one or more of the biological manifestations of the condition; (3) to alleviate one or more of the symptoms, effects or side effects associated with the condition or one or more of the symptoms, effects or side effects associated with the condition or treatment thereof; (4) to slow the progression of the condition or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, “prevention” is understood to refer to the prophylactic administration of a drug to substantially diminish the likelihood or severity of a condition or biological manifestation thereof, or to delay the onset of such condition or biological manifestation thereof. The skilled artisan will appreciate that “prevention” is not an absolute term. Prophylactic therapy is appropriate, for example, when a subject is considered at high risk for developing cancer, such as when a subject has a strong family history of cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The administration of a therapeutically effective amount of the combinations of the invention (or therapeutically effective amounts of each of the components of the combination) are advantageous over the individual component compounds in that the combinations provide one or more of the following improved properties when compared to the individual administration of a therapeutically effective amount of a component compound: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, or vi) an increase in the bioavailability of one or both of the component compounds.

The invention further provides pharmaceutical compositions, which include one or more of the components herein, and one or more pharmaceutically acceptable carriers, diluents, or excipients. The combination of the invention may comprise two pharmaceutical compositions, one comprising an anti-OX40 ABP of the invention, suitably an agonist anti-OX40 ABP, and the other comprising an anti-PD-1 ABP, suitably an antagonist anti-PD-1 ABP, each of which may have the same or different carriers, diluents or excipients. The carrier(s), diluent(s) or excipient(s) must be acceptable in the sense of being compatible with the other ingredients of the formulation, capable of pharmaceutical formulation, and not deleterious to the recipient thereof.

The components of the combination of the invention, and pharmaceutical compositions comprising such components may be administered in any order, and in different routes; the components and pharmaceutical compositions comprising the same may be administered simultaneously.

In accordance with another aspect of the invention there is also provided a process for the preparation of a pharmaceutical composition including admixing a component of the combination of the invention and one or more pharmaceutically acceptable carriers, diluents or excipients.

The components of the invention may be administered by any appropriate route. For some components, suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that the components may be compounded together or in separate pharmaceutical compositions.

In one embodiment, one or more components of a combination of the invention are administered intravenously. In another embodiment, one or more components of a combination of the invention are administered intratumorally. In another embodiment, one or more components of a combination of the invention are administered systemically, e.g. intravenously, and one or more other components of a combination of the invention are administered intratumorally. In another embodiment, all of the components of a combination of the invention are administered systemically, e.g. intravenously. In an alternative embodiment, all of the components of the combination of the invention are administered intratumorally. In any of the embodiments, e.g. in this paragraph, the components of the invention are administered as one or more pharmaceutical compositions.

Antigen Binding Proteins that Bind OX40

“Antigen Binding Protein (ABP)” means a protein that binds an antigen, including antibodies or engineered molecules that function in similar ways to antibodies. Such alternative antibody formats include triabody, tetrabody, miniantibody, and a minibody, Also included are alternative scaffolds in which the one or more CDRs of any molecules in accordance with the disclosure can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain. An ABP also includes antigen binding fragments of such antibodies or other molecules. Further, an ABP of a combination of the invention, or a method or use thereof, may comprise the VH regions formatted into a full length antibody, a (Fab′)2 fragment, a Fab fragment, a bi-specific or biparatopic molecule or equivalent thereof (such as scFV, bi- tri- or tetra-bodies, Tandabs etc.), when paired with an appropriate light chain. The ABP may comprise an antibody that is an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. The ABP may also be a chimeric antibody of the type described in WO86/01533 which comprises an antigen binding region and a non-immunoglobulin region.

Thus, herein an anti-OX40 ABP of a combination, or a method or use thereof, of the invention or protein is one that binds OX40, and in preferred embodiments does one or more of the following: modulate signaling through OX40, modulates the function of OX40, agonize OX40 signaling, stimulate OX40 function, or co-stimulate OX40 signaling. One of skill in the art would readily recognize a variety of well known assays to establish such functions.

The term “antibody” as used herein refers to molecules with an antigen binding domain, and optionally an immunoglobulin-like domain or fragment thereof and includes monoclonal (for example IgG, IgM, IgA, IgD or IgE and modified variants thereof), recombinant, polyclonal, chimeric, humanized, biparatopic, bispecific and heteroconjugate antibodies, or a closed conformation multispecific antibody. An “antibody” included xenogeneic, allogeneic, syngeneic, or other modified forms thereof. An antibody may be isolated or purified. An antibody may also be recombinant, i.e. produced by recombinant means; for example, an antibody that is 90% identical to a reference antibody may be generated by mutagenesis of certain residues using recombinant molecular biology techniques known in the art. Thus, the antibodies of the present invention may comprise heavy chain variable regions and light chain variable regions of a combination of the invention, or a method or use thereof, which may be formatted into the structure of a natural antibody or formatted into a full length recombinant antibody, a (Fab′)2 fragment, a Fab fragment, a bi-specific or biparatopic molecule or equivalent thereof (such as scFV, bi- tri- or tetra-bodies, Tandabs etc.), when paired with an appropriate light chain. The antibody may be an IgG1, IgG2, IgG3, or IgG4 or a modified variant thereof. The constant domain of the antibody heavy chain may be selected accordingly. The light chain constant domain may be a kappa or lambda constant domain. The antibody may also be a chimeric antibody of the type described in WO86/01533 which comprises an antigen binding region and a non-immunoglobulin region.

One of skill in the art will recognize that the anti-OX40 ABPs of a combination herein, or method or use thereof, of the invention bind an epitope of OX40; likewise an anti-PD-1 ABP of a combination herein, or a method or use thereof, of the invention binds an epitope of PD-1. The epitope of an ABP is the region of its antigen to which the ABP binds. Two ABPs bind to the same or overlapping epitope if each competitively inhibits (blocks) binding of the other to the antigen. That is, a 1×, 5×, 10×, 20× or 100× excess of one antibody inhibits binding of the other by at least 50% but preferably 75%, 90% or even 99% as measured in a competitive binding assay compared to a control lacking the competing antibody (see, e.g., Junghans et al., Cancer Res. 50:1495, 1990, which is incorporated herein by reference). Alternatively, two antibodies have the same epitope if essentially all amino acid mutations in the antigen that reduce or eliminate binding of one antibody reduce or eliminate binding of the other. Also the same epitope may include “overlapping epitopes” e.g. if some amino acid mutations that reduce or eliminate binding of one antibody reduce or eliminate binding of the other.

The strength of binding may be important in dosing and administration of an ABP of the combination, or method or use thereof, of the invention. In one embodiment, the ABP of the invention binds its target (e.g. OX40 or PD-1) with high affinity. For example, when measured by Biacore, the antibody binds to OX40, preferably human OX40, with an affinity of 1-1000 nM or 500 nM or less or an affinity of 200 nM or less or an affinity of 100 nM or less or an affinity of 50 nM or less or an affinity of 500 pM or less or an affinity of 400 pM or less, or 300 pM or less. In a further aspect the antibody binds to OX40, preferably human OX40, when measured by Biacore of between about 50 nM and about 200 nM or between about 50 nM and about 150 nM. In one aspect of the present invention the antibody binds OX40, preferably human OX40, with an affinity of less than 100 nM.

In a further embodiment, binding is measured by Biacore. Affinity is the strength of binding of one molecule, e.g. an antibody of a combination of the invention, or a method or use thereof, to another, e.g. its target antigen, at a single binding site. The binding affinity of an antibody to its target may be determined by equilibrium methods (e.g. enzyme-linked immunoabsorbent assay (ELISA) or radioimmunoassay (RIA)), or kinetics (e.g. BIACORE analysis). For example, the Biacore methods known in the art may be used to measure binding affinity.

Avidity is the sum total of the strength of binding of two molecules to one another at multiple sites, e.g. taking into account the valency of the interaction.

In an aspect, the equilibrium dissociation constant (KD) of the ABP of a combination of the invention, or a method or use thereof, and OX40, preferably human OX40, interaction is 100 nM or less, 10 nM or less, 2 nM or less or 1 nM or less. Alternatively the KD may be between 5 and 10 nM; or between 1 and 2 nM. The KD may be between 1 pM and 500 pM; or between 500 pM and 1 nM. A skilled person will appreciate that the smaller the KD numerical value, the stronger the binding. The reciprocal of KD (i.e. 1/KD) is the equilibrium association constant (KA) having units M-1. A skilled person will appreciate that the larger the KA numerical value, the stronger the binding.

The dissociation rate constant (kd) or “off-rate” describes the stability of the complex of the ABP on one hand and OX40, preferably human OX40 on the other hand, i.e. the fraction of complexes that decay per second. For example, a kd of 0.01 s-1 equates to 1% of the complexes decaying per second. In an embodiment, the dissociation rate constant (kd) is 1×10-3 s-1 or less, 1×10-4 s-1 or less, 1×10-5 s-1 or less, or 1×10-6 s-1 or less. The kd may be between 1×10-5 s-1 and 1×10-4 s-1; or between 1×10-4 s-1 and 1×10-3 s-1.

Competition between an anti-OX40 ABP of a combination of the invention, or a method or use thereof, and a reference antibody, e.g. for binding OX40, an epitope of OX40, or a fragment of the OX40, may be determined by competition ELISA, FMAT or Biacore. In one aspect, the competition assay is carried out by Biacore. There are several possible reasons for this competition: the two proteins may bind to the same or overlapping epitopes, there may be steric inhibition of binding, or binding of the first protein may induce a conformational change in the antigen that prevents or reduces binding of the second protein.

“Binding fragments” as used herein means a portion or fragment of the ABPs of a combination of the invention, or a method or use thereof, that include the antigen-binding site and are capable of binding OX40 as defined herein, e.g. but not limited to capable of binding to the same epitope of the parent or full length antibody.

Functional fragments of the ABPs of a combination of the invention, or a method or use thereof, are contemplated herein.

Thus, “binding fragments” and “functional fragments” may be an Fab and F(ab′)2 fragments which lack the Fc fragment of an intact antibody, clear more rapidly from the circulation, and may have less non-specific tissue binding than an intact antibody (Wahl et al., J. Nuc. Med. 24:316-325 (1983)). Also included are Fv fragments (Hochman, J. et al. (1973) Biochemistry 12:1130-1135; Sharon, J. et al. (1976) Biochemistry 15:1591-1594). These various fragments are produced using conventional techniques such as protease cleavage or chemical cleavage (see, e.g., Rousseaux et al., Meth. Enzymol., 121:663-69 (1986)).

“Functional fragments” as used herein means a portion or fragment of the ABPs of a combination of the invention, or a method or use thereof, that include the antigen-binding site and are capable of binding the same target as the parent ABP, e.g. but not limited to binding the same epitope, and that also retain one or more modulating or other functions described herein or known in the art.

As the ABPs of the present invention may comprise heavy chain variable regions and light chain variable regions of a combination of the invention, or a method or use thereof, which may be formatted into the structure of a natural antibody, a functional fragment is one that retains binding or one or more functions of the full length ABP as described herein. A binding fragment of an ABP of a combination of the invention, or a method or use thereof, may therefore comprise the VL or VH regions, a (Fab′)2 fragment, a Fab fragment, a fragment of a bi-specific or biparatopic molecule or equivalent thereof (such as scFV, bi- tri- or tetra-bodies, Tandabs etc.), when paired with an appropriate light chain.

The term “CDR” as used herein, refers to the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin.

It will be apparent to those skilled in the art that there are various numbering conventions for CDR sequences; Chothia (Chothia et al. (1989) Nature 342: 877-883), Kabat (Kabat et al., Sequences of Proteins of Immunological Interest, 4th Ed., U.S. Department of Health and Human Services, National Institutes of Health (1987)), AbM (University of Bath) and Contact (University College London). The minimum overlapping region using at least two of the Kabat, Chothia, AbM and contact methods can be determined to provide the “minimum binding unit”. The minimum binding unit may be a subportion of a CDR. The structure and protein folding of the antibody may mean that other residues are considered part of the CDR sequence and would be understood to be so by a skilled person. It is noted that some of the CDR definitions may vary depending on the individual publication used.

Unless otherwise stated and/or in absence of a specifically identified sequence, references herein to “CDR”, “CDRL1” (or “LC CDR1”), “CDRL2” (or “LC CDR2”), “CDRL3” (or “LC CDR3”), “CDRH1” (or “HC CDR1”), “CDRH2” (or “HC CDR2”), “CDRH3” (or “HC CDR3”) refer to amino acid sequences numbered according to any of the known conventions; alternatively, the CDRs are referred to as “CDR1,” “CDR2,” “CDR3” of the variable light chain and “CDR1,” “CDR2,” and “CDR3” of the variable heavy chain. In particular embodiments, the numbering convention is the Kabat convention.

The term “CDR variant” as used herein, refers to a CDR that has been modified by at least one, for example 1, 2 or 3, amino acid substitution(s), deletion(s) or addition(s), wherein the modified antigen binding protein comprising the CDR variant substantially retains the biological characteristics of the antigen binding protein pre-modification. It will be appreciated that each CDR that can be modified may be modified alone or in combination with another CDR. In one aspect, the modification is a substitution, particularly a conservative substitution, for example as shown in Table 1.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

For example, in a variant CDR, the amino acid residues of the minimum binding unit may remain the same, but the flanking residues that comprise the CDR as part of the Kabat or Chothia definition(s) may be substituted with a conservative amino acid residue.

Such antigen binding proteins comprising modified CDRs or minimum binding units as described above may be referred to herein as “functional CDR variants” or “functional binding unit variants”.

The antibody may be of any species, or modified to be suitable to administer to a cross species. For example the CDRs from a mouse antibody may be humanized for administration to humans. In any embodiment, the antigen binding protein is optionally a humanized antibody.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., Queen et al., Proc. Natl Acad Sci USA, 86:10029-10032 (1989), Hodgson et al., Bio/Technology, 9:421 (1991)). A suitable human acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies—see for example EP-A-0239400 and EP-A-054951.

In yet a further embodiment, the humanized antibody has a human antibody constant region that is an IgG. In another embodiment, the IgG is a sequence as disclosed in any of the above references or patent publications.

For nucleotide and amino acid sequences, the term “identical” or “identity” indicates the degree of identity between two nucleic acid or two amino acid sequences when optimally aligned and compared with appropriate insertions or deletions.

The percent sequence identity between two sequences is a function of the number of identical positions shared by the sequences (i.e., % identity=number of identical positions/total number of positions multiplied by 100), taking into account the number of gaps, and the length of each gap, which need to be introduced for optimal alignment of the two sequences. The comparison of sequences and determination of percent identity between two sequences can be accomplished using a mathematical algorithm, as described below.

Percent identity between a query nucleic acid sequence and a subject nucleic acid sequence is the “Identities” value, expressed as a percentage, which is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein.

Percent identity between a query amino acid sequence and a subject amino acid sequence is the “Identities” value, expressed as a percentage, which is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query amino acid sequence may be described by an amino acid sequence identified in one or more claims herein.

In any embodiment of a combination of the invention, or a method or use thereof, herein, the ABP may have any one or all CDRs, VH, VL, HC, LC, with 99, 98, 97, 96, 95, 94, 93, 92, 91, or 90, or 85, or 80, or 75, or 70 percent identity to the sequence shown or referenced, e.g. as defined by a SEQ ID NO disclosed herein.

ABPs that bind human OX40 receptor are provided herein (i.e. an anti-OX40 ABP and an anti-human OX40 receptor (hOX-40R) antibody, sometimes referred to herein as an “anti-OX40 ABP” or an “anti-OX40 antibody” and/or other variations of the same). These antibodies are useful in the treatment or prevention of acute or chronic diseases or conditions whose pathology involves OX40 signaling. In one aspect, an antigen binding protein, or isolated human antibody or functional fragment of such protein or antibody, that binds to human OX40R and is effective as a cancer treatment or treatment against disease is described, for example in combination with another compound such as an anti-PD-1 ABP, suitably an antagonist anti-PD1 ABP. Any of the antigen binding proteins or antibodies disclosed herein may be used as a medicament. Any one or more of the antigen binding proteins or antibodies may be used in the methods or compositions to treat cancer, e.g. those disclosed herein.

The isolated antibodies as described herein bind to OX40, and may bind to OX40 encoded from the following genes: NCBI Accession Number NP_003317, Genpept Accession Number P23510, or genes having 90 percent homology or 90 percent identity thereto. The isolated antibody provided herein may further bind to the OX40 receptor having one of the following GenBank Accession Numbers: AAB39944, CAE11757, or AAI05071.

Antigen binding proteins and antibodies that bind and/or modulate OX-40 receptor are known in the art. Exemplary anti-OX40 ABPs of a combination of the invention, or a method or use thereof, are disclosed, for example in International Publication No. WO2013/028231 (PCT/US2012/024570), international filing date 9 Feb. 2012, and WO2012/027328 (PCT/US2011/048752), international filing date 23 Aug. 2011, each of which is incorporated by reference in its entirety herein (To the extent any definitions conflict, this instant application controls).

In one embodiment, the OX-40 antigen binding protein is one disclosed in WO2012/027328 (PCT/US2011/048752), international filing date 23 Aug. 2011. In another embodiment, the antigen binding protein comprises the CDRs of an antibody disclosed in WO2012/027328 (PCT/US2011/048752), international filing date 23 Aug. 2011, or CDRs with at least 90% (e.g., 90%, 91%, 92%, 93%, 94%, 95%, 96%, 97%, 98%, or 99%, or 100%) sequence identity to the disclosed CDR sequences. In a further embodiment the antigen binding protein comprises a VH, a VL, or both of an antibody disclosed in WO2012/027328 (PCT/US2011/048752), international filing date 23 Aug. 2011, or a VH or a VL with at least 90% (e.g., 90%, 91%, 92%, 93%, 94%, 95%, 96%, 97%, 98%, or 99%, or 100%) sequence identity to the disclosed VH or VL sequences.

In another embodiment, the OX-40 antigen binding protein is one disclosed in WO2013/028231 (PCT/US2012/024570), international filing date 9 Feb. 2012. In another embodiment, the antigen binding protein comprises the CDRs of an antibody disclosed in WO2013/028231 (PCT/US2012/024570), international filing date 9 Feb. 2012, or CDRs with at least 90% (e.g., 90%, 91%, 92%, 93%, 94%, 95%, 96%, 97%, 98%, or 99%, or 100%) sequence identity to the disclosed CDR sequences. In a further embodiment the antigen binding protein comprises a VH, a VL, or both of an antibody disclosed in WO2013/028231 (PCT/US2012/024570), international filing date 9 Feb. 2012, or a VH or a VL with at least 90% (e.g., 90%, 91%, 92%, 93%, 94%, 95%, 96%, 97%, 98%, or 99%, or 100%) sequence identity to the disclosed VH or VL sequences.

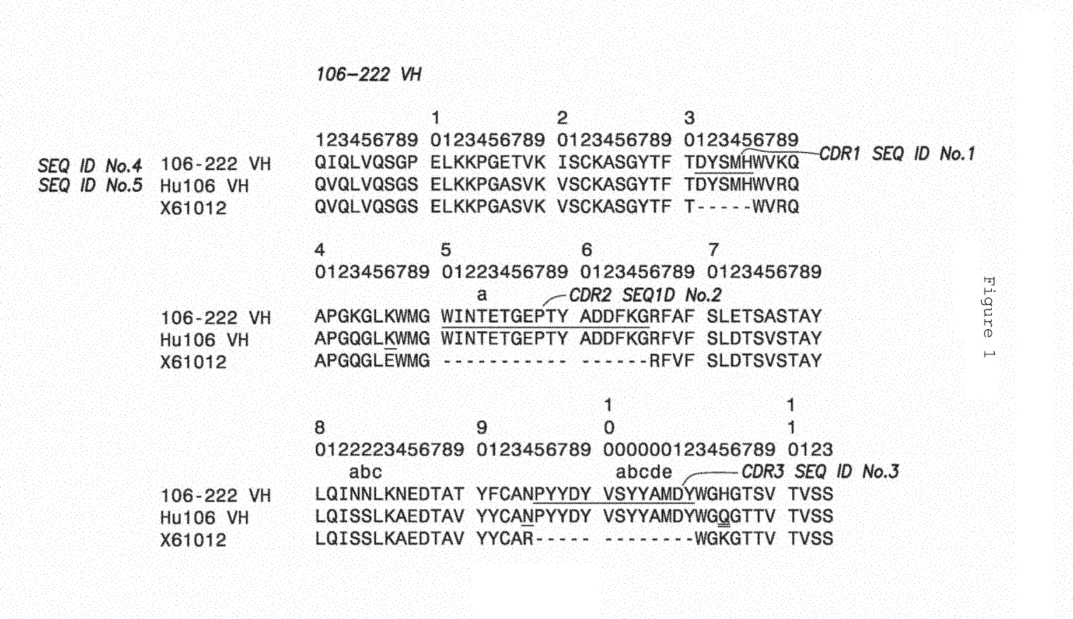

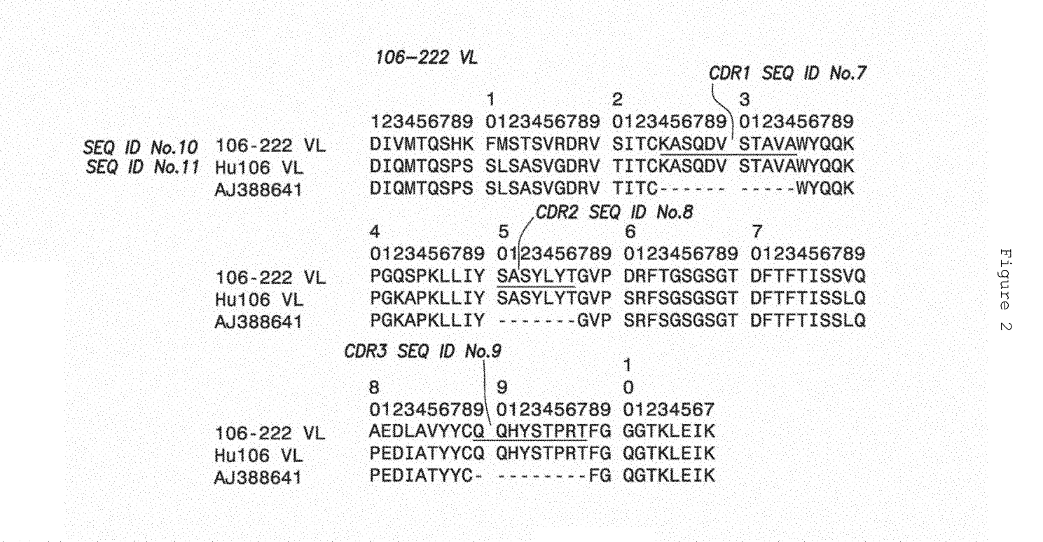

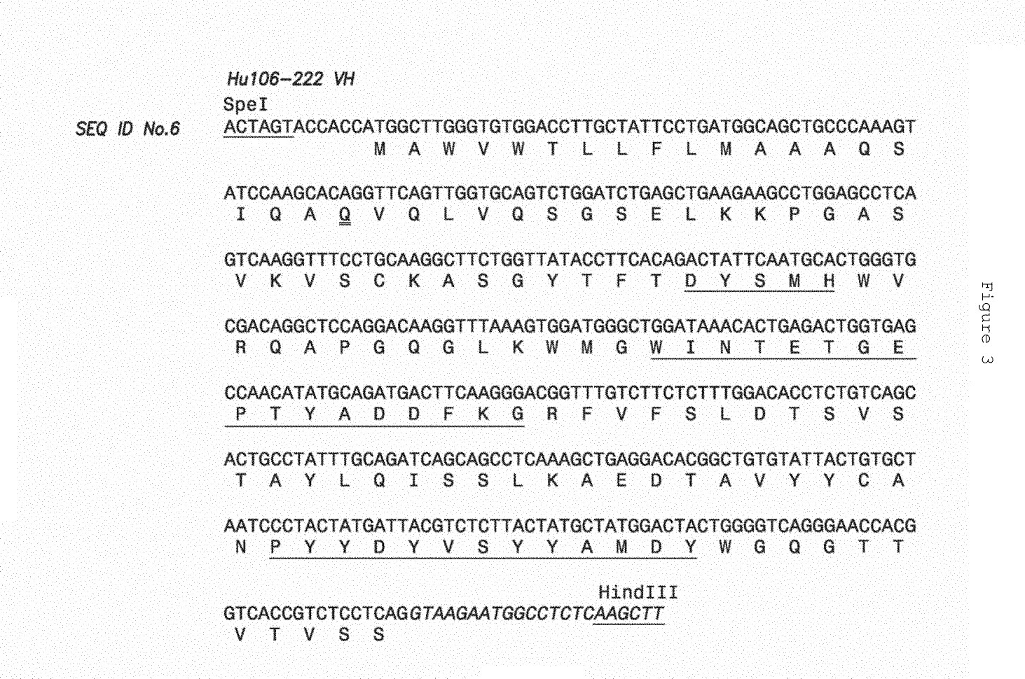

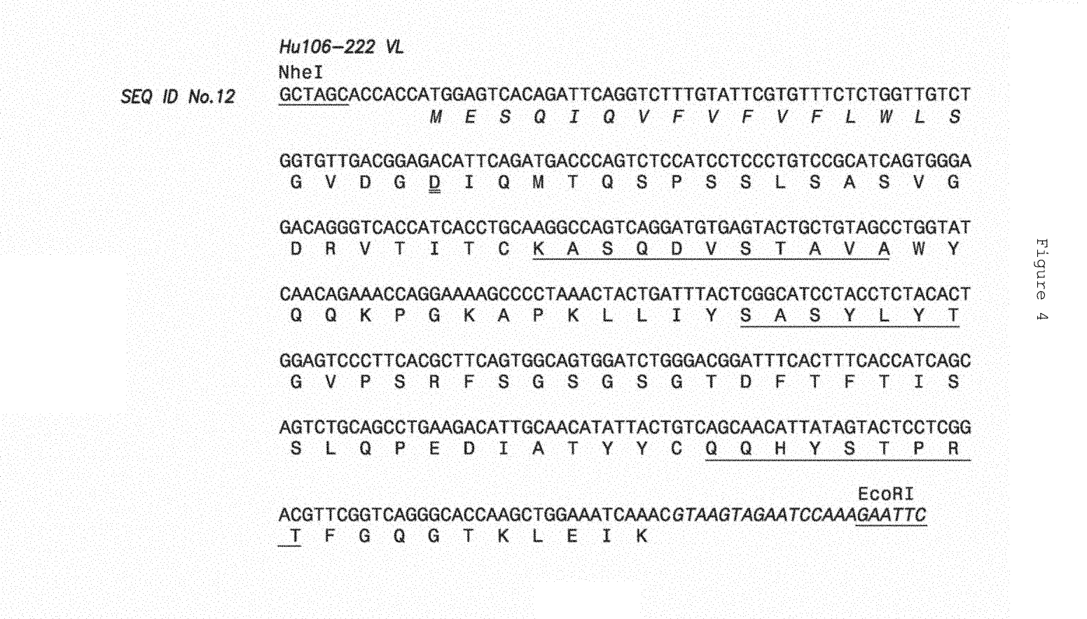

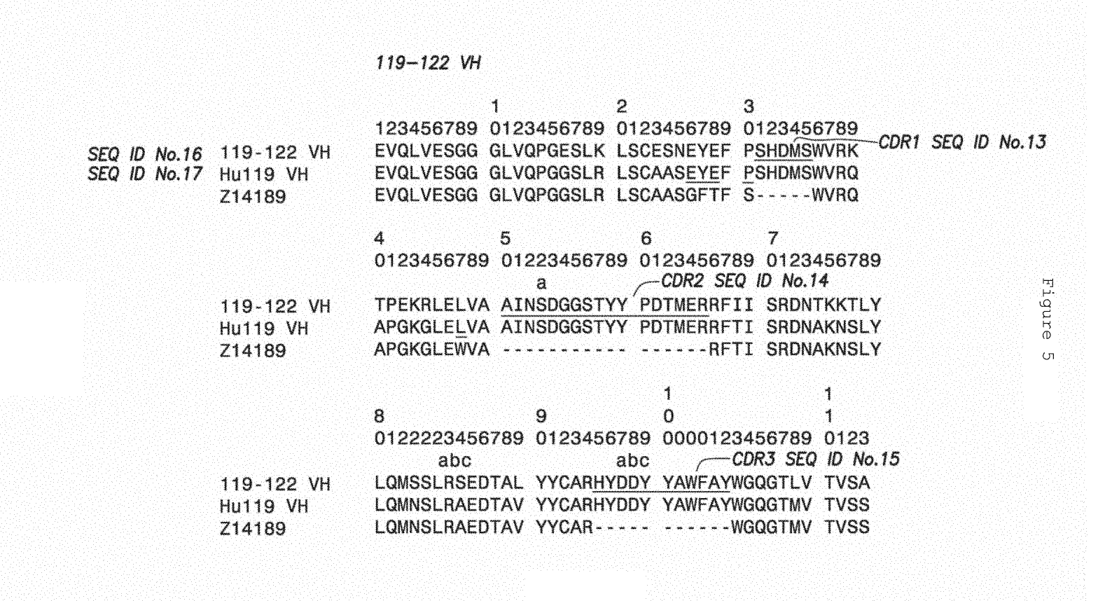

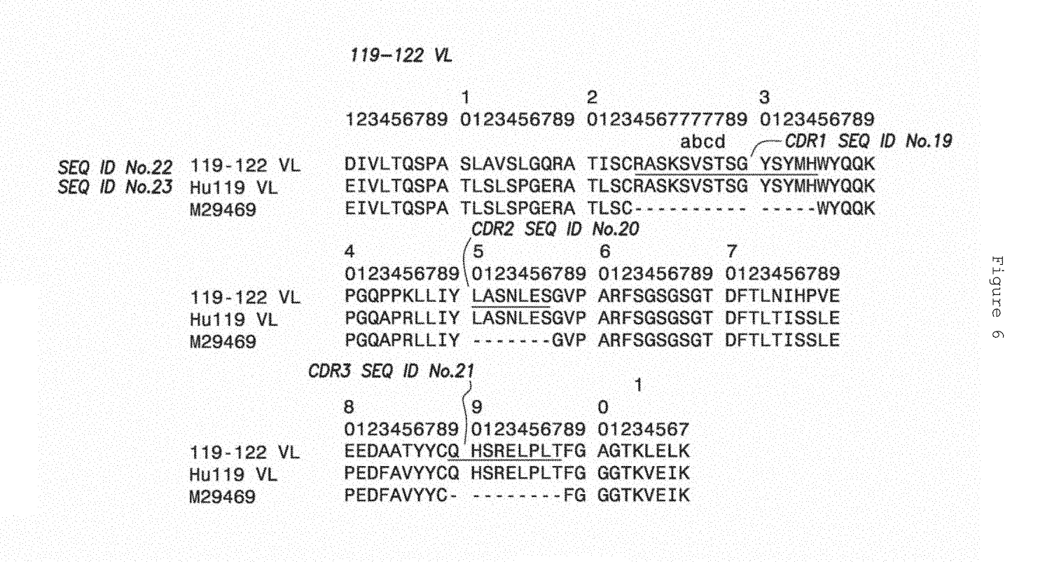

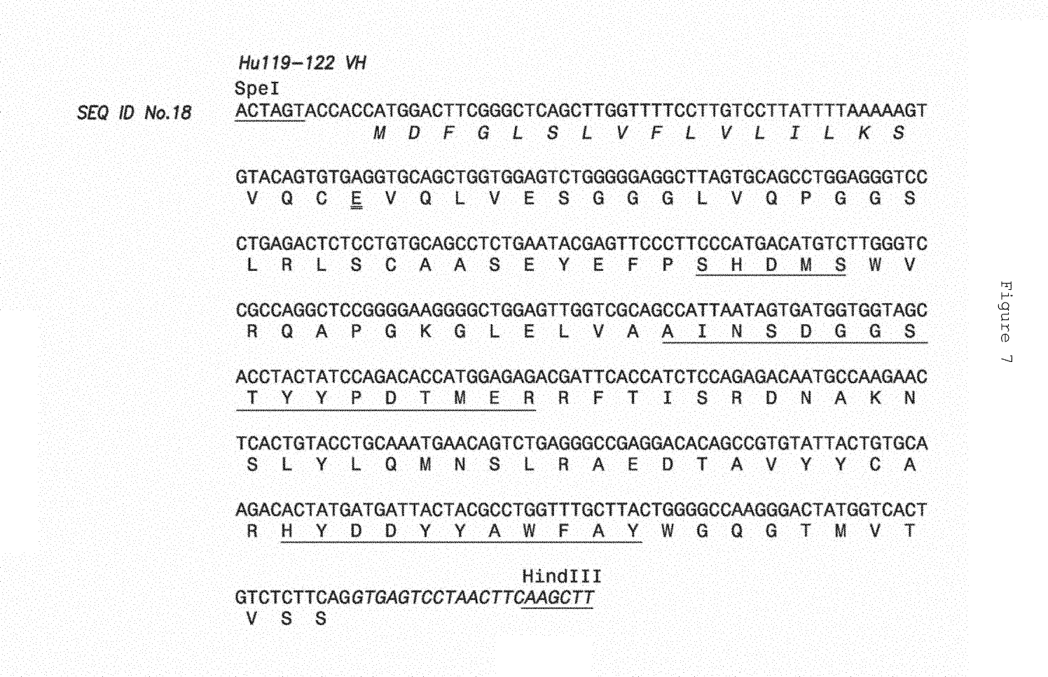

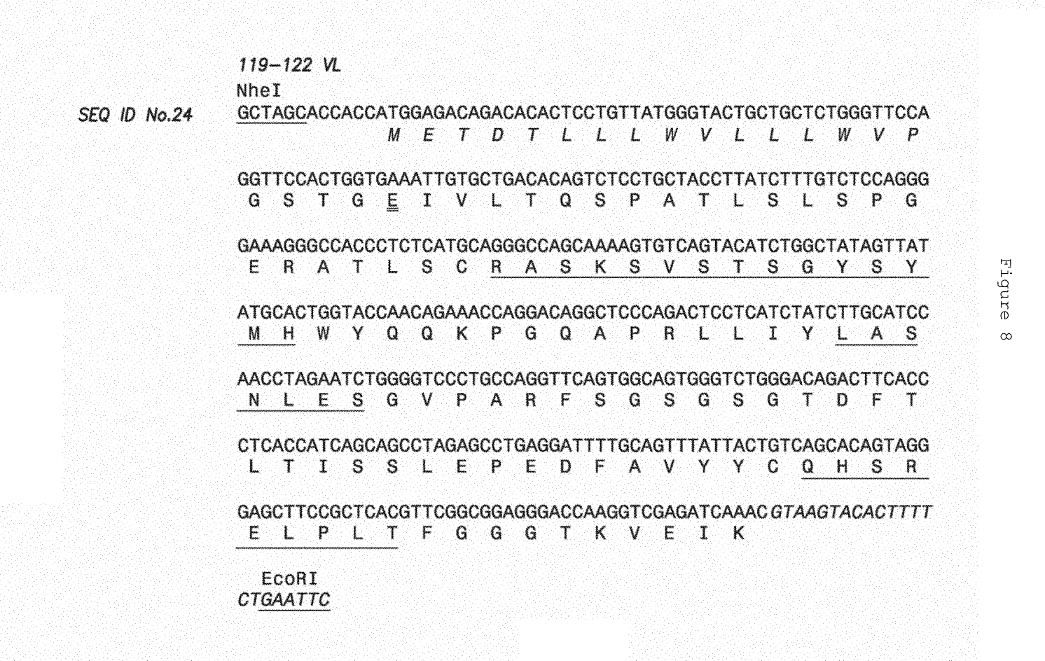

FIGS. 1-12 show sequences of the anti-OX40 ABPs of a combination of the invention, or a method or use thereof, e.g. CDRs and VH and VL sequences of the ABPs. In another embodiment, the anti-OX40 ABP of a combination of the invention, or a method or use thereof, comprises one or more of the CDRs or VH or VL sequences, or sequences with at least 90% (e.g., 90%, 91%, 92%, 93%, 94%, 95%, 96%, 97%, 98%, or 99%, or 100%) sequence identity thereto, shown in the Figures herein. FIG. 1 includes a disclosure of residues 1-30, 36-49, 67-98, and 121-131 of SEQ ID NO: 108. X61012 is disclosed as SEQ ID NO: 108. FIG. 2 includes a disclosure of residues 1-23, 35-49, 57-88, and 102-111 of SEQ ID NO: 109. AJ388641 is disclosed as SEQ ID NO: 109. FIG. 3 includes a disclosure of the amino acid sequence as SEQ ID NO: 110. FIG. 4 includes a disclosure of the amino acid sequence as SEQ ID NO: 111. FIG. 5 includes a disclosure of residues 17-46, 52-65, 83-114, and 126-136 of SEQ ID NO: 112. Z14189 is disclosed as SEQ ID NO: 112. FIG. 6 includes a disclosure of residues 21-43, 55-69, 77-108, and 118-127 of SEQ ID NO: 113. M29469 is disclosed as SEQ ID NO: 113. FIG. 7 protein is disclosed as SEQ ID NO: 114. FIG. 8 protein is disclosed as SEQ ID NO: 115.

FIG. 1 shows the alignment of the amino acid sequences of 106-222, humanized 106-222 (Hu106), and human acceptor X61012 (GenBank accession number) VH sequences are shown. Amino acid residues are shown in single letter code. Numbers above the sequences indicate the locations according to Kabat et al. (Sequences of Proteins of Immunological Interests, Fifth edition, NIH Publication No. 91-3242, U.S. Department of Health and Human Services, 1991). The same sequences as claimed herein are also provided in the Sequence Listing and the position numbers may be different. In FIG. 1, CDR sequences defined by Kabat et al. (1991) are underlined in 106-222 VH. CDR residues in X61012 VH are omitted in the figure. Human VH sequences homologous to the 106-222 VH frameworks were searched for within the GenBank database, and the VH sequence encoded by the human X61012 cDNA (X61012 VH) was chosen as an acceptor for humanization. The CDR sequences of 106-222 VH were first transferred to the corresponding positions of X61012 VH. Next, at framework positions where the three-dimensional model of the 106-222 variable regions suggested significant contact with the CDRs, amino acid residues of mouse 106-222 VH were substituted for the corresponding human residues. These substitutions were performed at positions 46 and 94 (underlined in Hu106 VH). In addition, a human framework residue that was found to be atypical in the corresponding V region subgroup was substituted with the most typical residue to reduce potential immunogenicity. This substitution was performed at position 105 (double-underlined in Hu106 VH).

FIG. 2 shows alignment of the amino acid sequences of 106-222, humanized 106-222 (Hu106), and human acceptor AJ388641 (GenBank accession number) VL sequences is shown. Amino acid residues are shown in single letter code. Numbers above the sequences indicate the locations according to Kabat et al. (1991). The same sequences as claimed herein are also provided in the Sequence Listing although the position numbers may be different. CDR sequences defined by Kabat et al. are underlined in 106-222 VH. CDR residues in AJ388641 VL are omitted in the figure. Human VL sequences homologous to the 106-222 VL frameworks were searched for within the GenBank database, and the VL sequence encoded by the human AJ388641 cDNA (AJ388641 VL) was chosen as an acceptor for humanization. The CDR sequences of 106-222 VL were transferred to the corresponding positions of AJ388641 VL. No framework substitutions were performed in the humanized form.

FIG. 3 shows the nucleotide sequence of the Hu106 VH gene flanked by SpeI and HindIII sites (underlined) is shown along with the deduced amino acid sequence. Amino acid residues are shown in single letter code. The signal peptide sequence is in italic. The N-terminal amino acid residue (Q) of the mature VH is double-underlined. CDR sequences according to the definition of Kabat et al. (1991) are underlined. The same sequences as claimed herein are also provided in the Sequence Listing and the position numbers may be different in the Sequence Listing. The intron sequence is in italic.

FIG. 4 shows the nucleotide sequence of the Hu106-222 VL gene flanked by NheI and EcoRI sites (underlined) is shown along with the deduced amino acid sequence. Amino acid residues are shown in single letter code. The signal peptide sequence is in italic. The N-terminal amino acid residue (D) of the mature VL is double-underlined. CDR sequences according to the definition of Kabat et al. (1991) are underlined. The intron sequence is in italic. The same sequences as claimed herein are also provided in the Sequence Listing although the position numbers may be different in the Sequence Listing.

FIG. 5 shows the alignment of the amino acid sequences of 119-122, humanized 119-122 (Hu119), and human acceptor Z14189 (GenBank accession number) VH sequences are shown. Amino acid residues are shown in single letter code. Numbers above the sequences indicate the locations according to Kabat et al. (Sequences of Proteins of Immunological Interests, Fifth edition, NIH Publication No. 91-3242, U.S. Department of Health and Human Services, 1991). CDR sequences defined by Kabat et al. (1991) are underlined in 119-122 VH. CDR residues in Z14189 VH are omitted in the figure. Human VH sequences homologous to the 119-122 VH frameworks were searched for within the GenBank database, and the VH sequence encoded by the human Z14189 cDNA (Z14189 VH) was chosen as an acceptor for humanization. The CDR sequences of 119-122 VH were first transferred to the corresponding positions of Z14189 VH. Next, at framework positions where the three-dimensional model of the 119-122 variable regions suggested significant contact with the CDRs, amino acid residues of mouse 119-122 VH were substituted for the corresponding human residues. These substitutions were performed at positions 26, 27, 28, 30 and 47 (underlined in the Hu119 VH sequence) as shown on the figure. The same sequences as claimed herein are also provided in the Sequence Listing although the position numbers may be different in the Sequence Listing.

FIG. 6 shows the alignment of the amino acid sequences of 119-122, humanized 119-122 (Hu119), and human acceptor M29469 (GenBank accession number) VL sequences are shown. Amino acid residues are shown in single letter code. Numbers above the sequences indicate the locations according to Kabat et al. (1991). CDR sequences defined by Kabat et al. (1) are underlined in 119-122 VL. CDR residues in M29469 VL are omitted in the sequence. Human VL sequences homologous to the 119-122 VL frameworks were searched for within the GenBank database, and the VL sequence encoded by the human M29469 cDNA (M29469 VL) was chosen as an acceptor for humanization. The CDR sequences of 119-122 VL were transferred to the corresponding positions of M29469 VL. No framework substitutions were needed in the humanized form. The same sequences as claimed herein are also provided in the Sequence Listing although the position numbers may be different in the Sequence Listing.

FIG. 7 shows the nucleotide sequence of the Hu119 VH gene flanked by SpeI and HindIII sites (underlined) is shown along with the deduced amino acid sequence. Amino acid residues are shown in single letter code. The signal peptide sequence is in italic. The N-terminal amino acid residue (E) of the mature VH is double-underlined. CDR sequences according to the definition of Kabat et al. (1991) are underlined. The intron sequence is in italic. The same sequences as claimed herein are also provided in the Sequence Listing although the position numbers may be different in the Sequence Listing.

FIG. 8 shows the nucleotide sequence of the Hu119 VL gene flanked by NheI and EcoRI sites (underlined) is shown along with the deduced amino acid sequence. Amino acid residues are shown in single letter code. The signal peptide sequence is in italic. The N-terminal amino acid residue (E) of the mature VL is double-underlined. CDR sequences according to the definition of Kabat et al. (1991) are underlined. The intron sequence is in italic. The same sequences as claimed herein are also provided in the Sequence Listing although the position numbers may be different in the Sequence Listing.

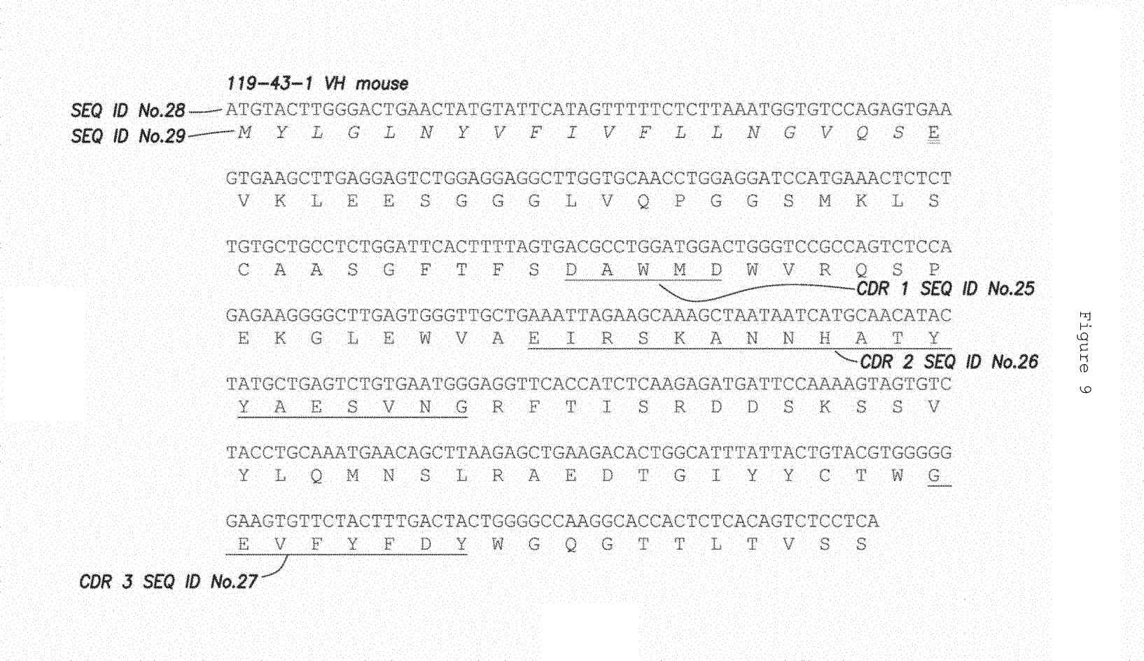

FIG. 9 shows the nucleotide sequence of mouse 119-43-1 VH cDNA along with the deduced amino acid sequence. Amino acid residues are shown in single letter code. The signal peptide sequence is in italic. The N-terminal amino acid residue (E) of the mature VH is double-underlined. CDR sequences according to the definition of Kabat et al. (Sequences of Proteins of Immunological Interests, Fifth edition, NIH Publication No. 91-3242, U.S. Department of Health and Human Services, 1991) are underlined.

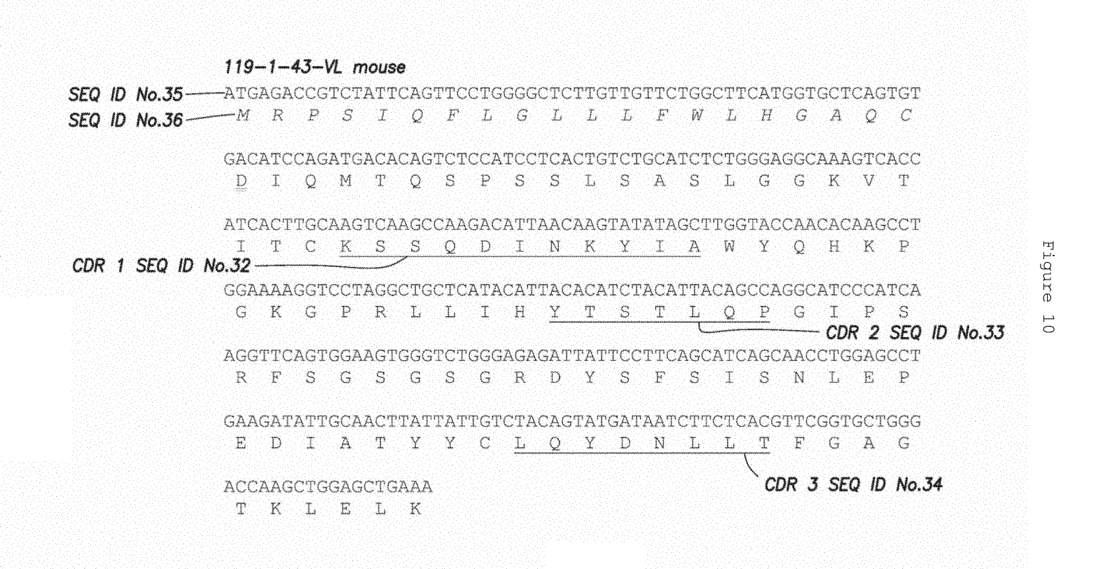

FIG. 10 shows the nucleotide sequence of mouse 119-43-1 VL cDNA is shown the deduced amino acid sequence. Amino acid residues are shown in single letter code. The signal peptide sequence is in italic. The N-terminal amino acid residue (D) of the mature VL is double-underlined. CDR sequences according to the definition of Kabat et al. (1991) are underlined.

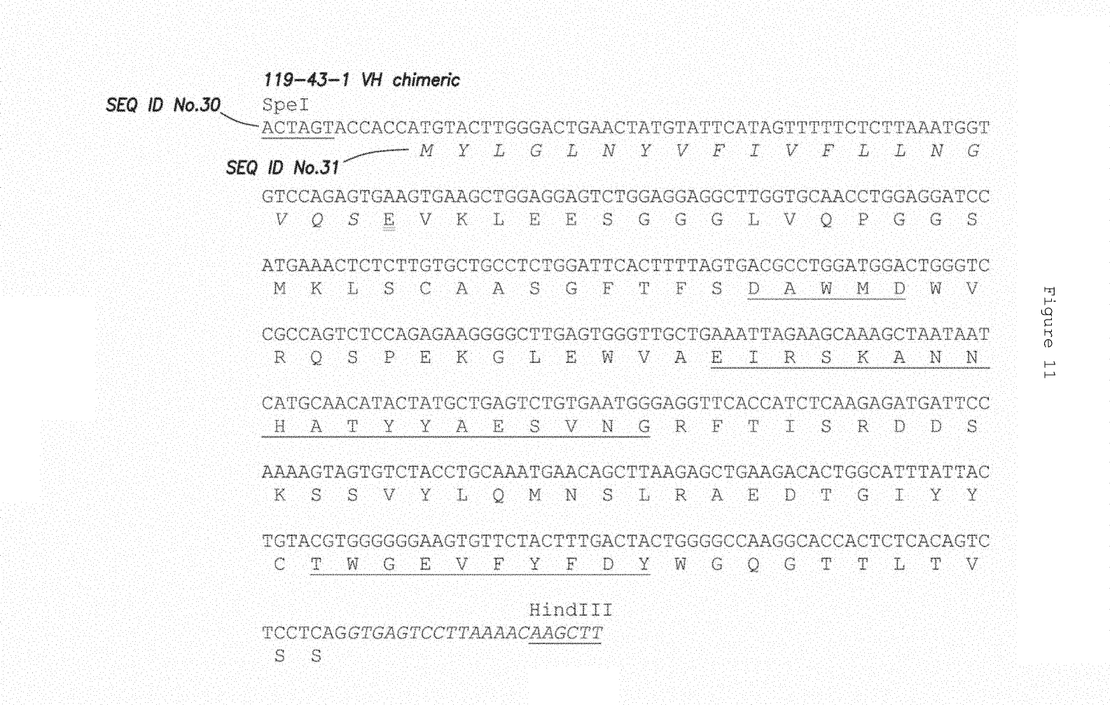

FIG. 11 shows the nucleotide sequence of the designed 119-43-1 VH gene flanked by SpeI and HindIII sites (underlined) along with the deduced amino acid sequence. Amino acid residues are shown in single letter code. The signal peptide sequence is in italic. The N-terminal amino acid residue (E) of the mature VH is double-underlined. CDR sequences according to the definition of Kabat et al. (1991) are underlined. The intron sequence is in italic.

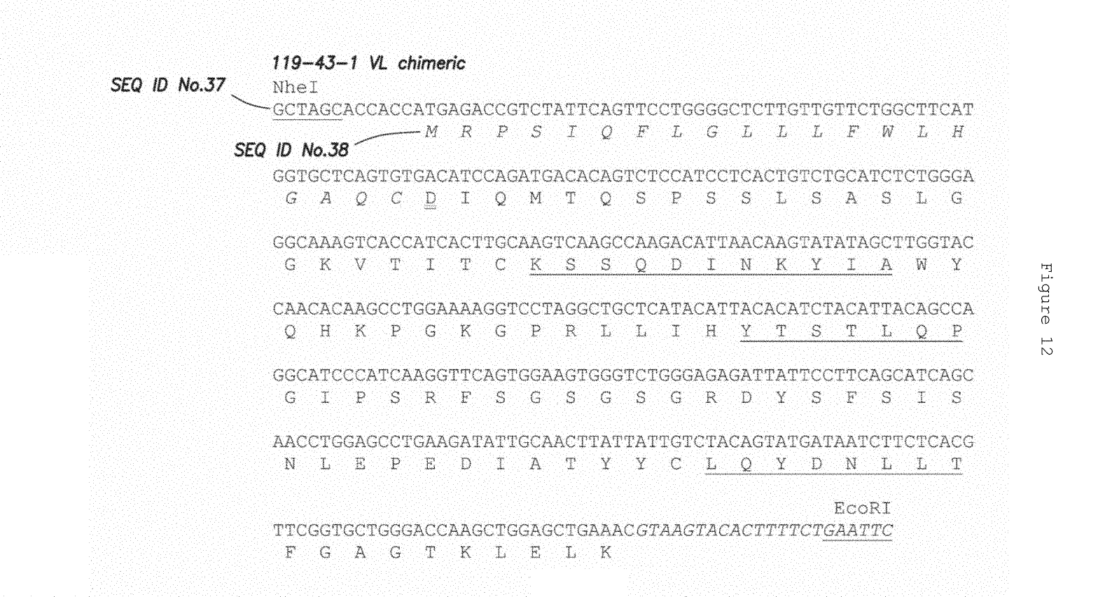

FIG. 12 shows the nucleotide sequence of the designed 119-43-1 VL gene flanked by NheI and EcoRI sites (underlined) along with the deduced amino acid sequence. Amino acid residues are shown in single letter code. The signal peptide sequence is in italic. The N-terminal amino acid residue (D) of the mature VL is double-underlined. CDR sequences according to the definition of Kabat et al. (1991) are underlined. The intron sequence is in italic.

In one embodiment, the anti-OX40 ABP of a combination of the invention, or a method or use thereof, comprises the CDRs of the 106-222 antibody e.g. CDRH1, CDRH2, and CDRH3 having the amino acid sequence as set forth in SEQ ID NOs 1, 2, and 3, and e.g. CDRL1, CDRL2, and CDRL3 having the sequences as set forth in SEQ ID NOs 7, 8, and 9 respectively. In one embodiment, the ABP of a combination of the invention, or a method or use thereof, comprises the CDRs of the 106-222, Hu106 or Hu106-222 antibody as disclosed in WO2012/027328 (PCT/US2011/048752), international filing date 23 Aug. 2011. As described herein, ANTIBODY 106-222 is a humanized monoclonal antibody that binds to human OX40 as disclosed in WO2012/027328 and described herein an antibody comprising CDRH1, CDRH2, and CDRH3 having the amino acid sequence as set forth in SEQ ID NOs 1, 2, and 3, and e.g. CDRL1, CDRL2, and CDRL3 having the sequences as set forth in SEQ ID NOs 7, 8, and 9, respectively and an antibody comprising VH having an amino acid sequence as set forth in SEQ ID NO:4 and a VL having an amino acid sequence as set forth in SEQ ID NO: 10. In a further embodiment, the anti-OX40 ABP of a combination of the invention, or a method or use thereof, comprises the VH and VL regions of the 106-222 antibody as shown in FIGS. 6-7 herein, e.g. a VH having an amino acid sequence as set forth in SEQ ID NO:4 and a VL having an amino acid sequence as set forth in SEQ ID NO: 10. In another embodiment, the ABP of a combination of the invention, or a method or use thereof, comprises a VH having an amino acid sequence as set forth in SEQ ID NO: 5, and a VL having an amino acid sequence as set forth in SEQ ID NO:11. In a further embodiment, the anti-OX40 ABP of a combination of the invention, or a method or use thereof, comprises the VH and VL regions of the Hu106-222 antibody or the 106-222 antibody or the Hu106 antibody as disclosed in WO2012/027328 (PCT/US2011/048752), international filing date 23 Aug. 2011. In a further embodiment, the anti-OX40 ABP of a combination of the invention, or a method or use thereof, is 106-222, Hu106-222 or Hu106, e.g. as disclosed in WO2012/027328 (PCT/US2011/048752), international filing date 23 Aug. 2011. In a further embodiment, the ABP of a combination of the invention, or a method or use thereof, comprises CDRs or VH or VL or antibody sequences with at least 90% (e.g., 90%, 91%, 92%, 93%, 94%, 95%, 96%, 97%, 98%, or 99%, or 100%) sequence identity to the sequences in this paragraph.

In another embodiment, the anti-OX40 ABP of a combination of the invention, or a method or use thereof, comprises the CDRs of the 119-122 antibody, e.g. CDRH1, CDRH2, and CDRH3 having the amino acid sequence as set forth in SEQ ID NOs 13, 14, and 15 respectively. In another embodiment, the anti-OX40 ABP of a combination of the invention, or a method or use thereof, comprises the CDRs of the 119-122 or Hu119 or Hu119-222 antibody as disclosed in WO2012/027328 (PCT/US2011/048752), international filing date 23 Aug. 2011. In a further embodiment, the anti-OX40 ABP of a combination of the invention, or a method or use thereof, comprises a VH having an amino acid sequence as set forth in SEQ ID NO: 16, and a VL having the amino acid sequence as set forth in SEQ ID NO: 22. In another embodiment, the anti-OX40 ABP of a combination of the invention, or a method or use thereof, comprises a VH having an amino acid sequence as set forth in SEQ ID NO: 17 and a VL having the amino acid sequence as set forth in SEQ ID NO: 23. In a further embodiment, the anti-OX40 ABP of a combination of the invention, or a method or use thereof, comprises the VH and VL regions of the 119-122 or Hu119 or Hu119-222 antibody as disclosed in WO2012/027328 (PCT/US2011/048752), international filing date 23 Aug. 2011. In a further embodiment, the ABP of a combination of the invention, or a method or use thereof, is 119-222 or Hu119 or Hu119-222 antibody, e.g. as disclosed in WO2012/027328 (PCT/US2011/048752), international filing date 23 Aug. 2011. In a further embodiment, the ABP comprises CDRs or VH or VL or antibody sequences with at least 90% (e.g., 90%, 91%, 92%, 93%, 94%, 95%, 96%, 97%, 98%, or 99%, or 100%) sequence identity to the sequences in this paragraph.

In another embodiment, the anti-OX40 ABP of a combination of the invention, or a method or use thereof, comprises the CDRs of the 119-43-1 antibody as disclosed in WO2013/028231 (PCT/US2012/024570), international filing date 9 Feb. 2012. In another embodiment, the anti-OX40 ABP of a combination of the invention, or a method or use thereof, comprises the CDRs of the 119-43-1 antibody as disclosed in WO2013/028231 (PCT/US2012/024570), international filing date 9 Feb. 2012. In a further embodiment, the anti-OX40 ABP of a combination of the invention, or a method or use thereof, comprises one of the VH and one of the VL regions of the 119-43-1 antibody. In a further embodiment, the anti-OX40 ABP of a combination of the invention, or a method or use thereof, comprises the VH and VL regions of the 119-43-1 antibody as disclosed in WO2013/028231 (PCT/US2012/024570), international filing date 9 Feb. 2012. In a further embodiment, the anti-OX40 ABP of a combination of the invention, or a method or use thereof, is 119-43-1 or 119-43-1 chimeric. In a further embodiment, the anti-OX40 ABP of a combination of the invention, or a method or use thereof, as disclosed in WO2013/028231 (PCT/US2012/024570), international filing date 9 Feb. 2012. In further embodiments, any one of the anti-OX40 ABPs described in this paragraph are humanized. In further embodiments, any one of the any one of the ABPs described in this paragraph are engineered to make a humanized antibody. In a further embodiment, the anti-OX40 ABP of a combination of the invention, or a method or use thereof, comprises CDRs or VH or VL or antibody sequences with at least 90% (e.g., 90%, 91%, 92%, 93%, 94%, 95%, 96%, 97%, 98%, or 99%, or 100%) sequence identity to the sequences in this paragraph.

In another embodiment, further embodiment, any mouse or chimeric sequences of any anti-OX40 ABP of a combination of the invention, or a method or use thereof, are engineered to make a humanized antibody.

In one embodiment, the anti-OX40 ABP of a combination of the invention, or a method or use thereof, comprises: (a) a heavy chain variable region CDR1 comprising the amino acid sequence of SEQ ID NO: 1; (b) a heavy chain variable region CDR2 comprising the amino acid sequence of SEQ ID NO: 2; (c) a heavy chain variable region CDR3 comprising the amino acid sequence of SEQ ID NO. 3; (d) a light chain variable region CDR1 comprising the amino acid sequence of SEQ ID NO. 7; (e) a light chain variable region CDR2 comprising the amino acid sequence of SEQ ID NO. 8; and (f) a light chain variable region CDR3 comprising the amino acid sequence of SEQ ID NO. 9.

In another embodiment, the anti-OX40 ABP of a combination of the invention, or a method or use thereof, comprises: (a) a heavy chain variable region CDR1 comprising the amino acid sequence of SEQ ID NO: 13; (b) a heavy chain variable region CDR2 comprising the amino acid sequence of SEQ ID NO: 14; (c) a heavy chain variable region CDR3 comprising the amino acid sequence of SEQ ID NO. 15; (d) a light chain variable region CDR1 comprising the amino acid sequence of SEQ ID NO. 19; (e) a light chain variable region CDR2 comprising the amino acid sequence of SEQ ID NO. 20; and (f) a light chain variable region CDR3 comprising the amino acid sequence of SEQ ID NO. 21.

In another embodiment, the anti-OX40 ABP of a combination of the invention, or a method or use thereof, comprises: a heavy chain variable region CDR1 comprising the amino acid sequence of SEQ ID NO: 1 or 13; a heavy chain variable region CDR2 comprising the amino acid sequence of SEQ ID NO: 2 or 14; and/or a heavy chain variable region CDR3 comprising the amino acid sequence of SEQ ID NO: 3 or 15, or a heavy chain variable region CDR having 90 percent identity thereto.

In another embodiment, the anti-OX40 ABP of a combination of the invention, or a method or use thereof, comprises: a light chain variable region CDR1 comprising the amino acid sequence of SEQ ID NO: 7 or 19; a light chain variable region CDR2 comprising the amino acid sequence of SEQ ID NO: 8 or 20 and/or a light chain variable region CDR3 comprising the amino acid sequence of SEQ ID NO: 9 or 21, or a heavy chain variable region having 90 percent identity thereto.

In another embodiment, the anti-OX40 ABP of a combination of the invention, or a method or use thereof, comprises: a light chain variable region (“VL”) comprising the amino acid sequence of SEQ ID NO: 10, 11, 22 or 23, or an amino acid sequence with at least 90% (e.g., 90%, 91%, 92%, 93%, 94%, 95%, 96%, 97%, 98%, or 99%, or 100%) sequence identity to the amino acid sequences of SEQ ID NO: 10, 11, 22 or 23. In another embodiment, the anti-OX40 ABP of a combination of the invention, or a method or use thereof, comprises a heavy chain variable region (“VH”) comprising the amino acid sequence of SEQ ID NO: 4, 5, 16 and 17, or an amino acid sequence with at least 90% (e.g., 90%, 91%, 92%, 93%, 94%, 95%, 96%, 97%, 98%, or 99%, or 100%) sequence identity to the amino acid sequences of SEQ ID NO: 4, 5, 16 and 17. In another embodiment, the anti-OX40 ABP of a combination of the invention, or a method or use thereof, comprises a variable heavy sequence of SEQ ID NO:5 and a variable light sequence of SEQ ID NO: 11, or a sequence having 90 percent identity thereto. In another embodiment, the anti-OX40 ABP of a combination of the invention, or a method or use thereof, comprises a variable heavy sequence of SEQ ID NO:17 and a variable light sequence of SEQ ID NO: 23 or a sequence having 90 percent identity thereto.

In another embodiment, the anti-OX40 ABP of a combination of the invention, or a method or use thereof, comprises a variable light chain encoded by the nucleic acid sequence of SEQ ID NO: 12, or 24, or a nucleic acid sequence with at least 90% (e.g., 90%, 91%, 92%, 93%, 94%, 95%, 96%, 97%, 98%, or 99%, or 100%) sequence identity to the nucleotide sequences of SEQ ID NO: 12 or 24. In another embodiment, the anti-OX40 ABP of a combination of the invention, or a method or use thereof, comprises a variable heavy chain encoded by a nucleic acid sequence of SEQ ID NO: 6 or 18, or a nucleic acid sequence with at least 90% (e.g., 90%, 91%, 92%, 93%, 94%, 95%, 96%, 97%, 98%, or 99%, or 100%) sequence identity to nucleotide sequences of SEQ ID NO: 6 or 18.

Also provided herein are monoclonal antibodies. In one embodiment, the monoclonal antibodies comprise a variable light chain comprising the amino acid sequence of SEQ ID NO: 10 or 22, or an amino acid sequence with at least 90% (e.g., 90%, 91%, 92%, 93%, 94%, 95%, 96%, 97%, 98%, or 99%, or 100%) sequence identity to the amino acid sequences of SEQ ID NO: 10 or 22. Further provided are monoclonal antibodies comprising a variable heavy chain comprising the amino acid sequence of SEQ ID NO: 4 or 16, or an amino acid sequence with at least 90% (e.g., 90%, 91%, 92%, 93%, 94%, 95%, 96%, 97%, 98%, or 99%, or 100%) sequence identity to the amino acid sequences of SEQ ID NO: 4 or 16.

Also provided herein are monoclonal antibodies. In one embodiment, the monoclonal antibodies comprise a variable light chain comprising the amino acid sequence of SEQ ID NO: 11 or 23, or an amino acid sequence with at least 90% (e.g., 90%, 91%, 92%, 93%, 94%, 95%, 96%, 97%, 98%, or 99%, or 100%) sequence identity to the amino acid sequences of SEQ ID NO: 11 or 23. Further provided are monoclonal antibodies comprising a variable heavy chain comprising the amino acid sequence of SEQ ID NO: 5 or 17, or an amino acid sequence with at least 90% (e.g., 90%, 91%, 92%, 93%, 94%, 95%, 96%, 97%, 98%, or 99%, or 100%) sequence identity to the amino acid sequences of SEQ ID NO: 5 or 17.

Another embodiment of a combination of the invention, or a method or use thereof, includes CDRs, VH regions, and VL regions, and antibodies and nucleic acids encoding the same as disclosed in the below Sequence Listing.

| Heavy Chain of ANTIBODY 106-222: | |
|---|---|
| (SEQ ID NO: 48) | |
| QVQLVQSGSELKKPGASVKVSCKASGYTFTDYSMHWVRQAPGQGLKWMGWINTE | |
| TGEPTYADDFKGRFVFSLDTSVSTAYLQISSLKAEDTAVYYCANPYYDYVSYYAMD | |
| YWGQGTTVTVSSASTKGPSVFPLAPSSKSTSGGTAALGCLVKDYFPEPVTVSWNS | |
| GALTSGVHTFPAVLQSSGLYSLSSVVTVPSSSLGTQTYICNVNHKPSNTKVDKKVEP | |
| KSCDKTHTCPPCPAPELLGGPSVFLFPPKPKDTLMISRTPEVTCVVVDVSHEDPEV | |
| KFNWYVDGVEVHNAKTKPREEQYNSTYRVVSVLTVLHQDWLNGKEYKCKVSNKAL | |
| PAPIEKTISKAKGQPREPQVYTLPPSRDELTKNQVSLTCLVKGFYPSDIAVEWESNG | |
| QPENNYKTTPPVLDSDGSFFLYSKLTVDKSRWQQGNVFSCSVMHEALHNHYTQKS | |
| LSLSPGK | |
| Light Chain of ANTIBODY 106-222: | |
| (SEQ ID NO: 49) | |
| DIQMTQSPSSLSASVGDRVTITCKASQDVSTAVAWYQQKPGKAPKLLIYSASYLYTG | |
| VPSRFSGSGSGTDFTFTISSLQPEDIATYYCQQHYSTPRTFGQGTKLEIKRTVAAPS | |
| VFIFPPSDEQLKSGTASVVCLLNNFYPREAKVQWKVDNALQSGNSQESVTEQDSK | |
| DSTYSLSSTLTLSKADYEKHKVYACEVTHQGLSSPVTKSFNRGEC | |
| Heavy Chain Variable Region of ANTIBODY 106-222: | |
| (SEQ ID NO: 5) | |
| QVQLVQSGSELKKPGASVKVSCKASGYTFTDYSMHWVRQAPGQGLKWMGWINTE | |
| TGEPTYADDFKGRFVFSLDTSVSTAYLQISSLKAEDTAVYYCANPYYDYVSYYAMD | |
| YWGQGTTVTVSS | |
| Light Chain Variable Region of ANTIBODY 106-222: | |
| (SEQ ID NO: 11) | |
| DIQMTQSPSSLSASVGDRVTITCKASQDVSTAVAWYQQKPGKAPKLLIYSASYLYTG | |
| VPSRFSGSGSGTDFTFTISSLQPEDIATYYCQQHYSTPRTFGQGTKLEIK | |
| CDR sequences of ANTIBODY 106-222: | |
| HC CDR1: | |
| (SEQ ID NO: 1) | |
| Asp Tyr Ser Met His | |
| HC CDR2: | |
| (SEQ ID NO: 2) | |
| Trp Ile Asn Thr Glu Thr Gly Glu Pro Thr Tyr Ala Asp Asp Phe Lys Gly | |
| HC CDR3: | |
| (SEQ ID NO: 3) | |
| Pro Tyr Tyr Asp Tyr Val Ser Tyr Tyr Ala Met Asp Tyr | |
| LC CDR1: | |
| (SEQ ID NO: 7) | |
| Lys Ala Ser Gln Asp Val Ser Thr Ala Val Ala | |
| LC CDR2: | |
| (SEQ ID NO: 8) | |
| Ser Ala Ser Tyr Leu Tyr Thr | |
| LC CDR3: | |
| (SEQ ID NO: 9) | |
| Gln Gln His Tyr Ser Thr Pro Arg Thr | |
| OX40 Antibody Sequence Listing | |
| <140> UNKNOWN | |
| <141> 2014-02-24 | |
| <150> PCT/US2012/024570 | |
| <151> 2012-02-09 | |
| <150> PCT/US2011/048752 | |
| <151> 2011-08-23 | |
| <160> 47 | |
| <170> PatentIn version 3.5 | |
| <210> 1 | |
| <211> 5 | |
| <212> PRT | |
| <213> Mus sp. | |
| <400> 1 | |
| Asp Tyr Ser Met His | |
| 1 5 | |
| <210> 2 | |
| <211> 17 | |
| <212> PRT | |
| <213> Mus sp. | |
| <400> 2 | |
| Trp Ile Asn Thr Glu Thr Gly Glu Pro Thr Tyr Ala Asp Asp Phe | |
| Lys | |
| 1 5 10 15 | |
| Gly | |
| <210> 3 | |
| <211> 13 | |
| <212> PRT | |
| <213> Mus sp. | |
| <400> 3 | |
| Pro Tyr Tyr Asp Tyr Val Ser Tyr Tyr Ala Met Asp Tyr | |
| 1 5 10 | |
| <210> 4 | |
| <211> 122 | |
| <212> PRT | |
| <213> Mus sp. | |
| <400> 4 | |
| Gln Ile Gln Leu Val Gln Ser Gly Pro Glu Leu Lys Lys Pro Gly | |
| Glu | |
| 1 5 10 15 | |
| Thr Val Lys Ile Ser Cys Lys Ala Ser Gly Tyr Thr Phe Thr Asp | |
| Tyr | |
| 20 25 30 | |
| Ser Met His Trp Val Lys Gln Ala Pro Gly Lys Gly Leu Lys Trp | |
| Met | |
| 35 40 45 | |
| Gly Trp Ile Asn Thr Glu Thr Gly Glu Pro Thr Tyr Ala Asp Asp | |
| Phe | |
| 50 55 60 | |
| Lys Gly Arg Phe Ala Phe Ser Leu Glu Thr Ser Ala Ser Thr Ala | |
| Tyr | |
| 65 70 75 80 | |
| Leu Gln Ile Asn Asn Leu Lys Asn Glu Asp Thr Ala Thr Tyr Phe | |
| Cys | |
| 85 90 95 | |
| Ala Asn Pro Tyr Tyr Asp Tyr Val Ser Tyr Tyr Ala Met Asp Tyr | |
| Trp | |
| 100 105 110 | |
| Gly His Gly Thr Ser Val Thr Val Ser Ser | |
| 115 120 | |
| <210> 5 | |
| <211> 122 | |
| <212> PRT | |
| <213> Artificial Sequence | |
| <220> | |
| <223> Description of Artificial Sequence: Synthetic | |
| polypeptide | |
| <400> 5 | |
| Gln Val Gln Leu Val Gln Ser Gly Ser Glu Leu Lys Lys Pro Gly | |
| Ala | |
| 1 5 10 15 | |
| Ser Val Lys Val Ser Cys Lys Ala Ser Gly Tyr Thr Phe Thr Asp | |
| Tyr | |
| 20 25 30 | |
| Ser Met His Trp Val Arg Gln Ala Pro Gly Gln Gly Leu Lys Trp | |
| Met | |
| 35 40 45 | |
| Gly Trp Ile Asn Thr Glu Thr Gly Glu Pro Thr Tyr Ala Asp Asp | |
| Phe | |
| 50 55 60 | |
| Lys Gly Arg Phe Val Phe Ser Leu Asp Thr Ser Val Ser Thr Ala | |
| Tyr | |
| 65 70 75 80 | |
| Leu Gln Ile Ser Ser Leu Lys Ala Glu Asp Thr Ala Val Tyr Tyr | |
| Cys | |
| 85 90 95 | |
| Ala Asn Pro Tyr Tyr Asp Tyr Val Ser Tyr Tyr Ala Met Asp Tyr | |
| Trp | |
| 100 105 110 | |
| Gly Gln Gly Thr Thr Val Thr Val Ser Ser | |
| 115 120 | |
| <210> 6 | |
| <211> 458 | |
| <212> DNA | |
| <213> Artificial Sequence | |
| <220> | |
| <223> Description of Artificial Sequence: Synthetic | |
| polynucleotide | |
| <400> 6 | |
| actagtacca ccatggcttg ggtgtggacc ttgctattcc tgatggcagc tgcccaaagt | 60 |
| atccaagcac aggttcagtt ggtgcagtct ggatctgagc tgaagaagcc tggagcctca | 120 |
| gtcaaggttt cctgcaaggc ttctggttat accttcacag actattcaat gcactgggtg | 180 |
| cgacaggctc caggacaagg tttaaagtgg atgggctgga taaacactga gactggtgag | 240 |
| ccaacatatg cagatgactt caagggacgg tttgtcttct ctttggacac ctctgtcagc | 300 |
| actgcctatt tgcagatcag cagcctcaaa gctgaggaca cggctgtgta ttactgtgct | 360 |
| aatccctact atgattacgt ctcttactat gctatggact actggggtca gggaaccacg | 420 |
| gtcaccgtct cctcaggtaa gaatggcctc tcaagctt | 458 |
| <210> 7 | |
| <211> 11 | |
| <212> PRT | |
| <213> Mus sp. | |
| <400> 7 | |
| Lys Ala Ser Gln Asp Val Ser Thr Ala Val Ala | |
| 1 5 10 | |
| <210> 8 | |
| <211> 7 | |
| <212> PRT | |
| <213> Mus sp. | |
| <400> 8 | |
| Ser Ala Ser Tyr Leu Tyr Thr | |
| 1 5 | |
| <210> 9 | |
| <211> 9 | |
| <212> PRT | |
| <213> Mus sp. | |
| <400> 9 | |
| Gln Gln His Tyr Ser Thr Pro Arg Thr | |
| 1 5 | |
| <210> 10 | |
| <211> 107 | |
| <212> PRT | |
| <213> Mus sp. | |
| <400> 10 | |
| Asp Ile Val Met Thr Gln Ser His Lys Phe Met Ser Thr Ser Val | |
| Arg | |
| 1 5 10 15 | |
| Asp Arg Val Ser Ile Thr Cys Lys Ala Ser Gln Asp Val Ser Thr | |
| Ala | |
| 20 25 30 | |
| Val Ala Trp Tyr Gln Gln Lys Pro Gly Gln Ser Pro Lys Leu Leu | |
| Ile | |
| 35 40 45 | |
| Tyr Ser Ala Ser Tyr Leu Tyr Thr Gly Val Pro Asp Arg Phe Thr | |
| Gly | |
| 50 55 60 | |
| Ser Gly Ser Gly Thr Asp Phe Thr Phe Thr Ile Ser Ser Val Gln | |
| Ala | |
| 65 70 75 80 | |
| Glu Asp Leu Ala Val Tyr Tyr Cys Gln Gln His Tyr Ser Thr Pro | |
| Arg | |
| 85 90 95 | |
| Thr Phe Gly Gly Gly Thr Lys Leu Glu Ile Lys | |
| 100 105 | |
| <210> 11 | |
| <211> 107 | |
| <212> PRT | |
| <213> Artificial Sequence | |
| <220> | |
| <223> Description of Artificial Sequence: Synthetic | |
| polypeptide | |
| <400> 11 | |
| Asp Ile Gln Met Thr Gln Ser Pro Ser Ser Leu Ser Ala Ser Val | |
| Gly | |
| 1 5 10 15 | |
| Asp Arg Val Thr Ile Thr Cys Lys Ala Ser Gln Asp Val Ser Thr | |
| Ala | |
| 20 25 30 | |
| Val Ala Trp Tyr Gln Gln Lys Pro Gly Lys Ala Pro Lys Leu Leu | |
| Ile | |
| 35 40 45 | |
| Tyr Ser Ala Ser Tyr Leu Tyr Thr Gly Val Pro Ser Arg Phe Ser | |
| Gly | |
| 50 55 60 | |
| Ser Gly Ser Gly Thr Asp Phe Thr Phe Thr Ile Ser Ser Leu Gln | |
| Pro | |
| 65 70 75 80 | |
| Glu Asp Ile Ala Thr Tyr Tyr Cys Gln Gln His Tyr Ser Thr Pro | |
| Arg | |
| 85 90 95 | |
| Thr Phe Gly Gln Gly Thr Lys Leu Glu Ile Lys | |
| 100 105 | |
| <210> 12 | |
| <211> 416 | |
| <212> DNA | |
| <213> Artificial Sequence | |
| <220> | |
| <223> Description of Artificial Sequence: Synthetic | |
| polynucleotide | |
| <400> 12 | |
| gctagcacca ccatggagtc acagattcag gtctttgtat tcgtgtttct ctggttgtct | 60 |
| ggtgttgacg gagacattca gatgacccag tctccatcct ccctgtccgc atcagtggga | 120 |
| gacagggtca ccatcacctg caaggccagt caggatgtga gtactgctgt agcctggtat | 180 |
| caacagaaac caggaaaagc ccctaaacta ctgatttact cggcatccta cctctacact | 240 |
| ggagtccctt cacgcttcag tggcagtgga tctgggacgg atttcacttt caccatcagc | 300 |
| agtctgcagc ctgaagacat tgcaacatat tactgtcagc aacattatag tactcctcgg | 360 |
| acgttcggtc agggcaccaa gctggaaatc aaacgtaagt agaatccaaa gaattc | 416 |
| <210> 13 | |
| <211> 5 | |
| <212> PRT | |
| <213> Mus sp. | |
| <400> 13 | |
| Ser His Asp Met Ser | |
| 1 5 | |
| <210> 14 | |
| <211> 17 | |
| <212> PRT | |
| <213> Mus sp. | |
| <400> 14 | |
| Ala Ile Asn Ser Asp Gly Gly Ser Thr Tyr Tyr Pro Asp Thr Met | |
| Glu | |
| 1 5 10 15 | |
| Arg | |
| <210> 15 | |
| <211> 11 | |
| <212> PRT | |
| <213> Mus sp. | |
| <400> 15 | |
| His Tyr Asp Asp Tyr Tyr Ala Trp Phe Ala Tyr | |
| 1 5 10 | |
| <210> 16 | |
| <211> 120 | |
| <212> PRT | |
| <213> Mus sp. | |
| <400> 16 | |
| Glu Val Gln Leu Val Glu Ser Gly Gly Gly Leu Val Gln Pro Gly | |
| Glu | |
| 1 5 10 15 | |
| Ser Leu Lys Leu Ser Cys Glu Ser Asn Glu Tyr Glu Phe Pro Ser | |
| His | |
| 20 25 30 | |
| Asp Met Ser Trp Val Arg Lys Thr Pro Glu Lys Arg Leu Glu Leu | |
| Val | |
| 35 40 45 | |
| Ala Ala Ile Asn Ser Asp Gly Gly Ser Thr Tyr Tyr Pro Asp Thr | |
| Met | |
| 50 55 60 | |
| Glu Arg Arg Phe Ile Ile Ser Arg Asp Asn Thr Lys Lys Thr Leu | |
| Tyr | |
| 65 70 75 80 | |
| Leu Gln Met Ser Ser Leu Arg Ser Glu Asp Thr Ala Leu Tyr Tyr | |
| Cys | |
| 85 90 95 | |
| Ala Arg His Tyr Asp Asp Tyr Tyr Ala Trp Phe Ala Tyr Trp Gly | |
| Gln | |
| 100 105 110 | |
| Gly Thr Leu Val Thr Val Ser Ala | |
| 115 120 | |
| <210> 17 | |
| <211> 120 | |
| <212> PRT | |
| <213> Artificial Sequence | |
| <220> | |
| <223> Description of Artificial Sequence: Synthetic | |
| polypeptide | |
| <400> 17 | |
| Glu Val Gln Leu Val Glu Ser Gly Gly Gly Leu Val Gln Pro Gly | |
| Gly | |
| 1 5 10 15 | |
| Ser Leu Arg Leu Ser Cys Ala Ala Ser Glu Tyr Glu Phe Pro Ser | |
| His | |
| 20 25 30 | |
| Asp Met Ser Trp Val Arg Gln Ala Pro Gly Lys Gly Leu Glu Leu | |
| Val | |
| 35 40 45 | |
| Ala Ala Ile Asn Ser Asp Gly Gly Ser Thr Tyr Tyr Pro Asp Thr | |
| Met | |
| 50 55 60 | |
| Glu Arg Arg Phe Thr Ile Ser Arg Asp Asn Ala Lys Asn Ser Leu | |
| Tyr | |
| 65 70 75 80 | |
| Leu Gln Met Asn Ser Leu Arg Ala Glu Asp Thr Ala Val Tyr Tyr | |
| Cys | |
| 85 90 95 | |
| Ala Arg His Tyr Asp Asp Tyr Tyr Ala Trp Phe Ala Tyr Trp Gly | |
| Gln | |
| 100 105 110 | |
| Gly Thr Met Val Thr Val Ser Ser | |
| 115 120 | |
| <210> 18 | |
| <211> 451 | |
| <212> DNA | |
| <213> Artificial Sequence | |
| <220> | |
| <223> Description of Artificial Sequence: Synthetic | |
| polynucleotide | |
| <400> 18 | |
| actagtacca ccatggactt cgggctcagc ttggttttcc ttgtccttat tttaaaaagt | 60 |
| gtacagtgtg aggtgcagct ggtggagtct gggggaggct tagtgcagcc tggagggtcc | 120 |
| ctgagactct cctgtgcagc ctctgaatac gagttccctt cccatgacat gtcttgggtc | 180 |
| cgccaggctc cggggaaggg gctggagttg gtcgcagcca ttaatagtga tggtggtagc | 240 |
| acctactatc cagacaccat ggagagacga ttcaccatct ccagagacaa tgccaagaac | 300 |
| tcactgtacc tgcaaatgaa cagtctgagg gccgaggaca cagccgtgta ttactgtgca | 360 |
| agacactatg atgattacta cgcctggttt gcttactggg gccaagggac tatggtcact | 420 |
| gtctcttcag gtgagtccta acttcaagct t | 451 |
| <210> 19 | |
| <211> 15 | |
| <212> PRT | |
| <213> Mus sp. | |
| <400> 19 | |
| Arg Ala Ser Lys Ser Val Ser Thr Ser Gly Tyr Ser Tyr Met His | |
| 1 5 10 15 | |
| <210> 20 | |
| <211> 7 | |
| <212> PRT | |
| <213> Mus sp. | |
| <400> 20 | |
| Leu Ala Ser Asn Leu Glu Ser | |
| 1 5 | |
| <210> 21 | |
| <211> 9 | |
| <212> PRT | |
| <213> Mus sp. | |
| <400> 21 | |
| Gln His Ser Arg Glu Leu Pro Leu Thr | |
| 1 5 | |
| <210> 22 | |
| <211> 111 | |
| <212> PRT | |
| <213> Mus sp. | |
| <400> 22 | |
| Asp Ile Val Leu Thr Gln Ser Pro Ala Ser Leu Ala Val Ser Leu | |
| Gly | |
| 1 5 10 15 | |
| Gln Arg Ala Thr Ile Ser Cys Arg Ala Ser Lys Ser Val Ser Thr | |
| Ser | |
| 20 25 30 | |
| Gly Tyr Ser Tyr Met His Trp Tyr Gln Gln Lys Pro Gly Gln Pro | |
| Pro | |
| 35 40 45 | |
| Lys Leu Leu Ile Tyr Leu Ala Ser Asn Leu Glu Ser Gly Val Pro | |
| Ala | |
| 50 55 60 | |
| Arg Phe Ser Gly Ser Gly Ser Gly Thr Asp Phe Thr Leu Asn Ile | |
| His | |
| 65 70 75 80 | |
| Pro Val Glu Glu Glu Asp Ala Ala Thr Tyr Tyr Cys Gln His Ser | |
| Arg | |
| 85 90 95 | |
| Glu Leu Pro Leu Thr Phe Gly Ala Gly Thr Lys Leu Glu Leu Lys | |
| 100 105 110 | |
| <210> 23 | |
| <211> 111 | |
| <212> PRT | |
| <213> Artificial Sequence | |
| <220> | |
| <223> Description of Artificial Sequence: Synthetic | |
| polypeptide | |
| <400> 23 | |
| Glu Ile Val Leu Thr Gln Ser Pro Ala Thr Leu Ser Leu Ser Pro | |
| Gly | |
| 1 5 10 15 | |
| Glu Arg Ala Thr Leu Ser Cys Arg Ala Ser Lys Ser Val Ser Thr | |
| Ser | |
| 20 25 30 | |
| Gly Tyr Ser Tyr Met His Trp Tyr Gln Gln Lys Pro Gly Gln Ala | |
| Pro | |
| 35 40 45 | |
| Arg Leu Leu Ile Tyr Leu Ala Ser Asn Leu Glu Ser Gly Val Pro | |
| Ala | |
| 50 55 60 | |
| Arg Phe Ser Gly Ser Gly Ser Gly Thr Asp Phe Thr Leu Thr Ile | |
| Ser | |
| 65 70 75 80 | |
| Ser Leu Glu Pro Glu Asp Phe Ala Val Tyr Tyr Cys Gln His Ser | |
| Arg | |
| 85 90 95 | |
| Glu Leu Pro Leu Thr Phe Gly Gly Gly Thr Lys Val Glu Ile Lys | |
| 100 105 110 | |
| <210> 24 | |
| <211> 428 | |
| <212> DNA | |
| <213> Artificial Sequence | |
| <220> | |
| <223> Description of Artificial Sequence: Synthetic | |
| polynucleotide | |
| <400> 24 | |
| gctagcacca ccatggagac agacacactc ctgttatggg tactgctgct ctgggttcca | 60 |
| ggttccactg gtgaaattgt gctgacacag tctcctgcta ccttatcttt gtctccaggg | 120 |
| gaaagggcca ccctctcatg cagggccagc aaaagtgtca gtacatctgg ctatagttat | 180 |
| atgcactggt accaacagaa accaggacag gctcccagac tcctcatcta tcttgcatcc | 240 |
| aacctagaat ctggggtccc tgccaggttc agtggcagtg ggtctgggac agacttcacc | 300 |
| ctcaccatca gcagcctaga gcctgaggat tttgcagttt attactgtca gcacagtagg | 360 |
| gagcttccgc tcacgttcgg cggagggacc aaggtcgaga tcaaacgtaa gtacactttt | 420 |
| ctgaattc | 428 |
| <210> 25 | |
| <211> 5 | |
| <212> PRT | |
| <213> Mus sp. | |
| <400> 25 | |
| Asp Ala Trp Met Asp | |
| 1 5 | |
| <210> 26 | |
| <211> 19 | |
| <212> PRT | |
| <213> Mus sp. | |
| <400> 26 | |
| Glu Ile Arg Ser Lys Ala Asn Asn His Ala Thr Tyr Tyr Ala Glu | |
| Ser | |
| 1 5 10 15 | |
| Val Asn Gly | |
| <210> 27 | |
| <211> 8 | |
| <212> PRT | |
| <213> Mus sp. | |
| <400> 27 | |
| Gly Glu Val Phe Tyr Phe Asp Tyr | |
| 1 5 | |
| <210> 28 | |
| <211> 414 | |
| <212> DNA | |
| <213> Mus sp. | |
| <400> 28 | |
| atgtacttgg gactgaacta tgtattcata gtttttctct taaatggtgt ccagagtgaa | 60 |
| gtgaagcttg aggagtctgg aggaggcttg gtgcaacctg gaggatccat gaaactctct | 120 |
| tgtgctgcct ctggattcac ttttagtgac gcctggatgg actgggtccg ccagtctcca | 180 |
| gagaaggggc ttgagtgggt tgctgaaatt agaagcaaag ctaataatca tgcaacatac | 240 |
| tatgctgagt ctgtgaatgg gaggttcacc atctcaagag atgattccaa aagtagtgtc | 300 |
| tacctgcaaa tgaacagctt aagagctgaa gacactggca tttattactg tacgtggggg | 360 |
| gaagtgttct actttgacta ctggggccaa ggcaccactc tcacagtctc ctca | 414 |
| <210> 29 | |
| <211> 138 | |
| <212> PRT | |
| <213> Mus sp. | |
| <400> 29 | |
| Met Tyr Leu Gly Leu Asn Tyr Val Phe Ile Val Phe Leu Leu Asn | |
| Gly | |
| 1 5 10 15 | |
| Val Gln Ser Glu Val Lys Leu Glu Glu Ser Gly Gly Gly Leu Val | |
| Gln | |
| 20 25 30 | |
| Pro Gly Gly Ser Met Lys Leu Ser Cys Ala Ala Ser Gly Phe Thr | |
| Phe | |
| 35 40 45 | |
| Ser Asp Ala Trp Met Asp Trp Val Arg Gln Ser Pro Glu Lys Gly | |
| Leu | |
| 50 55 60 | |
| Glu Trp Val Ala Glu Ile Arg Ser Lys Ala Asn Asn His Ala Thr | |
| Tyr | |
| 65 70 75 80 | |
| Tyr Ala Glu Ser Val Asn Gly Arg Phe Thr Ile Ser Arg Asp Asp | |
| Ser | |
| 85 90 95 | |
| Lys Ser Ser Val Tyr Leu Gln Met Asn Ser Leu Arg Ala Glu Asp | |
| Thr | |
| 100 105 110 | |
| Gly Ile Tyr Tyr Cys Thr Trp Gly Glu Val Phe Tyr Phe Asp Tyr | |
| Trp | |
| 115 120 125 | |
| Gly Gln Gly Thr Thr Leu Thr Val Ser Ser | |
| 130 135 | |
| <210> 30 | |
| <211> 448 | |
| <212> DNA | |
| <213> Artificial Sequence | |
| <220> | |
| <223> Description of Artificial Sequence: Synthetic | |
| polynucleotide | |
| <400> 30 | |
| actagtacca ccatgtactt gggactgaac tatgtattca tagtttttct cttaaatggt | 60 |
| gtccagagtg aagtgaagct ggaggagtct ggaggaggct tggtgcaacc tggaggatcc | 120 |
| atgaaactct cttgtgctgc ctctggattc acttttagtg acgcctggat ggactgggtc | 180 |
| cgccagtctc cagagaaggg gcttgagtgg gttgctgaaa ttagaagcaa agctaataat | 240 |
| catgcaacat actatgctga gtctgtgaat gggaggttca ccatctcaag agatgattcc | 300 |
| aaaagtagtg tctacctgca aatgaacagc ttaagagctg aagacactgg catttattac | 360 |
| tgtacgtggg gggaagtgtt ctactttgac tactggggcc aaggcaccac tctcacagtc | 420 |
| tcctcaggtg agtccttaaa acaagctt | 448 |
| <210> 31 | |
| <211> 138 | |
| <212> PRT | |
| <213> Artificial Sequence | |
| <220> | |
| <223> Description of Artificial Sequence: Synthetic | |
| polypeptide | |
| <400> 31 | |
| Met Tyr Leu Gly Leu Asn Tyr Val Phe Ile Val Phe Leu Leu Asn | |
| Gly | |
| 1 5 10 15 | |
| Val Gln Ser Glu Val Lys Leu Glu Glu Ser Gly Gly Gly Leu Val | |
| Gln | |
| 20 25 30 | |
| Pro Gly Gly Ser Met Lys Leu Ser Cys Ala Ala Ser Gly Phe Thr | |
| Phe | |
| 35 40 45 | |
| Ser Asp Ala Trp Met Asp Trp Val Arg Gln Ser Pro Glu Lys Gly | |
| Leu | |
| 50 55 60 | |
| Glu Trp Val Ala Glu Ile Arg Ser Lys Ala Asn Asn His Ala Thr | |
| Tyr | |
| 65 70 75 80 | |
| Tyr Ala Glu Ser Val Asn Gly Arg Phe Thr Ile Ser Arg Asp Asp | |
| Ser | |
| 85 90 95 | |
| Lys Ser Ser Val Tyr Leu Gln Met Asn Ser Leu Arg Ala Glu Asp | |
| Thr | |
| 100 105 110 | |
| Gly Ile Tyr Tyr Cys Thr Trp Gly Glu Val Phe Tyr Phe Asp Tyr | |
| Trp | |
| 115 120 125 | |
| Gly Gln Gly Thr Thr Leu Thr Val Ser Ser | |
| 130 135 | |
| <210> 32 | |
| <211> 11 | |
| <212> PRT | |
| <213> Mus sp. | |
| <400> 32 | |
| Lys Ser Ser Gln Asp Ile Asn Lys Tyr Ile Ala | |
| 1 5 10 | |
| <210> 33 | |
| <211> 7 | |
| <212> PRT | |
| <213> Mus sp. | |
| <400> 33 | |
| Tyr Thr Ser Thr Leu Gln Pro | |
| 1 5 | |
| <210> 34 | |
| <211> 8 | |
| <212> PRT | |
| <213> Mus sp. | |
| <400> 34 | |
| Leu Gln Tyr Asp Asn Leu Leu Thr | |
| 1 5 | |
| <210> 35 | |
| <211> 378 | |
| <212> DNA | |
| <213> Mus sp. | |
| <400> 35 | |
| atgagaccgt ctattcagtt cctggggctc ttgttgttct ggcttcatgg tgctcagtgt | 60 |
| gacatccaga tgacacagtc tccatcctca ctgtctgcat ctctgggagg caaagtcacc | 120 |
| atcacttgca agtcaagcca agacattaac aagtatatag cttggtacca acacaagcct | 180 |
| ggaaaaggtc ctaggctgct catacattac acatctacat tacagccagg catcccatca | 240 |
| aggttcagtg gaagtgggtc tgggagagat tattccttca gcatcagcaa cctggagcct | 300 |
| gaagatattg caacttatta ttgtctacag tatgataatc ttctcacgtt cggtgctggg | 360 |
| accaagctgg agctgaaa | 378 |
| <210> 36 | |
| <211> 126 | |
| <212> PRT | |
| <213> Mus sp. | |
| <400> 36 | |
| Met Arg Pro Ser Ile Gln Phe Leu Gly Leu Leu Leu Phe Trp Leu | |
| His | |
| 1 5 10 15 | |
| Gly Ala Gln Cys Asp Ile Gln Met Thr Gln Ser Pro Ser Ser Leu | |
| Ser | |
| 20 25 30 | |
| Ala Ser Leu Gly Gly Lys Val Thr Ile Thr Cys Lys Ser Ser Gln | |
| Asp | |
| 35 40 45 | |
| Ile Asn Lys Tyr Ile Ala Trp Tyr Gln His Lys Pro Gly Lys Gly | |
| Pro | |
| 50 55 60 | |
| Arg Leu Leu Ile His Tyr Thr Ser Thr Leu Gln Pro Gly Ile Pro | |
| Ser | |
| 65 70 75 80 | |
| Arg Phe Ser Gly Ser Gly Ser Gly Arg Asp Tyr Ser Phe Ser Ile | |
| Ser | |
| 85 90 95 | |
| Asn Leu Glu Pro Glu Asp Ile Ala Thr Tyr Tyr Cys Leu Gln Tyr | |
| Asp | |
| 100 105 110 | |
| Asn Leu Leu Thr Phe Gly Ala Gly Thr Lys Leu Glu Leu Lys | |
| 115 120 125 | |
| <210> 37 | |
| <211> 413 | |
| <212> DNA | |
| <213> Artificial Sequence | |
| <220> | |
| <223> Description of Artificial Sequence: Synthetic | |
| polynucleotide | |
| <400> 37 | |
| gctagcacca ccatgagacc gtctattcag ttcctggggc tcttgttgtt ctggcttcat | 60 |
| ggtgctcagt gtgacatcca gatgacacag tctccatcct cactgtctgc atctctggga | 120 |
| ggcaaagtca ccatcacttg caagtcaagc caagacatta acaagtatat agcttggtac | 180 |
| caacacaagc ctggaaaagg tcctaggctg ctcatacatt acacatctac attacagcca | 240 |
| ggcatcccat caaggttcag tggaagtggg tctgggagag attattcctt cagcatcagc | 300 |
| aacctggagc ctgaagatat tgcaacttat tattgtctac agtatgataa tcttctcacg | 360 |
| ttcggtgctg ggaccaagct ggagctgaaa cgtaagtaca cttttctgaa ttc | 413 |
| <210> 38 | |
| <211> 126 | |
| <212> PRT | |
| <213> Artificial Sequence | |
| <220> | |
| <223> Description of Artificial Sequence: Synthetic | |
| polypeptide | |
| <400> 38 | |
| Met Arg Pro Ser Ile Gln Phe Leu Gly Leu Leu Leu Phe Trp Leu | |
| His | |
| 1 5 10 15 | |
| Gly Ala Gln Cys Asp Ile Gln Met Thr Gln Ser Pro Ser Ser Leu | |
| Ser | |
| 20 25 30 | |
| Ala Ser Leu Gly Gly Lys Val Thr Ile Thr Cys Lys Ser Ser Gln | |
| Asp | |
| 35 40 45 | |
| Ile Asn Lys Tyr Ile Ala Trp Tyr Gln His Lys Pro Gly Lys Gly | |
| Pro | |
| 50 55 60 | |
| Arg Leu Leu Ile His Tyr Thr Ser Thr Leu Gln Pro Gly Ile Pro | |
| Ser | |
| 65 70 75 80 | |
| Arg Phe Ser Gly Ser Gly Ser Gly Arg Asp Tyr Ser Phe Ser Ile | |
| Ser | |
| 85 90 95 | |
| Asn Leu Glu Pro Glu Asp Ile Ala Thr Tyr Tyr Cys Leu Gln Tyr | |
| Asp | |
| 100 105 110 | |
| Asn Leu Leu Thr Phe Gly Ala Gly Thr Lys Leu Glu Leu Lys | |
| 115 120 125 | |
| <210> 39 | |
| <211> 20 | |
| <212> DNA | |
| <213> Artificial Sequence | |
| <220> | |
| <223> Description of Artificial Sequence: Synthetic | |
| primer | |
| <400> 39 | |
| cgctgttttg acctccatag | |
| 20 | |
| <210> 40 | |
| <211> 20 | |
| <212> DNA | |
| <213> Artificial Sequence | |
| <220> | |
| <223> Description of Artificial Sequence: Synthetic | |
| primer | |
| <400> 40 | |
| tgaaagatga gctggaggac | |
| 20 | |
| <210> 41 | |
| <211> 20 | |
| <212> DNA | |
| <213> Artificial Sequence | |
| <220> | |
| <223> Description of Artificial Sequence: Synthetic | |
| primer | |
| <400> 41 | |
| ctttcttgtc caccttggtg | |
| 20 | |
| <210> 42 | |
| <211> 19 | |
| <212> DNA | |
| <213> Artificial Sequence | |
| <220> | |
| <223> Description of Artificial Sequence: Synthetic | |
| primer | |
| <400> 42 | |
| gctgtcctac agtcctcag | |
| 19 | |
| <210> 43 | |
| <211> 18 | |
| <212> DNA | |
| <213> Artificial Sequence | |
| <220> | |
| <223> Description of Artificial Sequence: Synthetic | |
| primer | |
| <400> 43 | |
| acgtgccaag catcctcg | |
| 18 | |
| <210> 44 | |
| <211> 1407 | |
| <212> DNA | |
| <213> Artificial Sequence | |
| <220> | |
| <223> Description of Artificial Sequence: Synthetic | |
| polynucleotide | |
| <400> 44 | |
| atgtacttgg gactgaacta tgtattcata gtttttctct taaatggtgt ccagagtgaa | 60 |
| gtgaagctgg aggagtctgg aggaggcttg gtgcaacctg gaggatccat gaaactctct | 120 |
| tgtgctgcct ctggattcac ttttagtgac gcctggatgg actgggtccg ccagtctcca | 180 |
| gagaaggggc ttgagtgggt tgctgaaatt agaagcaaag ctaataatca tgcaacatac | 240 |
| tatgctgagt ctgtgaatgg gaggttcacc atctcaagag atgattccaa aagtagtgtc | 300 |
| tacctgcaaa tgaacagctt aagagctgaa gacactggca tttattactg tacgtggggg | 360 |
| gaagtgttct actttgacta ctggggccaa ggcaccactc tcacagtctc ctcagcctcc | 420 |
| accaagggcc catcggtctt ccccctggca ccctcctcca agagcacctc tgggggcaca | 480 |
| gcggccctgg gctgcctggt caaggactac ttccccgaac cggtgacggt gtcgtggaac | 540 |
| tcaggcgccc tgaccagcgg cgtgcacacc ttcccggctg tcctacagtc ctcaggactc | 600 |
| tactccctca gcagcgtggt gaccgtgccc tccagcagct tgggcaccca gacctacatc | 660 |
| tgcaacgtga atcacaagcc cagcaacacc aaggtggaca agaaagttga gcccaaatct | 720 |
| tgtgacaaaa ctcacacatg cccaccgtgc ccagcacctg aactcctggg gggaccgtca | 780 |
| gtcttcctct tccccccaaa acccaaggac accctcatga tctcccggac ccctgaggtc | 840 |
| acatgcgtgg tggtggacgt gagccacgaa gaccctgagg tcaagttcaa ctggtacgtg | 900 |
| gacggcgtgg aggtgcataa tgccaagaca aagccgcggg aggagcagta caacagcacg | 960 |
| taccgtgtgg tcagcgtcct caccgtcctg caccaggact ggctgaatgg caaggagtac | 1020 |
| aagtgcaagg tctccaacaa agccctccca gcccccatcg agaaaaccat ctccaaagcc | 1080 |
| aaagggcagc cccgagaacc acaggtgtac accctgcccc catcccggga tgagctgacc | 1140 |
| aagaaccagg tcagcctgac ctgcctggtc aaaggcttct atcccagcga catcgccgtg | 1200 |
| gagtgggaga gcaatgggca gccggagaac aactacaaga ccacgcctcc cgtgctggac | 1260 |
| tccgacggct ccttcttcct ctacagcaag ctcaccgtgg acaagagcag gtggcagcag | 1320 |
| gggaacgtct tctcatgctc cgtgatgcat gaggctctgc acaaccacta cacgcagaag | 1380 |
| agcctctccc tgtctccggg taaatga | 1407 |
| <210> 45 | |
| <211> 469 | |
| <212> PRT | |
| <213> Artificial Sequence | |
| <220> | |
| <223> Description of Artificial Sequence: Synthetic | |
| polypeptide | |
| <400> 45 | |
| Met Tyr Leu Gly Leu Asn Tyr Val Phe Ile Val Phe Leu Leu Asn | |
| Gly | |
| 1 5 10 15 | |
| Val Gln Ser Glu Val Lys Leu Glu Glu Ser Gly Gly Gly Leu Val | |
| Gln | |
| 20 25 30 | |
| Pro Gly Gly Ser Met Lys Leu Ser Cys Ala Ala Ser Gly Phe Thr | |
| Phe | |
| 35 40 45 | |
| Ser Asp Ala Trp Met Asp Trp Val Arg Gln Ser Pro Glu Lys Gly | |
| Leu | |
| 50 55 60 | |
| Glu Trp Val Ala Glu Ile Arg Ser Lys Ala Asn Asn His Ala Thr | |
| Tyr | |
| 65 70 75 80 | |
| Tyr Ala Glu Ser Val Asn Gly Arg Phe Thr Ile Ser Arg Asp Asp | |
| Ser | |
| 85 90 95 | |
| Lys Ser Ser Val Tyr Leu Gln Met Asn Ser Leu Arg Ala Glu Asp | |
| Thr | |
| 100 105 110 | |
| Gly Ile Tyr Tyr Cys Thr Trp Gly Glu Val Phe Tyr Phe Asp Tyr | |
| Trp | |
| 115 120 125 | |
| Gly Gln Gly Thr Thr Leu Thr Val Ser Ser Ala Ser Thr Lys Gly | |
| Pro | |
| 130 135 140 | |
| Ser Val Phe Pro Leu Ala Pro Ser Ser Lys Ser Thr Ser Gly Gly | |
| Thr | |
| 145 150 155 | |
| 160 | |
| Ala Ala Leu Gly Cys Leu Val Lys Asp Tyr Phe Pro Glu Pro Val | |
| Thr | |
| 165 170 175 | |
| Val Ser Trp Asn Ser Gly Ala Leu Thr Ser Gly Val His Thr Phe | |
| Pro | |
| 180 185 190 | |
| Ala Val Leu Gln Ser Ser Gly Leu Tyr Ser Leu Ser Ser Val Val | |
| Thr | |
| 195 200 205 | |
| Val Pro Ser Ser Ser Leu Gly Thr Gln Thr Tyr Ile Thr Cys Asn | |
| Val | |
| 210 215 220 | |
| Asn His Lys Pro Ser Asn Thr Lys Val Asp Lys Lys Val Glu Pro | |
| Lys | |
| 225 230 235 | |
| 240 | |
| Ser Cys Asp Lys Thr His Thr Cys Pro Pro Cys Pro Ala Pro Glu | |
| Leu | |
| 245 250 255 | |
| Leu Gly Gly Pro Ser Val Phe Leu Phe Pro Pro Lys Pro Lys Asp | |
| Thr | |
| 260 265 270 | |
| Leu Met Ile Ser Arg Thr Pro Glu Val Thr Cys Val Val Val Asp | |
| Val | |
| 275 280 285 | |
| Ser His Glu Asp Pro Glu Val Lys Phe Asn Trp Tyr Val Asp Gly | |
| Val | |
| 290 295 300 | |
| Glu Val His Asn Ala Lys Thr Lys Pro Arg Glu Glu Gln Tyr Asn | |
| Ser | |
| 305 310 315 | |
| 320 | |
| Thr Tyr Arg Val Val Ser Val Leu Thr Val Leu His Gln Asp Trp | |
| Leu | |
| 325 330 335 | |
| Asn Gly Lys Glu Tyr Lys Cys Lys Val Ser Asn Lys Ala Leu Pro | |
| Ala | |
| 340 345 350 | |
| Pro Ile Glu Lys Thr Ile Ser Lys Ala Lys Gly Gln Pro Arg Glu | |
| Pro | |
| 355 360 365 | |
| Gln Val Tyr Thr Leu Pro Pro Ser Arg Asp Glu Leu Thr Lys Asn | |
| Gln | |
| 370 375 380 | |
| Val Ser Leu Thr Cys Leu Val Lys Gly Phe Tyr Pro Ser Asp Ile | |
| Ala | |
| 385 390 395 | |
| 400 | |
| Val Glu Trp Glu Ser Asn Gly Gln Pro Glu Asn Asn Tyr Lys Thr | |
| Thr | |
| 405 410 415 | |
| Pro Pro Val Leu Asp Ser Asp Gly Ser Phe Phe Leu Tyr Ser Lys | |
| Leu | |
| 420 425 430 | |
| Thr Val Asp Lys Ser Arg Trp Gln Gln Gly Asn Val Phe Ser Cys | |
| Ser | |
| 435 440 445 | |
| Val Met His Glu Ala Leu His Asn His Tyr Thr Gln Lys Ser Leu | |
| Ser | |
| 450 455 460 | |
| Leu Ser Pro Gly Lys | |
| 465 | |
| <210> 46 | |
| <211> 702 | |
| <212> DNA | |
| <213> Artificial Sequence | |
| <220> | |
| <223> Description of Artificial Sequence: Synthetic | |
| polynucleotide | |
| <400> 46 | |
| atgagaccgt ctattcagtt cctggggctc ttgttgttct ggcttcatgg tgctcagtgt | 60 |
| gacatccaga tgacacagtc tccatcctca ctgtctgcat ctctgggagg caaagtcacc | 120 |
| atcacttgca agtcaagcca agacattaac aagtatatag cttggtacca acacaagcct | 180 |
| ggaaaaggtc ctaggctgct catacattac acatctacat tacagccagg catcccatca | 240 |
| aggttcagtg gaagtgggtc tgggagagat tattccttca gcatcagcaa cctggagcct | 300 |
| gaagatattg caacttatta ttgtctacag tatgataatc ttctcacgtt cggtgctggg | 360 |
| accaagctgg agctgaaacg aactgtggct gcaccatctg tcttcatctt cccgccatct | 420 |
| gatgagcagt tgaaatctgg aactgcctct gttgtgtgcc tgctgaataa cttctatccc | 480 |
| agagaggcca aagtacagtg gaaggtggat aacgccctcc aatcgggtaa ctcccaggag | 540 |
| agtgtcacag agcaggacag caaggacagc acctacagcc tcagcagcac cctgacgctg | 600 |
| agcaaagcag actacgagaa acacaaagtc tacgcctgcg aagtcaccca tcagggcctg | 660 |
| agctcgcccg tcacaaagag cttcaacagg ggagagtgtt ag | 702 |

```
<210> 47
<211> 233
<212> PRT
<213> Artificial Sequence
<220>
<223> Description of Artificial Sequence: Synthetic
            polypeptide
<400> 47
Met Arg Pro Ser Ile Gln Phe Leu Gly Leu Leu Leu Phe Trp Leu
His
1 5 10 15
Gly Ala Gln Cys Asp Ile Gln Met Thr Gln Ser Pro Ser Ser Leu
Ser
                        20 25 30
Ala Ser Leu Gly Gly Lys Val Thr Ile Thr Cys Lys Ser Ser Gln
Asp
                35 40 45
Ile Asn Lys Tyr Ile Ala Trp Tyr Gln His Lys Pro Gly Lys Gly
Pro
        50 55 60
Arg Leu Leu Ile His Tyr Thr Ser Thr Leu Gln Pro Gly Ile Pro
Ser
65 70 75 80
Arg Phe Ser Gly Ser Gly Ser Gly Arg Asp Tyr Ser Phe Ser Ile
Ser
                                85 90 95
Asn Leu Glu Pro Glu Asp Ile Ala Thr Tyr Tyr Cys Leu Gln Tyr
Asp
                        100 105 110
Asn Leu Leu Thr Phe Gly Ala Gly Thr Lys Leu Glu Leu Lys Arg
Thr
                115 120 125
Val Ala Ala Pro Ser Val Phe Ile Phe Pro Pro Ser Asp Glu Gln
Leu
        130 135 140
Lys Ser Gly Thr Ala Ser Val Val Cys Leu Leu Asn Asn Phe Tyr
Pro
145 150 155
160
Arg Glu Ala Lys Val Gln Trp Lys Val Asp Asn Ala Leu Gln Ser
Gly
                                165 170 175
Asn Ser Gln Glu Ser Val Thr Glu Gln Asp Ser Lys Asp Ser Thr
Tyr
                        180 185 190
Ser Leu Ser Ser Thr Leu Thr Leu Ser Lys Ala Asp Tyr Glu Lys
His
                195 200 205
Lys Val Tyr Ala Cys Glu Val Thr His Gln Gly Leu Ser Ser Pro
Val
        210 215 220
Thr Lys Ser Phe Asn Arg Gly Glu Cys
225 230
```

SEQ ID NOS:39-43 are the sequences of oligonucleotides used for PCR amplification and sequencing of Ch119-43-1 heavy and light chain cDNA.

SEQ ID NO:44 provides the nucleotide sequence of the coding region of gamma-1 heavy chain in pCh119-43-1 along with the deduced amino acid sequence (SEQ ID NO:45). Amino acid residues are shown in single letter code.

SEQ ID NO:46 provides the nucleotide sequence of the coding region of kappa light chain in pCh119-43-1 along with the deduced amino acid sequence (SEQ ID NO:47). Amino acid residues are shown in single letter code.

PD-1 Antigen Binding Proteins

The combinations, and methods and uses thereof, of the invention comprise anti-PD-1 antigen binding proteins that bind PD-1, such as antagonists molecules (such as antibodies) that block binding with a PD-1 ligand such as PD-L1 or PD-L2.

In an aspect, the equilibrium dissociation constant (KD) of the anti-PD-1 ABP of a combination of the invention, or a method or use thereof, and PD-1, preferably human PD-1, interaction is 100 nM or less, 10 nM or less, 2 nM or less or 1 nM or less. Alternatively the KD may be between 5 and 10 nM; or between 1 and 2 nM. The KD may be between 1 pM and 500 pM; or between 500 pM and 1 nM. A skilled person will appreciate that the smaller the KD numerical value, the stronger the binding. The reciprocal of KD (i.e. 1/KD) is the equilibrium association constant (KA) having units M-1. A skilled person will appreciate that the larger the KA numerical value, the stronger the binding.

The dissociation rate constant (kd) or “off-rate” describes the stability of the complex of the ABP on one hand and PD-1, preferably human PD-1 on the other hand, i.e. the fraction of complexes that decay per second. For example, a kd of 0.01 s-1 equates to 1% of the complexes decaying per second. In an embodiment, the dissociation rate constant (kd) is 1×10-3 s-1 or less, 1×10-4 s-1 or less, 1×10-5 s-1 or less, or 1×10-6 s-1 or less. The kd may be between 1×10-5 s-1 and 1×10-4 s-1; or between 1×10-4 s-1 and 1×10-3 s-1.

Competition between an anti-PD-1 ABP of a combination of the invention, or a method or use thereof, and a reference antibody, e.g. for binding PD-1, an epitope of PD-1, or a fragment of the PD-1, may be determined by competition ELISA, FMAT or Biacore. In one aspect, the competition assay is carried out by Biacore. There are several possible reasons for this competition: the two proteins may bind to the same or overlapping epitopes, there may be steric inhibition of binding, or binding of the first protein may induce a conformational change in the antigen that prevents or reduces binding of the second protein.

“Binding fragments” as used herein means a portion or fragment of the ABPs of a combination of the invention, or a method or use thereof, that include the antigen-binding site and are capable of binding PD-1 as defined herein, e.g. but not limited to capable of binding to the same epitope of the parent or full length antibody.

ABPs that bind human PD-1 receptor are provided herein (i.e. an anti-PD-1 ABP, sometimes referred to herein as an “anti-PD-1 ABP” or an “anti-PD-1 antibody” and/or other variations of the same). These antibodies are useful in the treatment or prevention of acute or chronic diseases or conditions whose pathology involves PD-1 signalling. In one aspect, an antigen binding protein, or isolated human antibody or functional fragment of such protein or antibody, that binds to human PD-1 and is effective as a cancer treatment or treatment against disease is described, for example in combination with another compound such as an anti-OX40 ABP, suitably an agonist anti-OX40 ABP. Any of the antigen binding proteins or antibodies disclosed herein may be used as a medicament. Any one or more of the antigen binding proteins or antibodies may be used in the methods or compositions to treat cancer, e.g. those disclosed herein.

The isolated antibodies as described herein bind to human PD-1, and may bind to human PD-1 encoded by the gene Pdcd1, or genes or cDNA sequences having 90 percent homology or 90 percent identity thereto. The complete hPD-1 mRNA sequence can be found under GenBank Accession No. U64863. The protein sequence for human PD-1 can be found at GenBank Accession No. AAC51773.

Antigen binding proteins and antibodies that bind and/or modulate PD-1 are known in the art. Exemplary anti-PD-1 ABPs of a combination of the invention, or a method or use thereof, are disclosed, for example in U.S. Pat. Nos. 8,354,509; 8,900,587; 8,008,449, each of which is incorporated by reference in its entirety herein (To the extent any definitions conflict, this instant application controls). PD-1 antibodies and methods of using in treatment of disease are described in U.S. Pat. No. 7,595,048; U.S. Pat. No. 8,168,179; U.S. Pat. No. 8,728,474; U.S. Pat. No. 7,722,868; U.S. Pat. No. 8,008,449; U.S. Pat. No. 7,488,802; U.S. Pat. No. 7,521,051; U.S. Pat. No. 8,088,905; U.S. Pat. No. 8,168,757; U.S. Pat. No. 8,354,509; and US Publication Nos. US20110171220; US20110171215; and US20110271358. Combinations of CTLA-4 and PD-1 antibodies are described in U.S. Pat. No. 9,084,776.

In another embodiment, further embodiment, any mouse or chimeric sequences of any anti-PD-1 ABP of a combination of the invention, or a method or use thereof, are engineered to make a humanized antibody.

In another embodiment, the anti-PD-1 ABP of a combination of the invention, or a method or use thereof, comprises one or more (e.g. all) of the CDRs or VH or VL or HC (heavy chain) or LC (light chain) sequences of pembrolizumab, or sequences with at least 90% (e.g., 90%, 91%, 92%, 93%, 94%, 95%, 96%, 97%, 98%, or 99%, or 100%) sequence identity thereto.

The HC and LC CDRs of permolizumab are provided below. In one embodiment, the anti-PD-1 ABP of a combination of the invention, or a method or use thereof, comprises: (a) a heavy chain variable region CDR1 of pembrolizumab; (b) a heavy chain variable region CDR2 of pembrolizumab; (c) a heavy chain variable region CDR3 of pembrolizumab; (d) a light chain variable region CDR1 of pembrolizumab; (e) a light chain variable region CDR2 of pembrolizumab; and (f) a light chain variable region CDR3 of pembrolizumab.

In another embodiment, the anti-PD-1 of a combination of the invention, or a method or use thereof, comprises: a heavy chain variable region CDR1 of pembrolizumab; a heavy chain variable region CDR2 of pembrolizumab and/or a heavy chain variable region CDR3 of pembrolizumab.

In another embodiment, the anti-PD-1 of a combination of the invention, or a method or use thereof, comprises: a light chain variable region CDR1 of pembrolizumab; a light chain variable region CDR2 of pembrolizumab and/or a light chain variable region CDR3 of pembrolizumab.

In another embodiment, the anti-PD-1 ABP of a combination of the invention, or a method or use thereof, comprises: a light chain variable region (“VL”) of pembrolizumab, or an amino acid sequence with at least 90% (e.g., 90%, 91%, 92%, 93%, 94%, 95%, 96%, 97%, 98%, or 99%, or 100%) sequence identity to the amino acid sequence of the VL of pembrolizumab.

In another embodiment, the anti-PD-1 ABP of a combination of the invention, or a method or use thereof, comprises a heavy chain variable region (“VH”) of pembrolizumab, or an amino acid sequence with at least 90% (e.g., 90%, 91%, 92%, 93%, 94%, 95%, 96%, 97%, 98%, or 99%, or 100%) sequence identity to the amino acid sequence of the VH of pembrolizumab.

In another embodiment, the anti-PD-1 ABP of a combination of the invention, or a method or use thereof, comprises: a light chain variable region (“VL”) of pembrolizumab, or an amino acid sequence with at least 90% (e.g., 90%, 91%, 92%, 93%, 94%, 95%, 96%, 97%, 98%, or 99%, or 100%) sequence identity to the amino acid sequence of the VL of pembrolizumab and the anti-PD-1 ABP of a combination of the invention, or a method or use thereof, comprises a heavy chain variable region (“VH”) of pembrolizumab, or an amino acid sequence with at least 90% (e.g., 90%, 91%, 92%, 93%, 94%, 95%, 96%, 97%, 98%, or 99%, or 100%) sequence identity to the amino acid sequence of the VH of pembrolizumab.

In another embodiment, the anti-PD-1 ABP of a combination of the invention, or a method or use thereof, comprises: a light chain (“LC”) of pembrolizumab, or an amino acid sequence with at least 90% (e.g., 90%, 91%, 92%, 93%, 94%, 95%, 96%, 97%, 98%, or 99%, or 100%) sequence identity to the amino acid sequence of the LC of pembrolizumab.

In another embodiment, the anti-PD-1 ABP of a combination of the invention, or a method or use thereof, comprises a heavy chain (“HC”) of pembrolizumab, or an amino acid sequence with at least 90% (e.g., 90%, 91%, 92%, 93%, 94%, 95%, 96%, 97%, 98%, or 99%, or 100%) sequence identity to the amino acid sequence of the HC of pembrolizumab.

In another embodiment, the anti-PD-1 ABP of a combination of the invention, or a method or use thereof, comprises: a light chain (“LC”) of pembrolizumab, or an amino acid sequence with at least 90% (e.g., 90%, 91%, 92%, 93%, 94%, 95%, 96%, 97%, 98%, or 99%, or 100%) sequence identity to the amino acid sequence of the LC of pembrolizumab and the anti-PD-1 ABP of a combination of the invention, or a method or use thereof, comprises a heavy chain (“HC”) of pembrolizumab, or an amino acid sequence with at least 90% (e.g., 90%, 91%, 92%, 93%, 94%, 95%, 96%, 97%, 98%, or 99%, or 100%) sequence identity to the amino acid sequence of the HC of pembrolizumab.

Another embodiment of a combination of the invention, or a method or use thereof, includes CDRs, VH regions, and VL regions, and antibodies and nucleic acids encoding the same as disclosed in the below Sequence Listing.

An anti-OX40 ABP (e.g., an agonist ABP, e.g. an anti-hOX40 ABP, e.g. antibody), e.g., an antibody described herein, can be used in combination with an ABP (e.g., antagonist ABP, e.g antagonist antibody) against PD-1 (e.g. human PD-1). For example, an anti-OX40 antibody can be used in combination with pembrolizumab.

While in development, pembrolizumab (KEYTRUDA®) was known as MK3475 and as lambrolizumab. Pembrolizumab (KEYTRUDA®) is a human programmed death receptor-1 (PD-1)-blocking antibody indicated for the treatment of patients with unresectable or metastatic melanoma and disease progression following ipilimumab and, if BRAF V600 mutation positive, a BRAF inhibitor. The recommended dose of pembrolizumab is 2 mg/kg administered as an intravenous infusion over 30 minutes every 3 weeks until disease progression or unacceptable toxicity.

Pembrolizumab is a humanized monoclonal antibody that blocks the interaction between PD-1 and its ligands, PD-L1 and PD-L2. Pembrolizumab is an IgG4 kappa immunoglobulin with an approximate molecular weight of 149 kDa.

Pembrolizumab for injection is a sterile, preservative-free, white to off-white lyophilized powder in single-use vials. Each vial is reconstituted and diluted for intravenous infusion. Each 2 mL of reconstituted solution contains 50 mg of pembrolizumab and is formulated in L-histidine (3.1 mg), polysorbate-80 (0.4 mg), sucrose (140 mg). May contain hydrochloric acid/sodium hydroxide to adjust pH to 5.5.

Pembrolizumab injection is a sterile, preservative-free, clear to slightly opalescent, colorless to slightly yellow solution that requires dilution for intravenous infusion. Each vial contains 100 mg of pembrolizumab in 4 mL of solution. Each 1 mL of solution contains 25 mg of pembrolizumab and is formulated in: L-histidine (1.55 mg), polysorbate 80 (0.2 mg), sucrose (70 mg), and Water for Injection, USP.

Binding of the PD-1 ligands, PD-L1 and PD-L2, to the PD-1 receptor found on T cells, inhibits T cell proliferation and cytokine production. Upregulation of PD-1 ligands occurs in some tumors and signaling through this pathway can contribute to inhibition of active T-cell immune surveillance of tumors. Pembrolizumab is a monoclonal antibody that binds to the PD-1 receptor and blocks its interaction with PD-L1 and PD-L2, releasing PD-1 pathway-mediated inhibition of the immune response, including the anti-tumor immune response. In syngeneic mouse tumor models, blocking PD-1 activity resulted in decreased tumor growth.

Pembrolizumab is described, e.g in U.S. Pat. Nos. 8,354,509 and 8,900,587.

The approved product is pembrolizumab (KEYTRUDA®) for injection, for intravenous infusion of the active ingredient pembrolizumab, available as a 50 mg lyophilized powder in a single-usevial for reconstitution. Pembrolizumab has been approved for the treatment of patients with unresectable or metastatic melanoma and disease progression following ipilimumab and, if BRAF V600 mutation positive, a BRAF inhibitor. Pembrolizumab (KEYTRUDA®) is a humanized monoclonal antibody that blocks the interaction between PD-I and its ligands, PD-LI and PD-L2. Pembrolizumab is an IgG4 kappa immunoglobulin with an approximate molecular weight of 149 kDa. The amino acid sequence for pembrolizumab is as follows, and is set forth using the same one-letter amino acid code nomenclature provided in the table at column 15 of the U.S. Pat. No. 8,354,509:

| Heavy Chain of pembrolizumab: | |
|---|---|
| (SEQ ID NO: 50) | |
| QVQLVQSGVE VKKPGASVKV SCKASGYTFT NYYMYWVRQA PGQGLEWMGG | 50 |
| INPSNGGTNF NEKFKNRVTL TTDSSTTTAY MELKSLQFDD TAVYYCARRD | 100 |
| YRFDMGFDYW GQGTTVTVSS ASTKGPSVFP LAPCSRSTSE STAALGCLVK | 150 |
| DYFPEPVTVS WNSGALTSGV HTFPAVLQSS GLYSLSSVVT VPSSSLGTKT | 200 |
| YTCNVDHKPS NTKVDKRVES KYGPPCPPCP APEFLGGPSV FLFPPKPKDT | 250 |
| LMISRTPEVT CVVVDVSQED PEVQFNWYVD GVEVHNAKTK PREEQFNSTY | 300 |
| VVSVLTVLH QDWLNGKEYK CKVSNKGLPS SIEKTISKAK GQPREPQVYT | 350 |
| LPPSQEEMTK NQVSLTCLVK GFYPSDIAVE WESNGQPENN YKTTPPVLDS | 400 |
| DGSFFLYSRL TVDKSRWQEG NVFSCSVMHE ALHNHYTQKS LSLSLGK | 447 |
| Light Chain of pembrolizumab: | |
| (SEQ ID NO: 51) | |
| EIVLTQSPAT LSLSPGERAT LSCRASKGVS TSGYSYLHWY QQKPGQAPRL | 50 |
| LIYLASYLES GVPARFSGSG SGTDFTLTIS SLEPEDFAVY YCQHSRDLPL | 100 |
| TFGGGTKVEI KRTVAAPSVF IFPPSDEQLK SGTASVVCLL NNFYPREAKV | 150 |
| QWKVDNALQS GNSQESVTEQ DSKDSTYSLS STLTLSKADY EKHKVYACEV | 200 |
| THQGLSSPVT KSFNRGEC | 218 |
| Heavy Chain Variable Region of pembrolizumab: | |
| (SEQ ID NO: 52) | |
| QVQLVQSGVE VKKPGASVKV SCKASGYTFT NYYMYWVRQA PGQGLEWMGG | 50 |
| INPSNGGTNF NEKFKNRVTL TTDSSTTTAY MELKSLQFDD TAVYYCARRD | 100 |
| YRFDMGFDYW GQGTTVTVSS | |
| Light Chain Variable Region of pembrolizumab: | |
| (SEQ ID NO: 53) | |
| EIVLTQSPAT LSLSPGERAT LSCRASKGVS TSGYSYLHWY QQKPGQAPRL | 50 |
| LIYLASYLES GVPARFSGSG SGTDFTLTIS SLEPEDFAVY YCQHSRDLPL | 100 |
| TFGGGTKVEI K | |
| CDR sequences of pembrolizumab: | |
| HC CDR1: | |

| (SEQ ID NO: 54) | |
|---|---|
| Asn Tyr Tyr Met Tyr | |
| HC CDR2: | |
| (SEQ ID NO: 55) | |
| Gly Ile Asn Pro Ser Asn Gly Gly Thr Asn Phe Asn Glu Lys Phe Lys Asn | |
| HC CDR3: | |
| (SEQ ID NO: 56) | |
| Arg Asp Tyr Arg Phe Asp Met Gly Phe Asp Tyr | |
| LC CDR1: | |
| (SEQ ID NO: 57) | |
| Arg Ala Ser Lys Gly Val Ser Thr Ser Gly Tyr Ser Tyr Leu His | |
| LC CDR2: | |
| (SEQ ID NO: 58) | |
| Leu Ala Ser Tyr Leu Glu Ser | |
| LC CDR3: | |
| (SEQ ID NO: 59) | |
| Gln His Ser Arg Asp Leu Pro Leu Thr | |
| Sequence Listing from U.S. Pat. No. 8,354,509 | |
| <210> | |
| <211> 435 | |
| <212> DNA | |
| <213> Artificial Sequence | |
| <220> 60 | |
| <223> hPD-1.08A heavy chain variable region | |
| <400> | |
| atgrgatgga gctgtatcat kctctttttg gtagcaacag ctacaggtgt ccactcccag | 60 |
| gtccaactgc agcagcctgg ggctgaactg gtgaagcctg gggcttcagt gaagttgtcc | 120 |
| tgcaaggcct ctggctacac cttcaccagt tattatctgt actggatgaa acagaggcct | 180 |
| ggacaaggcc ttgagtggat tgggggggtt aatcctagta atggtggtac taacttcagt | 240 |
| gagaagttca agagcaaggc cacactgact gtagacaaat cctccagcac agcctacatg | 300 |
| caactcagca gcctgacatc tgaggactct gcggtctatt actgtacaag aagggattct | 360 |
| aactacgacg ggggctttga ctactggggc caaggcacta ctctcacagt ctcctcagcc | 420 |
| aaaacgacac cccca | 435 |
| <210> 61 | |
| <211> 453 | |
| <212> DNA | |
| <213> Artificial Sequence | |
| <220> | |
| <223> hPD-1.08A light chain variable region | |
| <400> | |
| atggagacag acacactcct gctatgggta ctgctgctct gggttccagg ttccactggt | 60 |
| gacattgtgc tgacacagtc tcctacttcc ttagctgtat ctctggggca gagggccacc | 120 |
| atctcatgca gggccagcaa aagtgtcagt acatctggct ttagttattt gcactggtac | 180 |
| caacagaaac caggacagcc acccaaactc ctcatctttc ttgcatccaa cctagagtct | 240 |
| ggggtccctg ccaggttcag tggcagtggg tctgggacag acttcaccct caacatccat | 300 |
| cctgtggagg aggaggacgc tgcaacctat tattgtcagc acagttggga gcttccgctc | 360 |
| acgttcggtg ctgggaccaa gctggagctg aaacgggctg atgctgcacc aactgtatcc | 420 |
| atcttcccac catccagtaa gcttgggaag ggc | 453 |
| <210> 62 | |
| <211> 464 | |
| <212> DNA | |
| <213> Artificial Sequence | |
| <220> | |
| <223> hPD-1.09A heavy chain variable region | |
| <400> | |
| atgraatgca gctgggttat yctctttttg gtagcaacag ctacaggtgt ccactcccag | 60 |
| gtccaactgc agcagcctgg ggctgaactg gtgaagcctg ggacttcagt gaagttgtcc | 120 |
| tgcaaggctt ctggctacac cttcaccaac tactatatgt actgggtgaa gcagaggcct | 180 |
| ggacaaggcc ttgagtggat tggggggatt aatcctagca atggtggtac taacttcaat | 240 |
| gagaagttca agaacaaggc cacactgact gtagacagtt cctccagcac aacctacatg | 300 |
| caactcagca gcctgacatc tgaggactct gcggtctatt actgtacaag aagggattat | 360 |
| aggttcgaca tgggctttga ctactggggc caaggcacca ctctcacagt ctcctcagcc | 420 |
| aaaacgacac ccccatccgt ytatcccbtg gcccctggaa gctt | 464 |
| <210> 63 | |
| <211> 438 | |
| <212> DNA | |
| <213> Artificial Sequence | |
| <220> | |
| <223> hPD-1.09A light chain variable region | |
| <400> | |
| atggagwcag acacactsct gytatgggta ctgctgctct gggttccagg ttccactggc | 60 |
| gacattgtgc tgacacagtc tcctgcttcc ttagctgtat ctctgggaca gagggccgcc | 120 |
| atctcatgca gggccagcaa aggtgtcagt acatctggct atagttattt gcactggtac | 180 |
| caacagaaac caggacagtc acccaaactc ctcatctatc ttgcatccta cctagaatct | 240 |
| ggggtccctg ccaggttcag tggcagtggg tctgggacag acttcaccct caacatccat | 300 |
| cctgtggagg aggaggatgc tgcaacctat tactgtcagc acagtaggga ccttccgctc | 360 |
| acgttcggta ctgggaccaa gctggagctg aaacgggctg atgctgcacc aactgtatcc | 420 |
| atcttcccac catccagt | 438 |
| <210> 64 | |
| <211> 122 | |
| <212> PRT | |
| <213> Artificial Sequence | |
| <220> | |
| <223> hPD-1.08A heavy chain variable region | |
| <400> | |
| Gln Val Gln Leu Gln Gln Pro Gly Ala Glu Leu Val Lys Pro Gly Ala | |
| 1 5 10 15 | |
| Ser Val Lys Leu Ser Cys Lys Ala Ser Gly Tyr Thr Phe Thr Ser Tyr | |
| 20 25 30 | |
| Tyr Leu Tyr Trp Met Lys Gln Arg Pro Gly Gln Gly Leu Glu Trp Ile | |
| 35 40 45 | |
| Gly Gly Val Asn Pro Ser Asn Gly Gly Thr Asn Phe Ser Glu Lys Phe | |
| 50 55 60 | |
| Lys Ser Lys Ala Thr Leu Thr Val Asp Lys Ser Ser Ser Thr Ala Tyr | |
| 65 70 75 80 | |
| Met Gln Leu Ser Ser Leu Thr Ser Glu Asp Ser Ala Val Tyr Tyr Cys | |
| 85 90 95 | |
| Thr Arg Arg Asp Ser Asn Tyr Asp Gly Gly Phe Asp Tyr Trp Gly Gln | |
| 100 105 110 | |
| Gly Thr Thr Leu Thr Val Ser Ser Ala Lys | |
| 115 120 | |
| <210> 65 | |
| <211> 111 | |
| <212> PRT | |
| <213> Artificial Sequence | |
| <220> | |
| <223> hPD-1.08A light chain variable region | |
| <400> | |
| Asp Ile Val Leu Thr Gln Ser Pro Thr Ser Leu Ala Val Ser Leu Gly | |
| 1 5 10 15 | |
| Gln Arg Ala Thr Ile Ser Cys Arg Ala Ser Lys Ser Val Ser Thr Ser | |
| 20 25 30 | |
| Gly Phe Ser Tyr Leu His Trp Tyr Gln Gln Lys Pro Gly Gln Pro Pro | |
| 35 40 45 | |
| Lys Leu Leu Ile Phe Leu Ala Ser Asn Leu Glu Ser Gly Val Pro Ala | |
| 50 55 60 | |
| Arg Phe Ser Gly Ser Gly Ser Gly Thr Asp Phe Thr Leu Asn Ile His | |
| 65 70 75 80 | |
| Pro Val Glu Glu Glu Asp Ala Ala Thr Tyr Tyr Cys Gln His Ser Trp | |
| 85 90 95 | |
| Glu Leu Pro Leu Thr Phe Gly Ala Gly Thr Lys Leu Glu Leu Lys | |
| 100 105 110 | |
| <210> 66 | |
| <211> 122 | |
| <212> PRT | |
| <213> Artificial Sequence | |
| <220> | |
| <223> hPD-1.09A heavy chain variable region | |
| <400> | |
| Gln Val Gln Leu Gln Gln Pro Gly Ala Glu Leu Val Lys Pro Gly Thr | |
| 1 5 10 15 | |
| Ser Val Lys Leu Ser Cys Lys Ala Ser Gly Tyr Thr Phe Thr Asn Tyr | |
| 20 25 30 | |
| Tyr Met Tyr Trp Val Lys Gln Arg Pro Gly Gln Gly Leu Glu Trp Ile | |
| 35 40 45 | |
| Gly Gly Ile Asn Pro Ser Asn Gly Gly Thr Asn Phe Asn Glu Lys Phe | |
| 50 55 60 | |
| Lys Asn Lys Ala Thr Leu Thr Val Asp Ser Ser Ser Ser Thr Thr Tyr | |
| 65 70 75 80 | |
| Met Gln Leu Ser Ser Leu Thr Ser Glu Asp Ser Ala Val Tyr Tyr Cys | |
| 85 90 95 | |
| Thr Arg Arg Asp Tyr Arg Phe Asp Met Gly Phe Asp Tyr Trp Gly Gln | |
| 100 105 110 | |
| Gly Thr Thr Leu Thr Val Ser Ser Ala Lys | |
| 115 120 | |
| <210> 67 | |
| <211> 111 | |
| <212> PRT | |
| <213> Artificial Sequence | |
| <220> | |
| <223> hPD-1.09A light chain variable region | |
| <400> | |
| Asp Ile Val Leu Thr Gln Ser Pro Ala Ser Leu Ala Val Ser Leu Gly | |
| 1 5 10 15 | |
| Gln Arg Ala Ala Ile Ser Cys Arg Ala Ser Lys Gly Val Ser Thr Ser | |
| 20 25 30 | |
| Gly Tyr Ser Tyr Leu His Trp Tyr Gln Gln Lys Pro Gly Gln Ser Pro | |
| 35 40 45 | |
| Lys Leu Leu Ile Tyr Leu Ala Ser Tyr Leu Glu Ser Gly Val Pro Ala | |
| 50 55 60 | |
| Arg Phe Ser Gly Ser Gly Ser Gly Thr Asp Phe Thr Leu Asn Ile His | |
| 65 70 75 80 | |
| Pro Val Glu Glu Glu Asp Ala Ala Thr Tyr Tyr Cys Gln His Ser Arg | |
| 85 90 95 | |
| Asp Leu Pro Leu Thr Phe Gly Thr Gly Thr Lys Leu Glu Leu Lys | |
| 100 105 110 | |
| <210> 68 | |
| <211> 15 | |
| <212> PRT | |
| <213> Artificial Sequence | |
| <220> | |
| <223> hPD-1.08A light chain CDR1 | |
| <400> | |
| Arg Ala Ser Lys Ser Val Ser Thr Ser Gly Phe Ser Tyr Leu His | |
| 1 5 10 15 | |
| <210> 69 | |
| <211> 7 | |
| <212> PRT | |
| <213> Artificial Sequence | |
| <220> | |
| <223> hPD-1.08A light chain CDR2 | |
| <400> | |
| Leu Ala Ser Asn Leu Glu Ser | |
| 1 5 | |
| <210> 70 | |
| <211> 9 | |
| <212> PRT | |
| <213> Artificial Sequence | |
| <220> | |
| <223> hPD-1-08A light chain CDR3 | |
| <400> | |
| Gln His Ser Trp Glu Leu Pro Leu Thr | |
| 1 5 | |
| <210> 71 | |
| <211> 5 | |
| <212> PRT | |
| <213> Artificial Sequence | |
| <220> | |
| <223> hPD-1.08A heavy chain CDR1 | |
| <400> | |
| Ser Tyr Tyr Leu Tyr | |
| 1 5 | |
| <210> 72 | |
| <211> 17 | |
| <212> PRT | |
| <213> Artificial Sequence | |
| <220> | |
| <223> hPD-1.08A heavy chain CDR2 | |
| <400> | |
| Gly Val Asn Pro Ser Asn Gly Gly Thr Asn Phe Ser Glu Lys Phe Lys | |
| 1 5 10 15 | |
| Ser | |
| <210> 73 | |
| <211> 11 | |
| <212> PRT | |
| <213> Artificial Sequence | |
| <220> | |
| <223> hPD-1.08A heavy chain CDR3 | |
| <400> | |
| Arg Asp Ser Asn Tyr Asp Gly Gly Phe Asp Tyr | |
| 1 5 10 | |
| <210> 74 | |
| <211> 15 | |
| <212> PRT | |
| <213> Artificial Sequence | |
| <220> | |
| <223> hPD-1.09A light chain CDR1 | |
| <400> | |
| Arg Ala Ser Lys Gly Val Ser Thr Ser Gly Tyr Ser Tyr Leu His | |
| 1 5 10 15 | |
| <210> 75 | |
| <211> 7 | |
| <212> PRT | |
| <213> Artificial Sequence | |
| <220> | |
| <223> hPD-1.09A light chain CDR2 | |
| <400> | |
| Leu Ala Ser Tyr Leu Glu Ser | |
| 1 5 | |
| <210> 76 | |
| <211> 9 | |
| <212> PRT | |
| <213> Artificial Sequence | |
| <220> | |
| <223> hPD-1.09A light chain CDR3 | |
| <400> | |
| Gln His Ser Arg Asp Leu Pro Leu Thr | |
| 1 5 | |
| <210> 77 | |
| <211> 5 | |
| <212> PRT | |
| <213> Artificial Sequence | |
| <220> | |
| <223> hPD-1.09A heavy chain CDR1 | |
| <400> | |
| Asn Tyr Tyr Met Tyr | |
| 1 5 | |
| <210> 78 | |
| <211> 17 | |
| <212> PRT | |
| <213> Artificial Sequence | |
| <220> | |
| <223> hPD-1.09A heavy chain CDR2 | |
| <400> | |
| Gly Ile Asn Pro Ser Asn Gly Gly Thr Asn Phe Asn Glu Lys Phe Lys | |
| 1 5 10 15 | |
| Asn | |
| <210> 79 | |
| <211> 11 | |
| <212> PRT | |
| <213> Artificial Sequence | |
| <220> | |
| <223> hPD-1.09A heavy chain CDR3 | |
| <400> | |
| Arg Asp Tyr Arg Phe Asp Met Gly Phe Asp Tyr | |
| 1 5 10 | |
| <210> 80 | |
| <211> 417 | |
| <212> DNA | |
| <213> Artificial Sequence | |
| <220> | |
| <223> 109A-H heavy chain variable region | |
| <220> | |
| <221> sig_peptide | |
| <222> (1) . . . (57) | |
| <400> | |
| atggactgga cctggagcat ccttttcttg gtggcagcac caacaggagc ccactcccaa | 60 |
| gtgcagctgg tgcagtctgg agttgaagtg aagaagcccg gggcctcagt gaaggtctcc | 120 |
| tgcaaggctt ctggctacac ctttaccaac tactatatgt actgggtgcg acaggcccct | 180 |
| ggacaagggc ttgagtggat gggagggatt aatcctagca atggtggtac taacttcaat | 240 |
| gagaagttca agaacagagt caccttgacc acagactcat ccacgaccac agcctacatg | 300 |
| gaactgaaga gcctgcaatt tgacgacacg gccgtttatt actgtgcgag aagggattat | 360 |
| aggttcgaca tgggctttga ctactggggc caagggacca cggtcaccgt ctcgagc | 417 |
| <210> 81 | |
| <211> 417 | |
| <212> DNA | |
| <213> Artificial Sequence | |
| <220> | |
| <223> Codon optimized 109A-H heavy chain variable region | |
| <220> | |
| <221> sig_peptide | |
| <222> (1) . . . (57) | |
| <400> | |
| atggactgga cctggtctat cctgttcctg gtggccgctc ctaccggcgc tcactcccag | 60 |
| gtgcagctgg tgcagtccgg cgtggaggtg aagaagcctg gcgcctccgt caaggtgtcc | 120 |
| tgcaaggcct ccggctacac cttcaccaac tactacatgt actgggtgcg gcaggctccc | 180 |
| ggccagggac tggagtggat gggcggcatc aacccttcca acggcggcac caacttcaac | 240 |
| gagaagttca agaaccgggt gaccctgacc accgactcct ccaccaccac cgcctacatg | 300 |
| gagctgaagt ccctgcagtt cgacgacacc gccgtgtact actgcgccag gcgggactac | 360 |
| cggttcgaca tgggcttcga ctactggggc cagggcacca ccgtgaccgt gtcctcc | 417 |
| <210> 82 | |
| <211> 1398 | |
| <212> DNA | |
| <213> Artificial Sequence | |
| <220> | |
| <223> Codon optimized 409A-H heavy chain full length | |
| <220> | |
| <221> sig_peptide | |
| <222> (1) . . . (57) | |
| <400> | |
| atggccgtgc tgggcctgct gttctgcctg gtgaccttcc cttcctgcgt gctgtcccag | 60 |
| gtgcagctgg tgcagtccgg cgtggaggtg aagaagcctg gcgcctccgt caaggtgtcc | 120 |
| tgtaaggcct ccggctacac cttcaccaac tactacatgt actgggtgcg gcaggcccca | 180 |
| ggccagggac tggagtggat gggcggcatc aacccttcca acggcggcac caacttcaac | 240 |
| gagaagttca agaaccgggt gaccctgacc accgactcct ccaccacaac cgcctacatg | 300 |
| gaactgaagt ccctgcagtt cgacgacacc gccgtgtact actgcgccag gcgggactac | 360 |
| cggttcgaca tgggcttcga ctactggggc cagggcacca ccgtgaccgt gtcctccgct | 420 |
| agcaccaagg gcccttccgt gttccctctg gccccttgct cccggtccac ctccgagtcc | 480 |
| accgccgctc tgggctgtct ggtgaaggac tacttccctg agcctgtgac cgtgagctgg | 540 |
| aactctggcg ccctgacctc cggcgtgcac accttccctg ccgtgctgca gtcctccggc | 600 |
| ctgtactccc tgtcctccgt ggtgaccgtg ccttcctcct ccctgggcac caagacctac | 660 |
| acctgcaacg tggaccacaa gccttccaac accaaggtgg acaagcgggt ggagtccaag | 720 |
| tacggccctc cttgccctcc ctgccctgcc cctgagttcc tgggcggacc ctccgtgttc | 780 |
| ctgttccctc ctaagcctaa ggacaccctg atgatctccc ggacccctga ggtgacctgc | 840 |
| gtggtggtgg acgtgtccca ggaagatcct gaggtccagt tcaattggta cgtggatggc | 900 |
| gtggaggtgc acaacgccaa gaccaagcct cgggaggaac agttcaactc cacctaccgg | 960 |
| gtggtgtctg tgctgaccgt gctgcaccag gactggctga acggcaagga atacaagtgc | 1020 |
| aaggtcagca acaagggcct gccctcctcc atcgagaaaa ccatctccaa ggccaagggc | 1080 |
| cagcctcgcg agcctcaggt gtacaccctg cctcctagcc aggaagagat gaccaagaat | 1140 |
| caggtgtccc tgacatgcct ggtgaagggc ttctaccctt ccgatatcgc cgtggagtgg | 1200 |
| gagagcaacg gccagccaga gaacaactac aagaccaccc ctcctgtgct ggactccgac | 1260 |
| ggctccttct tcctgtactc caggctgacc gtggacaagt cccggtggca ggaaggcaac | 1320 |
| gtcttttcct gctccgtgat gcacgaggcc ctgcacaacc actacaccca gaagtccctg | 1380 |
| tccctgtctc tgggcaag | 1398 |
| <210> 83 | |
| <211> 393 | |
| <212> DNA | |
| <213> Artificial Sequence | |
| <220> | |
| <223> K09A-L-11 light chain variable region | |
| <220> | |
| <221> sig_peptide | |
| <222> (1) . . . (60) | |
| <400> | |
| atggaagccc cagctcagct tctcttcctc ctgctactct ggctcccaga taccaccgga | 60 |
| gaaattgtgt tgacacagtc tccagccacc ctgtctttgt ctccagggga aagagccacc | 120 |
| ctctcctgca gggccagcaa aggtgtcagt acatctggct atagttattt gcactggtat | 180 |
| caacagaaac ctggccaggc tcccaggctc ctcatctatc ttgcatccta cctagaatct | 240 |
| ggcgtcccag ccaggttcag tggtagtggg tctgggacag acttcactct caccatcagc | 300 |
| agcctagagc ctgaagattt tgcagtttat tactgtcagc acagcaggga ccttccgctc | 360 |
| acgttcggcg gagggaccaa agtggagatc aaa | 393 |
| <210> 84 | |
| <211> 393 | |
| <212> DNA | |
| <213> Artificial Sequence | |
| <220> | |
| <223> K09A-L-16 light chain variable region | |
| <220> | |
| <221> sig_peptide | |
| <222> (1) . . . (60) | |
| <400> | |
| atggaaaccc cagcgcagct tctcttcctc ctgctactct ggctcccaga taccaccgga | 60 |
| gaaattgtgc tgactcagtc tccactctcc ctgcccgtca cccctggaga gccggcctcc | 120 |
| atctcctgca gggccagcaa aggtgtcagt acatctggct atagttattt gcattggtac | 180 |
| ctccagaagc cagggcagtc tccacagctc ctgatctatc ttgcatccta cctagaatct | 240 |
| ggggtccctg acaggttcag tggcagtgga tcaggcacag attttacact gaaaatcagc | 300 |
| agagtggagg ctgaggatgt tggggtttat tactgccagc atagtaggga ccttccgctc | 360 |
| acgtttggcc aggggaccaa gctggagatc aaa | 393 |
| <210> 85 | |
| <211> 393 | |
| <212> DNA | |
| <213> Artificial Sequence | |
| <220> | |
| <223> K09A-L-17 light chain variable region | |
| <220> | |
| <221> sig_peptide | |
| <222> (1) . . . (60) | |
| <400> | |
| atgaggctcc ctgctcagct cctggggctg ctaatgctct gggtctctgg atccagtggg | 60 |
| gatattgtga tgacccagac tccactctcc ctgcccgtca cccctggaga gccggcctcc | 120 |
| atctcctgca gggccagcaa aggtgtcagt acatctggct atagttattt gcattggtat | 180 |
| ctgcagaagc cagggcagtc tccacagctc ctgatctatc ttgcatccta cctagaatct | 240 |
| ggagtcccag acaggttcag tggcagtggg tcaggcactg ctttcacact gaaaatcagc | 300 |
| agggtggagg ctgaggatgt tggactttat tactgccagc atagtaggga ccttccgctc | 360 |
| acgtttggcc aggggaccaa gctggagatc aaa | 393 |
| <210> 86 | |
| <211> 390 | |
| <212> DNA | |
| <213> Artificial Sequence | |
| <220> | |
| <223> Codon optimized K09A-L-11 light chain variable region | |
| <220> | |
| <221> sig_peptide | |
| <222> (1) . . . (57) | |
| <400> | |
| atggcccctg tgcagctgct gggcctgctg gtgctgttcc tgcctgccat gcggtgcgag | 60 |
| atcgtgctga cccagtcccc tgccaccctg tccctgagcc ctggcgagcg ggctaccctg | 120 |
| agctgcagag cctccaaggg cgtgtccacc tccggctact cctacctgca ctggtatcag | 180 |
| cagaagccag gccaggcccc tcggctgctg atctacctgg cctcctacct ggagtccggc | 240 |
| gtgcctgccc ggttctccgg ctccggaagc ggcaccgact tcaccctgac catctcctcc | 300 |
| ctggagcctg aggacttcgc cgtgtactac tgccagcact cccgggacct gcctctgacc | 360 |
| tttggcggcg gaacaaaggt ggagatcaag | 390 |
| <210> 87 | |
| <211> 390 | |
| <212> DNA | |
| <213> Artificial Sequence | |
| <220> | |
| <223> Codon optimized K09A-L-16 light chain variable region | |
| <220> | |
| <221> sig_peptide | |
| <222> (1) . . . (57) | |
| <400> | |
| atggcccctg tgcagctgct gggcctgctg gtgctgttcc tgcctgccat gcggtgcgag | 60 |
| atcgtgctga cccagtcccc tctgtccctg cctgtgaccc ctggcgagcc tgcctccatc | 120 |
| tcctgccggg cctccaaggg cgtgtccacc tccggctact cctacctgca ctggtatctg | 180 |
| cagaagcctg gccagtcccc ccagctgctg atctacctgg cctcctacct ggagtccggc | 240 |
| gtgcctgacc ggttctccgg ctccggcagc ggcaccgact tcaccctgaa gatctcccgg | 300 |
| gtggaggccg aggacgtggg cgtgtactac tgccagcact cccgggacct gcctctgacc | 360 |
| ttcggccagg gcaccaagct ggagatcaag | 390 |
| <210> 88 | |
| <211> 390 | |
| <212> DNA | |
| <213> Artificial Sequence | |
| <220> | |
| <223> Codon optimized K09A-L-17 light chain variable region | |
| <220> | |
| <221> sig_peptide | |
| <222> (1) . . . (57) | |
| <400> | |
| atggcccctg tgcagctgct gggcctgctg gtgctgttcc tgcctgccat gcggtgcgac | 60 |
| atcgtgatga cccagacccc tctgtccctg cctgtgaccc ctggcgagcc tgcctccatc | 120 |
| tcctgccggg cctccaaggg cgtgtccacc tccggctact cctacctgca ctggtatctg | 180 |
| cagaagcctg gccagtcccc tcagctgctg atctacctgg cctcctacct ggagtccggc | 240 |
| gtgcctgacc ggttctccgg ctccggaagc ggcaccgctt ttaccctgaa gatctccaga | 300 |
| gtggaggccg aggacgtggg cctgtactac tgccagcact cccgggacct gcctctgacc | 360 |
| ttcggccagg gcaccaagct ggagatcaag | 390 |
| <210> 89 | |
| <211> 139 | |
| <212> PRT | |
| <213> Artificial Sequence | |
| <220> | |
| <223> 109A-H heavy chain variable region | |
| <220> | |
| <221> sig_peptide | |
| <222> (1) . . . (19) | |
| <400> | |
| Met Asp Trp Thr Trp Ser Ile Leu Phe Leu Val Ala Ala Pro Thr Gly | |
| 1 5 10 15 | |
| Ala His Ser Gln Val Gln Leu Val Gln Ser Gly Val Glu Val Lys Lys | |
| 20 25 30 | |
| Pro Gly Ala Ser Val Lys Val Ser Cys Lys Ala Ser Gly Tyr Thr Phe | |
| 35 40 45 | |
| Thr Asn Tyr Tyr Met Tyr Trp Val Arg Gln Ala Pro Gly Gln Gly Leu | |
| 50 55 60 | |
| Glu Trp Met Gly Gly Ile Asn Pro Ser Asn Gly Gly Thr Asn Phe Asn | |
| 65 70 75 80 | |
| Glu Lys Phe Lys Asn Arg Val Thr Leu Thr Thr Asp Ser Ser Thr Thr | |
| 85 90 95 | |
| Thr Ala Tyr Met Glu Leu Lys Ser Leu Gln Phe Asp Asp Thr Ala Val | |
| 100 105 110 | |
| Tyr Tyr Cys Ala Arg Arg Asp Tyr Arg Phe Asp Met Gly Phe Asp Tyr | |
| 115 120 125 | |
| Trp Gly Gln Gly Thr Thr Val Thr Val Ser Ser | |
| 130 135 | |
| <210> 90 | |
| <211> 466 | |
| <212> PRT | |
| <213> Artificial Sequence | |
| <220> | |
| <223> 409A-H heavy chain full length | |
| <220> | |
| <221> sig_peptide | |
| <222> (1) . . . (19) | |
| <400> | |
| Met Ala Val Leu Gly Leu Leu Phe Cys Leu Val Thr Phe Pro Ser Cys | |
| 1 5 10 15 | |
| Val Leu Ser Gln Val Gln Leu Val Gln Ser Gly Val Glu Val Lys Lys | |
| 20 25 30 | |
| Pro Gly Ala Ser Val Lys Val Ser Cys Lys Ala Ser Gly Tyr Thr Phe | |
| 35 40 45 | |
| Thr Asn Tyr Tyr Met Tyr Trp Val Arg Gln Ala Pro Gly Gln Gly Leu | |
| 50 55 60 | |
| Glu Trp Met Gly Gly Ile Asn Pro Ser Asn Gly Gly Thr Asn Phe Asn | |
| 65 70 75 80 | |
| Glu Lys Phe Lys Asn Arg Val Thr Leu Thr Thr Asp Ser Ser Thr Thr | |
| 85 90 95 | |
| Thr Ala Tyr Met Glu Leu Lys Ser Leu Gln Phe Asp Asp Thr Ala Val | |
| 100 105 110 | |
| Tyr Tyr Cys Ala Arg Arg Asp Tyr Arg Phe Asp Met Gly Phe Asp Tyr | |
| 115 120 125 | |
| Trp Gly Gln Gly Thr Thr Val Thr Val Ser Ser Ala Ser Thr Lys Gly | |
| 130 135 140 | |
| Pro Ser Val Phe Pro Leu Ala Pro Cys Ser Arg Ser Thr Ser Glu Ser | |
| 145 150 155 160 | |
| Thr Ala Ala Leu Gly Cys Leu Val Lys Asp Tyr Phe Pro Glu Pro Val | |
| 165 170 175 | |
| Thr Val Ser Trp Asn Ser Gly Ala Leu Thr Ser Gly Val His Thr Phe | |
| 180 185 190 | |
| Pro Ala Val Leu Gln Ser Ser Gly Leu Tyr Ser Leu Ser Ser Val Val | |
| 195 200 205 | |
| Thr Val Pro Ser Ser Ser Leu Gly Thr Lys Thr Tyr Thr Cys Asn Val | |
| 210 215 220 | |
| Asp His Lys Pro Ser Asn Thr Lys Val Asp Lys Arg Val Glu Ser Lys | |
| 225 230 235 240 | |
| Tyr Gly Pro Pro Cys Pro Pro Cys Pro Ala Pro Glu Phe Leu Gly Gly | |
| 245 250 255 | |
| Pro Ser Val Phe Leu Phe Pro Pro Lys Pro Lys Asp Thr Leu Met Ile | |
| 260 265 270 | |
| Ser Arg Thr Pro Glu Val Thr Cys Val Val Val Asp Val Ser Gln Glu | |
| 275 280 285 | |
| Asp Pro Glu Val Gln Phe Asn Trp Tyr Val Asp Gly Val Glu Val His | |
| 290 295 300 | |
| Asn Ala Lys Thr Lys Pro Arg Glu Glu Gln Phe Asn Ser Thr Tyr Arg | |
| 305 310 315 320 | |
| Val Val Ser Val Leu Thr Val Leu His Gln Asp Trp Leu Asn Gly Lys | |
| 325 330 335 | |
| Glu Tyr Lys Cys Lys Val Ser Asn Lys Gly Leu Pro Ser Ser Ile Glu | |
| 340 345 350 | |
| Lys Thr Ile Ser Lys Ala Lys Gly Gln Pro Arg Glu Pro Gln Val Tyr | |
| 355 360 365 | |
| Thr Leu Pro Pro Ser Gln Glu Glu Met Thr Lys Asn Gln Val Ser Leu | |
| 370 375 380 | |
| Thr Cys Leu Val Lys Gly Phe Tyr Pro Ser Asp Ile Ala Val Glu Trp | |
| 385 390 395 400 | |
| Glu Ser Asn Gly Gln Pro Glu Asn Asn Tyr Lys Thr Thr Pro Pro Val | |
| 405 410 415 | |
| Leu Asp Ser Asp Gly Ser Phe Phe Leu Tyr Ser Arg Leu Thr Val Asp | |
| 420 425 430 | |
| Lys Ser Arg Trp Gln Glu Gly Asn Val Phe Ser Cys Ser Val Met His | |
| 435 440 445 | |
| Glu Ala Leu His Asn His Tyr Thr Gln Lys Ser Leu Ser Leu Ser Leu | |
| 450 455 460 | |
| Gly Lys | |
| 465 | |
| <210> 91 | |
| <211> 130 | |
| <212> PRT | |
| <213> Artificial Sequence | |
| <220> | |
| <223> K09A-L-11 light chain variable region | |
| <220> | |
| <221> sig_peptide | |
| <222> (1) . . . (19) | |
| <400> | |
| Met Ala Pro Val Gln Leu Leu Gly Leu Leu Val Leu Phe Leu Pro Ala | |
| 1 5 10 15 | |
| Met Arg Cys Glu Ile Val Leu Thr Gln Ser Pro Ala Thr Leu Ser Leu | |
| 20 25 30 | |
| Ser Pro Gly Glu Arg Ala Thr Leu Ser Cys Arg Ala Ser Lys Gly Val | |
| 35 40 45 | |
| Ser Thr Ser Gly Tyr Ser Tyr Leu His Trp Tyr Gln Gln Lys Pro Gly | |
| 50 55 60 | |
| Gln Ala Pro Arg Leu Leu Ile Tyr Leu Ala Ser Tyr Leu Glu Ser Gly | |
| 65 70 75 80 | |
| Val Pro Ala Arg Phe Ser Gly Ser Gly Ser Gly Thr Asp Phe Thr Leu | |
| 85 90 95 | |
| Thr Ile Ser Ser Leu Glu Pro Glu Asp Phe Ala Val Tyr Tyr Cys Gln | |
| 100 105 110 | |
| His Ser Arg Asp Leu Pro Leu Thr Phe Gly Gly Gly Thr Lys Val Glu | |
| 115 120 125 | |
| Ile Lys | |
| 130 | |
| <210> 92 | |
| <211> 130 | |
| <212> PRT | |
| <213> Artificial Sequence | |
| <220> | |
| <223> K09A-L-16 light chain variable region | |
| <220> | |
| <221> sig peptide | |
| <222> (1) . . . (19) | |
| <400> | |
| Met Ala Pro Val Gln Leu Leu Gly Leu Leu Val Leu Phe Leu Pro Ala | |
| 1 5 10 15 | |
| Met Arg Cys Glu Ile Val Leu Thr Gln Ser Pro Leu Ser Leu Pro Val | |
| 20 25 30 | |
| Thr Pro Gly Glu Pro Ala Ser Ile Ser Cys Arg Ala Ser Lys Gly Val | |
| 35 40 45 | |
| Ser Thr Ser Gly Tyr Ser Tyr Leu His Trp Tyr Leu Gln Lys Pro Gly | |
| 50 55 60 | |
| Gln Ser Pro Gln Leu Leu Ile Tyr Leu Ala Ser Tyr Leu Glu Ser Gly | |
| 65 70 75 80 | |
| Val Pro Asp Arg Phe Ser Gly Ser Gly Ser Gly Thr Asp Phe Thr Leu | |
| 85 90 95 | |
| Lys Ile Ser Arg Val Glu Ala Glu Asp Val Gly Val Tyr Tyr Cys Gln | |
| 100 105 110 | |
| His Ser Arg Asp Leu Pro Leu Thr Phe Gly Gln Gly Thr Lys Leu Glu | |
| 115 120 125 | |
| Ile Lys | |
| 130 | |
| <210> 93 | |
| <211> 130 | |
| <212> PRT | |
| <213> Artificial Sequence | |
| <220> | |
| <223> K09A-L-17 light chain variable region | |
| <220> | |
| <221> sig peptide | |
| <222> (1) . . . (19) | |
| <400> | |
| Met Ala Pro Val Gln Leu Leu Gly Leu Leu Val Leu Phe Leu Pro Ala | |
| 1 5 10 15 | |
| Met Arg Cys Asp Ile Val Met Thr Gln Thr Pro Leu Ser Leu Pro Val | |
| 20 25 30 | |
| Thr Pro Gly Glu Pro Ala Ser Ile Ser Cys Arg Ala Ser Lys Gly Val | |
| 35 40 45 | |
| Ser Thr Ser Gly Tyr Ser Tyr Leu His Trp Tyr Leu Gln Lys Pro Gly | |
| 50 55 60 | |
| Gln Ser Pro Gln Leu Leu Ile Tyr Leu Ala Ser Tyr Leu Glu Ser Gly | |
| 65 70 75 80 | |
| Val Pro Asp Arg Phe Ser Gly Ser Gly Ser Gly Thr Ala Phe Thr Leu | |
| 85 90 95 | |
| Lys Ile Ser Arg Val Glu Ala Glu Asp Val Gly Leu Tyr Tyr Cys Gln | |
| 100 105 110 | |
| His Ser Arg Asp Leu Pro Leu Thr Phe Gly Gln Gly Thr Lys Leu Glu | |
| 115 120 125 | |
| Ile Lys | |
| 130 | |
| <210> 94 | |
| <211> 469 | |
| <212> PRT | |
| <213> Artificial Sequence | |
| <220> | |
| <223> 109A-H heavy chain full length | |
| <220> | |
| <221> sig peptide | |
| <222> (1) . . . (19) | |
| <400> | |
| Met Ala Val Leu Gly Leu Leu Phe Cys Leu Val Thr Phe Pro Ser Cys | |
| 1 5 10 15 | |
| Val Leu Ser Gln Val Gln Leu Val Gln Ser Gly Val Glu Val Lys Lys | |
| 20 25 30 | |
| Pro Gly Ala Ser Val Lys Val Ser Cys Lys Ala Ser Gly Tyr Thr Phe | |
| 35 40 45 | |
| Thr Asn Tyr Tyr Met Tyr Trp Val Arg Gln Ala Pro Gly Gln Gly Leu | |
| 50 55 60 | |
| Glu Trp Met Gly Gly Ile Asn Pro Ser Asn Gly Gly Thr Asn Phe Asn | |
| 65 70 75 80 | |
| Glu Lys Phe Lys Asn Arg Val Thr Leu Thr Thr Asp Ser Ser Thr Thr | |
| 85 90 95 | |
| Thr Ala Tyr Met Glu Leu Lys Ser Leu Gln Phe Asp Asp Thr Ala Val | |
| 100 105 110 | |
| Tyr Tyr Cys Ala Arg Arg Asp Tyr Arg Phe Asp Met Gly Phe Asp Tyr | |
| 115 120 125 | |
| Trp Gly Gln Gly Thr Thr Val Thr Val Ser Ser Ala Ser Thr Lys Gly | |
| 130 135 140 | |
| Pro Ser Val Phe Pro Leu Ala Pro Ser Ser Lys Ser Thr Ser Gly Gly | |
| 145 150 155 160 | |
| Thr Ala Ala Leu Gly Cys Leu Val Lys Asp Tyr Phe Pro Glu Pro Val | |
| 165 170 175 | |
| Thr Val Ser Trp Asn Ser Gly Ala Leu Thr Ser Gly Val His Thr Phe | |
| 180 185 190 | |
| Pro Ala Val Leu Gln Ser Ser Gly Leu Tyr Ser Leu Ser Ser Val Val | |
| 195 200 205 | |
| Thr Val Pro Ser Ser Ser Leu Gly Thr Gln Thr Tyr Ile Cys Asn Val | |
| 210 215 220 | |
| Asn His Lys Pro Ser Asn Thr Lys Val Asp Lys Lys Val Glu Pro Lys | |
| 225 230 235 240 | |
| Ser Cys Asp Lys Thr His Thr Cys Pro Pro Cys Pro Ala Pro Glu Leu | |
| 245 250 255 | |
| Leu Gly Gly Pro Ser Val Phe Leu Phe Pro Pro Lys Pro Lys Asp Thr | |
| 260 265 270 | |
| Leu Met Ile Ser Arg Thr Pro Glu Val Thr Cys Val Val Val Asp Val | |
| 275 280 285 | |
| Ser His Glu Asp Pro Glu Val Lys Phe Asn Trp Tyr Val Asp Gly Val | |
| 290 295 300 | |
| Glu Val His Asn Ala Lys Thr Lys Pro Arg Glu Glu Gln Tyr Asn Ser | |
| 305 310 315 320 | |
| Thr Tyr Arg Val Val Ser Val Leu Thr Val Leu His Gln Asp Trp Leu | |
| 325 330 335 | |
| Asn Gly Lys Glu Tyr Lys Cys Lys Val Ser Asn Lys Ala Leu Pro Ala | |
| 340 345 350 | |
| Pro Ile Glu Lys Thr Ile Ser Lys Ala Lys Gly Gln Pro Arg Glu Pro | |
| 355 360 365 | |
| Gln Val Tyr Thr Leu Pro Pro Ser Arg Asp Glu Leu Thr Lys Asn Gln | |
| 370 375 380 | |
| Val Ser Leu Thr Cys Leu Val Lys Gly Phe Tyr Pro Ser Asp Ile Ala | |
| 385 390 395 400 | |
| Val Glu Trp Glu Ser Asn Gly Gln Pro Glu Asn Asn Tyr Lys Thr Thr | |
| 405 410 415 | |
| Pro Pro Val Leu Asp Ser Asp Gly Ser Phe Phe Leu Tyr Ser Lys Leu | |
| 420 425 430 | |
| Thr Val Asp Lys Ser Arg Trp Gln Gln Gly Asn Val Phe Ser Cys Ser | |
| 435 440 445 | |
| Val Met His Glu Ala Leu His Asn His Tyr Thr Gln Lys Ser Leu Ser | |
| 450 455 460 | |
| Leu Ser Pro Gly Lys | |
| 465 | |
| <210> 95 | |
| <211> 237 | |
| <212> PRT | |
| <213> Artificial Sequence | |
| <220> | |
| <223> K09A-L-11 light chain full length | |
| <220> | |
| <221> sig_peptide | |
| <222> (1) . . . (19) | |
| <400> | |
| Met Ala Pro Val Gln Leu Leu Gly Leu Leu Val Leu Phe Leu Pro Ala | |
| 1 5 10 15 | |
| Met Arg Cys Glu Ile Val Leu Thr Gln Ser Pro Ala Thr Leu Ser Leu | |
| 20 25 30 | |
| Ser Pro Gly Glu Arg Ala Thr Leu Ser Cys Arg Ala Ser Lys Gly Val | |
| 35 40 45 | |
| Ser Thr Ser Gly Tyr Ser Tyr Leu His Trp Tyr Gln Gln Lys Pro Gly | |
| 50 55 60 | |
| Gln Ala Pro Arg Leu Leu Ile Tyr Leu Ala Ser Tyr Leu Glu Ser Gly | |
| 65 70 75 80 | |
| Val Pro Ala Arg Phe Ser Gly Ser Gly Ser Gly Thr Asp Phe Thr Leu | |
| 85 90 95 | |
| Thr Ile Ser Ser Leu Glu Pro Glu Asp Phe Ala Val Tyr Tyr Cys Gln | |
| 100 105 110 | |
| His Ser Arg Asp Leu Pro Leu Thr Phe Gly Gly Gly Thr Lys Val Glu | |
| 115 120 125 | |
| Ile Lys Arg Thr Val Ala Ala Pro Ser Val Phe Ile Phe Pro Pro Ser | |
| 130 135 140 | |
| Asp Glu Gln Leu Lys Ser Gly Thr Ala Ser Val Val Cys Leu Leu Asn | |
| 145 150 155 160 | |
| Asn Phe Tyr Pro Arg Glu Ala Lys Val Gln Trp Lys Val Asp Asn Ala | |
| 165 170 175 | |
| Leu Gln Ser Gly Asn Ser Gln Glu Ser Val Thr Glu Gln Asp Ser Lys | |
| 180 185 190 | |
| Asp Ser Thr Tyr Ser Leu Ser Ser Thr Leu Thr Leu Ser Lys Ala Asp | |
| 195 200 205 | |
| Tyr Glu Lys His Lys Val Tyr Ala Cys Glu Val Thr His Gln Gly Leu | |
| 210 215 220 | |
| Ser Ser Pro Val Thr Lys Ser Phe Asn Arg Gly Glu Cys | |
| 225 230 235 | |
| <210> 96 | |
| <211> 237 | |
| <212> PRT | |
| <213> Artificial Sequence | |
| <220> | |
| <223> K09A-L-16 light chain full length | |
| <220> | |
| <221> sig_peptide | |
| <222> (1) . . . (19) | |
| <400> | |
| Met Ala Pro Val Gln Leu Leu Gly Leu Leu Val Leu Phe Leu Pro Ala | |
| 1 5 10 15 | |
| Met Arg Cys Glu Ile Val Leu Thr Gln Ser Pro Leu Ser Leu Pro Val | |
| 20 25 30 | |
| Thr Pro Gly Glu Pro Ala Ser Ile Ser Cys Arg Ala Ser Lys Gly Val | |
| 35 40 45 | |
| Ser Thr Ser Gly Tyr Ser Tyr Leu His Trp Tyr Leu Gln Lys Pro Gly | |
| 50 55 60 | |
| Gln Ser Pro Gln Leu Leu Ile Tyr Leu Ala Ser Tyr Leu Glu Ser Gly | |
| 65 70 75 80 | |
| Val Pro Asp Arg Phe Ser Gly Ser Gly Ser Gly Thr Asp Phe Thr Leu | |
| 85 90 95 | |
| Lys Ile Ser Arg Val Glu Ala Glu Asp Val Gly Val Tyr Tyr Cys Gln | |
| 100 105 110 | |
| His Ser Arg Asp Leu Pro Leu Thr Phe Gly Gln Gly Thr Lys Leu Glu | |
| 115 120 125 | |
| Ile Lys Arg Thr Val Ala Ala Pro Ser Val Phe Ile Phe Pro Pro Ser | |
| 130 135 140 | |
| Asp Glu Gln Leu Lys Ser Gly Thr Ala Ser Val Val Cys Leu Leu Asn | |
| 145 150 155 160 | |
| Asn Phe Tyr Pro Arg Glu Ala Lys Val Gln Trp Lys Val Asp Asn Ala | |
| 165 170 175 | |
| Leu Gln Ser Gly Asn Ser Gln Glu Ser Val Thr Glu Gln Asp Ser Lys | |
| 180 185 190 | |
| Asp Ser Thr Tyr Ser Leu Ser Ser Thr Leu Thr Leu Ser Lys Ala Asp | |
| 195 200 205 | |
| Tyr Glu Lys His Lys Val Tyr Ala Cys Glu Val Thr His Gln Gly Leu | |
| 210 215 220 | |
| Ser Ser Pro Val Thr Lys Ser Phe Asn Arg Gly Glu Cys | |
| 225 230 235 | |
| <210> 97 | |
| <211> 237 | |
| <212> PRT | |
| <213> Artificial Sequence | |
| <220> | |
| <223> K09A-L-17 light chain full length | |
| <220> | |
| <221> sig_peptide | |
| <222> (1) . . . (19) | |
| <400> | |
| Met Ala Pro Val Gln Leu Leu Gly Leu Leu Val Leu Phe Leu Pro Ala | |
| 1 5 10 15 | |
| Met Arg Cys Asp Ile Val Met Thr Gln Thr Pro Leu Ser Leu Pro Val | |
| 20 25 30 | |
| Thr Pro Gly Glu Pro Ala Ser Ile Ser Cys Arg Ala Ser Lys Gly Val | |
| 35 40 45 | |
| Ser Thr Ser Gly Tyr Ser Tyr Leu His Trp Tyr Leu Gln Lys Pro Gly | |
| 50 55 60 | |
| Gln Ser Pro Gln Leu Leu Ile Tyr Leu Ala Ser Tyr Leu Glu Ser Gly | |
| 65 70 75 80 | |
| Val Pro Asp Arg Phe Ser Gly Ser Gly Ser Gly Thr Ala Phe Thr Leu | |
| 85 90 95 | |
| Lys Ile Ser Arg Val Glu Ala Glu Asp Val Gly Leu Tyr Tyr Cys Gln | |
| 100 105 110 | |
| His Ser Arg Asp Leu Pro Leu Thr Phe Gly Gln Gly Thr Lys Leu Glu | |
| 115 120 125 | |
| Ile Lys Arg Thr Val Ala Ala Pro Ser Val Phe Ile Phe Pro Pro Ser | |
| 130 135 140 | |
| Asp Glu Gln Leu Lys Ser Gly Thr Ala Ser Val Val Cys Leu Leu Asn | |
| 145 150 155 160 | |
| Asn Phe Tyr Pro Arg Glu Ala Lys Val Gln Trp Lys Val Asp Asn Ala | |
| 165 170 175 | |
| Leu Gln Ser Gly Asn Ser Gln Glu Ser Val Thr Glu Gln Asp Ser Lys | |
| 180 185 190 | |
| Asp Ser Thr Tyr Ser Leu Ser Ser Thr Leu Thr Leu Ser Lys Ala Asp | |
| 195 200 205 | |
| Tyr Glu Lys His Lys Val Tyr Ala Cys Glu Val Thr His Gln Gly Leu | |
| 210 215 220 | |
| Ser Ser Pro Val Thr Lys Ser Phe Asn Arg Gly Glu Cys | |
| 225 230 235 | |

As another example, an anti-OX40 antibody can be used in combination with nivolumab (OPDIVO®). Nivolumab (OPDIVO®) is a programmed death receptor-1 (PD-1) blocking antibody indicated for the treatment of patients with:
unresectable or metastatic melanoma and disease progression following ipilimumab and, if BRAF V600 mutation positive, a BRAF inhibitor. This indication is approved under accelerated approval based on tumor response rate and durability of response. Continued approval for this indication may be contingent upon verification and description of clinical benefit in the confirmatory trials.
metastatic squamous non-small cell lung cancer with progression on or after platinum-based chemotherapy.

The recommended dose of nivolumab (OPDIVO®) is 3 mg/kg administered as an intravenous infusion over 60 minutes every 2 weeks unto disease progression or unacceptable toxicity.

Binding of the PD-1 ligands, PD-L1 and PD-L2, to the PD-1 receptor found on T cells, inhibits T-cell proliferation and cytokine production. Upregulation of PD-1 ligands occurs in some tumors and signaling through this pathway can contribute to inhibition of active T-cell immune surveillance of tumors.

Nivolumab is a human immunoglobulin G4 (IgG4) monoclonal antibody that binds to the PD-1 receptor and blocks its interaction with PD-L1 and PD-L2, releasing PD-1 pathway-mediated inhibition of the immune response, including the anti-tumor immune response. In syngeneic mouse tumor models, blocking PD-1 activity resulted in decreased tumor growth.

U.S. Pat. No. 8,008,449 exemplifies seven anti-PD-1 HuMAbs: 17D8, 2D3, 4H1, 5C4 (also referred to herein as nivolumab or BMS-936558), 4A1 1, 7D3 and 5F4. See also U.S. Pat. No. 8,779,105. Any one of these antibodies, or the CDRs thereof (or an amino acid sequence with at least 90% (e.g., 90%, 91%, 92%, 93%, 94%, 95%, 96%, 97%, 98%, or 99%, or 100%) sequence identity to any of these amino acid sequences), can be used in the compositions and methods described herein.

```
Heavy Chain of nivolumab:
(SEQ ID NO: 98)
QVQLVESGGGVVQPGRSLRLDCKASGITFSNSGMHWVRQAPGKGLEWVAVI
WYDGSKRYYADSVKGRFTISRDNSKNTLFLQMNSLRAEDTAVYYCATNDDY
WGQGTLVTVSSASTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVD
KRVESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGNVFS
CSVMHEALHNHYTQKSLSLSLGK
Light Chain of nivolumab:
(SEQ ID NO: 99)
EIVLTQSPATLSLSPGERATLSCRASQSVSSYLAWYQQKPGQAPRLLIYDASN
RATGIPARFSGSGSGTDFTLTISSLEPEDFAVYYCQQSSNWPRTFGQGTKVEIK
RTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRG
EC
Heavy Chain Variable region of nivolumab:
(SEQ ID NO: 100)
Gln Val Gln Leu Val Glu Ser Gly Gly Gly Val Val Gln Pro Gly Arg
1 5 10 15
Ser Leu Arg Leu Asp Cys Lys Ala Ser Gly Ile Thr Phe Ser Asn Ser
                        20 25 30
Gly Met His Trp Val Arg Gln Ala Pro Gly Lys Gly Leu Glu Trp Val
                35 40 45
Ala Val Ile Trp Tyr Asp Gly Ser Lys Arg Tyr Tyr Ala Asp Ser Val
        50 55 60
Lys Gly Arg Phe Thr Ile Ser Arg Asp Asn Ser Lys Asn Thr Leu Phe
65 70 75 80
Leu Gln Met Asn Ser Leu Arg Ala Glu Asp Thr Ala Val Tyr Tyr Cys
                                85 90 95
Ala Thr Asn Asp Asp Tyr Trp Gly Gln Gly Thr Leu Val Thr Val Ser
                        100 105 110
Ser
Light Chain Variable region of nivolumab:
(SEQ ID NO: 101)
Glu Ile Val Leu Thr Gln Ser Pro Ala Thr Leu Ser Leu Ser Pro Gly
1 5 10 15
Glu Arg Ala Thr Leu Ser Cys Arg Ala Ser Gln Ser Val Ser Ser Tyr
                        20 25 30
Leu Ala Trp Tyr Gln Gln Lys Pro Gly Gln Ala Pro Arg Leu Leu Ile
                35 40 45
Tyr Asp Ala Ser Asn Arg Ala Thr Gly Ile Pro Ala Arg Phe Ser Gly
        50 55 60
Ser Gly Ser Gly Thr Asp Phe Thr Leu Thr Ile Ser Ser Leu Glu Pro
65 70 75 80
Glu Asp Phe Ala Val Tyr Tyr Cys Gln Gln Ser Ser Asn Trp Pro Arg
                                85 90 95
Thr Phe Gly Gln Gly Thr Lys Val Glu Ile Lys
                        100 105
CDR sequences of nivolumab:
HC CDR1:
(SEQ ID NO: 102)
Asn Ser Gly Met His
HC CDR2:
(SEQ ID NO: 103)
Val Ile Trp Tyr Asp Gly Ser Lys Arg Tyr Tyr Ala Asp Ser Val Lys
Gly
HC CDR3:
(SEQ ID NO: 104)
Asn Asp Asp Tyr
LC CDR1:
(SEQ ID NO: 105)
Arg Ala Ser Gln Ser Val Ser Ser Tyr Leu Ala
LC CDR2:
(SEQ ID NO: 106)
Asp Ala Ser Asn Arg Ala Thr
LC CDR3:
(SEQ ID NO: 107)
Gln Gln Ser Ser Asn Trp Pro Arg Thr
```

In another embodiment, the anti-PD-1 ABP of a combination of the invention, or a method or use thereof, comprises one or more (e.g. all) of the CDRs or VH or VL or HC (heavy chain) or LC (light chain) sequences of nivolumab, or sequences with at least 90% (e.g., 90%, 91%, 92%, 93%, 94%, 95%, 96%, 97%, 98%, or 99%, or 100%) sequence identity thereto.

The HC and LC CDRs of nivolumab are provided herein. In one embodiment, the anti-PD-1 ABP of a combination of the invention, or a method or use thereof, comprises: (a) a heavy chain variable region CDR1 of nivolumab; (b) a heavy chain variable region CDR2 of nivolumab; (c) a heavy chain variable region CDR3 of nivolumab; (d) a light chain variable region CDR1 of nivolumab; (e) a light chain variable region CDR2 of nivolumab; and (f) a light chain variable region CDR3 of nivolumab.

In another embodiment, the anti-PD-1 of a combination of the invention, or a method or use thereof, comprises: a heavy chain variable region CDR1 of nivolumab; a heavy chain variable region CDR2 of nivolumab and/or a heavy chain variable region CDR3 of nivolumab.

In another embodiment, the anti-PD-1 of a combination of the invention, or a method or use thereof, comprises: a light chain variable region CDR1 of nivolumab; a light chain variable region CDR2 of nivolumab and/or a light chain variable region CDR3 of nivolumab.

In another embodiment, the anti-PD-1 ABP of a combination of the invention, or a method or use thereof, comprises: a light chain variable region (“VL”) of nivolumab, or an amino acid sequence with at least 90% (e.g., 90%, 91%, 92%, 93%, 94%, 95%, 96%, 97%, 98%, or 99%, or 100%) sequence identity to the amino acid sequence of the VL of nivolumab.

In another embodiment, the anti-PD-1 ABP of a combination of the invention, or a method or use thereof, comprises a heavy chain variable region (“VH”) of nivolumab, or an amino acid sequence with at least 90% (e.g., 90%, 91%, 92%, 93%, 94%, 95%, 96%, 97%, 98%, or 99%, or 100%) sequence identity to the amino acid sequence of the VH of nivolumab.

In another embodiment, the anti-PD-1 ABP of a combination of the invention, or a method or use thereof, comprises: a light chain variable region (“VL”) of nivolumab, or an amino acid sequence with at least 90% (e.g., 90%, 91%, 92%, 93%, 94%, 95%, 96%, 97%, 98%, or 99%, or 100%) sequence identity to the amino acid sequence of the VL of nivolumab and the anti-PD-1 ABP of a combination of the invention, or a method or use thereof, comprises a heavy chain variable region (“VH”) of nivolumab, or an amino acid sequence with at least 90% (e.g., 90%, 91%, 92%, 93%, 94%, 95%, 96%, 97%, 98%, or 99%, or 100%) sequence identity to the amino acid sequence of the VH of nivolumab.

In another embodiment, the anti-PD-1 ABP of a combination of the invention, or a method or use thereof, comprises: a light chain (“LC”) of nivolumab, or an amino acid sequence with at least 90% (e.g., 90%, 91%, 92%, 93%, 94%, 95%, 96%, 97%, 98%, or 99%, or 100%) sequence identity to the amino acid sequence of the LC of nivolumab.

In another embodiment, the anti-PD-1 ABP of a combination of the invention, or a method or use thereof, comprises a heavy chain (“HC”) of nivolumab, or an amino acid sequence with at least 90% (e.g., 90%, 91%, 92%, 93%, 94%, 95%, 96%, 97%, 98%, or 99%, or 100%) sequence identity to the amino acid sequence of the HC of nivolumab.

In another embodiment, the anti-PD-1 ABP of a combination of the invention, or a method or use thereof, comprises: a light chain (“LC”) of nivolumab, or an amino acid sequence with at least 90% (e.g., 90%, 91%, 92%, 93%, 94%, 95%, 96%, 97%, 98%, or 99%, or 100%) sequence identity to the amino acid sequence of the LC of nivolumab and the anti-PD-1 ABP of a combination of the invention, or a method or use thereof, comprises a heavy chain (“HC”) of nivolumab, or an amino acid sequence with at least 90% (e.g., 90%, 91%, 92%, 93%, 94%, 95%, 96%, 97%, 98%, or 99%, or 100%) sequence identity to the amino acid sequence of the HC of nivolumab.

Another embodiment of a combination of the invention, or a method or use thereof, includes CDRs, VH regions, and VL regions, HC, and LC, and antibodies and nucleic acids encoding the same as disclosed in the below Sequence Listing.

An anti-OX40 ABP (e.g., an agonist ABP, e.g. an anti-hOX40 ABP, e.g. antibody), e.g., an antibody described herein, can be used in combination with an ABP (e.g., antagonist ABP, e.g antagonist antibody) against PD-1 (e.g. human PD-1). For example, an anti-OX40 antibody can be used in combination with nivolumab.

Thus, in one embodiment, methods are provided for treating cancer in a human in need thereof comprising administering a therapeutically effective amount of a monoclonal antibody that binds to human OX40 comprising a VH region comprising an amino acid sequence with at least 90%, 91%, 92%, 93%, 94%, 95%, 96%, 97%, 98%, 99% or 100% sequence identity to the amino acid sequence as set forth in SEQ ID NO: 4 and a VL comprising and amino acid sequence with at least 90%, 91%, 92%, 93%, 94%, 95%, 96%, 97%, 98%, 99% or 100% sequence identity to the amino acid sequence as set forth in SEQ ID NO:10, and pembrolizumab, or an antibody having 90%, 91%, 92%, 93%, 94%, 95%, 96%, 97%, 98%, 99% or 100% sequence identity thereto. In one embodiment, the monoclonal antibody that binds OX40 comprises a heavy chain variable region comprising the amino acid sequence set forth in SEQ ID NO:4 and a light chain variable region comprising the amino acid sequence set forth in SEQ ID NO:10.

In one aspect, the cancer is a solid tumor. In one aspect, the cancer is selected from: melanoma, lung cancer, kidney cancer, breast cancer, head and neck cancer, colon cancer, ovarian cancer, pancreatic cancer, liver cancer, prostate cancer, bladder cancer, gastric cancer. In another aspect, the cancer is selected from: NSCLC, squamous cell carcinoma of the head and neck (SCCHN), renal cell carcinoma (RCC), melanoma, bladder cancer, soft tissue sarcoma (STS), triple-negative breast cancer (TNBC), and colorectal carcinoma displaying microsatellite instability (MSI CRC).

In one embodiment, the monoclonal antibody that binds to OX40 is ANTIBODY 106-222. In one embodiment, the monoclonal antibody that binds to OX40 and pembrolizumab are administered at the same time. In one embodiment, the monoclonal antibody that binds to OX40 and pembrolizumab are administered sequentially, in any order. In one embodiment, the monoclonal antibody that binds to OX40 and/or the pembrolizumab are administered intravenously. Suitably, the monoclonal antibody that binds to OX40 and/or the pembrolizumab are administered intratumorally.

In one embodiment, the monoclonal antibody that binds to OX40 is administered at a dose of about 0.1 mg/kg to about 10 mg/kg. The monoclonal antibody that binds to OX40 can be administered at a frequency selected from: once daily, once weekly, once every two weeks (Q2W) and once every three weeks (Q3W). Suitably, the antibody that binds to OX40 is administered once every three weeks. In one aspect, the pembrolizumab is administered at a dose of 200 mg Q3W.

In one embodiment pharmaceutical compositions are provided comprising a therapeutically effective amount of an a monoclonal antibody that bind to OX40 comprising a VH region comprising an amino acid sequence with at least 90%, 91%, 92%, 93%, 94%, 95%, 96%, 97%, 98%, 99% or 100% sequence identity to the amino acid sequence as set forth in SEQ ID NO:5 and comprising a VL comprising an amino acid sequence with at least 90%, 91%, 92%, 93%, 94%, 95%, 96%, 97%, 98%, 99% or 100% sequence identity to the amino acid sequence as set forth in SEQ ID NO:11, and pembrolizumab, or an antibody comprising 90%, 91%, 92%, 93%, 94%, 95%, 96%, 97%, 98%, 99% or 100% sequence identity thereto. Suitably, the monoclonal antibody that binds OX40 comprises a variable heavy chain comprising the amino acid sequence set forth in SEQ ID NO:5 and a variable light chain comprising the amino acid sequence set forth in SEQ ID NO: 11. Suitably, the pharmaceutical composition comprises pembrolizumab.

Also provided as part of the instant invention are combination kits comprising a pharmaceutical compositions of the present invention together with one or more pharmaceutically acceptable carriers.

The present invention also provides use of the pharmaceutical compositions or kit of the invention in the manufacture of a medicament for the treatment of cancer.

In one embodiment, the present invention provides methods of reducing tumor size in a human having cancer comprising administering a therapeutically effective amount of ANTIBODY 106-222 and a therapeutically effective amount of pembrolizumab to said human. In one aspect, the human demonstrates complete response or partial response according to RECIST version 1.1.

In some embodiments of the methods presented herein (e.g., methods of treating cancer and/or reducing tumor size in a human), the monoclonal antibody that binds to human OX40 (e.g., a monoclonal antibody that binds to human OX40 comprising a VH region comprising an amino acid sequence with at least 90%, 91%, 92%, 93%, 94%, 95%, 96%, 97%, 98%, 99% or 100% sequence identity to the amino acid sequence as set forth in SEQ ID NO: 5 and a VL comprising and amino acid sequence with at least 90%, 91%, 92%, 93%, 94%, 95%, 96%, 97%, 98%, 99% or 100% sequence identity to the amino acid sequence as set forth in SEQ ID NO:11) (e.g., ANTIBODY 106-222) (e.g., a therapeutically effective amount thereof) is administered to the human first and pembrolizumab (or an antibody having 90%, 91%, 92%, 93%, 94%, 95%, 96%, 97%, 98%, 99% or 100% sequence identity thereto) (e.g., a therapeutically effective amount thereof) is administered second. In some embodiments, the monoclonal antibody that binds to human OX40 (e.g., a monoclonal antibody that binds to human OX40 comprising a VH region comprising an amino acid sequence with at least 90%, 91%, 92%, 93%, 94%, 95%, 96%, 97%, 98%, 99% or 100% sequence identity to the amino acid sequence as set forth in SEQ ID NO: 5 and a VL comprising and amino acid sequence with at least 90%, 91%, 92%, 93%, 94%, 95%, 96%, 97%, 98%, 99% or 100% sequence identity to the amino acid sequence as set forth in SEQ ID NO:11) (e.g., ANTIBODY 106-222) is administered intravenously (e.g., intravenous infusion). In some embodiments, pembrolizumab (or an antibody having 90%, 91%, 92%, 93%, 94%, 95%, 96%, 97%, 98%, 99% or 100% sequence identity thereto) is administered intravenously. In some embodiments, pembrolizumab (or an antibody having 90%, 91%, 92%, 93%, 94%, 95%, 96%, 97%, 98%, 99% or 100% sequence identity thereto) is administered to subjects starting at least 1 hour and no more than 2 hours following the end of administration of the monoclonal antibody that binds to human OX40 (e.g., a monoclonal antibody that binds to human OX40 comprising a VH region comprising an amino acid sequence with at least 90%, 91%, 92%, 93%, 94%, 95%, 96%, 97%, 98%, 99% or 100% sequence identity to the amino acid sequence as set forth in SEQ ID NO: 5 and a VL comprising and amino acid sequence with at least 90%, 91%, 92%, 93%, 94%, 95%, 96%, 97%, 98%, 99% or 100% sequence identity to the amino acid sequence as set forth in SEQ ID NO:11) (e.g., ANTIBODY 106-222). In some embodiments, pembrolizumab (or an antibody having 90%, 91%, 92%, 93%, 94%, 95%, 96%, 97%, 98%, 99% or 100% sequence identity thereto) is intravenously administered to the human starting at least 1 hour and no more than 2 hours following the end of infusion (e.g., intravenous infusion) of the monoclonal antibody that binds to human OX40 (e.g., a monoclonal antibody that binds to human OX40 comprising a VH region comprising an amino acid sequence with at least 90%, 91%, 92%, 93%, 94%, 95%, 96%, 97%, 98%, 99% or 100% sequence identity to the amino acid sequence as set forth in SEQ ID NO: 5 and a VL comprising and amino acid sequence with at least 90%, 91%, 92%, 93%, 94%, 95%, 96%, 97%, 98%, 99% or 100% sequence identity to the amino acid sequence as set forth in SEQ ID NO:11) (e.g., ANTIBODY 106-222). In some embodiments, the monoclonal antibody that binds to OX40 is administered at a dose of about 0.003 mg/kg to about 10 mg/kg (e.g., 0.1 mg/kg to about 10 mg/kg) (e.g., Q3W). In some embodiments, the pembrolizumab is administered at a dose of 200 mg (e.g., Q3W).

In some embodiments of the methods presented herein (e.g., methods of treating cancer and/or reducing tumor size in a human), the monoclonal antibody that binds to human OX40 (e.g., a monoclonal antibody that binds to human OX40 comprising a VH region comprising an amino acid sequence with at least 90%, 91%, 92%, 93%, 94%, 95%, 96%, 97%, 98%, 99% or 100% sequence identity to the amino acid sequence as set forth in SEQ ID NO: 5 and a VL comprising and amino acid sequence with at least 90%, 91%, 92%, 93%, 94%, 95%, 96%, 97%, 98%, 99% or 100% sequence identity to the amino acid sequence as set forth in SEQ ID NO:11) (e.g., ANTIBODY 106-222) (e.g., a therapeutically effective amount thereof) is administered intravenously (e.g., by intravenous infusion) (to the human) first and pembrolizumab (or an antibody having 90%, 91%, 92%, 93%, 94%, 95%, 96%, 97%, 98%, 99% or 100% sequence identity thereto) (e.g., a therapeutically effective amount thereof) is administered intravenously (to the human) second, wherein pembrolizumab (or an antibody having 90%, 91%, 92%, 93%, 94%, 95%, 96%, 97%, 98%, 99% or 100% sequence identity thereto) is intravenously administered to the human starting at least 1 hour and no more than 2 hours following the end of infusion (e.g., intravenous infusion) of the monoclonal antibody that binds to human OX40 (e.g., a monoclonal antibody that binds to human OX40 comprising a VH region comprising an amino acid sequence with at least 90%, 91%, 92%, 93%, 94%, 95%, 96%, 97%, 98%, 99% or 100% sequence identity to the amino acid sequence as set forth in SEQ ID NO: 5 and a VL comprising and amino acid sequence with at least 90%, 91%, 92%, 93%, 94%, 95%, 96%, 97%, 98%, 99% or 100% sequence identity to the amino acid sequence as set forth in SEQ ID NO:11) (e.g., ANTIBODY 106-222). In some embodiments, the monoclonal antibody that binds to OX40 is administered at a dose of about 0.003 mg/kg to about 10 mg/kg (e.g., 0.1 mg/kg to about 10 mg/kg) (e.g., Q3W). In some embodiments, the pembrolizumab is administered at a dose of 200 mg (e.g., Q3W).

Methods of Treatment

The combinations of the invention are believed to have utility in disorders wherein the engagement of OX40 (e.g., agonistic engagement) and/or PD-1 (e.g., antagonistic engagement), is beneficial.

The present invention thus also provides a combination of the invention, for use in therapy, particularly in the treatment of disorders wherein the engagement of OX40 (e.g., agonistic engagement) and/or PD-1 (e.g., antagonistic engagement), is beneficial, particularly cancer.

A further aspect of the invention provides a method of treatment of a disorder wherein engagement of OX40 (e.g., agonistic engagement) and/or PD-1 (e.g., antagonistic engagement), comprising administering a combination of the invention.

A further aspect of the present invention provides the use of a combination of the invention in the manufacture of a medicament for the treatment of a disorder engagement of OX40 (e.g., agonistic engagement) and/or PD-1 (e.g., antagonistic engagement), is beneficial. In preferred embodiments the disorder is cancer.

Examples of cancers that are suitable for treatment with combination of the invention include, but are limited to, both primary and metastatic forms of head and neck, breast, lung, colon, ovary, and prostate cancers. Suitably the cancer is selected from: brain (gliomas), glioblastomas, astrocytomas, glioblastoma multiforme, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor) and testicular cancer.

Additionally, examples of a cancer to be treated include Barret's adenocarcinoma; biliary tract carcinomas; breast cancer; cervical cancer; cholangiocarcinoma; central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system); colorectal cancer including large intestinal colon carcinoma; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer and non-small cell lung cancer; ovarian cancer; endometrial cancer; pancreatic cancer; pituitary adenoma; prostate cancer; renal cancer; sarcoma; skin cancers including melanomas; and thyroid cancers.

Suitably, the present invention relates to a method for treating or lessening the severity of a cancer selected from: brain (gliomas), glioblastomas, astrocytomas, glioblastoma multiforme, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, breast, pancreatic and prostate.

Suitably, the present invention relates to a method for treating or lessening the severity of NSCLC (non small cell lung cancer), squamous cell carcinoma of the head and neck (SCCHN), renal cell carcinoma (RCC), melanoma, bladder cancer, soft tissue sarcoma (STS), triple-negative breast cancer (TNBC), and colorectal carcinoma displaying microsatellite instability (MSI CRC).

Suitably, the present invention relates to a method for treating or lessening the severity of melanoma, e.g. metastatic melanoma.

Suitably, the present invention relates to a method for treating or lessening the severity of squamous non-small cell lung cancer, e.g. metastatic squamous non-small cell lung cancer.

Suitably the present invention relates to a method for treating or lessening the severity of pre-cancerous syndromes in a mammal, including a human, wherein the pre-cancerous syndrome is selected from: cervical intraepithelial neoplasia, monoclonal gammapathy of unknown significance (MGUS), myelodysplastic syndrome, aplastic anemia, cervical lesions, skin nevi (pre-melanoma), prostatic intraepithelial (intraductal) neoplasia (PIN), Ductal Carcinoma in situ (DCIS), colon polyps and severe hepatitis or cirrhosis.

The combination of the invention may be used alone or in combination with one or more other therapeutic agents. The invention thus provides in a further aspect a further combination comprising a combination of the invention with a further therapeutic agent or agents, compositions and medicaments comprising the combination and use of the further combination, compositions and medicaments in therapy, in particular in the treatment of diseases susceptible engagement of OX40, e.g. agonism of OX40, and/or PD-1, e.g. antagonism of PD-1.

In the embodiment, the combination of the invention may be employed with other therapeutic methods of cancer treatment. In particular, in anti-neoplastic therapy, combination therapy with other chemotherapeutic, hormonal, antibody agents as well as surgical and/or radiation treatments other than those mentioned above are envisaged. Combination therapies according to the present invention thus include the administration of an anti-OX40 ABP of a combination, or method or use thereof, of the invention and/or an anti-PD-1 ABP of a combination, or method or use thereof, of the invention as well as optional use of other therapeutic agents including other anti-neoplastic agents. Such combination of agents may be administered together or separately and, when administered separately this may occur simultaneously or sequentially in any order, both close and remote in time. In one embodiment, the pharmaceutical combination includes an anti-OX40 ABP, suitably an agonist anti-OX40 ABP and an anti-PD-1 ABP, suitably an antagonist anti-PD1 ABP, and optionally at least one additional anti-neoplastic agent.

In one embodiment, the further anti-cancer therapy is surgical and/or radiotherapy.

In one embodiment, the further anti-cancer therapy is at least one additional anti-neoplastic agent.

Any anti-neoplastic agent that has activity versus a susceptible tumor being treated may be utilized in the combination. Typical anti-neoplastic agents useful include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine angiogenesis inhibitors; immunotherapeutic agents; proapoptotic agents; and cell cycle signaling inhibitors.

Anti-microtubule or anti-mitotic agents: Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. Paclitaxel has been approved for clinical use in the treatment of refractory ovarian cancer in the United States (Markman et al., Yale Journal of Biology and Medicine, 64:583, 1991; McGuire et al., Ann. Intern, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes: Platinum coordination complexes are non-phase specific anti-cancer agents, which are interactive with DNA. The platinum complexes enter tumor cells, undergo, equation and form intra- and interstrand crosslinks with DNA causing adverse biological effects to the tumor. Examples of platinum coordination complexes include, but are not limited to, oxaliplatin, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma.

Alkylating agents: Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease.

Busulfan, 1,4-butanediol dimethanesulfonate, is commercially available as MYLERAN® TABLETS. Busulfan is indicated for the palliative treatment of chronic myelogenous leukemia.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease.

Antibiotic anti-neoplastics: Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma.

Doxorubicin, (8S, 10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas.

Topoisomerase II inhibitors: Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children.

Antimetabolite neoplastic agents: Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine).

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Other purine analogs include pentostatin, erythrohydroxynonyladenine, fludarabine phosphate, and cladribine.

Gemcitabine, 2′-deoxy-2′, 2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer.

Methotrexate, N-[4[[(2,4-diamino-6-pteridinyl) methyl]methylamino] benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder.

Topoisomerase I inhibitors: Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®. Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. Hormones and hormonal analogues: Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors: Signal transduction pathway inhibitors are those inhibitors, which block or inhibit a chemical process which evokes an intracellular change. As used herein this change is cell proliferation or differentiation. Signal transduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, ret, vascular endothelial growth factor receptor (VEGFr), tyrosine kinase with immunoglobulin-like and epidermal growth factor identity domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-lacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kinases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Anti-angiogenic agents: Anti-angiogenic agents including non-receptor MEKngiogenesis inhibitors may also be useful. Anti-angiogenic agents such as those which inhibit the effects of vascular endothelial growth factor, (for example the anti-vascular endothelial cell growth factor antibody bevacizumab [Avastin™], and compounds that work by other mechanisms (for example linomide, inhibitors of integrin αvβ3 function, endostatin and angiostatin);

Immunotherapeutic agents: Agents used in immunotherapeutic regimens may also be useful in combination with the compounds of formula (I). Immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies

Proapoptotoc agents: Agents used in proapoptotic regimens (e.g., bcl-2 antisense oligonucleotides) may also be used in the combination of the present invention.

Cell cycle signalling inhibitors: Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the combination of the present invention comprises an anti-OX40 ABP and a PD-1 modulator (e.g. anti-PD-1 ABP) and at least one anti-neoplastic agent selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine MEKngiogenesis inhibitors, immunotherapeutic agents, proapoptotic agents, and cell cycle signaling inhibitors.

In one embodiment, the combination of the present invention comprises an anti-OX40 ABP and a PD-1 modulator (e.g. anti-PD-1 ABP) and at least one anti-neoplastic agent which is an anti-microtubule agent selected from diterpenoids and vinca alkaloids.

In a further embodiment, the at least one anti-neoplastic agent agent is a diterpenoid.

In a further embodiment, the at least one anti-neoplastic agent is a vinca alkaloid.

In one embodiment, the combination of the present invention comprises an anti-OX40 ABP and a PD-1 modulator (e.g. anti-PD-1 ABP) and at least one anti-neoplastic agent, which is a platinum coordination complex.

In a further embodiment, the at least one anti-neoplastic agent is paclitaxel, carboplatin, or vinorelbine.

In a further embodiment, the at least one anti-neoplastic agent is carboplatin.

In a further embodiment, the at least one anti-neoplastic agent is vinorelbine.

In a further embodiment, the at least one anti-neoplastic agent is paclitaxel.

In one embodiment, the combination of the present invention comprises an anti-OX40 ABP and a PD-1 modulator (e.g. anti-PD-1 ABP) and at least one anti-neoplastic agent which is a signal transduction pathway inhibitor.

In a further embodiment the signal transduction pathway inhibitor is an inhibitor of a growth factor receptor kinase VEGFR2, TIE2, PDGFR, BTK, erbB2, EGFr, IGFR-1, TrkA, TrkB, TrkC, or c-fms.

In a further embodiment the signal transduction pathway inhibitor is an inhibitor of a serine/threonine kinase rafk, akt, or PKC-zeta.

In a further embodiment the signal transduction pathway inhibitor is an inhibitor of a non-receptor tyrosine kinase selected from the src family of kinases.

In a further embodiment the signal transduction pathway inhibitor is an inhibitor of c-src.

In a further embodiment the signal transduction pathway inhibitor is an inhibitor of Ras oncogene selected from inhibitors of farnesyl transferase and geranylgeranyl transferase.

In a further embodiment the signal transduction pathway inhibitor is an inhibitor of a serine/threonine kinase selected from the group consisting of PI3K.

In a further embodiment the signal transduction pathway inhibitor is a dual EGFr/erbB2 inhibitor, for example N-{3-Chloro-4-[(3-fluorobenzyl) oxy]phenyl}-6-[5-({[2-(methanesulphonyl) ethyl]amino}methyl)-2-furyl]-4-quinazolinamine (structure below):

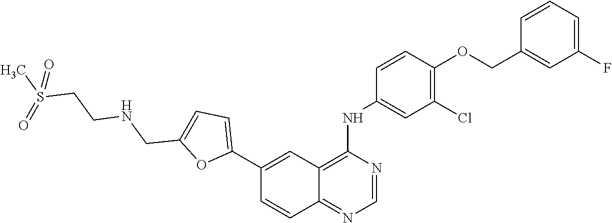 [SMILES: CS(=O)(=O)CCNCc1ccc(-c2ccc3ncnc(Nc4ccc(OCc5cccc(F)c5)c(Cl)c4)c3c2)o1]

In one embodiment, the combination of the present invention comprises a compound of formula I or a salt or solvate thereof and at least one anti-neoplastic agent which is a cell cycle signaling inhibitor.

In further embodiment, cell cycle signaling inhibitor is an inhibitor of CDK2, CDK4 or CDK6.

In one embodiment the mammal in the methods and uses of the present invention is a human.

As indicated, therapeutically effective amounts of the combinations of the invention (an anti-OX40 ABP and a PD-1 modulator (e.g. anti-PD-1 ABP)) are administered to a human. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attendant physician.

The following examples are intended for illustration only and are not intended to limit the scope of the invention in any way.

### EXAMPLES

### Example 1

An Open Label Dose Escalating Study of ANTIBODY 106-222 Administered Alone and in Combination with Anticancer Agents Including Pembrolizumab in Subjects with Selected Advanced Solid Tumors

The stimulation of antitumor T-cell activity, through inhibition of negative T-cell regulatory pathways with immunotherapeutic checkpoint inhibitors, has been very successful in the treatment of melanoma and non-small cell lung cancer (NSCLC). Another approach that provides an attractive target for the development of immunotherapy anticancer agents is the modulation of costimulatory pathways to enhance T-cell function. OX40 is a potent costimulatory receptor expressed primarily on activated CD4+ and CD8+ T cells. OX40 agonists have been shown to increase antitumor immunity and improve tumor-free survival in non-clinical models and OX40 agonist monoclonal antibodies (mAbs) are currently being evaluated in Phase I clinical trials. ANTIBODY 106-222 (described herein an antibody comprising CDRH1, CDRH2, and CDRH3 having the amino acid sequence as set forth in SEQ ID NOs 1, 2, and 3, and e.g. CDRL1, CDRL2, and CDRL3 having the sequences as set forth in SEQ ID NOs 7, 8, and 9, respectively and an antibody comprising VH region having an amino acid sequence as set forth in SEQ ID NO:5 and a VL region having an amino acid sequence as set forth in SEQ ID NO: 11) is a humanized wild-type immunoglobulin G1 (IgG1) anti-OX40 agonistic mAb (anti-human OX40 agonistic monoclonal antibody) and will be evaluated as a single-agent treatment in Part 1 of the current study. ANTIBODY 106-222 is also described, e.g. in WO 2012/027328 and in the Figures of the present application.

The anticancer immune response is a multistep process and it is expected that tumors may utilize redundant mechanisms to block the antitumor response; in these instances, combination therapies will likely be required. Combining an OX40 agonist with a programmed death receptor-1 (PD-1) inhibitor targets two different steps in the cancer-immunity cycle; OX40 agonism is expected to increase priming/activation of T cells, while inhibition of PD-1 blocks its interaction with programmed death ligand 1 (PD-L1) and programmed death ligand 2 (PD-L2), releasing the PD-1 pathway-mediated inhibition of the immune response. Based on non-clinical data, combination treatment with an OX40 agonist and a PD-1 inhibitor is anticipated to have synergistic antitumor activity, compared with single-agent treatment. The combination of ANTIBODY 106-222 with the PD-1 inhibitor pembrolizumab will be evaluated in Part 2 of the current study.

Objectives/Endpoints

The primary objectives of the study are to evaluate the safety and tolerability and to identify the maximum tolerated dose (MTD) or maximum administered dose (MAD) of ANTIBODY 106-222 when administered intravenously as monotherapy (Part 1) or in combination with pembrolizumab (Part 2) to subjects with selected advanced or recurrent solid tumors. Secondary objectives include: the evaluation of antitumor activity; characterization of pharmacokinetics (PK) for ANTIBODY 106-222 when administered alone; characterization of PK for ANTIBODY 106-222 and pembrolizumab when administered in combination; evaluation of pharmacodynamic activity in the blood and tumor microenvironment; and determination of the immunogenicity of ANTIBODY 106-222 when administered alone or for ANTIBODY 106-222 and pembrolizumab when administered in combination.
Safety endpoints: Adverse events (AEs), serious adverse events (SAEs), dose-limiting toxicity (DLT), withdrawals due to AEs, dose reductions or delays, and changes in safety assessments (e.g., laboratory parameters, vital signs, and cardiac parameters).
Antitumor activity endpoints: Objective response rate (ORR) and Disease Control Rate (DCR) (complete response [CR]+partial response [PR]+stable disease [SD]≥weeks), time to response, duration of response, progression-free survival (PFS), and overall survival (OS). Unless otherwise specified, all response endpoints will be assessed by Response Evaluation Criteria in Solid Tumors (RECIST) v1.1 and by irRECIST (immune-related RECIST).
PK endpoints: Plasma ANTIBODY 106-222 and serum pembrolizumab concentrations and PK parameters including maximum observed concentration (Cmax), area under the concentration-time curve over the dosing interval (AUC(0-τ), and minimum observed concentration (Cmin).
Pharmacodynamic endpoints: Assessment of lymphocyte OX40 receptor membrane expression and occupancy by ANTIBODY 106-222, along with the phenotype, quantity, and activation state of T cells in the periphery. Assessment of tumor biopsies via immunohistochemistry (IHC) for the numbers of tumor-infiltrating lymphocytes and other immune cells expressing key phenotypic markers.
Immunogenicity endpoints: Number and percentage of subjects who develop detectable antidrug antibodies (ADA).

Overall Design

This is a first time in human (FTIH), open-label, non-randomized, multicenter study designed to evaluate the safety, tolerability, PK, pharmacodynamics, and preliminary clinical activity of ANTIBODY 106-222 administered intravenously to subjects with selected advanced or recurrent solid tumors. The study will be conducted in 2 parts, each part consisting of a dose-escalation phase followed by a cohort expansion phase. Part 1 will evaluate ANTIBODY 106-222 monotherapy, while Part 2 will evaluate ANTIBODY 106-222 in combination with pembrolizumab. ANTIBODY 106-222 will first be evaluated as monotherapy in escalating doses. Once a dose of ANTIBODY 106-222 has been identified that is both tolerable and demonstrates pharmacodynamic activity, enrollment of Part 2 may begin. In Part 2, escalating doses of ANTIBODY 106-222 will be evaluated with fixed doses of pembrolizumab. The transition of the study from dose-escalation to cohort expansion and from monotherapy (Part 1) to combination therapy with pembrolizumab (Part 2) will be performed under the guidance of a Protocol Steering Committee. The remit, membership, roles, and responsibilities of the Steering Committee are described in a Steering Committee Charter. Pending a review of emerging data from this study and under the guidance of the Steering Committee, the protocol may be subsequently amended to include investigation of additional anticancer agent combinations with ANTIBODY 106-222.

Treatment Arms and Duration

The study includes a screening period, a treatment period, and a follow-up period. Subjects will be screened for eligibility beginning approximately 4 weeks before the start of treatment. The maximum duration of treatment with ANTIBODY 106-222 will be 48 weeks; the maximum duration of treatment with pembrolizumab will be 2 years. The follow-up period for safety assessments will be a minimum of 3 months from the date of the last dose. The post-treatment follow-up period includes disease assessments every 12 weeks until confirmed disease progression (PD). Following PD, subjects will be contacted every 3 months to assess survival status.

In Part 1, dose escalation for ANTIBODY 106-222 monotherapy will begin with a starting dose of 0.003 mg/kg ANTIBODY 106-222 administered once every 3 weeks (Q3W). In Part 2, dose escalation for ANTIBODY 106-222+pembrolizumab combination therapy will begin with a fixed dose of 200 mg pembrolizumab administered Q3W and a starting dose of ANTIBODY 106-222 that is two dose levels below a tolerated dose of ANTIBODY 106-222 monotherapy that has also demonstrated pharmacodynamic activity in Part 1A of the study. Dose adjustments are allowed to address tolerability and safety issues.

Type and Number of Subjects

The study will enroll up to approximately 180 subjects with tumor types that may include NSCLC, squamous cell carcinoma of the head and neck (SCCHN), renal cell carcinoma (RCC), melanoma, bladder cancer, soft tissue sarcoma (STS), triple-negative breast cancer (TNBC), and colorectal carcinoma displaying microsatellite instability (MSI CRC).

Analysis

During the dose-escalation phases of the study, safety, PK, and pharmacodynamic marker data will be examined while the study is being conducted in order to determine subsequent dosing levels. After each dosing cohort, a continual reassessment method (CRM) analysis may be used to recommend the next dose level based on observed DLTs.

In each expansion cohort, clinical activity, safety, PK, and pharmacodynamic marker data will be examined on an on-going basis and enrollment within each cohort may be curtailed or expanded in response to unfavorable or favorable outcomes. Tumor response data will be monitored and a tumor cohort may be terminated if there is insufficient evidence of clinical activity. The futility stopping rules are based on the methodology of Lee & Liu [Lee, 2008]. CRM-recommended dose-escalation levels, futility stopping rules, and posterior probabilities are only guidelines and the totality of the data will be considered by the team in decision making.

Study Rationale

The stimulation of antitumor T-cell activity, through inhibition of negative T-cell regulatory pathways with immunotherapeutic checkpoint inhibitors, has been very successful in the treatment of melanoma and NSCLC. Another approach that provides an attractive target for the development of immunotherapy anticancer agents is the modulation of costimulatory pathways to enhance T-cell function. OX40 is a potent costimulatory receptor expressed primarily on activated CD4+ and CD8+ T cells. OX40 agonists have been shown to increase antitumor immunity and improve tumor-free survival in non-clinical models and OX40 agonist mAbs are currently being evaluated in Phase I clinical trials. ANTIBODY 106-222 is a humanized wild-type IgG1 anti-OX40 agonistic mAb and will be evaluated as a single-agent treatment in Part 1 of the current study.

The anticancer immune response is a multistep process and it is expected that tumors may utilize redundant mechanisms to block the antitumor response; in these instances, combination therapies will likely be required. Combining an OX40 agonist with a PD-1 inhibitor targets two different steps in the cancer-immunity cycle; OX40 agonism is expected to increase priming/activation of T cells, while inhibition of PD-1 blocks its interaction with PD-L1 and PD-L2, releasing the PD-1 pathway-mediated inhibition of the immune response. Based on non-clinical data, combination treatment with an OX40 agonist and a PD-1 inhibitor is anticipated to have synergistic antitumor activity, compared with single-agent treatment. The combination of ANTIBODY 106-222 with the PD-1 inhibitor pembrolizumab will be evaluated in Part 2 of the current study.

This FTIH, open-label, dose-escalation study will assess the safety, PK, pharmacodynamics, and preliminary clinical activity of ANTIBODY 106-222 in subjects with selected advanced or recurrent solid tumors as monotherapy (Part 1), in combination with pembrolizumab (Part 2), and potentially in combination with additional therapies.

Brief Background

Immunotherapy has emerged as a transformative anticancer therapeutic strategy over the past few years. In particular, the inhibition of negative T-cell regulatory pathways with the checkpoint inhibitors has been very successful, first in the treatment of melanoma and, more recently, expanding to additional indications, including NSCLC. Ipilimumab and pembrolizumab are examples of these initial checkpoint inhibitors, which are mAbs that block the activity of the cytotoxic T-lymphocyte-associated antigen 4 (CTLA-4) and PD-1 pathways, respectively, thereby freeing the T-cell priming and T-cell effector functions from their negative regulatory effects.

In addition to regulatory mechanisms that negatively regulate effector T-cell function, costimulatory pathways are also attractive targets to modulate for the development of anticancer agents. OX40 (CD134) is a member of the tumor necrosis factor receptor (TNFR) family of transmembrane receptors, which is induced following antigen-dependent stimulation of both CD4+ and CD8+ T cells and following interaction with its cognate ligand (OX40L, expressed on activated antigen presenting cells) generally functions to transduce a costimulatory signal during the process of T-cell activation. In blood and peripheral tissues, OX40 expression is limited to the small subset of recently activated CD4+ and CD8+ cells; however, in tumors, infiltrating T lymphocytes are enriched for OX40 positive cells, where it functions to augment T-cell activation, proliferation, and survival through direct and indirect (e.g., cytokine release) mechanisms [Betting, 2009; Croft, 2010]. In addition to its function on effector T cells, OX40 is also expressed on tumor infiltrating regulatory T cells (Tregs), which tend to have an inhibitory effect on the immune response. Indeed, the efficacy of anti-OX40 antibodies in animal tumor models relies to some extent also on the depletion of tumor-specific Tregs residing in the tumor microenvironment [Bulliard, 2014; Marabelle, 2013]. As has been shown for anti-CTLA4 and anti-GITR (Glucocorticoid-induced TNFR family related gene) antibodies, this intratumoral Treg depletion is critical for the in vivo antitumor activity of immune checkpoint antibodies and is mediated by activatory FcγR positive myeloid cells residing in the tumor microenvironment [Bulliard, 2013; Selby, 2013; Simpson, 2013]. OX40 signaling has been shown to block the activity of induced Tregs, in part by blocking the release of the inhibitory cytokine interleukin-10 (IL-10), thereby further promoting effector T-cell immune responses [Ito, 2006]. Finally, OX40 can also be found on natural killer (NK) cells where it appears to stimulate NK-mediated antibody-dependent cellular cytotoxicity (ADCC) [Liu, 2008]. Together, these potential mechanisms of action make the stimulation of OX40 with agonistic agents an attractive target for a novel anti-cancer immunotherapy.

ANTIBODY 106-222

Background

ANTIBODY 106-222 is a humanized wild-type IgG1 anti-OX40 agonistic mAb. ANTIBODY 106-222 demonstrated several mechanisms of action in vitro including promoting effector CD4+ T-cell proliferation, inhibiting the induction of IL-10-producing CD4+ Type 1 regulatory (Tr1) cells and blocking the suppressive function of natural Tregs (nTregs), and binding to FcR, which is anticipated to augment OX40 signaling via cross-linking of the antibody via the Fc domain on FcR positive cells. Importantly, it has been shown that OX40 activation gives a costimulatory signal to T cells, dependent on a T-cell receptor (TCR) engagement, suggesting that ANTIBODY 106-222 is not a super agonist in the models tested.

ANTIBODY 106-222 is suitably cross-reactive to cynomolgus monkey OX40 to evaluate the pharmacology, pharmacodynamics, PK, and toxicology in this species. Single and repeat dose studies in cynomolgus monkeys demonstrated that ANTIBODY 106-222 bound to OX40 positive cells. ANTIBODY 106-222 is not cross-reactive with rodent OX40; however, a surrogate mAb to murine OX40 (OX86), was used to generate in vivo nonclinical evidence for both single agent efficacy and combination synergy with a variety of other immunotherapy agents in a range of syngeneic tumor models.

Nonclinical Pharmacokinetics of ANTIBODY 106-222

The nonclinical PK of ANTIBODY 106-222 has been investigated in mice following a single intraperitoneal (IP) administration and in cynomolgus monkeys following single and repeated intravenous (IV) administration.

The PK of ANTIBODY 106-222 in male mice following a single-dose IP administration had concentration profiles typical for mAbs (very slow plasma clearance and low volume of distribution at steady state) [Wang, 2008], suggesting that ANTIBODY 106-222 was mainly confined to the systemic circulation. Due to lack of cross-reactivity with murine OX40 the impact on PK expression of target is not evaluable in this species.

Similar PK profiles typical for mAbs were observed in monkeys. Following a single IV administration in male cynomolgus monkeys (n=3) at 2 mg/kg, all ANTIBODY 106-222-treated animals showed similar Cₘₐₓ values ranging from 31.8 to 39.9 μg/mL and similar systemic exposures (AUC₀₋₁₆₈ₕ) through Day 5. There was a dramatic change in clearance observed from about 7 to 14 days in all treated animals, typical of an immunogenicity response; all animals were confirmed to be positive for ADA in an ADA bridging assay.

Following repeat dose IV administration in cynomolgus monkeys at 10 or 100 mg/kg/week for 4 weeks, the mean AUC₀₋₁₆₈ₕ and Cₘₐₓ values for ANTIBODY 106-222 were similar between males and females at both doses during Weeks 1 and 4. The systemic exposure to ANTIBODY 106-222 (as defined by gender-averaged AUC₀₋₁₆₈ₕ and Cₘₐₓ values) increased dose-proportionally (n=3/group). The increases in the gender-averaged AUC₀₋₁₆₈ₕ and Cₘₐₓ values of ANTIBODY 106-222 from Week 1 to 4 ranged from 1.9- to 2.9 fold at both doses. Instances of decreased plasma concentrations were observed after the fourth dose in monkeys at 10 mg/kg/week due to primate ADA formation.

Nonclinical Safety of ANTIBODY 106-222

The toxicology program was conducted in cynomolgus monkeys. These monkeys were shown to be a suitable species based on OX40 receptor expression in tissues, orthologous protein sequence homology, and similar dose-dependent binding of ANTIBODY 106-222 for both human and monkey OX40 receptor on CD4+ T cells. IHC assessment of OX40 distribution in normal human tissues showed positive staining in cells or lymphoid cell aggregates, considered likely to be a subset of T cells, in a number of the tissues. These results are in general agreement with results from the evaluation of a microarray gene expression database (Gene Logic, Ocimum Biosolutions, LLC, Houston, Tex., USA).

ANTIBODY 106-222 was well tolerated in monkeys following weekly IV dosing for 4 weeks at doses up to 100 mg/kg/week.

ADA were observed in monkeys given 0 mg/kg/week; the incidence occurred inversely to dose. In a PK/pharmacodynamic study, all monkeys (n=3) or two of three monkeys given 2 or 10 mg/kg/week, respectively, demonstrated a dramatic increase in clearance as early as 7 days post-dose (2 mg/kg/week) and were confirmed to be positive for ADA. In monkeys given ANTIBODY 106-222 weekly for 4 weeks followed by a 6 week off-dose period, ADA were detected in two of ten monkeys given 10 mg/kg/week, in which one monkey (titer >10000) demonstrated a decrease in exposure following the fourth weekly dose, and the other monkey (titer=1000), which did not have detectable ADA until the off-dose period, did not demonstrate a clear association to decreased exposure. As ADA were only noted in animals maintained throughout the off-dose period, the ability to determine toxicity in the terminal necropsy animals at this dose on this study was not compromised by ADA. The generation of ADA in animals administered humanized protein is generally not predictive of a potential for ADA formation in humans.

In an in vivo syngeneic efficacy study with BALB/c mice bearing 4T1 mammary carcinoma cells, mortality and clinical observations consistent with anaphylaxis were observed in the majority of mice given ≥20 μg OX86 (rat wild-type IgG1 mAb against OX40 receptor) at 3 weeks of twice-weekly dosing (5^{th} dose). This effect appears to be unique to this specific model, as other efficacy studies using either BALB/c or C57BL/6 mice with the same batch of OX86 given at comparable doses and duration with other syngeneic cell types demonstrated no tolerability issues. Similar effects have been observed by others using 4T1 tumor-bearing mice given rat anti-PD-1 or hamster PD-L1 [Mall, 2014] or with tumor administration only [duPre, 2007]. To date, anaphylaxis has not been reported in subjects receiving OX40 agonist mAbs or approved PD-1 therapies [Curti, 2013; KEYTRUDA® Prescribing Information, 2014; OPDIVO® Prescribing Information, 2014]. Therefore, based upon the data and literature findings described above, it suggests that the anaphylaxis seen in 4T1 tumor-bearing mice given OX86 is model specific and that the proposed safety monitoring strategy (real time monitoring of ADA and acute hypersensitivity reactions, see Dose and Safety Management Guidelines Section) adequately addresses the potential risk of this effect.

The potential for ANTIBODY 106-222 to induce cytokine release has been investigated. In a human in vitro assay, using whole blood or isolated peripheral blood mononuclear cells (PBMCs) (with and without prior anti-CD3 and anti-CD28 stimulation), no release of cytokines in response to soluble or immobilized ANTIBODY 106-222 was observed. However, to further explore the potential for cytokine release, human PBMCs were incubated at higher (10×) cell density, incubated with immobilized ANTIBODY 106-222 and stimulated instead with submaximal levels of immobilized anti-CD3 (10 and 100× lower), to provide more sensitive assay conditions. In these assays increased cytokine production (IL-2, IFNγ, TNFα) was observed compared to anti-CD3 alone. Similar cytokine increases, along with proliferation, were also observed using isolated human CD4+ T cells with immobilized ANTIBODY 106-222, prior anti-CD3 and anti-CD28 stimulation to upregulate OX40 expression and longer incubation periods (48-72 hours compared with 24 hours). In repeat dose monkey studies up to 4 weeks in duration of dosing over a dose range of 0.03 to 100 mg/kg/week, there were no ANTIBODY 106-222-related changes in plasma cytokine levels at either 4 or 24 hours of the first dose or 4 hours after the second weekly dose. While low levels of cytokine release in subjects given ANTIBODY 106-222 is expected by activated T cells as part of the pharmacodynamics, the response may not be fully predicted by these in vitro assays, and close clinical monitoring is planned.

Single-dose safety pharmacology studies have not been conducted with ANTIBODY 106-222. Evaluations of cardiovascular function were performed on the 3^{rd} week of the 4-week monkey toxicology study, which evaluated heart rate, electrocardiogram (ECG) waveform evaluation, and corrected QT interval duration (QTc) evaluation. There were no ANTIBODY 106-222-related effects on these cardiovascular measurements nor were there any clinical observations of respiratory or general behavior effects of the antibody.

The no observed adverse effect level (NOAEL) was determined to be 100 mg/kg/week, the highest dose tested (Week 4 gender average mean AUC₀₋₁₆₈ₕ: 594 mg·h/mL, range 548 to 634 mg·h/mL; Cmax: 4.88 mg/mL, range 4.27 to 5.46 mg/mL).

Nonclinical Activity and Pharmacodynamics of ANTIBODY 106-222

In Vitro Studies

ANTIBODY 106-222 demonstrated several mechanisms of action in vitro, including promoting effector CD4+ T-cell proliferation, inhibiting the induction of IL-10 producing CD4+ Tr1 cells and blocking the suppressive function of nTregs, and binding to FcR, which is anticipated to augment OX40 signaling via cross-linking of the antibody via the Fc domain on FcR positive cells.

ANTIBODY 106-222 bound specifically to the recombinant OX40 extracellular domain from cynomolgus monkeys (Kd 408 nM) and humans (Kd 4.9 nM), but not to the related human receptors DcR3 and CD40. ANTIBODY 106-222 bound to both activated cynomolgus monkey and human CD4+ T cells with similar EC₅₀ values (0.35 and 0.30 μg/mL, respectively). These data suggest that affinity differences observed for binding to recombinant OX40 are not truly reflective of the binding to cell-surface OX40.

Many anti-TNFR family antibodies (including anti-OX40) appear to require the formation of high-density antibody complexes and costimulation which may occur in vivo during cell:cell interactions in tissues expressing various FcγRs [White, 2013]. In the case of OX40 antibodies, this can be mimicked in vitro by immobilizing the antibody to the surface of plastic tissue-culture plates and incubating cells on this plate-bound antibody. Importantly, it was shown that OX40 activation gives a costimulatory signal to T cells dependent on TCR engagement (e.g., CD3 ligation), suggesting that ANTIBODY 106-222 is not a super agonist in the in vitro systems tested in absence of TCR signal. ANTIBODY 106-222 (immobilized) stimulated proliferation of immobilized anti-CD3 activated cynomolgus monkey CD4+ T cells with a mean EC₅₀ value of 0.72 μg/mL (4.8 nM) and anti-CD3 induced proliferation of activated human CD4+ T cells with a mean EC₅₀ value of 0.19 μg/mL (1.3 nM).

OX40 agonist antibodies have been shown to reduce the suppressive function of human Tregs. Human purified CD4+ T cells were differentiated into induced Tregs using vitamin D3 and dexamethasone and cultured with human CD32a (FcγRIIA)-expressing L-cells (which could facilitate antibody crosslinking via the FcγRIIA). Addition of ANTIBODY 106-222 in solution during the differentiation phase was able to prevent naïve T cells from differentiating into IL-10⁺ Tr1 cells.

As expected for an IgG1 antibody, ANTIBODY 106-222 bound to cynomolgus monkey and human FcRγs (and to human complement C1q) and showed low but measurable levels of reporter FcγRIIIA engagement in a reporter assay system. In ADCC assays, some cell lysis of an OX40+ target cell line was observed with ANTIBODY 106-222 treatment. However, in primary human PBMC assays, ANTIBODY 106-222 generally did not impact the viability of CD4+ and CD8+ T cells. Statistical analysis of the PBMC ADCC data did not support a robust effect on viability with ANTIBODY 106-222. In all cases the reduction in viability with ANTIBODY 106-222 was less than observed for anti-CD52 or anti-CD20 positive control antibodies. Overall these in vitro findings suggest that ANTIBODY 106-222 may have the potential to cause ADCC of OX40+ target cells in vivo, however effects were not consistent across donors and these in vitro assays may not fully reflect the immune microenvironments in vivo.

In Vivo Studies

In cynomolgus monkeys given a single IV dose (2 mg/kg) of ANTIBODY 106-222 there was a transient decrease in the percentage of OX40+/CD4+ T cells on Day 2, recovering by Day 7, suggesting that ANTIBODY 106-222 appears to bind to OX40+ cells in peripheral blood and that these cells and may reflect margination rather than depletion of these cells. In a repeat-dose IV study, cynomolgus monkeys were given ANTIBODY 106-222 (10 mg/kg/week) for 4 weeks. As observed in the single-dose study, the percentage of free OX40 on peripheral blood T cells was reduced in groups treated with ANTIBODY 106-222 compared with the vehicle group, which was sustained for the duration of the study. This indirectly suggests that ANTIBODY 106-222 was bound to OX40+ cells following dosing. OX40-positive cells did not appear to be depleted as they could be detected using a non-competitive anti-OX40 antibody. No clear evidence of changes in T-cell activation markers were observed in peripheral blood, spleen, or lymph nodes in treated groups compared with non-treated groups in either study. OX40 positive cells were also detected in these tissues in both treated and non-treated groups suggesting cells were not depleted in tissues by ANTIBODY 106-222. There were no clinical observations considered related to treatment in either study.

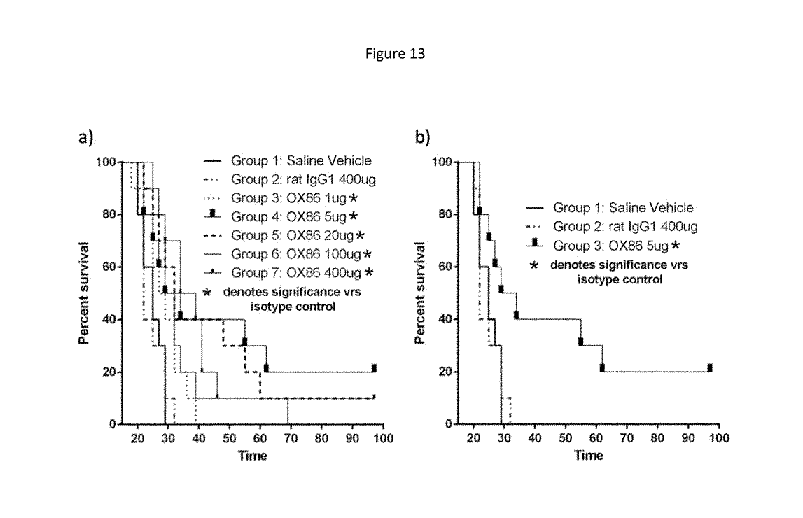

A surrogate mAb to murine OX40 (OX86) was used to generate in vivo nonclinical evidence for monotherapy activity in syngeneic tumor models. In a series of experiments, female BALB/c mice bearing CT26 mouse colon carcinoma tumors (n=10/group), were given twice weekly IP doses of OX86 ranging from 1 to 400 μg/mouse in phosphate-buffered saline for 3 weeks. All doses showed a significant increase in survival compared to control groups (FIGS. 13 a and b). Assuming similar potency between anti-OX86 and ANTIBODY 106-222, exposures that have demonstrated efficacy preclinically are predicted to be achievable in human subjects.

Additional in vivo experiments were performed with BALB/c mice bearing A20 mouse lymphoma cell line tumors and showed modest tumor reduction and with C57BL/6 mice bearing B16F10 mouse melanoma cell line tumors with no significant effect on tumor reduction or survival noted. Additionally, the mouse adoptive cell transfer (ACT) model, MC38/gp100 was utilized to evaluate ANTIBODY 106-222 in vivo since a humanized mouse model is unavailable. Overall the ACT model was not robust and produced highly variable results.

In vivo studies in syngeneic tumor models were also performed with ANTIBODY 106-222 in combination with anti-PD-1 antibodies and anti-CTLA-4 antibodies; the anti-PD-1 combination studies are briefly described below.

Pembrolizumab

Refer to the pembrolizumab IB/approved labeling for detailed background information on pembrolizumab [KEYTRUDA® Prescribing Information, 2014; Merck Sharp & Dohme Corp, 2014].

PD-1 as a Therapeutic Target

The PD-1 receptor-ligand interaction is a major pathway hijacked by tumors to suppress immune control [Pedoeem, 2014]. The normal function of PD-1, expressed on the cell surface of activated T cells under healthy conditions, is to down-modulate unwanted or excessive immune responses, including autoimmune reactions. PD-1 (encoded by the gene Pdcd1) is an Ig superfamily member related to CD28 and CTLA-4, which has been shown to negatively regulate antigen receptor signaling upon engagement of its ligands (PD-L1 and/or PD-L2). The structures of murine PD-1 alone [Zhang, 2004] and in complex with its ligands were first resolved [Lazar-Molnar, 2008; Lin, 2008], and more recently the NMR-based structure of the human PD-1 extracellular region and analyses of its interactions with its ligands were also reported [Cheng, 2013]. PD-1 and family members are type I transmembrane glycoproteins containing an Ig Variable-type (V-type) domain responsible for ligand binding and a cytoplasmic tail which is responsible for the binding of signaling molecules. The cytoplasmic tail of PD-1 contains 2 tyrosine-based signaling motifs, an immunoreceptor tyrosine-based inhibition motif (ITIM) and an immunoreceptor tyrosine-based switch motif (ITSM). Following T-cell stimulation, PD-1 recruits the tyrosine phosphatases SHP-1 and SHP-2 to the ITSM motif within its cytoplasmic tail, leading to the dephosphorylation of effector molecules, such as CD3, PKC and ZAP70, which are involved in the CD3 T-cell signaling cascade [Sheppard, 2004]. The mechanism by which PD-1 down-modulates T-cell responses is similar to, but distinct from that of CTLA-4 [Ott, 2013]. PD-1 was shown to be expressed on activated lymphocytes, including peripheral CD4+ and CD8+ T cells, B cells, Tregs and NK cells [Yao, 2014]. Expression has also been shown during thymic development on CD4-CD8− (double negative) T cells [Nishimura, 1996], as well as subsets of macrophages [Huang, 2009] and dendritic cells [P\tilde{e}a-Cruz, 2010]. The ligands for PD-1 (PD-L1 and PD-L2) are constitutively expressed or can be induced in a variety of cell types [Keir, 2008]. PD-L1 is expressed at low levels on various non-hematopoietic tissues, most notably on vascular endothelium, whereas PD-L2 protein is only detectably expressed on antigen-presenting cells found in lymphoid tissue or chronic inflammatory environments [Keir, 2008]. Both ligands are type I transmembrane receptors containing both IgV- and IgC-like domains in the extracellular region and short cytoplasmic regions with no known signaling motifs. Binding of either PD-1 ligand to PD-1 inhibits T-cell activation triggered through the T-cell receptor. PD-L2 is thought to control immune T-cell activation in lymphoid organs, whereas PD-L1 serves to dampen unwarranted T-cell function in peripheral tissues. Although healthy organs express little (if any) PD-L1, a variety of cancers were demonstrated to express abundant levels of this T-cell inhibitor [Karim, 2009, Taube, 2012], which, via its interaction with the PD-1 receptor on tumor-specific T cells, plays a critical role in immune evasion by tumors [Sanmamed, 2014]. As a consequence, the PD-1/PD-L1 pathway is an attractive target for therapeutic intervention in cancer [Topalian, 2012].

Pembrolizumab Background and Clinical Trials

Pembrolizumab [KEYTRUDA® (US); previously known as lambrolizumab, MK-3475 and SCH 9000475] is a potent and highly selective humanized mAb of the IgG4/kappa isotype designed to directly block the interaction between PD-1 and its ligands, PD-L1 and PD-L2. Pembrolizumab was recently approved in the US and is indicated for the treatment of subjects with unresectable or metastatic melanoma and disease progression following ipilimumab and, if BRAF V600 mutation positive, a BRAF inhibitor [KEYTRUDA® Prescribing Information, 2014; Poole, 2014]. It is the first anti-PD-1 therapy to receive regulatory approval in the US, and is currently under regulatory review in the EU.

Ongoing clinical trials are being conducted in advanced melanoma, NSCLC, head and neck cancer, urothelial cancer, gastric cancer, TNBC, Hodgkin's lymphoma and a number of other advanced solid tumor indications and hematologic malignancies. For study details please refer to the IB [Merck Sharp & Dohme Corp, 2014].

Rationale for Pembrolizumab Dose Selection

An open-label Phase I trial (KEYNOTE-001) is being conducted to evaluate the safety and clinical activity of single agent pembrolizumab. The dose escalation portion of this trial evaluated three dose levels, 1 mg/kg, 3 mg/kg, and 10 mg/kg, administered every 2 weeks (Q2W) in subjects with advanced solid tumors. All three dose levels were well tolerated and no dose-limiting toxicities were observed. This first in human study of pembrolizumab showed evidence of target engagement and objective evidence of tumor size reduction at all dose levels (1 mg/kg, 3 mg/kg and 10 mg/kg Q2W). No MTD has been identified.

In KEYNOTE-001, two randomized cohort evaluations of melanoma subjects receiving pembrolizumab at a dose of 2 mg/kg versus 10 mg/kg Q3W have been completed, and one randomized cohort evaluating of 10 mg/kg Q3W versus 10 mg/kg Q2W has also been completed. The clinical efficacy and safety data demonstrate a lack of clinically important differences in efficacy response or safety profile at these doses. For example, in Cohort B2, advanced melanoma subjects who had received prior ipilimumab therapy were randomized to receive pembrolizumab at 2 mg/kg versus 10 mg/kg Q3W. The ORR was 26% (21/81) in the 2 mg/kg group and 26% (20/76) in the 10 mg/kg group [Robert, 2014]. The proportion of subjects with drug-related AE, grade 3-5 drug-related AE, serious drug-related AE, death or discontinuation due to an AE was comparable between groups or lower in the 10 mg/kg group. In Cohort B3, advanced melanoma subjects (irrespective of prior ipilimumab therapy) were randomized to receive pembrolizumab at 10 mg/kg Q2W versus 10 mg/kg Q3W. The ORR was 30.9% (38/123) in the 10 mg/kg Q2W group and 24.8% (30/121) in the 10 mg/kg Q3W group. The proportion of subjects with drug-related AE, grade 3-5 drug-related AE, serious drug-related AE, death or discontinuation due to an AE was comparable between groups.

PK data analysis of pembrolizumab administered Q2W and Q3W showed slow systemic clearance, limited volume of distribution, and a long half-life [Merck Sharp & Dohme Corp, 2014]. Pharmacodynamic data (IL-2 release assay) suggested that peripheral target engagement is durable (>21 days). This early PK and pharmacodynamic data provides scientific rationale for testing a Q3W dosing schedule. Because Q3W dosing is more convenient for subjects, Q3W dosing will be further studied.

The rationale for further exploration of 2 mg/kg and comparable doses of pembrolizumab in solid tumors is based on: 1) similar efficacy and safety of pembrolizumab when dosed at either 2 mg/kg or 10 mg/kg Q3W in melanoma subjects, 2) the flat exposure-response relationships of pembrolizumab for both efficacy and safety in the dose ranges of 2 mg/kg Q3W to 10 mg/kg Q3W, 3) the lack of effect of tumor burden or indication on distribution behavior of pembrolizumab (as assessed by the population PK model) and 4) the assumption that the dynamics of pembrolizumab target engagement will not vary meaningfully with tumor type.

The choice of the 200 mg Q3W fixed dosing regimen is based on simulations performed using the population PK model of pembrolizumab showing that the fixed dose of 200 mg every 3 weeks will provide exposures that 1) are optimally consistent with those obtained with the 2 mg/kg dose every 3 weeks, 2) will maintain individual subject exposures in the exposure range established in melanoma as associated with maximal efficacy response and 3) will maintain individual subjects exposure in the exposure range established in melanoma that are well tolerated and safe.

A fixed dose regimen will also be simpler and more convenient for physicians and to reduce potential for dosing errors. A fixed dosing scheme will reduce complexity in the logistical chain at treatment facilities and reduce wastage.

Rationale for OX40 Agonist and PD-1 Inhibitor Combination

The anticancer immune response is a multistep process that includes antigen processing and presentation, T-cell priming and activation, tumor infiltration, and subsequent destruction by activated effector T cells [Chen, 2013]. Each of these steps can be negatively regulated, which provides the malignant tumor with redundant mechanisms by which to block an anticancer immune response. In some cases, tumors will be highly dependent on a single mechanism, and in these cases, there is the potential to achieve significant clinical activity with a single immunomodulatory therapy. However, it is expected that tumors will often utilize redundant mechanisms to block the antitumor immune response. In these instances, combination therapies will likely be required. A recently described example of the benefit of combination immunotherapy is the clinical data generated by the combination of ipilimumab (anti-CTLA-4) and nivolumab (anti-PD-1) in subjects with metastatic melanoma [Wolchok, 2013].

The rationale for combining an OX40 agonist with an anti-PD-1 agent is based on the fact that these two agents target different steps in the cancer-immunity cycle. Similar to the ipilimumab/nivolumab combination, ANTIBODY 106-222 is expected to increase the priming/activation of antitumor T cells while the anti-PD-1 agent pembrolizumab prevents the inhibitory effect of the PD-1/PD-L1 pathway on effector T cells in the tumor. Recently, Guo et al reported synergistic antitumor activity for the combination of PD-1 blockade and OX40 agonism in a murine ID8 ovarian cancer model. The activity of the combination treatment was associated with increased CD4+ and CD8+ cells and decreased CD4+FoxP3+ Tregs and CD11b+Gr-1+ myeloid suppressor cells [Guo, 2014].

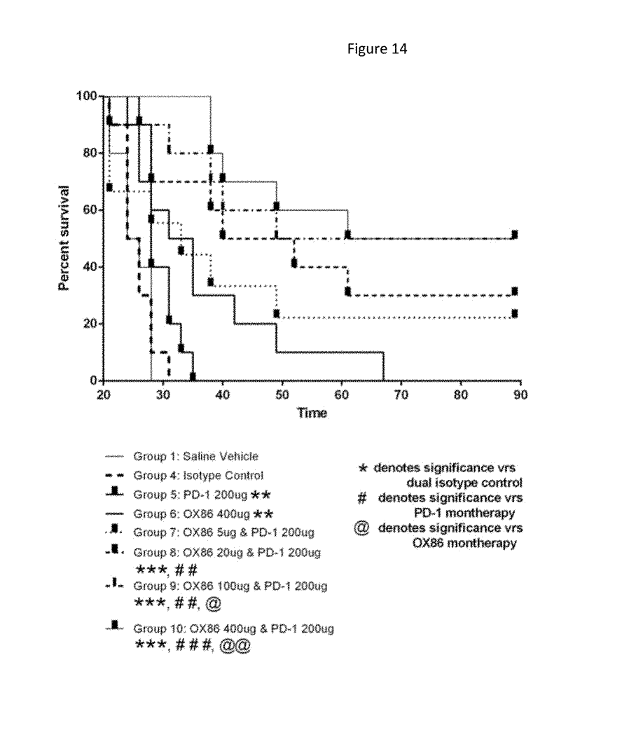

A surrogate mAb to murine OX40 (OX86) was used to generate in vivo nonclinical evidence for combination synergy with a PD-1 inhibitor in syngeneic tumor models. In a series of experiments, female BALB/c mice bearing CT26 mouse colon carcinoma cell line tumors (n=10/group) were given twice weekly IP dosing with OX86 at 1 to 400 μg/mouse and in combination with anti-PD-1 mAb at 20 or 200 μg/mouse for 4 weeks. Both OX86 and anti-PD-1 mAb monotherapy decreased tumor volume and increased survival compared with saline and isotype controls; however, the combination of OX86/anti-PD-1 significantly increased survival compared with monotherapy and was well tolerated (FIG. 14). Similar combination efficacy and survival outcomes were reported for female BALB/c mice bearing A20 mouse lymphoma cell line tumors; however, no synergy or additive effects were reported in female C57BL/6 mice bearing B16F10 mouse melanoma cell line tumors.

Objectives and Endpoints

| | |
|---|---|
| Objectives | Endpoints |

Part 1: ANTIBODY 106-222 Monotherapy
Primary

| | |
|---|---|
| To evaluate the safety and | AEs, SAEs, DLT, withdrawals |
| tolerability and identify the | due to AEs, dose reductions |
| MTD^{a} or the MAD of ANTIBODY 106- | or delays, and changes in |
| 222 administered intravenously | safety assessments (e.g., |
| to subjects with selected advanced | laboratory parameters, vital |
| or recurrent solid tumors. | signs, and cardiac parameters). |

Secondary

| | |
|---|---|
| To evaluate the antitumor activity | ORR and DCR (CR+ PR+ |
| of ANTIBODY 106-222 in subjects | SD ≥12 weeks), time to response, |
| with selected advanced or | duration of response, PFS, and OS.^{b} |
| recurrent solid tumors. | ANTIBODY 106-222 concentrations |
| To characterize the PK of | in plasma and PK parameters |
| ANTIBODY 106-222 monotherapy. | including Cmax, AUC(0-τ), and |
| | Cmin. |
| To evaluate the pharmacodynamic | Assessment of lymphocyte OX40 |
| activity of ANTIBODY 106-222 in | receptor membrane expression |
| the periphery, i.e., blood and in the | and occupancy by ANTIBODY |
| tumor microenvironment. | 106-222, along with the |
| | phenotype, quantity, and |
| | activation state of T cells in |
| | the periphery. |
| | Assessment of tumor biopsies |
| | via IHC for the numbers of |
| | tumor-infiltrating lymphocytes |
| | and other immune cells |
| | expressing key phenotypic markers. |
| To determine the immunogenicity of | Number and percentage of subjects |
| ANTIBODY 106-222. | who develop detectable ADA. |

Exploratory

| | |
|---|---|
| To explore the relationship between | Evaluation of antitumor activity |
| antitumor activity, PK parameters, | (CR, PR, SD, PD), PK parameters, |
| and pharmacodynamic response | and pharmacodynamic activity. |
| after treatment with ANTIBODY | |
| 106-222. | |
| To explore the association between | Correlation between antitumor |
| treatment with ANTIBODY 106-222 | activity and expression of immune |
| and changes in genomic DNA, | related genes, e.g., but not |
| gene expression (RNA and | limited to TCR sequences and gene |
| protein), measures of immune | signatures, along with the |
| function in tissue and blood and | expression of circulating soluble |
| antitumor activity. | factors such as cytokines and |
| | stress-related proteins in both |
| | tissues and blood. |
| To explore the immune response | Determination of the correlation |
| biomarkers in tumor tissue and their | between the immune response in |
| association with the antitumor | tumor samples and antitumor |
| activity with ANTIBODY 106-222. | activity of ANTIBODY 106-222 to |
| | identify potential selection |
| | biomarkers for subject enrichment. |
| Pharmacogenetics (PGx): To | Germline genetic evaluations may |
| evaluate the association of genetic | be conducted for: |

| | |
|---|---|
| variations in the host DNA and | Medicine response, including |
| response to therapy. | ANTIBODY 106-222 or any |
| | concomitant medicines. |
| | Disease susceptibility, |
| | severity, and progression and |
| | related conditions. |

Hypothesis
For the primary objective, no formal statistical hypothesis will be tested;
analysis will be descriptive and exploratory.
^{a}In the final determination of the MTD, all available safety and tolerability data will be considered
^{b}Unless otherwise specified, all response endpoints will be assessed by Response Evaluation Criteria in Solid Tumors (RECIST) v1.1 and by irRECIST (immune-related RECIST); irRECIST will be used to determine treatment decisions.
RNA = Ribonucleic acid;
DNA = Deoxyribonucleic acid

| | |
|---|---|
| Objectives | Endpoints |

PART 2: Combination ANTIBODY 106-222 plus pembrolizumab
Primary

| | |
|---|---|
| To evaluate the safety and | AEs, SAEs, DLTs, withdrawals |
| tolerability and identify the | due to AEs, dose reductions or |
| MTD^{a} or the MAD of ANTIBODY 106- | delays, and changes in safety |
| 222 administered intravenously | assessments (e.g., laboratory |
| in combination with IV | parameters, vital signs, and |
| pembrolizumab to subjects with | cardiac parameters). |
| selected advanced or recurrent | |
| solid tumors. | |

Secondary

| | |
|---|---|
| To evaluate the antitumor | ORR and DCR (CR+ PR+ |
| activity of ANTIBODY 106-222 in | SD ≥12 weeks), time to |
| combination with pembrolizumab | response, duration of response, |
| in subjects with selected | PFS, and OS.^{b} |
| advanced or recurrent solid | |
| tumors. | |
| To characterize the PK of | Plasma ANTIBODY 106-222 and |
| ANTIBODY 106-222 and | serum pembrolizumab |
| pembrolizumab when | concentrations and PK |
| administered in combination. | parameters including Cmax, |
| | AUC(0-τ), and Cmin. |
| To evaluate the | Assessment of lymphocyte OX40 |
| pharmacodynamic activity of | receptor membrane expression |
| ANTIBODY 106-222 in combination | and occupancy by ANTIBODY |
| with pembrolizumab in the | 106-222, along with the |
| periphery, i.e., blood and | phenotype, quantity, and |
| in the tumor microenvironment. | activation state of T cells in |
| | the periphery. |
| | Assessment of tumor biopsies via |
| | IHC for the numbers of tumor- |
| | infiltrating lymphocytes and |
| | other immune cells expressing |
| | key phenotypic markers. |
| To determine the immunogenicity | Number and percentage of |
| of ANTIBODY 106-222 and | subjects who develop detectable |
| pembrolizumab when | ADA. |
| administered in combination. | |

Exploratory

| | |
|---|---|
| To explore the relationship | Evaluation of antitumor activity |
| between antitumor activity, PK | (CR, PR, SD, PD), PK |
| parameters, and | parameters, and |
| pharmacodynamic response after | pharmacodynamic activity. |
| treatment with ANTIBODY 106- | |
| 222 in combination with | |
| pembrolizumab. | |
| To explore the association | Correlation between antitumor |
| between treatment with | activity and expression of |
| ANTIBODY 106-222 in | immune-related genes, e.g., but |
| combination with pembrolizumab | not limited to TCR sequences and |
| and changes in genomic DNA, | gene signatures, along with the |
| gene expression (RNA and | expression of circulating soluble |
| protein), measures of immune | factors such as cytokines and |
| function in tissue and blood | stress related proteins in both |
| and antitumor activity. | tissues and blood. |
| To explore the immune response | Determination of the correlation |
| biomarkers in tumor tissue and | between the immune response in |
| their association with the | tumor samples and antitumor |
| antitumor activity with | activity of ANTIBODY 106-222 to |
| ANTIBODY 106-222 in combination | identify potential selection |
| with pembrolizumab. | biomarkers for subject |
| | enrichment. |
| PGx: To evaluate the association | Germline genetic evaluations may |
| of genetic variations in the host | be conducted for: |

| | |
|---|---|
| DNA and response to therapy. | Medicine response, including |
| | GSK317499 and |
| | pembrolizumab or any |
| | concomitant medicines. |
| | Disease susceptibility, |
| | severity, and progression and |
| | related conditions. |

Hypothesis
For the primary objective, no formal statistical hypothesis will be tested;
analysis will be descriptive and exploratory.
^{a}In the final determination of the MTD, all available safety and tolerability data will be considered
^{b}Unless otherwise specified, all response endpoints will be assessed by RECIST v1.1 and by irRECIST; irRECIST will be used to determine treatment decisions.
RNA = Ribonucleic acid;
DNA = Deoxyribonucleic acid

Study Design

Overall Design

This is a FTIH, open-label, non-randomized, multicenter study designed to evaluate the safety, tolerability, PK, pharmacodynamics, and preliminary clinical activity of ANTIBODY 106-222 administered intravenously to subjects with selected advanced or recurrent solid tumors.

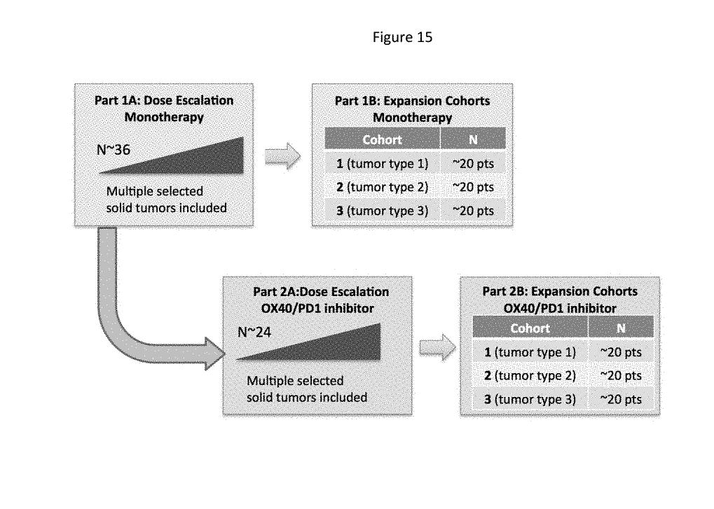

The study will be conducted in 2 parts, each part consisting of a dose-escalation phase followed by a cohort expansion phase (see FIG. 15). Part 1 will evaluate ANTIBODY 106-222 monotherapy, while Part 2 will evaluate ANTIBODY 106-222 in combination with pembrolizumab. As shown in FIG. 15, ANTIBODY 106-222 will first be evaluated as monotherapy in escalating doses. Once a dose of ANTIBODY 106-222 has been identified that is both tolerable and demonstrates pharmacodynamic activity, enrollment of Part 2 may begin. In Part 2, escalating doses of ANTIBODY 106-222 will first be evaluated with fixed doses of pembrolizumab. Each part will also include expansion cohorts for up to three different tumor types.

The study will enroll up to approximately 180 subjects with tumor types that may include NSCLC, SCCHN, RCC, melanoma, bladder cancer, STS, TNBC, and MSI CRC. In the dose-escalation phase of the study, subjects with any of the aforementioned tumor types may be included; whereas in the cohort expansion phase of the study, each expansion cohort will enroll subjects with one specific tumor type selected from the aforementioned list. Up to three expansion cohorts may be included for each part of the study.

A subject's disease status and determination of disease progression at postbaseline visits will be evaluated by the local investigators' assessments of radiology by RECIST v1.1 and irRECIST; a decision to discontinue treatment due to disease progression will be based upon irRECIST; however, the primary endpoint analysis will use RECIST v1.1. Scans will be collected centrally and stored to allow for the option of central radiologic audit or review.

A Steering Committee will be established to review safety, PK, and other clinical data during the course of the study, to provide objective interpretation of study results, and guidance for key decisions. The remit of the Steering Committee will include guidance for the transition of the study from dose-escalation to cohort expansion and from Part 1 to Part 2, the selection of specific tumor types to include in the expansion cohorts, and the selection of the recommended Phase 2 dose (RP2D); the study team will also seek endorsement from GSK Medical Governance for the transition of the study from one part to another. In the final determination of the MTD and RP2D, all available safety and tolerability data will be considered. Pending a review of emerging data from this study and under the guidance of the Steering Committee, the protocol may be subsequently amended to include investigation of additional anticancer agent combinations with ANTIBODY 106-222. The remit, membership, roles and responsibilities of the Steering Committee are described in a Steering Committee Charter. Key decisions of the Steering Committee will be documented and reported to all participating principal investigators (PIs) and Institutional Review Boards (IRBs)/Independent Ethics Committees (IECs).

Dose Escalation

For the first two dose levels (see Table 2), an accelerated titration design is planned with one subject enrolled at each of these dose levels. Each subject must complete the 4 week DLT evaluation period and the available safety data must be reviewed before a decision is made on whether to proceed to the next dose level. If a subject experiences a DLT, then this will trigger the implementation of the modified 3+3 design as shown in Table 2. If a subject withdraws from the study before the completion of the 4 week DLT evaluation period for reasons other than DLT, the subject will be replaced.

For subsequent dose levels, a modified 3+3 design will be used for dose escalation as shown in Table 2. The first three subjects treated at the third dose level will begin treatment 1 week apart to allow assessment of initial safety data in each subject before beginning the next subject's treatment. Evaluation of the available safety data over the first 4 weeks of treatment is required from at least 3 subjects before a decision is made whether to enroll additional subjects at the same, or the next higher dose level. Subjects who withdraw from the study before the completion of 4 weeks treatment for reasons other than DLT may be replaced. After the third dose level cohort is completed, subsequent dose levels may initially enroll up to 4 subjects and subjects will begin treatment at least 24 hours apart.

If 1 of 3 (or 1 of 4) subjects experiences a DLT at a particular dose level, additional subjects will be enrolled at that dose level so that a total of 6 subjects are treated at that dose level. If at least 2 of 6 subjects experience a DLT at a particular dose level, a lower (or intermediate) dose level may be explored to better define the MTD. The Steering Committee may propose that a given dose-escalation cohort be expanded up to a total of 12 subjects if (i) further evaluation of the frequency of a given toxicity is warranted, based upon the observed safety profile in the 6 subjects already recruited in the cohort or (ii) further evaluation of pharmacodynamic markers to aid dose selection is warranted; in either case, the incidence of confirmed DLT must not exceed 33%. Dose-escalation decisions will be documented in writing with copies maintained at each site and the study files.

TABLE 2
3 + 3 Dose-Escalation Guidelines

| Number of Subjects | |
|---|---|
| with DLT at a | |
| Given Dose Level | Action^{a} |
| 0 out of 3 subjects | Escalate to next dose level and enter 3 subjects |
| 1 out of 3 subjects | Accrue 3 additional evaluable subjects at current |
| | dose level for a total of 6 evaluable subjects |

If 0 of the 3 additional subjects experience
a DLT, proceed to next dose level.
If 1 or more of the additional subjects
experience a DLT, the dose escalation is
stopped and this dose is declared the MTD.

| | |
|---|---|
| 1 out of 6 subjects | Escalate to next dose level |
| 2 or more subjects in a | Dose escalation will be stopped. At this dose |
| dosing cohort (up to 6 | level, the MTD has been exceeded (highest dose |
| subjects) | administered). |

^{a}The Steering Committee may propose that a given dose-escalation cohort be expanded up to a total of 12 subjects if (i) further evaluation of the frequency of a given toxicity is warranted, based upon the observed safety profile in the 6 subjects already recruited in the cohort or (ii) further evaluation of pharmacodynamic markers to aid dose selection is warranted; in either case, the incidence of confirmed DLT must not equal or exceed 33%.
DLT = Dose-limiting toxicity;
MTD = Maximum tolerated dose

Part 1A: Monotherapy Dose Escalation

Dose escalation for ANTIBODY 106-222 monotherapy will begin with a starting dose of 0.003 mg/kg ANTIBODY 106-222 administered Q3W (see Dose Justification Section). Table 3 illustrates the maximum dose that may be selected for each dose level increase. The maximum increase in dose is 3.33-fold or less. Dose levels intermediate to those in Table 3, or schedules other than once every three weeks may be explored if exposure is significantly higher than predicted, if there is excessive toxicity, or if further evaluation of pharmacodynamic markers to aid dose selection is warranted.

TABLE 3
Part 1A Dose Levels

| Dose Level | ANTIBODY 106-222 (mg/kg)^{a} |
|---|---|
| 1 | 0.003 |
| 2 | 0.01 |
| 3 | 0.03 |
| 4 | 0.1 |
| 5 | 0.3 |
| 6 | 1.0 |
| 7 | 3.0 |
| 8 | 10.0 |

^{a}Lower dose intensities may be explored if exposure is significantly higher than predicted, if there is excessive toxicity, or if further evaluation of pharmacodynamic markers to aid dose selection is warranted. This may be achieved by reducing the dose or by alternate dosing schedules.

Part 2A: Combination Dose Escalation (ANTIBODY 106-222+Pembrolizumab)

Dose escalation for ANTIBODY 106-222+pembrolizumab combination therapy will begin with a fixed dose of 200 mg pembrolizumab administered Q3W and a starting dose of ANTIBODY 106-222 that is at least 2 dose levels below a tolerated dose of ANTIBODY 106-222 monotherapy that has also demonstrated pharmacodynamic activity in Part 1A of the study. An example of potential combinations of ANTIBODY 106-222 and pembrolizumab is described in Table 4. In this example, a dose of 1 mg/kg ANTIBODY 106-222 alone was tolerated in at least 3 subjects in Part 1A of the study.

TABLE 4
Example of Part 2A Dose Levels

| Dose Level | ANTIBODY 106-222 (mg/kg) | Pembrolizumab (mg) |
|---|---|---|
| 1 | 0.1 | 200 |
| 2 | 0.3 | 200 |
| 3 | 1.0 | 200 |
| 4 | 3.0 | 200 |
| 5 | 10.0 | 200 |

If the combination doses in the starting dose cohort of Part 2A are not tolerable, lower doses of ANTIBODY 106-222 may be evaluated in combination with 200 mg pembrolizumab. The dose of pembrolizumab will remain fixed at 200 mg throughout the study.

Dose escalation will proceed until the MTD of the combination regimen is identified, as described in Dose Escalation Section. Dose-escalation decisions will take into account all available data, including the safety profile of prior cohorts throughout the time subjects are on study, which will be reviewed by the investigator(s), GSK Medical Monitor, pharmacokineticist, and statistician. The dose-escalation decision for the subsequent cohort and rationale will be documented in writing with copies maintained at each site and the study files.

Any cohort may be expanded beyond the 3 to 6 subjects enrolled during dose escalation, to a maximum of 12 to facilitate collection of additional safety, PK, and pharmacodynamic data. A total of up to 12 subjects may be treated at the dose of ANTIBODY 106-222 selected for Parts 1B and 2B to better characterize the safety, PK, and pharmacodynamic data at that dose, before opening the Dose-Expansion phase.

Dose-Limiting Toxicity

All toxicities will be graded using National Cancer Institute-Common Toxicity Criteria for Adverse Events (NCI-CTCAE) (version 4.0).

An AE is considered to be a DLT if it is considered by the investigator to be clinically relevant and attributed (definitely, probably, or possibly) to the study treatment during the first 4 weeks (i.e., 28 days) of treatment and meets at least one of the criteria listed in Table 5. If an AE is considered related to the underlying disease it is not a DLT. For Part 2, ≥Grade 3 toxicities that are known to occur with pembrolizumab and are controlled within 2 weeks using the recommended supportive measures may not be considered dose-limiting.

TABLE 5
Dose-Limiting Toxicity Criteria

| Toxicity | DLT Definition |
|---|---|
| Hematologic | Febrile neutropenia |
| | Grade 4 neutropenia of >7 days' duration or requiring G-CSF |
| | Grade 4 anemia of any duration |
| | Grade 4 thrombocytopenia of any duration or Grade 3 |
| | thrombocytopenia with bleeding |
| Non- | Grade 4 toxicity |
| hematologic | Grade 3 toxicity that does not downgrade to ≤Grade 2 within 3 |
| | days despite optimal supportive care^{a}, with the following |
| | exceptions: |

Laboratory abnormality that is not clinically significant
according to the investigators (for example, isolated Grade 3
elevation of amylase or lipase not associated with clinical or
radiographic evidence of pancreatitis)
Grade 3 endocrinopathy that is adequately controlled by
hormonal replacement

| | |
|---|---|
| | Any Grade 2 ocular toxicity requiring systemic steroids, or |
| | any ≥Grade 3 ocular toxicity |
| Other | Toxicity that results in permanent discontinuation of ANTIBODY |
| | 106-222 or ANTIBODY 106-222 and pembrolizumab during the |
| | first 4 weeks of treatment |
| | Any other toxicity considered to be dose-limiting that occurs |
| | beyond 4 weeks will be considered in the selection of the dose |
| | for expansion cohorts |
| | Any other event which in the judgment of the investigator and |
| | GSK Medical Monitor is considered to be a DLT |

^{a}Suggested management guidelines described in Dose and Safety Management Guidelines Section for toxicity and may include systemic corticosteroids for immune-related toxicities; if use of systemic corticosteroids delays administration of the second dose of study treatment but the event does not otherwise meet the DLT criteria for non-hematologic toxicity, the dose delay will not be considered a DLT.
DLT = Dose-limiting toxicity;
G-CSF = Granulocyte colony-stimulating factor;
AST = Aspartate aminotransferase;
ALT = Alanine aminotransferase;
ULN = Upper limit of normal;
GSK = GlaxoSmithKline

If a subject experiences a DLT in the first 4 weeks of treatment, the subject will be discontinued from study therapy unless the investigator considers it in the best interest of the subject to continue on study (e.g., in case of tumor regression, symptomatic disease improvement, and/or if the type of DLT is viewed as preventable in subsequent cycles, e.g., by pre-medication). Such cases will require approval by the Sponsor before continuation on study treatment at the same or lower dose.

Guidance for the management of toxicity, including dose modification algorithms, is provided in Dose and Safety Management Guidelines Section and is based on the experience of management of immune-related adverse events (irAEs) since the development of ipilimumab and PD-1 inhibitors such as pembrolizumab. Dose and Safety Management Guidelines Section includes general guidance for the management of non-hematologic AEs, general guidance for the management of irAEs (General Guidelines for Immune-Related Adverse Events Section), general principles of irAE identification and evaluation (General Principles of Immune-Related Adverse Events Identification and Evaluation Section), and specific guidance for: hepatotoxicity (Management of Hepatotoxicity Section), gastrointestinal events (Management of Gastrointestinal Events (Diarrhea or Colitis) Section), skin toxicity (Management of Skin Toxicity Section), endocrine events (Management of Endocrine Events Section), pneumonitis (Management of Pneumonitis Section), hematologic events (Management of Hematologic Events Section), uveitis/iritis (Uveitis/Iritis Section), infusion reactions or severe cytokine release (Management of Infusion Reactions or Severe Cytokine Release Syndrome (sCRS) Section) and dose delay (Dose Delay Section).

Cohort Expansion

Any dose level(s) in Parts 1A and 2A (dose escalation) may be selected for cohort expansion in Parts 1B and 2B of the study in order to collect additional data on safety, PK, pharmacodynamic activity, and clinical activity.

Each expansion cohort will include subjects with a single tumor type and will enroll up to approximately 20 subjects who will be treated at the selected dose level. In both Part 1B and Part 2B, up to three expansion cohorts will be enrolled with one indication per cohort. Selection of tumor indications will be based in part on data generated in Part 1A and Part 2A, respectively. The Steering Committee will review the available preliminary safety, PK, pharmacodynamic, and clinical activity data before selecting the dose level indications for all 3 cohorts. Criteria that may be considered in the determination of which dose level(s) to expand and which tumor types to select for cohort expansion may include:
Target engagement and pharmacodynamic activity: Observed OX40 receptor occupancy and pharmacodynamic activity. Pharmacodynamic activity will be determined by an evaluation of markers of T-cell activation and proliferation in whole blood. The changes in numbers and activation state of lymphocytes will also be assessed and correlated with individual responses as well as immune cell populations within the tumor (see Biomarkers/Pharmacodynamic Markers Section).
Tolerability: The frequency of DLT, AEs of special interest (AESI), and the extent of dose modifications for either ANTIBODY 106-222 or the combination agent.
Clinical activity: Evidence of clinical response, including SD of at least 12 weeks and/or minor responses.

After 10 subjects have been enrolled in a given expansion cohort, the Steering Committee may recommend continued accrual in that expansion cohort up to a total of approximately 20 subjects. While it is anticipated that the additional 10 subjects in each cohort will be treated at the same dose level as the initial 10 subjects, the Steering Committee may recommend exploration of a different dose level on the basis of emerging data.

The selection of dose level(s) and tumor types selected for cohort expansion will be communicated to the sites in writing.

Intra-Subject Dose Escalation

There will be no intra-subject dose escalation, except as follows. Upon determination of the dose selected for Part 1B, subjects being treated at lower doses in Part 1A may be considered for escalation/titration to the Part 1B dose. For such subjects, the decision whether to dose escalate will be made on a case-by-case basis after agreement by the investigator and the GSK Medical Monitor.

There will be no intra-subject dose escalation in Part 2 of the study.

Treatment Arms and Duration

The study includes a screening period, a treatment period, and a follow-up period. Subjects will be screened for eligibility beginning approximately 4 weeks before the start of treatment. The maximum duration of treatment with ANTIBODY 106-222 will be 48 weeks; the maximum duration of treatment with pembrolizumab will be 2 years (Table 6). The follow-up period for safety assessments will be a minimum of 3 months from the date of the last dose. The post-treatment follow-up period includes disease assessments every 12 weeks until documented PD. Following PD, subjects will be contacted every 3 months to assess survival status.

Subjects with confirmed PR or CR will be followed for response duration and may be eligible for additional treatment with ANTIBODY 106-222 at the time of relapse/progression. The decision whether a subject will receive additional treatment will be discussed and agreed upon by the treating investigator and the Sponsor/Medical Monitor on a case-by-case basis.

TABLE 6
Study Treatments

| | |
|---|---|
| Study Part | Study Treatment |

Part 1: ANTIBODY 106-222 Monotherapy

| | |
|---|---|
| 1A - Dose | ANTIBODY 106-222 IV^{a} Q3W for up to 48 weeks |
| escalation | |
| 1B - Cohort | ANTIBODY 106-222 IV^{b} Q3W for up to 48 weeks |
| expansion | |

Part 2: ANTIBODY 106-222 in combination with pembrolizumab

| | |
|---|---|
| 2A - Dose | ANTIBODY 106-222 IV^{c} Q3W for up to 48 weeks |
| escalation | Pembrolizumab 200 mg IV Q3W for up to 2 years |
| 2B - Cohort | ANTIBODY 106-222 IV^{b} Q3W for up to 48 weeks |
| expansion | Pembrolizumab 200 mg IV Q3W for up to 2 years |

^{a}For dose levels see Table 3.
^{b}At one or two dose levels shown to be tolerable in dose escalation of each part.
^{c}For dose levels see Table 4.
IV = Intravenous;
Q3W = Every 3 weeks

Type and Number of Subjects

The number of dose levels and the level at which the MTD is reached cannot be determined in advance. An adequate number of subjects will be enrolled into the study to establish the recommended dose(s) for further study. It is estimated that a total of up to approximately 180 subjects will be enrolled in this two-part study (approximately 60 subjects in Parts 1A and 2A [dose escalation]; approximately 120 subjects in Parts 1B and 2B [cohort expansion]).

In Parts 1A and 2A, if a subject prematurely discontinues before the completion of 4 weeks treatment, for reasons other than DLT, a replacement subject may be enrolled at the discretion of the Sponsor in consultation with the investigator. Subjects will not be replaced in Parts 1B and 2B of the study.

Design Justification

This study evaluates the safety, tolerability, pharmacodynamic effects, and preliminary clinical activity of ANTIBODY 106-222 as a monotherapy and in combination with anti-PD-1, pembrolizumab. The safety, tolerability, and pharmacodynamics of monotherapy ANTIBODY 106-222 will be evaluated in a modified 3+3 dose escalation that includes an accelerated titration design for the first two dose levels. The dose escalation will be followed by expansion cohorts in defined subject populations. Upon demonstration of clear pharmacodynamic immune activation, exploration of the combination of ANTIBODY 106-222 with pembrolizumab may commence, in parallel with the continuing monotherapy exploration. Dose escalation of ANTIBODY 106-222 in combination with a 200 mg fixed dose of pembrolizumab will begin at a dose of ANTIBODY 106-222 that is at least 2 dose levels below a dose of ANTIBODY 106-222 that has been demonstrated to be safe at that point in time.

In the dose escalation phase, subjects will be enrolled with selected solid tumors that are likely to respond to anti-OX40 therapy (e.g., indications previously reported to have a response to immunotherapies, predicted immunogenicity, and/or expression of OX40). The tumor types to be evaluated in dose escalation are as follows: NSCLC, SCCHN, RCC, melanoma, bladder cancer, STS, TNBC, and MSI CRC.

Almost all of these histologies have demonstrated prior response to anti-CTLA-4 and/or anti-PD-1/PD-L1 therapies [Zamarin, 2015]. In addition, gene expression data [TCGA, 2014] suggest that all of these tumor types have at least moderate expression of OX40.

The inclusion of the combination with pembrolizumab is based on the preference to identify potential transformational activity early in development. Although ANTIBODY 106-222 is expected to have meaningful clinical activity as a monotherapy, the full potential of the molecule is likely to be discovered in combination with other agents, particularly immunotherapies. Pembrolizumab is an ideal combination partner for ANTIBODY 106-222 because it targets a different aspect of the cancer-immunity cycle, has a toxicity profile of mainly Grade 1 or 2 events, and preclinical data strongly supports the potential for synergy.

In order to ensure sufficient safety and pharmacodynamic data are available before beginning enrollment to the ANTIBODY 106-222/pembrolizumab combination (Part 2), available clinical data, including safety, pharmacodynamics and efficacy, will be reviewed by the Steering Committee. The study team will also seek endorsement from GSK Medical Governance in order to initiate Part 2 of the study. Upon deciding to open Part 2, the decision will be documented and reported to all participating PIs and IRBs/IECs.

Dose Justification

Part 1: Starting Dose

According to the International Council on Harmonization of Technical Requirements for Registration of Pharmaceuticals for Human Use (ICH)S9 guidance, calculating a starting dose based on ⅙ of the highest non-severely toxic dose (HNSTD) in the most relevant species (cynomolgus monkey) yields a starting dose of ˜16 mg/kg/week. By determining the minimum anticipated biological effect level (MABEL), a more conservative starting dose of 0.003 mg/kg ANTIBODY 106-222 Q3W was selected as a safe starting dose for this FTIH study. The projected human ANTIBODY 106-222 exposure predicted by cynomolgus monkey PK, ANTIBODY 106-222 binding characteristics, and OX86 efficacy data in mouse, as well as consideration of prior clinical experience with agonizing the OX40 pathway also factored into the selection of the starting dose.

Predicted Exposure:

The PK of ANTIBODY 106-222 was assessed in several cynomolgus monkey studies, with single and repeat doses ranging from 0.03 mg/kg to 100 mg/kg; ANTIBODY 106-222 exposure was approximately dose-proportional. Across the dose levels tested, PK profiles did not demonstrate any evidence of target-mediated disposition. These results suggest that allometric methods are appropriate to predict human PK; furthermore, human PK of ANTIBODY 106-222 is expected to be similar to PK of mAbs of the same isotype. Assuming a plasma volume of 3 L for a 70 kg subject, a Cmax of 0.07 μg/mL is predicted for a dose of 0.003 mg/kg.

Nonclinical Safety:

In repeat-dose toxicology studies, ANTIBODY 106-222 was well tolerated in cynomolgus monkeys following weekly IV dosing for up to 4 weeks at doses ranging from 0.03 to 100 mg/kg/week. In these studies, there were no test article related changes, including those associated with T-cell modulation. The NOAEL was determined to be 100 mg/kg/week, the highest dose tested, which is well above the proposed clinical starting dose regardless of the method for computing the human equivalent dose (HED).

Potential for Severe Cytokine Release:

Several lines of investigation were followed to assess the potential of ANTIBODY 106-222 to induce an excessive cytokine response as observed for super agonist TGN1412. In vitro and ex vivo data show that OX40 is expressed on only a small proportion of T cells, i.e., recently activated effector and regulatory T cells in blood and tissues such as lymph node and spleen. Moreover, stimulation through the TCR and CD28 pathways is required for optimal T-cell activation with OX40 agonism. A range of conditions were explored in vitro to assess the potential of ANTIBODY 106-222 to induce cytokine release. In some assays, no evidence of cytokine release was observed; however, under the most sensitive assay conditions using immobilized ANTIBODY 106-222 in pre-activated CD4+ T cells, increased cytokine production (IL-2, IFNγ, and tumor necrosis factor alpha [TNF-α]) was observed. In vivo cytokine monitoring in repeat dose monkey toxicology studies (doses of 0.03-100 mg/kg) and the CT26 mouse tumor model did not demonstrate excessive cytokine release. These data suggest that ANTIBODY 106-222 is not a super agonist and has a low potential for severe cytokine release syndrome (sCRS).

MABEL Determination:

The MABEL assessment is based on receptor occupancy as characterized by in vitro binding experiments with primary human target cells. Using receptor occupancy as a surrogate for biological activity is appropriate as receptor occupancy provides a general characterization of ANTIBODY 106-222 signaling in target cells and since individual biological effects of ANTIBODY 106-222 are not yet prioritized in terms of their impact on subject safety. The binding of ANTIBODY 106-222 to its ligand and target cells was characterized in several experiments yielding different binding coefficients depending on the degree of cellular activation and OX40 expression.

OX40 expression in blood is limited to the small subset of recently activated CD4+ and CD8+ cells [Croft, 2010]. In ex vivo studies, the frequency of OX40+ T-cells in human blood or PBMC cultures from healthy volunteers ranged from <1%. Also in cancer patients lower OX40 expression was observed in peripheral blood compared to tumor sites and draining lymph nodes [Vetto, 1997]. Based on these findings, the unstimulated human whole-blood binding assay, which exhibited a low but quantifiable level of OX40 binding, was considered most representative for the OX40 response in peripheral blood and was selected to determine the MABEL. ANTIBODY 106-222 was shown to bind to lymphocytes in whole blood in a concentration-dependent manner with an EC50 value of 1.45 μg/mL (pooled data derived from 4 donors). Using the Cmax of 0.07 μg/mL predicted for the first 0.003 mg/kg dose, a receptor occupancy of 5% is predicted based on the binding to lymphocytes in human whole blood (the MABEL dose corresponding to 10% receptor occupancy in this experiment is 0.007 mg/kg). Note, that the receptor occupancy calculations here and below assume that the difference between free and total ANTIBODY 106-222 is negligible, i.e. receptor occupancy=Cmax/(EC50+Cmax). This approach yields higher receptor occupancy and more conservative dose estimates compared to an approach which assumes a specific level of target expression.

OX40 expression in certain tissues may be higher than observed in peripheral blood; for example, in the spleen and additionally in the microenvironment of a tumor. In normal cynomolgus monkeys, where ANTIBODY 106-222 was well tolerated, a frequency of 2-30% of OX40+ lymphocytes was detected in lymphoid tissue (spleen). In the tumor and draining lymph nodes of cancer patients the frequency of activated CD4+/OX40+ T-cells was reported to be up to 30% in tumor and draining lymph node samples compared with 0% in peripheral blood [Vetto, 1997]. Data from stimulated OX40 binding experiments, in which cells are activated and OX40 expression is highly upregulated, are therefore expected to be more representative for the OX40 response to ANTIBODY 106-222 in tumor and draining lymph node tissues. For stimulated PBMC half-maximal binding was typically achieved between 0.1 μg/mL to 0.3 μg/mL and similarly, ANTIBODY 106-222 bound to activated human CD4+ T cells with a mean EC50 value of 0.19 μg/mL. Monoclonal antibody concentrations in peripheral tissues are expected to be substantially lower than time-matched concentrations in serum [Tabrizi, 2010; Shah, 2013]. Assuming an antibody biodistribution coefficient of 25% [Shah, 2013] for a given tumor tissue, the peak concentrations are expected be ≤0.018 μg/mL resulting in ≤8% receptor occupancy when using a binding EC50 of 0.2 μg/mL for the stimulated PBMC or CD4+ T cell assays. Applying the stimulated binding EC50 of 0.2 μg/mL to the Cmax of 0.07 μg/mL in peripheral blood yields predicted receptor occupancy of 26%. However, this prediction is not considered representative of the clinical OX40 response as the stimulated assays had much larger degrees of cellular activation than would be expected in patient blood.

In summary, the starting dose of 0.003 mg/kg is expected to result in less than 10% occupancy of OX40 receptors in blood (based on unstimulated binding experiments) and tumor tissues and draining lymph nodes (based on stimulated binding experiments), which is generally assumed a safe level of receptor engagement for immune agonists.

Clinical Experience with OX40 Agonism:

Clinical experience with MEDI6469 did not show cytokine release syndrome (CRS) or other severe toxicity in subjects dosed with single cycles of 0.1 to 2 mg/kg of the antibody [Curti, 2013]. MEDI6469 did not show a significant dose-dependent difference in efficacy for single cycles of 0.1, 0.4, and 2 mg/kg dose levels. Maximal biological activity as defined by stimulation of T-cell proliferation measured by changes in Ki-67 expression in response to MEDI6469 dosing was achieved at the 0.4 mg/kg dose level. With an EC50 of 0.048 μg/mL for binding as measured by ELISA [Curti, 2013], the potency of MEDI6469 appears to be comparable to (or possibly higher than) that of ANTIBODY 106-222. Using the same approach as specified above to predict Cmax and binding, the starting dose of 0.1 mg/kg MEDI6469 with a binding constant of 0.048 μg/mL [Curti, 2013] leads to a predicted receptor occupancy of 98% in the central circulation at Cmax, further supporting the starting dose of ANTIBODY 106-222 of 0.003 mg/kg.

Potential for Clinical Benefit:

Efficacy for agonizing the OX40 pathway has been assessed with an anti-OX86 antibody, a surrogate mAb to murine OX40. In the CT26 mouse colon cancer model, robust efficacy was observed at doses as low as 5 μg per mouse in the most sensitive experiments (FIG. 13b). Assuming similar potency between anti-OX86 and ANTIBODY 106-222, the HED is estimated to be 0.015 or 0.027 mg/kg assuming that AUC drives efficacy and using a clearance based scaling approach with allometric scaling exponents of 0.67 (body surface area normalization) or 0.75 (quarter power scaling) for systemic clearance and mouse and human weights of 0.025 kg and 70 kg, respectively. Assuming that Cmax drives efficacy and using a volume of distribution based scaling approach the HED is estimated to be 0.2 mg/kg assuming an allometric scaling exponent of 1 for volume of distribution. The starting dose is therefore predicted to lie at the lower end of the predicted therapeutic dose range in subjects.

Dosing Frequency:

In a clinical study with MEDI6469 a key biomarker, Ki-67 expression on T cells exhibited maximal stimulation at about 14 days after the first dose [Curti, 2013]. The Ki-67 stimulation declined by about 28 days after the first dose (or 23 days after the last dose in the cycle). Guided by these biomarker dynamics and the expectation of standard IgG1 mAb PK (terminal elimination half-life longer than 2 weeks), a dosing frequency of Q3W for ANTIBODY 106-222 was chosen. This dosing frequency also increases subject convenience for administration with the planned combination partner pembrolizumab which is also dosed Q3W per label.

In summary, the proposed 0.003 mg/kg starting dose of ANTIBODY 106-222 Q3W in study 201212 is anticipated to be safe and tolerable. Subjects will undergo extended clinical observation following ANTIBODY 106-222 dosing and other measures to monitor and treat all subjects for any possible excessive cytokine release.

Part 2: Starting Dose

No drug-drug interaction affecting the PK for the combination is expected for ANTIBODY 106-222 and pembrolizumab. As a checkpoint inhibitor rather than a direct immune-stimulator pembrolizumab is not expected to substantially increase the potential for excessive cytokine release in response to ANTIBODY 106-222, but specific synergies cannot be excluded a priori. In the CT26 mouse efficacy experiments, no differences with regard to indicators of excessive immune stimulation were noted in the anti-PD-1 combination versus the OX86 monotherapy groups at all the dose levels tested. Similar to monotherapy, robust efficacy was seen for OX86 doses as low as 5 μg per mouse (in combination with 200 μg anti-PD-1 mouse homolog) and combination dosing was well tolerated.

The starting dose of ANTIBODY 106-222 for the Part 2/Combination Dose-Escalation phase will be at least 2 dose levels below a dose that has been shown to be tolerated during the monotherapy dose escalation. This determination is based on an allowance that a 10-fold lower dose of ANTIBODY 106-222 should provide a sufficient safety margin when pembrolizumab is added.

The dose of pembrolizumab will be 200 mg IV Q3W.

Benefit:Risk Assessment

The following section outlines the risk assessment and mitigation strategy for this protocol.

In toxicology studies performed in monkeys with ANTIBODY 106-222, no adverse effects were observed. Additionally, nonclinical data with ANTIBODY 106-222 and a rat surrogate antibody in mouse models do not suggest CRS as a significant concern (limitations of nonclinical models are recognized).

Another agonistic anti-OX40 antibody was previously administered in subjects without evidence of severe cytokine release [Curti, 2013]. MEDI6469 (mouse IgG1 mAb currently being developed by Medimmune/AZ) was very well tolerated for doses of 0.1 to 2 mg/kg (single cycle of 3 doses per week), with transient lymphopenia and Grade 1/2 flu-like symptoms as primary toxicities. The proposed starting dose for ANTIBODY 106-222 is well below those administered in the study using MEDI6469.

In addition, OX40 is expressed on a small proportion of T cells, primarily recently activated effector T cells and Tregs. This significantly limits the potential for sCRS. OX40 is not a super agonist and requires stimulation through TCR and CD28 for optimal T-cell activation.

Due to the mechanism of action of ANTIBODY 106-222, toxicities commonly associated with other immune-modulating agents such as checkpoint inhibitors may also occur after administration of ANTIBODY 106-222. However, these toxicities were not seen in nonclinical models. Table 7 outlines the risk assessment and mitigation strategy for this protocol.

Risk Assessment

TABLE 7
Risk Assessment

| | | |
|---|---|---|
| Potential Risk | | |
| of Clinical | Summary of Data/Rationale | |
| Significance | for Risk | Mitigation Strategy |

| Hypersensitivity | Risk for infusion reactions | Subjects Severe |
|---|---|---|
| reaction | and hypersensitivity is | hypersensitivity to another |
| | inherent to many mAbs | mAb are not eligible for |
| | [Brennan, 2010] | participation in this study |
| | | See Management of Infusion |
| | | Reactions or Severe |
| | | Cytokine Release Syndrome |
| | | (sCRS) Section |
| Severe cytokine | OX40 is a costimulatory | See Management of Infusion |
| release | receptor that can stimulate | Reactions or Severe |
| syndrome | proliferation and activation | Cytokine Release Syndrome |
| (sCRS) | of T cells | (sCRS)Section |
| | ANTIBODY 106-222 is an | |
| | OX40 agonist that can | |
| | costimulate T-cell activation | |
| | in the context of TCR signal | |
| | and CD28 cosignal. | |
| | An anti-CD28 super agonist | |
| | (TGN1412) induced rapid- | |
| | onset catastrophic CRS in 6 | |
| | healthy volunteers | |

| | | |
|---|---|---|
| Other immune- | Inflammatory AEs such as | Subjects with the |
| related AEs | diarrhea/colitis, pneumonitis, | following medical history |
| | and hepatotoxicity are well | are not eligible for |
| | established after treatment with | participation in this study |

| | |
|---|---|
| immune-modulating agents, | Toxicity (≥Grade 3) |
| and are consistent with the | related to prior |
| immune-stimulatory mechanism | immunotherapy |
| of action of these agents. | leading to study |
| | treatment |
| | discontinuation |
| | Severe |
| | hypersensitivity to |
| | another mAb |

TCR = T-cell receptor;
mAb = Monoclonal antibody;
AEs = Adverse Events

Overall Benefit:Risk Conclusion

This is an open-label, dose escalation study and the FTIH study of this agent to be conducted in subjects with relapsed/refractory solid tumors for which no standard therapies are anticipated to result in a durable remission. ANTIBODY 106-222 has nonclinical activity in vivo, however it is unknown whether ANTIBODY 106-222 will have clinical activity, thus any potential beneficial effect for an individual subject attributable to ANTIBODY 106-222 is unknown. Data obtained in this study may help identify individuals more likely to benefit or have side effects from ANTIBODY 106-222. Study participants may benefit from the medical tests and screening performed during the study.

Selection of Study Population and Withdrawal Criteria

Deviations from inclusion and exclusion criteria are not allowed because they can potentially jeopardize the scientific integrity of the study, regulatory acceptability, or subject safety. Therefore, adherence to the criteria as specified in the protocol is essential.

Inclusion Criteria

Subjects eligible for enrollment in the study must meet all of the following criteria:
1. Provide signed, written informed consent.
2. Male and female subjects, age ≥18 years (at the time consent is obtained).
3. Histological documentation of locally advanced, recurrent or metastatic solid malignancy that has progressed after standard therapy appropriate for the specific tumor type, or for which standard therapy has proven to be ineffective, intolerable, or is considered inappropriate. Subjects should not have received more than 5 prior lines of therapy for advanced disease including both standards of care and investigational therapies. Subjects whose cancers harbor molecular alterations for which targeted therapy is standard of care should have received health authority-approved appropriate targeted therapy for their tumor types before enrollment.
4. Subjects with the following solid tumors are eligible for screening: NSCLC, SCCHN, RCC, melanoma, bladder, STS, TNBC, and MSI CRC.
5. A biopsy of the tumor tissue obtained at anytime from the initial diagnosis to study entry. Although a fresh biopsy obtained during screening is preferred, archival tumor specimen is acceptable if it is not feasible to obtain a fresh biopsy. For Part 1B and Part 2B, any archival tumor specimen must have been obtained within 3 months of starting study drug.
6. Measurable disease per RECIST version 1.1. Palpable lesions that are not measurable by radiologic or photographic evaluations may not be utilized as the only measurable lesion.
7. Eastern Cooperative Oncology Group (ECOG) performance status (PS) 0-1.
8. Life expectancy of at least 12 weeks.
9. Adequate organ function (see Table 8):

TABLE 8
Definitions for Adequate Organ Function

| System | Laboratory Values | |
|---|---|---|
| Hematologic | | |
| ANC | ≥1.5 × 10⁹/L | |
| Lymphocyte count | >1,000/mm³ | |
| Hemoglobin | ≥9 | g/dL |
| Platelets | ≥100 × 10⁹/L | |
| Hepatic | | |
| Total bilirubin | ≤1.5 × ULN | |
| For subjects with Gilbert's Syndrome (only if direct | ≤3.0 × ULN | |
| bilirubin ≤35%) | | |
| ALT | ≤1.5 × ULN | |
| Renal | | |
| Serum Creatinine | ≤1.5 × ULN | |
| OR | | |
| Calculated CrCl^{a} | >50 | mL/min |

| | |
|---|---|
| Endocrine | |
| TSH^{b} | WNL |

ANC = Absolute neutrophil count;
ALT = alanine aminotransferase;
CrCl = creatinine clearance;
TSH = thyroid-stimulating hormone;
ULN = upper limit of normal;
WNL = within normal limits
^{a}Estimated CrCl should be calculated using the Chronic Kidney Disease Epidemiology Collaboration (CKD-EPI) formula
^{b}If TSH is not within normal limits at baseline, the subject may still be eligible if total T3 or free T3 and free T4 are within the normal limits.

10. QT duration corrected for heart rate by Fridericia's formula (QTcF) <450 msec or QTcF <480 msec for subjects with bundle branch block.
The QTcF is the QT interval corrected for heart rate according to Fridericia's formula, machine-read or manually over-read.
11. In France, a subject will be eligible for inclusion in this study only if either affiliated to or a beneficiary of a social security category.
12. Female subject: is eligible to participate if she is not pregnant (as confirmed by a negative serum beta-human chorionic gonadotrophin (β-hCG) test), not lactating, and at least one of the following conditions applies:
   a. Non-reproductive potential defined as:
      Pre-menopausal females with one of the following:
         Documented tubal ligation
         Documented hysteroscopic tubal occlusion procedure with follow-up confirmation of bilateral tubal occlusion
         Hysterectomy
         Documented Bilateral Oophorectomy
      Postmenopausal defined as 12 months of spontaneous amenorrhea [in questionable cases a blood sample with simultaneous follicle stimulating hormone (FSH) and estradiol levels consistent with menopause (refer to laboratory reference ranges for confirmatory levels)]. Females on hormone replacement therapy (HRT) and whose menopausal status is in doubt will be required to use one of the highly effective contraception methods if they wish to continue their HRT during the study. Otherwise, they must discontinue HRT to allow confirmation of post-menopausal status prior to study enrolment.
   b. Reproductive potential and agrees to follow one of the options listed below in the GSK Modified List of Highly Effective Methods for Avoiding Pregnancy in Females of Reproductive Potential (FRP) requirements from 30 days prior to the first dose of study medication and until 120 days after the last dose of study medication and completion of the follow-up visit.
      GSK Modified List of Highly Effective Methods for Avoiding Pregnancy in Females of Reproductive Potential (FRP)
      This list does not apply to FRP with same sex partners, when this is their preferred and usual lifestyle or for subjects who are and will continue to be abstinent from penile-vaginal intercourse on a long term and persistent basis.
         Contraceptive subdermal implant with a <1% rate of failure per year, as stated in the product label
         Intrauterine device or intrauterine system with a <1% rate of failure per year, as stated in the product label [Hatcher, 2007]
         Oral Contraceptive, either combined or progestogen alone [Hatcher, 2007]
         Injectable progestogen [Hatcher, 2007]
         Contraceptive vaginal ring [Hatcher, 2007]
         Percutaneous contraceptive patches [Hatcher, 2007]
         Male partner sterilization with documentation of azoospermia prior to the female subject's entry into the study, and this male is the sole partner for that subject [Hatcher, 2007].
      These allowed methods of contraception are only effective when used consistently, correctly and in accordance with the product label. The investigator is responsible for ensuring that subjects understand how to properly use these methods of contraception.
13. Male subjects with female partners of child bearing potential must comply with the following contraception requirements from the time of first dose of study medication until 120 days after the last dose of study medication.
   a. Vasectomy with documentation of azoospermia.
   b. Male condom plus partner use of one of the contraceptive options below:
      Contraceptive subdermal implant with a <1% rate of failure per year, as stated in the product label [Hatcher, 2007]
      Intrauterine device or intrauterine system with a <1% rate of failure per year, as stated in the product label [Hatcher, 2007]
      Oral Contraceptive, either combined or progestogen alone [Hatcher, 2007] Injectable progestogen [Hatcher, 2007]
      Contraceptive vaginal ring [Hatcher, 2007]
      Percutaneous contraceptive patches [Hatcher, 2007]
   These allowed methods of contraception are only effective when used consistently, correctly and in accordance with the product label. The investigator is responsible for ensuring that subjects understand how to properly use these methods of contraception.

Exclusion Criteria

A subject will not be eligible for inclusion in this study if any of the following criteria apply:
1. Prior treatment with the following agents (from last dose of prior treatment to first dose of ANTIBODY 106-222):
   TNFR agonists, including OX40, CD27, CD137 (4-1BB), CD357 (GITR): at any time.
   Checkpoint inhibitors, including PD-1, PD-L1, and CTLA-4 inhibitors: within 8 weeks.
   Other anticancer therapy, including chemotherapy, targeted therapy, and biological therapy: within 4 weeks or 5 half lives of the drug, whichever is shorter. Prior radiation therapy is permissible if at least one unirradiated measurable lesion is available for assessment via RECIST version 1.1. A wash out of at least two weeks before start of study drug for palliative radiation to the extremities for osseous bone metastases and 4 weeks for radiation to the chest, brain, or visceral organs is required.
   Investigational therapy: if the subject has participated in a clinical trial and has received an investigational product: within 30 days or 5 half-lives of the investigational product (whichever is shorter). At least 14 days must have passed between the last dose of prior investigational agent and the first dose of study drug. Note: if the agent is a TNFR agonist or a checkpoint inhibitor, the above exclusions take precedence.
2. Prior treatment with Receptor activator of nuclear factor-kappaB ligand (RANKL) inhibitors (e.g., denosumab) within 4 weeks of the start of study drug.
3. Prior allogeneic or autologous bone marrow transplantation or other solid organ transplantation.
4. Toxicity from previous treatment:
   Subjects with ≥Grade 3 toxicity related to prior immunotherapy leading to study treatment discontinuation are not eligible.
   Subjects whose toxicity related to prior treatment has not resolved to ≤Grade 1 (except alopecia, or endocrinopathy managed with replacement therapy) are not eligible.
5. Malignancy other than disease under study, except as noted below:
   Any other malignancy from which the subject has been disease-free for more than 2 years and, in the opinion of the principal investigators and GSK Medical Monitor, will not affect the evaluation of the effects of this clinical trial treatment on currently targeted malignancy, can be included in this clinical trial.
6. Central nervous system (CNS) metastases, with the following exception:
   Subjects who have previously-treated CNS metastases, are asymptomatic, and have had no requirement for steroids or anti-seizure medication for 2 weeks prior to first dose of study drug.
Note: Subjects with carcinomatous meningitis are excluded regardless of clinical stability.
7. Has received transfusion of blood products (including platelets or red blood cells) or administration of colony stimulating factors (including granulocyte colony-stimulating factor [G-CSF], granulocyte-macrophage colony-stimulating factor [GM-CSF], recombinant erythropoietin) within 2 weeks before the first dose of study drug.
8. Major surgery ≤4 weeks before the first dose of study treatment. Subjects must have also fully recovered from any surgery (major or minor) and/or its complications before initiating study treatment.
9. Active autoimmune disease that has required systemic treatment within the last 2 years (i.e., with use of disease modifying agents, corticosteroids or immunosuppressive drugs). Replacement therapy (e.g., thyroxine or physiologic corticosteroid replacement therapy for adrenal or pituitary insufficiency, etc.) is not considered a form of systemic treatment.
10. Concurrent medical condition requiring the use of systemic immunosuppressive medications within 28 days before the first dose of study treatment. Physiologic doses of corticosteroids for treatment of endocrinopathies or steroids with minimal systemic absorption, including topical, inhaled, or intranasal corticosteroids may be continued if the subject is on a stable dose.
11. Active infection, known human immunodeficiency virus infection, or positive test for hepatitis B surface antigen or hepatitis C.
12. Current active liver or biliary disease (with the exception of Gilbert's syndrome or asymptomatic gallstones, liver metastases, or otherwise stable chronic liver disease per investigator assessment).
NOTE: Stable chronic liver disease should generally be defined by the absence of ascites, encephalopathy, coagulopathy, hypoalbuminemia, esophageal or gastric varices, persistent jaundice, or cirrhosis.
13. Known drug or alcohol abuse.
14. Recent history (within the past 6 months) of acute diverticulitis, inflammatory bowel disease, intra-abdominal abscess, or gastrointestinal obstruction
15. Receipt of any live vaccine within 4 weeks.
16. Recent history of allergen desensitization therapy within 4 weeks of starting study treatment.
17. History of severe hypersensitivity to other mAbs.
18. History or evidence of cardiovascular risk including any of the following:
   Recent (within the past 6 months) history of serious uncontrolled cardiac arrhythmia or clinically significant ECG abnormalities including second degree (Type II) or third degree atrioventricular block.
   Documented cardiomyopathy, myocardial infarction, acute coronary syndromes (including unstable angina pectoris), coronary angioplasty, stenting, or bypass grafting within the past 6 months before enrollment.
   Documented congestive heart failure (Class II, III, or IV) as defined by the New York Heart Association functional classification system (NYHA, 1994).
   Recent (within the past 6 months) history of symptomatic pericarditis.
19. History of idiopathic pulmonary fibrosis, pneumonitis, interstitial lung disease, or organizing pneumonia, or evidence of active, non-infectious pneumonitis. Note: post-radiation changes in the lung related to prior radiotherapy and/or asymptomatic radiation-induced pneumonitis not requiring treatment may be permitted if agreed by the investigator and Medical Monitor.
20. Recent history (within 6 months) of uncontrolled symptomatic ascites or pleural effusions.
21. Any serious and/or unstable pre-existing medical, psychiatric disorder, or other condition that could interfere with the subject's safety, obtaining informed consent, or compliance to the study procedures.
22. Is or has an immediate family member (e.g., spouse, parent/legal guardian, sibling or child) who is investigational site or sponsor staff directly involved with this trial, unless prospective IRB approval (by chair or designee) is given allowing exception to this criterion for a specific subject.

Screening Failures

In order to ensure transparent reporting of screen failure subjects, meet the Consolidated Standards of Reporting Trials (CONSORT) publishing requirements, and respond to queries from Regulatory authorities, a minimal set of screen failure information is required including Demography, Screen Failure details, Eligibility Criteria, and any SAE.

Withdrawal/Stopping Criteria

Subjects will receive study treatment for the scheduled time period, unless one of the following occurs earlier: disease progression (as determined by irRECIST), death, or unacceptable toxicity, including meeting stopping criteria for liver chemistry defined in Liver Chemistry Stopping Criteria Section. In addition, study treatment might be permanently discontinued for any of the following reasons:
Deviation(s) from the protocol
Request of the subject or proxy (withdrawal of consent by subject or proxy)
Investigator's discretion
Subject is lost to follow-up
Study is closed or terminated
Subjects with infusion delays >49 days (i.e., 2 missed doses+7 days) should discontinue study drug(s) unless the treating investigator and Sponsor/Medical Monitor agree there is strong evidence supporting continued treatment.
Note: Subjects who require permanent discontinuation of one of the study treatments due to toxicity in a given treatment combination must permanently discontinue both treatments (unless continued treatment with the remaining agent is agreed upon by the treating investigator and Sponsor/Medical Monitor) in that combination and the reason for discontinuation must be recorded. The treatment discontinuation visit (TDV) should be conducted within 30 days of the decision to discontinue study drug(s).
Intercurrent illness that prevents further administration of study treatment(s)
Criteria for discontinuation of study drug(s) as described in Dose and Safety Management Guidelines Section (Safety Management Guidelines) have been met
Criteria described in QTcF Stopping Criteria Section have been met
Criteria described in Stopping Rules for Clinical Deterioration Section have been met

The primary reason study treatment was permanently discontinued must be documented in the subject's medical records and electronic case report form (eCRF).

All subjects who permanently discontinue study treatment without disease progression will be followed for disease progression according to the protocol schedule until;
New anticancer therapy is initiated
Disease progression
Death

A subject with a CR requires confirmation of response via imaging at least 4 weeks after the first imaging showed a CR. Early discontinuation of ANTIBODY 106-222 and/or pembrolizumab may be considered for subjects who have attained a confirmed complete response per RECIST 1.1 that have been treated for at least 6 months and had at least two treatments beyond the date when the initial CR was declared.

Once a subject has permanently discontinued from study treatment, the subject will not be allowed to be re-treated, except as described in Study Treatment Restart or Rechallenge Section and in the following scenario. Re-treatment of subjects who progress after a best overall response of PR or CR may be considered on a case-by-case basis after discussion between the treating investigator and the Sponsor/Medical Monitor.

All subjects who permanently discontinue study treatment will be followed for a minimum of 6 months from the date of the last dose. The follow-up period for safety assessments will be a minimum of 3 months from the date of the last dose. The post treatment follow-up period includes disease assessments every 12 weeks until documented PD. Following PD, subjects will be contacted every 3 months to assess survival status.

If the subject voluntarily discontinues from treatment due to toxicity, ‘AE’ will be recorded as the primary reason for permanent discontinuation on the eCRF.

All subjects who discontinue from study treatment will undergo safety assessments at the time of discontinuation and during post-study treatment follow-up as specified in the Time and Events Table.

Liver Chemistry Stopping Criteria

Liver chemistry stopping and increased monitoring criteria have been designed to assure subject safety and evaluate liver event etiology (in alignment with the Food and Drug Administration [FDA] pre-marketing clinical liver safety guidance). http://www.fda.gov/downloads/Drugs/GuidanceComplianceRegulatoryInformation/Guidances/UCM174090.pdf

If any of the following criteria are met, study treatment must be discontinued:

Liver Chemistry Stopping Criteria -Liver Stopping Event

| | |
|---|---|
| ALT-absolute | ALT ≥ 5 × ULN |
| ALT Increase | ALT ≥ 3 × ULN but <5 × ULN persists for ≥4 weeks |
| Bilirubin^{1,2} | ALT ≥ 3 × ULN and bilirubin ≥ 2 × ULN |
| | (>35% direct bilirubin) |
| INR² | ALT ≥ 3 × ULN and INR > 1.5, if INR measured |
| Cannot | ALT ≥ 3 × ULN but <5 × ULN and cannot be |
| Monitor | monitored weekly for ≥4 weeks |
| Symptomatic³ | ALT ≥ 3 × ULN associated with symptoms |
| | (new or worsening) believed to be related to liver |
| | injury or hypersensitivity |

1. Serum bilirubin fractionation should be performed if testing is available. If serum bilirubin fractionation is not immediately available, discontinue study treatment for that subject if ALT ≥3×ULN and bilirubin ≥2×ULN. Additionally, if serum bilirubin fractionation testing is unavailable, record presence of detectable urinary bilirubin on dipstick, indicating direct bilirubin elevations and suggesting liver injury.
2. All events of ALT ≥3×ULN and bilirubin ≥2×ULN (>35% direct bilirubin) or ALT ≥3×ULN and INR >1.5, if INR measured which may indicate severe liver injury (possible ‘Hy's Law’), must be reported as an SAE (excluding studies of hepatic impairment or cirrhosis); INR measurement is not required and the threshold value stated will not apply to subjects receiving anticoagulants
3. New or worsening symptoms believed to be related to liver injury (such as fatigue, nausea, vomiting, right upper quadrant pain or tenderness, or jaundice) or believed to be related to hypersensitivity (such as fever, rash or eosinophilia)

For additional guidance on the management of hepatotoxicity, please see Management of Hepatotoxicity Section.

Study Treatment Restart or Rechallenge

If a subject meets liver chemistry stopping criteria do not restart/rechallenge the subject with study treatment unless:
GSK Medical Governance approval is granted
Ethics and/or IRB approval is obtained, if required, and
Separate consent for treatment restart/rechallenge is signed by the subject

QTcF Stopping Criteria
The QTcF correction formula must be used for each individual subject to determine eligibility for and discontinuation from the study. This formula may not be changed or substituted once the subject has been enrolled.
The QTcF should be based on single or averaged QTcF values of triplicate ECGs obtained over a brief (e.g., 5-10 minute) recording period.

If a subject meets either of the following criteria, they must be discontinued.
QTcF >500 msec

OR
Change from baseline of QTcF >60 msec

For subjects with underlying bundle branch block follow the discontinuation criteria listed below:

| | |
|---|---|
| Baseline QTcF with | Discontinuation QTcF with |
| Bundle Branch Block | Bundle Branch Block |

| | | |
|---|---|---|
| <450 | msec | ≥500 msec |
| 450-480 | msec | ≥530 msec |

QTcF = QT duration corrected for heart rate by Fridericia's formula

Stopping Rules for Clinical Deterioration

Accumulating clinical evidence indicates that the emergence of objective responses to agents that activate antitumor immune responses may follow delayed kinetics of weeks or months, and can be preceded by initial apparent progression with appearance of new lesions or some enlarging lesions while certain index lesions are regressing (“mixed response”). Therefore, it is reasonable to allow a subject experiencing apparent progression to continue to receive treatment until progression is confirmed at the next imaging assessment at least 4 weeks later. These considerations should be balanced by clinical judgment as to whether the subject is clinically deteriorating and unlikely to receive any benefit from continued treatment.

Such deterioration will be assessed to have occurred after a clinical event that, in the investigator's opinion, is attributable to disease progression, is unlikely to reverse with continued study treatment and therefore indicates that the subject is not benefiting from study treatment and cannot be managed by the addition of supportive care (e.g., bisphosphonates and/or bone directed radiotherapy, thoracentesis, or paracentesis for accumulating effusions). The decision to stop treatment should be discussed with the Sponsor's Medical Monitor. Examples of events that may, in the investigator's opinion, indicate a lack of clinical benefit include, but are not limited to, the following:
ECOG PS decrease of at least 2 points from baseline
Skeletal related events defined by the following:
   pathologic bone fracture in the region of cancer involvement
   cancer related surgery to bone, and/or
   spinal cord or nerve root compression
Development of new CNS metastases
Any setting where the initiation of new antineoplastic therapy has been deemed beneficial to the subject even in the absence of any such documented clinical events

Subject and Study Completion

A subject will be considered to have completed the study if they complete screening assessments, received at least one dose of study treatment(s), and the TDV, or are receiving ongoing study treatment at the time of the Sponsor's decision to close the study.

For both Part 1 (dose-escalation phase) and Part 2 (expansion cohort), a completed subject is one who has discontinued study treatment for reasons listed in Withdrawal/Stopping Criteria Section and completed a TDV or has died while receiving study treatment.

The end of the study is defined as the last subject's last visit.

Study Treatment

Investigational Product and Other Study Treatment

The term ‘study treatment’ is used throughout the protocol to describe any combination of products received by the subject as per the protocol design. Study treatment may therefore refer to the individual study treatments or the combination of those study treatments.

ANTIBODY 106-222 will be intravenously administered to subjects at each study site under medical supervision of an investigator or designee. When administered in combination with pembrolizumab in Part 2 of the study, ANTIBODY 106-222 will be administered first. The date and time of administration will be documented in the source documents and reported in the eCRF.

In Part 2 of the study, pembrolizumab (Table 9) will be intravenously administered to subjects starting at least 1 hour and no more than 2 hours following the end of the ANTIBODY 106-222 infusion under medical supervision of an investigator or designee. The date and time of administration will be documented in the source documents and reported in the eCRF.

For drug administered by an investigator or designee, the dose of study treatment and study participant identification will be confirmed at the time of dosing by a member of the study site staff other than the person administering the study treatment. The specific time of study treatment administration (e.g., time of the week for first administration; time of the day for each administration) should take into consideration PK sampling time points and study visit procedures. Infusions may be administered up to 72 hours before or after the planned date of treatment for administrative reasons only (e.g., scheduling an infusion around a holiday).

The Study Reference Manual (SRM) contains specific instructions for the calculation of ANTIBODY 106-222 doses, and for the preparation of both ANTIBODY 106-222 and pembrolizumab infusions, and administration of these infusions.

TABLE 9
Investigational Product Dosage/Administration

| | Study Ttreatment | |
|---|---|---|
| Product name: | ANTIBODY 106-222 | Pembrolizumab |
| Dosage form: | Lyophilized powder for | Solution for infusion or |
| | reconstitution | Lyophilized powder for |
| | | reconstitution^{c} |
| Unit dose | 40 mg lyophilized | 100 mg/4 mL solution or |
| strength(s)/ | powder | 50 mg lyophilized powder |
| Dosage | Dose range: 0.003 | Dose range: 200 mg |
| level(s): | to ≤10 mg/kg | |
| Route of | IV infusion - 30 min^{a,b} | IV infusion - 30 min^{a} |
| Administration | | |
| Frequency of | Q3W^{b} | Q3W^{b} |
| Administration | | |
| Dosing | Make every effort to | Make every effort to target |
| instructions: | target infusion timing to | infusion timing to be as |
| | be as close to 30 min as | close to 30 min as |
| | possible. However, given | possible. However, given |
| | the variability of infusion | the variability of infusion |
| | pumps from site to site, | pumps from site to site, a |
| | a window of −5 min | window of −5 min and +10 |
| | and +10 min is permitted | min is permitted (i.e., |
| | (i.e., infusion time is 30 | infusion time is 30 min: −5 |
| | min: −5 min/+10 min or | min/+10 min or 25 to 40 |
| | 25 to 40 min). | min). |
| Manufacturer/ | GSK | Merck |
| source of | | |
| procurement | | |

^{a}Infusions may be prolonged in the event of an infusion reaction. If multiple subjects experience clinically significant infusion reactions, the infusion rate may be slowed for all future administrations of study drug(s) for all subjects. Should this global change in infusion rate be required, it will be communicated to the sites in writing.
^{b}Dose levels 1 and 2 will be administered less than 30 min, please refer to the SRM for infusion directions.
^{c}The dosage form of pembrolizumab will be either solution or lyophilized powder for all subjects/doses. This will be decided and communicated prior to start of the combination phase of the study.
Q3W = Every 3 weeks;
GSK = GlaxoSmithKline

Treatment Assignment

Subjects will be identified by a unique subject number that will remain consistent for the duration of the study.

Upon completion of all the required screening assessments, eligible subjects will be registered into a GSK designated registration and medication ordering system, by the investigator or authorized site staff.

Subjects will be assigned to study treatment in the order in which they complete screening assessments (i.e., the study is not randomized).

Planned Dose Adjustments

Dose and Safety Management Guidelines

Distinct safety management guidelines, including dose modification algorithms, are provided in this section for subjects treated with:
ANTIBODY 106-222
Pembrolizumab

Please note: In instances where the investigator is directed to permanently discontinue study treatment, these instructions are mandatory as described in Withdrawal/Stopping Criteria Section.

An overview of the available dose modification guidelines is presented in Table 10.

All AEs are to be graded according to NCI-CTCAE (version 4.0) (http://ctep.cancer.gov). All dose modifications and the reason(s) for the dose modification must be documented in the eCRF.

TABLE 10
General Dose Modification and Management Guidelines for Drug-
related Non-Hematologic Adverse Events Not Otherwise Specified

| Severity | Management | Follow-up |
|---|---|---|
| Grade 1 | Administer | Symptoms resolve to baseline within 7 days: |

| | |
|---|---|
| symptomatic | Provide close follow-up to evaluate for |
| treatment as | increased severity |

| | |
|---|---|
| appropriate | Symptoms ongoing >7 days: |

| | |
|---|---|
| Continue study | Consider following algorithm for Grade |
| drug(s)^{a} | 2 events |

| | | |
|---|---|---|
| Grade 2 | Administer | Symptoms ongoing >7 days or worsening |

| | |
|---|---|
| symptomatic | Consider interruption of study drug(s)^{a} |

| | |
|---|---|
| treatment | Resume study drug(s) at the same |
| Investigate etiology | dose if symptoms have improved to |
| Consider consulting | Grade 1 and steroid dose is 10 mg |
| subspecialist, | prednisone/day or less |

| | |
|---|---|
| biopsy, and/or | Consider starting moderate dose |
| diagnostic | systemic corticosteroids (e.g., 0.5 |
| procedure | mg/kg/day of prednisone or equivalent) |

| | |
|---|---|
| Discuss with | Continue steroids until improvement |
| Sponsor/Medical | to Grade 1 or resolution; taper |
| Monitor | steroids as medically appropriate |

If symptoms continue or worsen to
Grade 3-4, see below

| | | |
|---|---|---|
| Grade | Interrupt study | Symptoms improve to ≤Grade 2: |

| | | |
|---|---|---|
| 3-4 | drug(s)^{a} | Continue steroids until improvement |
| | Consult | to ≤Grade 1 or baseline; taper steroids |
| | subspecialist | over at least 1 month, then if symptoms |
| | Administer 1- | have improved to Grade 1 and steroid |
| | 2 mg/kg/day IV | dose is 10 mg prednisone/day or less, |
| | methylprednisolone | consider resumption of study drug(s) at |
| | Discuss with | the next lower dose level |

| | |
|---|---|
| Sponsor/Medical | Symptoms ongoing: |

| | |
|---|---|
| Monitor | Discuss further management with |
| | consultant and Sponsor/Medical |
| | Monitor |
| | Consider alternative |
| | immunosuppressive therapy |

^{a}If multiple study drugs are administered per protocol, guidance may apply to one or more study agents; discuss management on a case-by-case basis with the Sponsor/Medical Monitor.

General Guidelines for Immune-Related Adverse Events

An irAE is defined as a clinically significant AE of any organ that is associated with study treatment exposure, is of unknown etiology, and is consistent with an immune-related mechanism. Special attention should be paid to AEs that may be suggestive of potential irAEs. An irAE can occur shortly after the first dose or several months after the last dose of treatment.

Early recognition of irAEs and initiation of treatment are critical to reduce the risk of complications, since the majority of irAEs are reversible with the use of steroids and other immune suppressants [Pardoll, 2012; Weber, 2012]. If an irAE is suspected, the subject should return to the study site as soon as possible instead of waiting for his/her next scheduled visit. Subjects who experience a new or worsening irAE should be contacted and/or evaluated at the study site more frequently.

If an irAE is suspected, a thorough evaluation should be conducted in an effort to possibly rule out neoplastic, infectious, metabolic, toxin, or other etiologic causes before diagnosing an irAE. Serological, immunological, and histological (biopsy) data should be considered to support the diagnosis of an immune-related toxicity. Consultation with the appropriate medical specialist should be considered when investigating a possible irAE.

Organs most frequently affected by irAEs include the skin and the colon due to their rapid regeneration rate. Less frequently affected tissues are lung, liver, and the pituitary and thyroid glands. Mild irAEs are usually treated symptomatically and do not require dosing delays or discontinuation. Higher grade and persistent lower grade irAEs typically necessitate interrupting or discontinuing treatment and administration of systemic steroids or other immunosuppressive agents (such as TNF blockers) when systemic steroids are not effective.

General Principles of Immune-Related Adverse Events Identification and Evaluation

Before administration of study treatment, investigators are to review a subject's AEs, concomitant medications, and clinical evaluation results e.g., vital signs, lab results, ECGs, ECOG PS, physical exam findings, responses, etc. as outlined in the Time and Events Table to monitor for new or worsening irAEs and ensure continued dosing is appropriate.

AESI are defined as events of potential immunologic etiology. Such events recently reported after treatment with other immune modulatory therapy include ≥2 Grade colitis, uveitis, hepatitis, pneumonitis, ≥Grade 3 diarrhea, endocrine disorders, and specific cutaneous toxicities, as well as other events that may be immune mediated, including but not limited to demyelinating polyneuropathy, myasthenia gravis-like syndrome, non-infectious myocarditis, or non-infectious pericarditis.

For subjects who experience signs or symptoms that may be consistent with an AESI, sites are strongly encouraged to immediately notify the GSK Medical Monitor of the event via email and/or phone. Documentation of events potentially qualifying for AESI should occur after discussion between the investigator and the Sponsor/Medical Monitor. Even events without clear confirmation of their immunologic etiology may qualify for AESI. Many of these events may also qualify as an SAE. See the SRM for details.

General Guidelines for Clinically Significant Toxicities not Otherwise Specified

While specific guidance is provided for AESI, it is possible that other clinically significant drug-related toxicities that are not specifically described may occur and warrant dose modification.

Investigators must contact the GSK Medical Monitor for all Grade 3 or greater clinically significant non-hematological drug-related toxicities where interruption or permanent discontinuation of study treatment may be warranted according to the guidelines provided in Dose and Safety Management Guidelines Section. Otherwise, investigators are encouraged to contact the GSK Medical Monitor as needed to discuss any case that warrants separate discussion outside of the scope of current guidelines.

In case toxicity does not resolve to Grade 0 to 1 within 12 weeks after the last infusion, study treatment should be permanently discontinued after consultation with the Sponsor. With investigator and Sponsor agreement, subjects with a laboratory AE still at Grade 2 after 12 weeks may continue treatment in the trial only if asymptomatic and controlled.

For subjects who experience a recurrence of the same AE(s) at the same grade or greater with rechallenge of study treatment, a consultation between the GSK Medical Monitor and investigator should occur to determine whether the subject should continue in the study. Recurrence of an SAE at the same grade or greater with rechallenge of study treatment must result in permanent discontinuation of the study treatment.

Management of Hepatotoxicity

In the event of treatment-emergent hepatotoxicity, potential contributing factors such as concomitant medications, viral hepatitis and other infectious causes, choledocholithiasis, and hepatic metastases, and myositis should be investigated. Concomitant medications known to be hepatotoxic which may be contributing to liver dysfunction should be discontinued or replaced with alternative medications to allow for recovery of liver function. As generally understood, AST or ALT >3×ULN and concomitant bilirubin ≥2.0×ULN (>35% direct bilirubin), in the absence of elevated alkaline phosphatase or biliary injury, suggests significant liver injury. Record alcohol use on the liver event alcohol intake form in the eCRF. Liver dysfunction must be fully evaluated even if clinical signs and symptoms indicate progression of liver tumor lesions. Imaging studies must be obtained to document potential progression of malignancy. Guidelines for management of emergent hepatotoxicity are shown in Table 11.

TABLE 11
Guidelines for Dose Modification and Management of Hepatotoxicity

| Severity | Management | Follow-up |
|---|---|---|
| Grade1 | MONITOR | Monitor the subject at least weekly until |
| ALT > ULN to | Assess liver function at | liver chemistries resolve, stabilize, or |
| 3x ULN | least weekly | return to within baseline |
| OR | | Hepatotoxicity improves to ≤ Grade 1 |
| Total bilirubin > | | or baseline or remains stable: |
| ULN to 1.5x | | Provide close follow-up to evaluate |
| ULN | | for increased severity. |
| | | Hepatotoxicity worsens to ≥ Grade 2: |
| | | see below |
| Grade 2 | INTERRUPT/HOLD | Monitor the subject at least twice |
| ALT > 3-5x ULN | Consider interruption of | weekly until liver chemistries resolve, |
| OR | study drug(s) after | stabilize, or return to within baseline |
| Total | discussion with | Hepatotoxicity improves to ≤ Grade 1 |
| bilirubin > | Sponsor/Medical Monitor^{a} | or baseline within 7 days: |
| 1.5-3xULN | Assess for infection and | Resume study drug(s) (note: |
| | liver metastases | requirements specified in Study |
| | Repeat liver chemistries | Treatment Restart or Rechallenge |
| | (include ALT, AST, | Section must be met before |
| | alkaline phosphatase, | treatment can restart) |
| | bilirubin) and perform liver | Provide close follow-up to evaluate |
| | event follow-up | for recurrence |
| | assessments within 24-72 | Hepatotoxicity ongoing > 7 days: |
| | hours (see below) | Start systemic corticosteroids (e.g., |
| | Assess liver function at | 0.5 mg/kg/day of prednisone or |
| | least twice weekly | equivalent) |
| | Discuss with | Continue steroids until improvement |
| | Sponsor/Medical Monitor | to ≤ Grade 1 or baseline or |
| | within 24 hours | resolution; taper steroids over at least |
| | Consider consultation with | one month |
| | a hepatologist | Resume study drug(s) if |
| | | hepatotoxicity improves to ≤ Grade 1 |
| | | or baseline and steroid dose is 10 mg |
| | | prednisone/day or less |
| | | Provide close follow-up to evaluate |
| | | for recurrence |
| | | Discontinue study treatment if: |
| | | Hepatotoxicity continues or worsens |
| | | to ALT > 5x ULN or total bilirubin to > 3x |
| | | ULN (follow instructions below) |
| | | ALT ≥ 3xULN but < 5xULN persists |
| | | for ≥ 4 weeks |
| | | ALT ≥ 3xULN but < 5xULN and |
| | | cannot be monitored weekly for ≥ 4 |
| | | weeks |
| | | Unable to reduce corticosteroid dose |
| | | to 10 mg or less of prednisone or |
| | | equivalent per day within 12 weeks |
| | | Recurrence after rechallenge: |
| | | Discontinue study drug(s) |
| | | permanently |
| | | Monitor subject closely for clinical |
| | | signs and symptoms |
| | | Perform full panel LFTs a weekly or |
| | | more frequently if clinically indicated |
| | | until ALT decreases to ≤ Grade 1 |
| | | At the time of the recurrence, |
| | | complete the eCRF liver event forms. |
| Grade 3 | DISCONTINUE | Monitor the subject at least twice |
| ALT > 5 x ULN | Immediately discontinue | weekly until liver chemistries resolve, |
| OR | study drug(s)^{a} | stabilize, or return to within baseline |
| Total | Assess for infection and | If ALT or bilirubin have not decreased |
| bilirubin > 3x | liver metastases | within 72 hours in the absence of |
| ULN | Repeat liver chemistries | other etiologies and steroid treatment |
| | (include ALT, AST, | has not been administered, initiate |
| | alkaline phosphatase, | treatment with 1-2 mg/kg/day IV |
| | bilirubin) and perform liver | methylprednisolone |
| | event follow-up | Continue steroids until improvement |
| | assessment within 24 | to ≤ Grade 1 or baseline or |
| | hours (see below) | resolution; taper steroids over at least |
| | Assess liver function at | one month |
| | least twice weekly | If serum transaminase levels do not |
| | Consider administration of | decrease 48 hours after initiation of |
| | 1-2 mg/kg/day IV | systemic steroids, oral |
| | methylprednisolone | mycophenolate mofetil 500 mg every |
| | Discuss with | 12 hours may be given. Infliximab is |
| | Sponsor/Medical Monitor | not recommended due to its potential |
| | within 24 hours | for hepatotoxicity. |
| | Consider consultation with | |
| | a hepatologist | |
| Grade 4 | DISCONTINUE | Monitor the subject at least twice |
| ALT > 20 x ULN | Immediately discontinue | weekly until liver chemistries resolve, |
| OR | study drug(s)^{a} | stabilize, or return to within baseline |
| Total | Assess for infection and | Continue steroids until improvement |
| bilirubin > 10x | liver metastases | to ≤ Grade 1 or baseline or |
| ULN | Repeat liver chemistries | resolution; taper steroids over at least |
| Additional | (include ALT, AST, | one month |
| Stopping | alkaline phosphatase, | If serum transaminase levels do not |
| Criteria: ALT ≥ | bilirubin) and perform liver | decrease 48 hours after initiation of |
| 3x ULN AND | event follow-up | systemic steroids, oral |
| Total bilirubin ≥ | assessment within 24 | mycophenolate mofetil 500 mg every |
| 2x ULN ( > 35% | hours (see below) | 12 hours may be given. Infliximab is |
| direct bilirubin)^{b} | Assess liver function at | not recommended due to its potential |
| ALT ≥ 3xULN | least twice weekly | for hepatotoxicity. |
| and INR > 1.5, if | Administer 1-2 mg/kg/day | |
| INR measured^{c} | IV methylprednisolone | |
| ALT ≥ 3xULN | Discuss with | |
| associated with | Sponsor/Medical Monitor | |
| symptoms (new | within 24 hours | |
| or worsening) | Consider consultation with | |
| believed to be | a hepatologist | |
| related to liver | | |
| injury or | | |
| hypersensitivity^{d} | | |

^{a}If multiple study drugs are administered per protocol, guidance may apply to one or more study agents; discuss management on a case-by-case basis with the Sponsor/Medical Monitor.
^{b}Serum bilirubin fractionation should be performed if testing is available. If serum bilirubin fractionation is not immediately available, discontinue study treatment for that subject if ALT ≥ 3xULN and bilirubin ≥ 2xULN. Additionally, if serum bilirubin fractionation testing is unavailable, record presence of detectable urinary bilirubin on dipstick, indicating direct bilirubin elevations and suggesting liver injury.
^{c}All events of ALT ≥ 3xULN and bilirubin ≥ 2xULN (>35% direct bilirubin) or ALT ≥ 3xULN and INR > 1.5, if INR measured which may indicate severe liver injury (possible ‘Hy's Law’), must be reported as an SAE (excluding studies of hepatic impairment or cirrhosis); INR measurement is not required and the threshold value stated will not apply to subjects receiving anticoagulants
^{d}New or worsening symptoms believed to be related to liver injury (such as fatigue, nausea, vomiting, right upper quadrant pain or tenderness, or jaundice) or believed to be related to hypersensitivity (such as fever, rash or eosinophilia)
ALT = Alanine aminotransferase;
ULN = Upper limit of normal;
AST = Aspartate aminotransferase;
IV = Intravenous
LFT = liver function tests;
INR = International Normalized Ratio

Liver Event Follow-Up Assessments
Viral hepatitis serology: Hepatitis A IgM antibody; Hepatitis B surface antigen and Hepatitis B Core Antibody (IgM); Hepatitis C RNA; Cytomegalovirus IgM antibody; Epstein-Barr viral capsid antigen IgM antibody (or if unavailable, obtain heterophile antibody or monospot testing); Hepatitis E IgM antibody
Quantitative hepatitis B DNA and hepatitis delta antibody: Only in those with underlying chronic hepatitis B at study entry (identified by positive hepatitis B surface antigen). If hepatitis delta antibody assay cannot be performed, it can be replaced with a polymerase chain reaction of Hepatitis D RNA virus (where needed) [Le Gal, 2005].
Blood sample for PK analysis, obtained within 28 days after last dose of study drug: Record the date/time of the PK blood sample draw and the date/time of the last dose of study treatment prior to blood sample draw on the eCRF. If the date or time of the last dose is unclear, provide the subject's best approximation. If the date/time of the last dose cannot be approximated OR a PK sample cannot be collected in the time period indicated above, do not obtain a PK sample
Serum creatine phosphokinase (CPK) and lactate dehydrogenase (LDH)
Fractionate bilirubin, if total bilirubin 2×ULN
Obtain complete blood count with differential to assess eosinophilia
Record the appearance or worsening of clinical symptoms of liver injury, or hypersensitivity, on the AE report form
Record use of concomitant medications on the concomitant medications report form including acetaminophen, herbal remedies, other over the counter medications
Record alcohol use on the liver event alcohol intake case report form
For bilirubin or INR criteria:
   Anti-nuclear antibody, anti-smooth muscle antibody, Type 1 anti-liver kidney microsomal antibodies, and quantitative total IgG (or gamma globulins).
   Serum acetaminophen adduct high-performance liquid chromatography (HPLC) assay (quantifies potential acetaminophen contribution to liver injury in subjects with definite or likely acetaminophen use in the preceding week [James, 2009]).
Liver imaging (ultrasound, magnetic resonance, or computerized tomography) and/or liver biopsy to evaluate liver disease; complete Liver Imaging and/or Liver Biopsy eCRF forms.

Management of Gastrointestinal Events (Diarrhea or Colitis)

Signs/symptoms may include, but are not limited to: diarrhea, constipation, abdominal pain, cramping and/or bloating, nausea and/or vomiting, blood and/or mucus in stool with or without fever, rectal bleeding, peritoneal signs consistent with bowel perforation, and ileus.

Differential diagnosis: All attempts should be made to rule out other causes such as metastatic disease, bacterial or parasitic infection, viral gastroenteritis, or the first manifestation of an inflammatory bowel disease by examination for stool leukocytes, stool cultures, and a Clostridium difficile titer. Dose modification guidelines for gastrointestinal events are provided in Table 12.

TABLE 12
Guidelines for Dose Modification and Management
of Gastrointestinal Events (Diarrhea or Colitis)

| Severity | Management | Follow-up |
|---|---|---|
| Grade 1 | Administer anti- | Symptoms resolve to baseline within 7 days: |
| | diarrheal and | Provide close follow-up to evaluate for |
| | symptomatic treatment | increased severity. |
| | as appropriate | Symptoms ongoing > 7 days: |
| | | Consider following algorithm for Grade 2 |
| | | events |
| Grade 2 | Interrupt study drug(s)^{a} | Symptoms resolve to ≤ Grade 1 or baseline within |
| | Administer | 3 days: |
| | antidiarrheal and | resume study drug(s) |
| | symptomatic treatment | Symptoms ongoing > 3 days, blood or mucus in |
| | Discuss with | stool, or ulceration/bleeding on endoscopy. |
| | Sponsor/Medical | consider GI consultation and endoscopy to |
| | Monitor | confirm or rule out colitis |
| | | Start systemic corticosteroids (e.g. |
| | | 0.5 mg/kg/day of prednisone or equivalent) |
| | | Continue steroids until improvement to |
| | | Grade 1 or resolution; taper steroids as |
| | | medically appropriate |
| | | Resume study drug(s) if symptoms have |
| | | improved to Grade 1 and steroid dose is 10 |
| | | mg prednisone/day or less |
| | | If symptoms continue or worsen to Grade 3- |
| | | 4, see below |
| Grade 3 | Interrupt study drug(s)^{a} | When symptoms improve to ≤ Grade 1, |
| | Assess for bowel | taper steroids over at least 1 month. |
| | perforation; do not | If corticosteroid therapy does not reduce |
| | administer | initial symptoms within 48 to 72 hours, treat |
| | corticosteroids if | with additional anti-inflammatory measures. |
| | present | Discontinue additional anti-inflammatory |
| | Consult | measures upon symptom relief and initiate |
| | gastrointestinal (GI) | a prolonged steroid taper over 45-60 days. |
| | service, perform | If symptoms worsen during steroid taper, |
| | endoscopy with biopsy | retaper starting at a higher dose followed by |
| | Administer 1- | a more prolonged taper. |
| | 2 mg/kg/day IV | Recurrence after rechallenge: |
| | methylprednisolone | Discontinue study drug(s) permanently |
| | Discuss with | unless otherwise agreed upon by the |
| | Sponsor/Medical | Sponsor/Medical Monitor and |
| | Monitor | Investigators |
| Grade 4 | Discontinue study | Management as per Grade 3 |
| | drug(s) | |
| | Immediately inform | |
| | Sponsor/Medical | |
| | Monitor | |

^{a}If multiple study drugs are administered per protocol, guidance may apply to one or more study agents; discuss management on a case-by-case basis with the Sponsor/Medical Monitor.
IV = Intravenous

Management of Skin Toxicity

Differential diagnosis: All attempts should be made to rule out other causes such as metastatic disease, infection, or allergic dermatitis. Dose modification guidelines for skin toxicity are provided in Table 13.

TABLE 13
Guidelines for Dose Modification and Management of Skin Toxicity

| Severity | Management | Follow-up |
|---|---|---|
| Localized rash | Symptomatic | Provide close follow-up |
| | management | |
| Non-localized | Interrupt study | Symptoms resolve to baseline within 7 days: |
| rash | drug(s)^{a} | resume study drug(s) |
| (diffuse, ≤ 50% | Discuss with | Symptoms ongoing > 7 days: |
| of skin) | Sponsor/Medical | Start topical or systemic corticosteroids |
| | Monitor | (e.g. 0.5-1 mg/kg/day of prednisone or |
| | Consider | equivalent) |
| | dermatology | Continue steroids until improvement to |
| | consultation and | Grade 1 or resolution; taper steroids as |
| | biopsy | medically appropriate |
| | | Resume study drug(s) if symptoms have |
| | | improved to Grade 1 and steroid dose is |
| | | 10 mg prednisone/day or less |
| | | If symptoms continue or worsen, see |
| | | below |
| Stevens- | Permanently | When dermatitis is controlled, taper |
| Johnson | discontinue study | steroids over at least 1 month |
| syndrome, toxic | drug(s)^{a} | |
| epidermal | Administer 1-2 | |
| necrolysis, or | mg/kg/day IV | |
| rash | methylprednisolone | |
| complicated by | Discuss with | |
| full thickness | Sponsor/Medical | |
| dermal | Monitor | |
| ulceration, or | Consider | |
| necrotic, | dermatology | |
| bullous, or | consultation and | |
| hemorrhagic | biopsy | |
| manifestations | | |

^{a}If multiple study drugs are administered per protocol, guidance may apply to one or more study agents; discuss management on a case-by-case basis with the Sponsor/Medical Monitor.
IV = Intravenous

Management of Endocrine Events

Signs/symptoms may include, but are not limited to: fatigue, weakness, headache, mental status and/or behavior changes, fever, vision disturbances, cold intolerance, abdominal pain, unusual bowel habits, loss of appetite, nausea and/or vomiting, and hypotension. Endocrine events may include the following AE terms: adrenal insufficiency, hyperthyroidism, hypophysitis, hypopituitarism, hypothyroidism, thyroid disorder, and thyroiditis.

Dose modification guidelines for endocrine events are provided in Table 14.

TABLE 14
Guidelines for Dose Modification and Management of Endocrine Events
Hypophysitis with clinically significant adrenal insufficiency
and hypotension, dehydration, and/or electrolyte abnormalities
(such as hyponatremia and hyperkalemia) constitutes an adrenal
crisis and must be considered a medical emergency.

| Severity | Management | Follow-up |
|---|---|---|
| Moderate | Consider interruption | Consider resuming study |
| Signs and/or | of study drug(s)^{a} if | agent(s) when: |
| symptoms of | symptomatic | Subject is stable (on |
| dysfunction | Assess endocrine | hormone-replacement |
| Endocrinopathies | function | therapy if indicated) and |
| requiring | Consider pituitary | symptoms have resolved |
| hormone | imaging | or return to baseline |
| replacement or | Administer up to 1-2 | Subject is receiving ≤ 10 |
| medical | mg/kg/day IV | mg prednisone or |
| intervention | methylprednisolone if | equivalent per day |
| | clinically indicated | |
| | Initiate appropriate | |
| | hormone- | |
| | replacement therapy | |
| | Consider consultation | |
| | with endocrinology | |
| | Discuss with | |
| | Sponsor/Medical | |
| | Monitor | |
| Severe | Consider interruption | Consider resuming study |
| Adrenal crisis or | of study drug(s)^{a} | agent(s) when: |
| other adverse | Discuss with | Subject is stable (on |
| reactions | Sponsor/Medical | hormone-replacement |
| requiring | Monitor | therapy if indicated) and |
| hospitalization, | Consider immediate | symptoms have resolved |
| urgent medical | initiation of 1- | or return to baseline |
| intervention. | 2 mg/kg/day IV | Subject is receiving ≤ 10 |
| | methylprednisolone | mg prednisone or |
| | Consult | equivalent per day |
| | endocrinology | |
| | Other management | |
| | as above | |

^{a}If multiple study drugs are administered per protocol, guidance may apply to one or more study agents; discuss management on a case-by-case basis with the Sponsor/Medical Monitor.
IV = Intravenous

1.1.1.1. Management of Pneumonitis

Signs/symptoms may include, but are not limited to: dyspnea, dry cough, hemoptysis, fever, chest pain and/or tightness, abnormal breath sounds, and fatigue. If symptoms indicate possible new or worsening cardiac abnormalities additional testing and/or a cardiology consultation should be considered. Pneumonitis events may include the following AE terms: pneumonitis, interstitial lung disease, and acute interstitial pneumonitis.

If symptoms indicate possible new or worsening cardiac abnormalities additional testing and/or a cardiology consultation should be considered.

Differential diagnosis: All attempts should be made to rule out other causes such as metastatic disease, and bacterial or viral infection. It is important that subjects with a suspected diagnosis of pneumonitis be managed as per the guidance below until treatment-related pneumonitis is excluded. Treatment of both a potential infectious etiology and pneumonitis in parallel may be warranted. Management of the treatment of suspected pneumonitis with steroid treatment should not be delayed for a therapeutic trial of antibiotics. If an alternative diagnosis is established, the subject does not require management as below; however the AE should be reported regardless of etiology. Dose modification guidelines for pneumonitis are provided in Table 15.

TABLE 15
Guidelines for Dose Modification and Management of Pneumonitis

| Severity | Management | Follow-up |
|---|---|---|
| Grade 1 | Discuss continued | Serial imaging |
| (asymptomatic | treatment with study | |
| with radiographic | drug(s) with | |
| findings only) | Sponsor/Medical | |
| | Monitor | |
| | Consider pulmonary | |
| | consultation and/or | |
| | bronchoscopy if | |
| | clinically indicated | |
| Grade 2 | First episode: | When symptoms improve to ≤ |
| | Interrupt study drug(s)^{a} | Grade 1, taper steroids over at |
| | Consider pulmonary | least 1 month. Permanently |
| | consultation with | discontinue study drug(s) if unable |
| | bronchoscopy and | to reduce corticosteroid dose to ≤ 10 |
| | bronchoalveolar | mg prednisone or equivalent |
| | lavage (BAL) | daily. |
| | Administer 1- | Rechallenge with study drug(s) at |
| | 2 mg/kg/day IV | the same dose(s) may be |
| | methylprednisolone | considered if a first event improves |
| | Discuss with | to Grade 1 or resolves within 12 |
| | Sponsor/Medical | weeks of onset. Repeat chest |
| | Monitor | imaging monthly as clinically |
| | Second episode: | indicated. |
| | Permanently | |
| | discontinue study | |
| | drug(s)^{a} | |
| Grade 3 and 4 | Permanently discontinue | When symptoms improve to ≤ Grade |
| | study drug(s)^{a} | 1, taper steroids over at |
| | Bronchoscopy with | least 1 month. |
| | biopsy and BAL is | If corticosteroid therapy does not |
| | recommended | reduce initial symptoms within 48 |
| | Administer 1- | to 72 hours, treat with additional |
| | 2 mg/kg/day IV | anti-inflammatory measures. |
| | methylprednisolone | Discontinue additional anti- |
| | Discuss with | inflammatory measures upon |
| | Sponsor/Medical | symptom relief and initiate a |
| | Monitor | prolonged steroid taper over 45- |
| | | 60 days. |
| | | If symptoms worsen during steroid |
| | | taper, retaper starting at a higher |
| | | dose followed by a more prolonged |
| | | taper. |
| | | Add anti-infective prophylaxis as |
| | | appropriate. |

^{a}If multiple study drugs are administered per protocol, guidance may apply to one or more study agents; discuss management on a case-by-case basis with the Sponsor/Medical Monitor.
IV = Intravenous

1.1.1.2. Management of Hematologic Events

Dose modification guidelines for hematologic events are provided in Table 16.

TABLE 16
Guidelines for Dose Modification and
Management of Hematologic Events

| Severity | Management | Follow-up |
|---|---|---|
| Grade | As clinically indicated | Provide close follow-up to evaluate for |
| 1-2 | | increased severity |
| Grade 3 | Consider interruption | As clinically indicated |
| | of study drug(s) | Retreatment with study drug(s) may be |
| | Discuss with Medical | considered on a case-by-case basis if |
| | Monitor | agreed upon by investigator and |
| | Obtain flow cytometry | Sponsor/Medical Monitor. |
| | study of T and B | |
| | lymphocytes | |
| | Consult hematology | |
| | Further management | |
| | as clinically indicated | |
| Grade 4 | Consider | As clinically indicated |
| | discontinuation of | Retreatment with study drug(s) may be |
| | study drug(s) for any | considered on a case-by-case basis if |
| | severe or life | agreed upon by investigator and |
| | threatening event ^{a} | Sponsor/Medical Monitor. |
| | Consult hematology | |
| | Obtain flow cytometry | |
| | study of T and B | |
| | lymphocytes. | |
| | Discuss with | |
| | Sponsor/Medical | |
| | Monitor | |

^{a} If multiple study drugs are administered per protocol, guidance may apply to one or more study agents; discuss management on a case-by-case basis with the Sponsor/Medical Monitor.
IV = Intravenous

Uveitis/Iritis

All attempts should be made to rule out other causes such as metastatic disease, infection or other ocular disease (e.g. glaucoma or cataracts). However the AE should be reported regardless of etiology. Dose modification guidelines for uveitis/iritis are provided in Table 17.

TABLE 17
Guidelines for Dose Modification and Management of Uveitis/Iritis

| Severity | Management | Follow-up |
|---|---|---|
| Grade 1 | Symptomatic treatment as | Symptoms resolve to baseline |
| | appropriate | within 7 days: |
| | | Provide close follow-up to |
| | | evaluate for increased severity. |
| | | Symptoms ongoing > 7 days: |
| | | Consider following algorithm |
| | | for Grade 2 events |
| Grade 2 | Interrupt study drug(s)^{a} | Symptoms resolve to baseline |
| | Consultation with | within 7 days: |
| | ophthalmologist is | resume study drug(s) |
| | strongly recommended | Symptoms ongoing > 7 days: |
| | Treat with topical | Discontinue study drugs |
| | steroids such as 1% | If symptoms continue or |
| | prednisolone acetate | worsen to Grade 3-4, see |
| | suspension and | below |
| | iridocyclitics | |
| | Discuss with | |
| | Sponsor/Medical Monitor | |
| Grade 3 | Interrupt study drug(s)^{a} | Symptoms improve to ≤ |
| | Administer 1-2 | Grade 2: |
| | mg/kg/day IV | continue steroids until |
| | methylprednisolone | improvement to ≤ Grade |
| | (local administration of | 1 or baseline; taper steroids |
| | corticosteroids may be | over at least one month |
| | considered after | Symptoms ongoing ≥ 12 |
| | consultation with an | weeks |
| | ophthalmologist) | permanently discontinue |
| | Consultation with | study drugs |
| | ophthalmologist is | |
| | strongly recommended | |
| | Discuss with | |
| | Sponsor/Medical Monitor | |
| Grade 4 | Discontinue study | Management as per Grade 3 |
| | drug(s) | |
| | Immediately inform | |
| | Sponsor/Medical Monitor | |

^{a}If multiple study drugs are administered per protocol, guidance may apply to one or more study agents; discuss management on a case-by-case basis with the Sponsor/Medical Monitor.
IV = Intravenous

Management of Infusion Reactions or Severe Cytokine Release Syndrome (sCRS)

Infusion reactions are a well-documented AE associated with the administration of mAbs. Infusion reactions typically develop within 30 minutes to 2 hours after initiation of drug infusion, although symptoms may be delayed for up to 48 hours. The incidence of infusion reactions varies by mAb agent, and there are multiple mechanisms known to lead to infusion-related reactions including both IgE-dependant anaphylactic and non-IgE dependent anaphylactoid hypersensitivities. Cytokine release syndrome, and when severe, cytokine “storm”, has been identified as a sequelae of the immune system activation associated with infusion reactions.

Infusion Reaction

Infusion reactions may affect any organ system in the body. Most are mild in severity, although severe and even fatal reactions occur. As a group, infusion reactions (including both cytokine mediated and allergic) usually occur during or within a few hours of drug infusion. Occasionally, a reaction may occur one to two days after administration. The NCI-CTCAE (version 4.0) for grading adverse reactions during chemotherapy administration has a scale for grading the severity of infusion reactions and separate grading scales for allergic reactions and anaphylaxis. While use of these separate grading scales may be useful for classifying the nature of an infusion reaction for research purposes, they are less useful for clinical care, since it may not be obvious if the subject is having an allergic infusion reaction or a non allergic infusion reaction.

Clinically infusion reaction may present with flushing, itching, urticaria, and/or angioedema, repetitive cough, sudden nasal congestion, shortness of breath, chest tightness, wheeze, sensation of throat closure or choking, and/or change in voice quality, faintness, tachycardia (or less often bradycardia), hypotension, hypertension and/or loss of consciousness, nausea, vomiting, abdominal cramping, and/or diarrhea, sense of impending doom, tunnel vision, dizziness, and/or seizure, severe back, chest, and pelvic pain.

Cytokine Release Syndrome

Cytokine-associated toxicity, also known as CRS, is a non-antigen-specific toxicity that occurs as a result of strong immune activation. The magnitude of immune activation typically required to mediate clinical benefit using modern immunotherapies exceeds levels of immune activation that occurs in more natural settings. As immune-based therapies have become more potent, CRS is becoming increasingly recognized.

Symptomatology associated with CRS and the severity of symptoms varies greatly, and management can be complicated by intercurrent conditions in these subjects. Fever is a hallmark, and many features of CRS mimic infection. It is not uncommon for subjects to experience temperatures exceeding 40° C.

Potentially life-threatening complications of CRS include cardiac dysfunction, adult respiratory distress syndrome, neurologic toxicity, renal and/or hepatic failure, and disseminated intravascular coagulation. Of particular concern is cardiac dysfunction, which can be rapid onset and severe, but is typically reversible.

It is difficult to determine the exact etiology of this AE in the clinical setting proximate to its occurrence, which makes it difficult to differentiate between a typical infusion reaction and CRS. Since there is a wide commonality in the clinical presentation of these events, the immediate treatment does not vary with respect to the etiology.

In order to better understand the underlying etiology of these events, serum tryptase, C-reactive protein (CRP), ferritin, and a cytokine panel should be drawn during the occurrence of an infusion reaction/CRS of any grade. The serum tryptase, CRP and ferritin panels should be performed at the PI's designated local laboratory. The serum cytokine panel will be performed at a GSK designated laboratory. These results will help us better understand (albeit retrospectively) the etiology of the AE, as outlined in Table 18.

TABLE 18
Biomarker Panel

| Biomarker | Relationship to Adverse Event |
|---|---|
| Serum tryptase^{a} | IgE-related infusion reaction |
| | (Allergic/anaphylaxis) |
| | [Schwartz, 2006] |
| Serum CRP^{a} | Elevated in CRS [Lee, 2014] |
| Serum ferritin^{a} | Elevated in CRS [Lee, 2014] |
| Serum cytokine panel^{b} | *Reported to be elevated in |
| (IFN-γ*\hat{ }, TNF-α*\hat{ }, IL-2*, IL- | CRS [Lee, 2014] |
| 4, IL-5*, IL-6*\hat{ }, IL-8*, IL- | \hat{ }consistently reported as elevated |
| 10*, IL-12p70, IL-13, and | in CRS [Lee, 2014] |
| IL-17) | |

CRP = C-reactive protein;
CRS = Cytokine release syndrome;
IFN-γ = Interferon gamma;
TNF-α = Tumor necrosis factor alpha;
IL = Interleukin.
^{a}Performed by PI designated local laboratory
^{b}Performed by GSK designated laboratory

Treatment of Study Treatment Overdose

ANTIBODY 106-222 Overdose

An overdose is defined as administration of a dose that is at least 50% greater than the intended dose. In the event of an overdose the investigator should:
Contact the Medical Monitor immediately.
Closely monitor the subject for AEs/SAEs and laboratory abnormalities for at least 130 days.
Obtain a plasma sample for PK analysis within 28 days from the date of the last dose of study treatment if requested by the Medical Monitor (determined on a case-by-case basis).
Document the quantity of the excess dose as well as the duration of the overdosing in the eCRF.

Decisions regarding dose interruptions or modifications will be made by the investigator in consultation with the Medical Monitor based on the clinical evaluation of the subject.

There is no specific antidote for overdose with ANTIBODY 106-222. In the event of a suspected overdose, it is recommended that the appropriate supportive clinical care should be instituted, as dictated by the subject's clinical status.

Pembrolizumab Overdose

An overdose of pembrolizumab will be defined as 1000 mg of pembrolizumab. No specific information is available on the treatment of overdose of pembrolizumab. In the event of overdose, the subject should be observed closely for signs of toxicity. Appropriate supportive treatment should be provided if clinically indicated.

Treatment after the End of the Study

The investigator is responsible for ensuring that consideration has been given to the post-study care of the subject's medical condition.

Refer to Withdrawal/Stopping Criteria Section and Time and Events Table Section for follow-up assessments of subjects who are to be followed for disease progression and/or survival after they permanently discontinue from study treatment.

Concomitant Medications and Non-Drop Therapies

Subjects will be instructed to inform the investigator before starting any new medications from the time of first dose of study treatment until the end of the study (Final Study Visit). Any concomitant medication(s), including non-prescription medication(s) and herbal product(s), taken during the study will be recorded in the eCRF. The minimum requirement is that drug name, dose, and the dates of administration are to be recorded. Additionally, a complete list of all prior anticancer therapies will be recorded in the eCRF.

Questions regarding concomitant medications should be directed to the GSK Medical Monitor for clarification.

If future changes are made to the list of permitted/prohibited medications, formal documentation will be provided by GSK and stored in the study file. Any such changes will be communicated to the investigative sites in the form of a letter.

Time and Events Table

TABLE 19
Time and Events Table - Monotherapy and Combination Therapy

| | Scrn^{a/b} | | | Treatment^{a} | | | | | | | | | | Follow-up | |
|---|---|---|---|---|---|---|---|---|---|---|---|---|---|---|---|
| | Week^{a} | | | | | | | | | | | | | | |
| | ≤4 | 0 | 1 | 2 | 3 | 4 | 5 | 6 | 9 | 12 | >12-48^{c} | ≥49-105^{c} | TDV^{d} | DFS FU^{e} | SFU^{f} |
| | Day | | | | | | | | | | | | | | |
| | | | | | | | | | | | | | 30 d after | | |
| | ≤28 | 1 | 8 | 15 | 22 | 29 | 36 | 43 | 64 | 85 | 106-337 | ≥344-736 | last dose | Q12 W | Q12 W |
| | Dose | | | | | | | | | | | | | | |
| | | 1 | | | 2 | | | 3 | 4 | 5 | 6-17 | 18-36 | | | |
| Informed Consent | X | | | | | | | | | | | | | | |
| Inclusion/Exclusion | X | X | | | | | | | | | | | | | |
| Demographics, Medical | X | | | | | | | | | | | | | | |
| History, Prior Medications | | | | | | | | | | | | | | | |
| Concomitant Medications | | | | Assess at each visit from first dose until the TDV visit | | | | | | | | | | | |
| Subject Registration | | X | | | | | | | | | | | | | |
| Anti-Cancer Treatment | | | | | | | | | | | | | X | X | X |

Part I Study Treatment Monotherapy

| | | | | | | |
|---|---|---|---|---|---|---|
| Administer ANTIBODY | X | X | X | X | X | X |
| 106-222 (±2 days)^{g} | | | | | | |

Part 2 Study Treatment Combination Therapy (note: administer pembrolizumab
1 hour after the end of the ANTIBODY 106-222 infusion)

| | | | | | | | |
|---|---|---|---|---|---|---|---|
| Administer ANTIBODY | X | X | X | X | X | X | |
| 106-222^{g} | | | | | | | |
| Administer | X | X | X | X | X | X | X |
| Pembrolizumab^{g} | | | | | | | |

Safety

| AE/SAE Assessment | | Assess at each visit from first dose until the TDV visit for | | | | | | | | | | | |
|---|---|---|---|---|---|---|---|---|---|---|---|---|---|
| | | AEs and until 90 days after last dose for AESI and SAEs^{d} | | | | | | | | | | | |
| ECOG PS | X | X | X | X | X | X | X | X | X | X | Q6 w | Q6 w | X |
| Physical Examination | X | X | | | X | | | X | X | X | Q6 w | Q6 w | |
| Vital Signs and Weight | X | X | X | X | X | X | X | X | X | X | X | X | X |
| 12-lead ECG ^{k} | X | X ^{k} | | | X ^{k} | | | X | X | X ^{k} | Q12 w | Q12 w | X |

Laboratory Assessments (Safety) - perform
assessments pre-dose on each dosing day

| Hepatitis B and C | X | | | | | | | | | | | | |
|---|---|---|---|---|---|---|---|---|---|---|---|---|---|
| Pregnancy Test: Serum β- | ≤3 d | As clinically indicated | | | | | | | | | | | |
| hCG | | | | | | | | | | | | | |
| Clinical Chemistry | X | X | X | X | X | X | X | X | X | X | X | X | X |
| Hematology | X | X | X | X | X | X | X | X | X | X | X | X | X |
| Thyroid function tests | X | | | | | | | X | | X | X | Q6 W | X |
| Calculated CrCl | X | | | | X | | | X | X | X | X | X | X |
| Urinalysis | X | | X | X | X | X | X | X | X | X | X | X | X |

Disease Assessments

| Tumor imaging ^{h} | X | X | Q12 W | |
|---|---|---|---|---|
| Telephone call for survival | | | | X |
| status^{f} | | | | |

Tumor Biopsies

| | | | |
|---|---|---|---|
| Archived tumorⁱ | X | | |
| Fresh tissue sampleⁱ | X | X | X |

| | |
|---|---|
| PD tissue sample^{j} | X^{j} |

Visit Abbreviations:
Scrn = Screen;
TDV = Treatment Discontinuation Visit;
DFSFU = Disease-Free Survival Follow-up;
SFU = Survival Follow-up;
PD = Progressive Disease;
AE = Adverse Event;
SAE = Serious Adverse Event;
AESI = Adverse Events of Special Interest;
ECOG PS = Eastern Cooperative Oncology Group performance status;
ECG = Electrocardiogram;
β-hCG = Beta-human chorionic gonadotropin;
CrCl = Creatinine clearance
^{a}Visit Windows: With the exception of Screening/baseline and Day 1 visits and unless otherwise specified, assessments performed at ≤3-week intervals will have a ±3 day window and assessments performed at >3 week intervals will have a ±1 week window. For Screening/Baseline and Day 1 visits, all procedures must be completed before first dose.
^{b}Screening assessments or procedures to be performed within 4 weeks (28 days) of the first dose (unless otherwise specified, with the exception of the serum pregnancy test which must be performed within 3 days of the first dose of study treatment).
^{c}The frequency of safety assessments from week 12 onwards will be as follows (unless stated otherwise in the table):
Every ANTIBODY 106-222 or pembrolizumab dosing day (pre-dose): Clinical chemistry, hematology, urinalysis, vital signs and weight
Every 6 weeks: Thyroid function tests, ECOG PS assessments, physical examination
Every 12 weeks: 12-lead ECG,
^{d}The treatment discontinuation visit should be completed 30 days from the last dose of study treatment. The window for this visit is +10 days. All AEs and concurrent medications will be collected until at least 30 days after the last dose of study treatment. All AESIs and SAEs and any concurrent medications relevant to the reported AESIs and SAEs will be collected until at least 90 days after the last dose of study treatment or until the start of new anticancer therapy, whichever occurs first. Any drug or study-related SAEs occurring after the 90-day window will be reported according to directions provided in Time Period and Frequency for Collecting Adverse Events and Serious Adverse Events Information Section.
^{e}If study treatment has been permanently discontinued in the absence of PD, the subject will return for disease assessments every 12 weeks until PD is documented (by irRECIST), another anticancer treatment is initiated, or death, whichever occurs first. These visits are described as Disease Free Survival Follow-up (DFS FU) visits.
^{f}The Survival FU visit should be completed every 12 weeks after documented disease progression (or after initiation of another anticancer treatment). Subjects should be contacted every 12 weeks (±2 weeks) until death occurs.
^{g}Dosing of ANTIBODY 106-222 and pembrolizumab at every 3-week intervals is shown in the Time and Events Table; however, dosing of ANTIBODY 106-222 may be delayed due to toxicity. During the combination phase, ANTIBODY 106-222 should be administered first, and pembrolizumab should be administered at least 1 hour and no more than 2 hours following the end of the ANTIBODY 106-222 infusion. ANTIBODY 106-222 will be dosed for a maximum of 48 weeks. Pembrolizumab will be dosed for a maximum of 2 years.
^{h} Screening tumor imaging must be obtained within 28 days of the first dose. Tumor imaging will be performed every 12 weeks (±1 week) until disease progression has been confirmed by irRECIST; additional scans may be obtained if PD is suspected but not confirmed. Immune-related RECIST will be used to determine treatment decisions for PD. If a subject has achieved a CR or PR in the previous radiologic assessment, a repeat scan should be performed as a part of the confirmation of response, within 4-6 weeks to confirm the response. At the TDV, tumor imaging is only required if the last disease assessment did not show PD and was performed ≥6 weeks before TDV. During the DFS FU visits (performed when a subject has permanently discontinued study treatment before disease progression has been documented), tumor imaging will be obtained every 12 weeks (±1 week) until PD, initiation of a new anticancer treatment, or death, whichever comes first.
ⁱA fresh tumor biopsy should be attempted at screening (before first dose) and at Week 6 (after the 3^{rd} dose of study treatment ±1 week). Once evaluable paired tumor biopsies are collected for up to 10 subjects in the dose expansion phases, this requirement may be waived. Tumor lesions planned for biopsy must not be used as indicator lesions for assessment of disease. Although a fresh biopsy obtained during screening is preferred, a recent archival tumor specimen is acceptable if it is not feasible to obtain a fresh biopsy.
^{j}Progressive Disease Tissue Sample: An optional fresh tumor biopsy should be attempted at the time of disease progression.
^{k} For Part 1 only: On Day 1 ECG measurements will be performed in triplicate predose and at the following times after the infusion: EOI + 30 m, EOI + 4 h, EOI + 24 h, on Day 22 ECG measurements will be performed in triplicate predose and on Day 85 ECG measurements will be performed in triplicate predose and at the following times after the infusion: EOI + 30 m.

TABLE 20
Time and Events Table - Pharmacokinetics, Antidrug Antibodies, and Pharmacodynamics (Parts 1 and 2)

| | | Treatment | | | | | | | | | | | | | |
|---|---|---|---|---|---|---|---|---|---|---|---|---|---|---|---|
| | Day | | | | | | | | | | | | | | |
| | | | | | | | | | | | | | | 30 D | 12 Wks |
| | | | | | | | | | | | | | | after | Post- |
| | | | | | | | | | | | | | | Last | Treatment ± |
| | 1 | 2 | 8 | 15 | 22 | 23 | 29 | 36 | 43 | 64 | 85 | 106 | ≥148 | Dose | 1 week |
| | Dose | | | | | | | | | | | | | | |
| | 1 | | | | 2 | | | | 3 | 4 | 5 | 6 | ≥8 | | |
| Pharmacogenetics | X | | | | | | | | | | | | | | |
| (6 mL)^{a} | | | | | | | | | | | | | | | |
| Receptor | Pre | EOI + | X | X | Pre | | | | Pre | | | | | X | |
| occupancy and | EOI + | 24 h | | | EOI + | | | | EOI + | | | | | | |
| phenotyping | 4 h | | | | 4 h | | | | 4 h | | | | | | |
| panels(10 mL) | | | | | | | | | | | | | | | |
| Plasma + PBMC | Pre | | X | | Pre | | | | Pre | | | | | X | |
| prep (20 mL)^{b} | | | | | | | | | | | | | | | |
| Cytokines (5 mL) | Pre | EOI + | X | | Pre | | | | Pre | | | | | | |
| | EOI + | 24 h | | | EOI + | | | | EOI + | | | | | | |
| | 4 h | | | | 4 h | | | | 4 h | | | | | | |
| ANTIBODY 106- | Pre | EOI + | X | X | Pre | EOI + | X | X | Pre | Pre | Pre | Pre | Pre^{e} | X | X |
| 222 | EOI + | 24 h^{d} | | | EOI + | 24 | | | EOI + | EOI + | EOI + | EOI + | | | |
| Pharmacokinetics | 30 m | | | | 30 m | | | | 30 m | 30 m | 30 m | 30 m | | | |
| (1 mL) | EOI + | | | | EOI + | | | | | | | | | | |
| | 4 h | | | | 4 h | | | | | | | | | | |
| Part 2 only: | Pre | EOI + | X | X | Pre | | | | | Pre | | Pre | Pre^{e} | X | X |
| Pembrolizumab | EOPI + | 24 h | | | | | | | | | | | | | |
| Pharmacokinetics | 30 m | | | | | | | | | | | | | | |
| (3 mL) | | | | | | | | | | | | | | | |
| Anti- ANTIBODY | Pre | | | | Pre | | | | Pre | | Pre | | Pre^{e} | X | X |
| 106-222 | | | | | | | | | | | | | | | |
| antibodies^{c} | | | | | | | | | | | | | | | |
| (5 mL) | | | | | | | | | | | | | | | |
| Part 2 only: Anti- | Pre | | | | Pre | | | | | Pre | | Pre | Pre^{e} | X | X |
| Pembrolizumab | | | | | | | | | | | | | | | |
| antibodies^{c} | | | | | | | | | | | | | | | |
| (5 mL) | | | | | | | | | | | | | | | |

Timepoint Definitions:
X = Anytime during visit
Pre = within 60 min before the start of the ANTIBODY 106-222 infusion
EOI + 30 m = within 30 minutes of the end of the ANTIBODY 106-222 infusion
EOPI + 30 m = within 30 minutes of the end of the pembrolizumab infusion
EOI + 4 h = within 4 hours ±10 minutes of the end of the ANTIBODY 106-222 infusion
EOI + 24 h = 24 hours ±4 hours alter the end of the ANTIBODY 106-222 infusion
^{a}Informed consent for optional pharmacogenetics research must be obtained before collecting a sample. It is recommended that the blood sample be taken at the first opportunity after a subject has met all eligibility requirements, and can be done up to 28 days before Day 1.
^{b}If the baseline/pre-dose sample is not viable, samples for PBMCs are not needed at subsequent visits.
^{c}In addition to these scheduled immunogenicity assessments, “event-driven” testing will also be employed for those subjects that experience anaphylaxis, serious hypersensitivity, or AEs related to study drug administration that led to withdrawal from the study.
^{d}Day 2 PK samples are only required during the dose-escalation phases of the study.
^{e}Dose 8 and every 4 dosing cycles
D = Days;
PBMC = Peripheral blood mononuclear cell;
EOI = End of infusion;
Procedures for blood sample collection, processing, storage, and shipping are described in the SRM.

Screening and Critical Baseline Assessments

Demographic and Baseline Assessments

The following demographic parameters will be captured: year of birth, sex, race, and ethnicity.

Medical/medication/family history will be assessed as related to the inclusion/exclusion criteria listed in Selection of Study Population and Withdrawal Criteria Section.

Procedures conducted as part of the subject's routine clinical management (e.g., blood counts, ECG, scans, etc) and obtained prior to signing of informed consent may be utilized for screening or baseline purposes provided the procedure meets the protocol-defined criteria and has been performed in the timeframe of the study.

Critical Baseline Assessments

Cardiovascular medical history/risk factors (as detailed in the eCRF) will be assessed at screening.

Baseline Documentation of Target and Non-Target Lesions
All baseline lesion assessments must be performed within 28 days before the first dose.
Lymph nodes that have a short axis of <10 mm are considered non-pathological and should not be recorded or followed.
Pathological lymph nodes with <15 mm, but ≥10 mm short axis are considered non-measurable.
Pathological lymph nodes with ≥15 mm short axis are considered measurable and can be selected as target lesions; however, lymph nodes should not be selected as target lesions when other suitable target lesions are available.
Measurable lesions up to a maximum of 2 lesions per organ and 5 lesions in total, representative of all involved organs, should be identified as target lesions, and recorded and measured at baseline. These lesions should be selected on the basis of their size (lesions with the longest diameter) and their suitability for accurate repeated measurements (either by imaging techniques or clinically).

Note: Cystic lesions thought to represent cystic metastases should not be selected as target lesions when other suitable target lesions are available.

Note: Measurable lesions that have been previously irradiated and have not been shown to be progressing following irradiation should not be considered as target lesions.
Lytic bone lesions or mixed lytic-blastic lesions, with identifiable soft tissue components, that can be evaluated by computed tomography (CT) or magnetic resonance imaging (MRI) can be considered measurable. Bone scans, fluorodeoxyglucose-positron-emission tomography (FDG-PET) scans or X-rays are not considered adequate imaging techniques to measure bone lesions.
All other lesions (or sites of disease) should be identified as non-target and should also be recorded at baseline. Non-target lesions will be grouped by organ. Measurements of these lesions are not required, but the presence or absence of each should be noted throughout follow-up.

The following are required at baseline: A CT scan with contrast of the chest, abdomen, and pelvis, other areas as indicated by the subject's underlying disease, and clinical disease assessment for palpable lesions. For subjects with head and neck cancer, a CT or MRI of the head and neck area is required. At each post-baseline assessment, evaluations of the sites of disease identified by these scans are required.

NOTE: Although CT scan is preferred, MRI may be used as an alternative method of baseline disease assessment, especially for those subjects where a CT scan is contraindicated due to allergy to contrast, provided that the method used to document baseline status is used consistently throughout study treatment to facilitate direct comparison.

Confirmation of CR and PR are required per protocol. Confirmation assessments must be performed within 4 to 6 weeks after the criteria for response have initially been met and may be performed at the next protocol scheduled assessment. If a confirmation assessment is performed prior to the next protocol schedule assessment, the next protocol scheduled evaluation is still required (e.g., evaluations must occur at each protocol scheduled time point regardless of unscheduled assessments).

Efficacy

Evaluation of Anticancer Activity
Lesion assessment method and timing, evaluation of disease, disease progression and response criteria will be conducted according to RECIST (version 1.1) [Eisenhauer, 2009] and irRECIST as outlined below. irRECIST will be used to determine treatment decisions and will be used for the primary analysis of anticancer activity.
Disease assessment modalities may include imaging (e.g., CT scan, MRI, bone scan, plain radiography) and physical examination (as indicated for palpable/superficial lesions).
The baseline disease assessment will be completed within 4 weeks prior to the first dose of ANTIBODY 106-222, then every 12 weeks thereafter, and at the final study visit. See the Time and Events Table for the schedule of assessments of anticancer activity.
Assessments must be performed on a calendar schedule and should not be affected by dose interruptions/delays.
For post-baseline assessments, a window of ±7 days is permitted to allow for flexible scheduling. If the last radiographic assessment was more than 12 weeks prior to the subject's withdrawal from study and PD has not been documented, a disease assessment should be obtained at the time of withdrawal from the study.
Subjects whose disease responds (either CR or PR) should have a confirmatory disease assessment performed 4 weeks after the date of assessment during which the response was demonstrated. More frequent disease assessments may be performed at the discretion of the investigator.
To ensure comparability between the baseline and subsequent assessments, the same method of assessment and the same technique will be used when assessing response.

Safety

Planned time points for all safety assessments are listed in the Time and Events Table.

Adverse Events and Serious Adverse Events

The investigator and their designees are responsible for detecting, documenting, and reporting events that meet the definitions of an AE or SAE.

Time Period and Frequency for Collecting Adverse Events and Serious Adverse Events Information
AEs and SAEs will be collected from the start of study treatment until the follow-up contact at the time points specified in the Time and Events Table
Medical occurrences that begin prior to the start of study treatment but after obtaining informed consent may be recorded on the Medical History/Current Medical Conditions section of the eCRF.
Any AESI and SAEs assessed as related to study participation (e.g., protocol-mandated procedures, invasive tests, or change in existing therapy) or related to a GSK product will be recorded from the time a subject consents to participate in the study up to 90 days after the last dose of study drug(s). If another anti-cancer agent is started during this time, AESI and SAEs should be recorded until 30 days after the last dose, or initiation of other anti-cancer agent (whichever is later). SAEs must be reported within 24 hours to the Sponsor either by electronic media or paper
All AESI and SAEs will be recorded and reported to GSK within 24 hours.
Investigators are not obligated to actively seek AEs or SAEs in former study subjects. However, if the investigator learns of any SAE, including a death, at any time after a subject has been discharged from the study, and he/she considers the event reasonably related to the study treatment or study participation, the investigator must promptly notify GSK.

NOTE: The method of recording, evaluating, and assessing causality of AEs and SAEs plus procedures for completing and transmitting SAE reports to GSK.

Before each ECG test, the subject should be at rest for approximately 10 minutes. The subject should be in the semi-recumbent or supine position; the same position must be used for all subsequent ECG tests.

For Part 1 of the study, ECG measurements will be performed in triplicate at specified times. All other measurements may be performed as single ECG measurements.

All laboratory tests with values that are considered clinically significantly abnormal during participation in the study or within 30 days after the last dose of study treatment should be repeated until the values return to normal or baseline. If such values do not return to normal within a period judged reasonable by the investigator, the etiology should be identified and the Sponsor notified.

Pharmacokinetics

Blood Sample Collection

Blood samples for PK analysis of ANTIBODY 106-222 and pembrolizumab will be collected at the time points described in Time and Events Table Section (Table 20). The actual date and time of each blood sample collection will be recorded in the eCRF. The timing of PK samples may be altered and/or PK samples may be obtained at additional time points to ensure thorough PK monitoring. Details on PK blood sample collection, processing, storage, and shipping procedures are provided in the SRM.

Blood samples (1 mL) for analysis of plasma ANTIBODY 106-222 concentrations and blood samples (3 mL) for analysis of serum pembrolizumab concentrations will be collected from all subjects at the times indicated in Table 20.

Processing, storage and shipping procedures are provided in the SRM.

Blood Sample Analysis

Plasma or serum analysis for ANTIBODY 106-222 and pembrolizumab will be performed under the control of PTS-DMPK/Scinovo, GSK or Merck Sharp & Dohme Corp the details of which will be included in the SRM. Concentrations of ANTIBODY 106-222 and pembrolizumab will be determined in plasma and serum samples, respectively, using the currently approved bioanalytical methodology. Raw data will be archived at the bioanalytical site (detailed in the SRM). Once the plasma or serum has been analyzed for ANTIBODY 106-222 and pembrolizumab any remaining plasma may be analyzed for other compound-related metabolites and the results reported under a separate PTS-DMPK/Scinovo, GSK or Merck Sharp & Dohme protocol.

Biomarkers/Pharmacodynamic Markers

Blood Biomarkers

Blood samples will be collected and analyzed by flow cytometry to evaluate the binding of ANTIBODY 106-222 to the OX40 receptor, and its pharmacodynamic effect on lymphocytes. OX40 receptor occupancy will be determined prior to dosing of ANTIBODY 106-222, after treatment, and at selected treatment intervals. The numbers of T cells, B cells, and NK cells as well as subsets of T cells will be simultaneously evaluated in whole blood by flow cytometry. The activation and proliferation status of T cells will also be simultaneously assessed in the same sample.

Blood samples will also be collected for isolation of PBMC and plasma. Plasma samples will be used for an analysis of circulating soluble factors in relation to T-cell activation and may be analyzed for soluble OX40 and soluble OX40-drug complex depending on the availability of the assays. Factors to be analyzed may include but are not limited to: the presence of IFN-γ, TNF-α, IL-2, IL-4, IL-5, IL-6, IL 10, IL-8, IL-12p70, IL-13, and IL-17 as well as antibodies against tumor, self, or viral antigens.

PBMCs isolated from whole blood will be preserved and stored for flow cytometry of additional cell types such as immune regulatory populations which may include but are not limited to myeloid derived suppressor cells, subsequent functional analysis or assessment of the diversity of the T-cell repertoire, its relationship to clinical responses, and changes in response to treatment with ANTIBODY 106-222. The functional state of PBMCs may be analyzed for expression of cytokines which may include but not limited to IFN-γ, IL-2, TNFα, IL-17, Granzyme B, and CD107a. PBMCs may also be evaluated for genomic (DNA) and gene expression (RNA or protein) alterations to determine treatment-related changes in immune-related signatures.

Tumor Tissue

Archival tumor tissue as well as fresh pre- and post-treatment biopsies in at least 10 subjects of the dose-expansion cohorts and if possible in the dose escalation cohorts will be evaluated by IHC for expression of phenotypic and functional immune cell markers on tumor infiltrating lymphocytes (TILs) and other immune cells and as well as immune signaling markers on the surface of tumor cells, to understand antitumor immune responses. In addition, when possible, similar analyses will be performed on tumor tissue samples obtained upon progression. Additionally, tumor tissue may be sequenced to assess TCR diversity as well as evaluated for any DNA/RNA/protein changes correlating with response.

Other biomarkers may be evaluated as determined by additional data. Details for sample collection, processing, storage, and shipment will be provided in the SRM.

Genetics

Information regarding genetic research is included in Appendix 7.

Data Management

For this study subject data will be entered into GSK defined eCRFs, transmitted electronically to GSK or designee, and combined with data provided from other sources in a validated data system.

Management of clinical data will be performed in accordance with applicable GSK standards and data cleaning procedures to ensure the integrity of the data, e.g., removing errors and inconsistencies in the data.

AEs and concomitant medications terms will be coded using MedDRA (Medical Dictionary for Regulatory Activities) and an internal validated medication dictionary, GSKDrug.

eCRFs (including queries and audit trails) will be retained by GSK, and copies will be sent to the investigator to maintain as the investigator copy. Subject initials will not be collected or transmitted to GSK according to GSK policy.

Data Analysis Considerations

In the dose escalation cohorts, the dose will be escalated based on all available data, including biomarker and PK data and the safety profile of prior cohorts. In addition, the recommended dose from a Continuous Reassessment Method (N-CRM) analysis [Neuenschwander, 2008] may be calculated. The N-CRM is a type of Bayesian adaptive dose-escalation scheme. The method is fully adaptive and makes use of all the DLT information available at the time of each dose assignment. The Fixed and Adaptive Clinical Trial Simulator (FACTS) will be used to conduct the N-CRM analysis. The DLT information on all subjects enrolled in the trial are used to update the estimated dose-toxicity relationship and provide supportive information in addition to the 3+3 design in the next escalation/de-escalation decision; the 3+3 algorithm is expected to be used as the primary criteria for dose escalation.

The expansion phases are designed to evaluate preliminary efficacy. A futility assessment will be conducted and enrollment may be paused in order to evaluate accumulating data including safety, responses and pharmacodynamic data. The methodology is based on the predictive probability of success if enrollment continues until all planned subjects are recruited [Lee, 2008].

For Part 1: Monotherapy Dose Expansion, after 10 subjects have been enrolled in each cohort, the number of observed responses may be used to guide further enrollment according to the rules summarized in Table 21. However, all available data will be considered in making enrollment decisions.

Starting with 10 subjects and allowing for a maximum sample size of 20, this design will have a type I error rate (α) of 0.128 and 88% power when the true response rate is 30%. The trial is not designed to stop early for efficacy but is designed to assess futility if the predictive probability of success is 1% or less. The type I error rate, power, and predictive probability of success to assess futility were derived from explicitly stating the minimum and maximum sample size, futility stopping rate, and selection of the optimizing criterion as the maximization of power under the alternative hypothesis. The Bayesian prior used in determining the design was Beta (0.1, 0.9), a relatively non-informative distribution with a mean response rate of 10%. Under the null hypothesis, if the true response rate is 10%, the expected sample size of the design is 16 subjects per expansion cohort and the probability of early termination is 73%. Under the alternative hypothesis, if the true response rate is 30%, the expected sample size of the design is 20 subjects per expansion cohort and the probability of early termination is 6%.

These operating characteristics assume that the futility assessment rules are followed. If not and the trial continues to enroll until 20 subjects are evaluated, the overall type 1 error rate increases from 0.128 to 0.133, with an increase in power from 88% to 89%.

The statistical approach for creating futility assessment rules for the expansion phase of the combination cohorts will be similar to that of the monotherapy phase, determined according to which tumor types are selected for study. In addition, a Bayesian hierarchical model may be used to share information across cohorts if PK and biomarker data suggest a strong similarity in clinical activity among cohorts.

CRM-recommended dose-escalation levels, futility assessment rules, and posterior probabilities are only guidelines and the totality of the data will be considered by the team in decision making.

Analysis Populations

The All Treated Population is defined as all subjects who receive at least one dose of ANTIBODY 106-222. Safety and anticancer activity will be evaluated based on this analysis population.

The PK Population will consist of all subjects from the All Treated Population for whom a PK sample is obtained and analyzed.

Anticancer Activity Analyses

The All Treated Population will be used for anticancer activity analyses. Since this is a Phase I study, anticancer activity will be evaluated based on clinical evidence and response criteria. If data warrant, the response data will be summarized by dose level.

If the data warrant, PFS and duration of response will be calculated and listed for each subject. PFS is defined as time from the date of first dose to the date of disease progression according to clinical or radiological assessment or death due to any causes, whichever occurs earliest. Duration of response is defined as the time from first documented evidence of CR or PR until disease progression or death due to any cause among subjects who achieve an overall response (i.e., unconfirmed or confirmed CR or PR). If the subject does not have a documented date of event, PFS will be censored at the date of the last adequate assessment. Further details on rules of censoring will be provided in the RAP. PFS will be summarized using the Kaplan-Meier method if the data warrant.

Secondary Analyses

Pharmacokinetic Analyses

Pharmacokinetic Parameters

PK analysis of ANTIBODY 106-222 and pembrolizumab will be the responsibility of the Clinical Pharmacology Modeling and Simulation (CPMS) Department, GSK or Merck Sharp and Dohme Corp.

PK analysis of drug concentration-time data will be conducted by non-compartmental methods under the direction of CPMS, Quantitative Sciences, GSK. The following PK parameters will be determined if data permit:
Cmax
time to Cmax (tmax)
Cmin
area under the plasma concentration-time curve (AUC(0-t), AUC(0-τ) (repeat dosing) and/or AUC(0-∞) (single dose)
apparent terminal phase elimination rate constant (λz) (single dose)
apparent terminal phase half-life (t½) (single dose)
systemic clearance of parent drug (CL)

Statistical Analysis of Pharmacokinetic Data

Statistical analyses of the PK parameters data will be the responsibility of Clinical Statistics, GSK.

Drug concentration-time data will be listed for each subject and summarized by descriptive statistics at each time point by cohort. PK parameter data will be listed for each subject and summarized by descriptive statistics by cohort.

The data from this study may be combined with the data from other studies for a population PK analysis, which will be reported separately.

Pharmacokinetic/Pharmacodynamic Analyses

Data obtained from the pharmacodynamic samples will be descriptively and/or graphically summarized, and if warranted, exploratory PK/Pharmacodynamic analyses will be conducted to inform dose selection decisions.

Immunogenicity Analyses

Serum samples will be collected and tested for the presence of antibodies that bind to ANTIBODY 106-222 and pembrolizumab. Serum samples for testing anti-ANTIBODY 106-222 and anti-pembrolizumab antibodies will be collected as described in the Time and Events schedule (Time and Events Table Section). The actual date and time of each blood sample collection will be recorded. Details of blood sample collection (including volume to be collected), processing, storage, and shipping procedures are provided in the SRM.

The timing and number of planned immunogenicity samples may be altered during the course of the study, based on newly-available data to ensure appropriate safety monitoring. In the event of a hypersensitivity reaction that is either 1) clinically-significant in the opinion of the investigator, or 2) leads to the subject withdrawing from the study, blood samples should be taken from the subject for immunogenicity testing at the time of the event and again 30 days, 12 weeks, and 24 weeks after. For subjects who prematurely withdraw from the study, immunogenicity testing will occur at withdrawal and at follow-up 30 days, 12 weeks, and 24 weeks after the last dose.

Serum will be tested for the presence of anti-ANTIBODY 106-222 antibodies using the currently approved analytical methodology using a tiered testing schema: screening, confirmation and titration steps. The presence of treatment emergent ADA will be determined using a ANTIBODY 106-222 bridging style ADA assay with a bio-analytically determined cut-point determined during assay validation. Samples taken after dosing with ANTIBODY 106-222 that have a value at or above the cut-point will be considered treatment-emergent ADA-positive. These ADA positive samples will be further evaluated in a confirmatory assay, and confirmed positive samples will be further characterized by assessment of titer. Results of anti-ANTIBODY 106-222 antibody testing will be reported at the end of the study and will include incidence and titer. The presence or absence of antibodies to ANTIBODY 106-222 in dosed subjects will be analyzed, then summarized descriptively and/or graphically presented.

Other Analyses

Translational Research Analyses

The results of translational research investigations may be reported in the main clinical study report (CSR). All endpoints of interest from all comparisons will be descriptively and/or graphically summarized as appropriate to the data.

Further details on the translational research analyses will be addressed in the RAP.

Novel Biomarker(s) Analyses

The results of these biomarker investigations may be reported separately from the main clinical study report. All endpoints of interest from all comparisons will be descriptively and/or graphically summarized as appropriate to the data.

Additional exploratory analyses may be performed to further characterize the novel biomarker.

Pharmacogenetic Analyses

Further details on PGx analyses will be addressed in

Appendix 7 and the PGx RAP.

### REFERENCES

Andrade R J, Robles M, Lucena M I. Rechallenge in drug-induced liver injury: the attractive hazard. Expert Opin Drug Saf. 2009; 8:709-714.
Betting D J, Yamada R E, Kafi K, Said J, van Rooijen N, Timmerman J M. Intratumoral But Not Systemic Delivery of CpG Oligodeoxynucleotide Augments the Efficacy of Anti-CD20 Monoclonal Antibody Therapy Against B Cell Lymphoma. J Immunother. 2009; 32:622-631.
Brennan F R, Morton L D, Spindeldreher S, Kiessling A, Allenspach R, Hey A, et al. Safety and immunotoxicity assessment of immunomodulatory monoclonal antibodies. mAbs. 2010; 2:233-255.
Bulliard, Y., Jolicoeur, R., Windman, M., Rue, S. M., Ettenberg, S., Knee, D. A., Brogdon, J. L. (2013). Activating Fcγ receptors contribute to the antitumor activities of immunoregulatory receptor-targeting antibodies. The Journal of Experimental Medicine, 210(9), 1685-93. http://doi.org/10.1084/jem.20130573
Bulliard, Y., Jolicoeur, R., Zhang, J., Dranoff, G., Wilson, N. S., & Brogdon, J. L. (2014). OX40 engagement depletes intratumoral Tregs via activating FcγRs, leading to antitumor efficacy. Immunology and Cell Biology. http://doi.org/10.1038/icb.2014.26
Chen D S, Mellman I. Oncology meets immunology: The cancer-immunity cycle. Immunity. 2013; 39:1-10.
Cheng X, Veverka V, Radhakrishnan A, Waters L C, Muskett F W, Morgan S H, et al. Structure and interactions of the human programmed cell death 1 receptor. J Biol Chem. 2013; 288:11771-11785.
Croft M. Control of Immunity by the TNFR-Related Molecule OX40 (CD134). Ann Rev Immunol. 2010; 28:57-78.
Curti B D, Kovacsovics-Bankowski M, Morris N, Walker E, Chisholm L, Floyd K, et al. OX40 is a potent immune-stimulating target in late-stage cancer patients. Cancer Res. 2013; 73:7189-7198.
duPre' S A, Hunter Jr. K W. Murine Mammary carcinoma 4T1 induces a leukemoid reaction with spelnomegaly: Association with tumor-derived growth factors. Exp Mol Pathol. 2007; 82:12-24.
Eisenhauer E A, Therasse P, Bogaerts J, Schwartz L H, Sargent D, Ford R, et al. New response evaluation criteria in solid tumors: Revised RECIST guidelines (version 1.1). Eur J Cancer. 2009; 45:228-247.
Guo Z, Wang X, Cheng, D, et al. PD-1 blockade and OX40 triggering synergistically protects against tumor growth in a murine model of ovarian cancer. PLOS ONE. 2014; 9:1-10
Hatcher R A, Trussell J, Nelson A L, Cates W Jr, Stewart F, Kowal D, editors. Contraceptive Technology. 19th edition. New York: Ardent Media, 2007; 24. Table 3-2.
Huang X, Venet F, Wang Y L, Lepape A, Yuan Z, Chen Y, et al. PD-1 expression by macrophages plays a pathologic role in altering microbial clearance and the innate inflammatory response to sepsis. Proc Natl Acad Sci USA. 2009; 106:6303-6308.
Hunt, C M. Mitochondrial and immunoallergic injury increase risk of positive drug rechallenge after drug-induced liver injury: A systematic review. Hepatol. 2010; 52:2216-2222.
Ito T, Wang Y H, Duramad O, Hanabuchi S, Perng O A, et al. OX40 ligand shuts down IL-10-producing regulatory T cells. PNAS USA. 2006; 103:13138-43.
James L P, Letzig L, Simpson P M, Capparelli E, Roberts D W, Hinson J A, et al. Pharmacokinetics of acetaminophen-adduct in adults with acetaminophen overdose and acute liver failure. Drug Metab Dispos. 2009; 37:1779-1784.
Karim R, Jordanova E S, Piersma S J, Kenter G G, Chen L, Boer J M, et al. Tumor-expressed B7-H1 and B7-D C in relation to PD-1+ T-cell infiltration and survival of patients with cervical carcinoma. Clin Cancer Res. 2009; 15:6341-6347.
Keir M E, Butte M J, Freeman G J, Sharpe A H. PD-1 and its ligands in tolerance and immunity. Ann Rev Immunol. 2008; 26:677-704.
KEYTRUDA (pembrolizumab) prescribing information. Merck Sharp & Dohme Corporation, Whitehouse Station, N.J., USA, September 2014.
Lazar-Molnar E, Yan Q, Cao E, Ramagopal U, Nathenson S G, Almo S C. Crystal structure of the complex between programmed death-1 (PD-1) and its ligand PD-L2. Proc Nati Aced Sci USA. 2008; 105:10483-10488.
Le Gal F, Gordien E, Affolabi D, Hanslik T, Alloui C, Dény P, et al. Quantification of hepatitis delta virus RNA in serum by consensus real-time per indicates different patterns of virological response to interferon therapy in chronically infected patients. J Clin Microbiol. 2005; 43:2363-2369.
Lee D W, Gardner R, Porter D L, Louis C U, Ahmed N, Jensen M, et al. Current concepts in the diagnosis and management of cytokine release syndrome. Blood. 2014; 124:188-195.
Lee J J, Liu D D. A predictive probability design for Phase II cancer clinical trials. Clin Trials. 2008; 5:93-106.
Levey A S, Stevens L A, Schmid C H, Zhang Y, Castro A F, Feldman H I, et al. A New Equation to Estimate Glomerular Filtration Rate. Ann Intern Med. 2009; 150:604-612.
Lin D Y, Tanaka Y, Iwasaki M, Gittis A G, Su H.-P, Mikami B, et al. The PD-1/PD-L1 complex resembles the antigen-binding Fv domains of antibodies and T cell receptors. Proc Natl Acad Sci USA. 2008; 105:3011-3016.
Liu C, Lou Y, Lizee G, Qin H, Liu S, et al. Plasmacytoid dendritic cells induce NK cell-dependent, tumor antigen-specific T cell cross-priming and tumor regression in mice. J Clin Invest. 2008; 118:1165-75.
Mall C, Sckisel G D, Mirsoian A, Grossenbacher S K, Murphy W J. Monoclonal antibody therapies targeting immune checkpoints induce fatal anaphylactic reactions in a murine model of breast cancer. J Immunother Cancer. 2014; 2:P111.
Marabelle, A., Kohrt, H., Sagiv-Barfi, I., Ajami, B., Axtell, R., Zhou, G., Levy, R. (2013). Depleting tumor-specific Tregs at a single site eradicates disseminated tumors. Journal of Clinical Investigation, June 3; 123(6), 2447-2463. http://doi.org/10.1172/JCI64859
Maude S L, Barrett D, Teachey D T, Grupp S A. Managing cytokine release syndrome associated with novel T cell-engaging therapies. Cancer J. 2014; 20:119-222.
Merck Sharp & Dohme Corp. Investigator's brochure for pembrolizumab. 19 Dec. 2014
Neuenschwander B, Branson M, Gsponer T. Critical aspects of the Bayesian approach to phase I cancer trials. Statistics Med. 2008; 27:2420-2439.
Nishimura H, Agata Y, Kawasaki A, Sato M, Imamura S, Minato N, et al. Developmentally regulated expression of the PD-1 protein on the surface of double-negative (CD4-CD8−) thymocytes. Int Immunol. 1996; 8:773-780.
NYHA: The Criteria Committee of the New York Heart Association (NYHA). Nomenclature and criteria for diagnosis of diseases of the heart and great vessels. 9th Ed. Boston, Mass.: Little, Brown & Co.; 1994:253-256.
Oken, M M, Creech, R H, Tormey, D C, Horton, J, Davis, T E, McFadden, E T, et al. Toxicity and response criteria of the Eastern Cooperative Oncology Group. Am J Clin Oncol. 1982; 5:649-655.
OPDIVO® (nivolumab) prescribing information. Britol-Myers Squibb Company, Princeton, N.J., USA, December 2014.
Ott P A, F S Hodi, Robert C. CTLA-4 and PD-1/PD-L1 blockade: new immunotherapeutic modalities with durable clinical benefit in melanoma patients. Clin Cancer Res. 2013; 19:5300-5309.
Papay J I, Clines D, Rafi R, Yuen N, Britt S D, Walsh J S, et al. Drug-induced liver injury following positive drug rechallenge. Regul Tox Pharm. 2009; 54:84-90.
Pardoll D M. The blockade of immune checkpoints in cancer immunotherapy. Nature Rev Cancer. 2012; 12:252-264.
P\tilde{e}a-Cruz V, McDonough S M, Diaz-Griffero F, Crum C P, Carrasco R D, Freeman G J. PD-1 on immature and PD-1 ligands on migratory human Langerhans cells regulate antigen-presenting cell activity. J Invest Dermatol. 2010; 130:2222-2230.
Pedoeem A, Azoulay-Alfaguter I, Strazza M, Silverman G J, Mor A. Programmed death-1 pathway in cancer and autoimmunity. Clin Immunol. 2014; 153:145-52.
Poole R M. Pembrolizumab: First Global Approval. Drugs. 2014.
Robert C, Ribas A, Wolchok J D, Hodi F S, Hamid O, Kefford R, et al. Anti-programmed-death-receptor-1 treatment with pembrolizumab in ipilimumab-refractory advanced melanoma: A randomised dose-comparison cohort of a phase 1 trial. Lancet. 2014; 384:1109-1117.
Sanmamed M F, Chen L. Inducible expression of B7-H1 (PD-L1) and its selective role in tumor site immune modulation. Cancer J. 2014; 20:256-261.
Schwartz L B. Diagnostic value of tryptase in anaphylaxis and mastocytosis. Immunol Allergy Clin. 2006; 26:451-463.
Selby, M. J., Engelhardt, J. J., Quigley, M., Henning, K. A., Chen, T., Srinivasan, M., & Korman, A. J. (2013). Anti-CTLA-4 Antibodies of IgG2a Isotype Enhance Antitumor Activity through Reduction of Intratumoral Regulatory T Cells. Cancer Immunology Research. http://doi.org/10.1158/2326-6066.CIR-13-0013
Shah, D K, Betts, A M. Antibody biodistribution coefficients: Inferring tissue concentrations of monoclonal antibodies based on the plasma concentrations in several preclinical species and human. mAbs. 2013; 5:2, 297-305.
Sheppard K A, Fitz L J, Lee J M, Benander C, George J A, Wooters J, et al. PD-1 inhibits T-cell receptor induced phosphorylation of the ZAP70/CD3zeta signalosome and downstream signaling to PKCtheta. FEBS Lett. 2004; 574:37-41.
Simpson, T. R., Li, F., Montalvo-Ortiz, W., Sepulveda, M. A., Bergerhoff, K., Arce, F., Quezada, S. A. (2013). Fc-dependent depletion of tumor-infiltrating regulatory T cells co-defines the efficacy of anti-CTLA-4 therapy against melanoma. The Journal of Experimental Medicine, 210(9), 1695-710. http://doi.org/10.1084/jem.20130579
Tabrizi, M, Bornstein, G G, Suria H. Biodistribution Mechanisms of Therapeutic Monoclonal Antibodies in Health and Disease. The AAPS Journal. 2010, Vol. 12, No. 1, March 2010.
Taube J M, Anders R A, Young G D, Xu H, Sharma R, McMiller T L, et al. Colocalization of inflammatory response with B7-h1 expression in human melanocytic lesions supports an adaptive resistance mechanism of immune escape. Sci Transl Med. 2012; 4:Article 127ra37.
TCGA Research Network: http://cancergenome.nih.gov. Accessed August 2014.
Topalian S L, Drake C G, Pardoll D M. Targeting the PD-1/B7-H1(PD-L1) pathway to activate anti-tumor immunity. Curr Opin Immunol. 2012; 24:207-212.
Vetto J T, Lum S, Morris A, Sicotte M, Davis J, Lemon M, Weinberg A. Presence of the T-cell activation marker OX-40 on tumor infiltrating lymphocytes and draining lymph node cells from patients with melanoma and head and neck cancers. Am J Surg. 1997 September; 174(3):258-65
Wang W, Wang E Q, Balthasar J P. Monoclonal Antibody Pharmacokinetics and Pharmacodynamics. Clin Pharmacol Ther. 2008; 84:548-558.
Weber J S, Kahler K C, Hauschild A. Management of immune-related adverse events and kinetics of response with ipilimumab. J Clin Oncol. 2012; 30:2691-2697.
White A L, Chan H T, French R R, Beers S A, Cragg M S, Johnson P W, Glennie M J. FcγRIIB controls the potency of agonistic anti-TNFR mAbs. Cancer Immunol Immunother. 2013 May; 62(5):941-8.
Wolchok J D, Hoos A, O'Day S, Weber J S, Hamid O, Lebbe C, et al. Guidelines for the Evaluation of Immune Therapy Activity in Solid Tumors: Immune-Related Response Criteria. Clin Cancer Res 2009; 15(23): 7412-20.
Wolchok J D, Kluger H, Callahan M K, Postow M A, Rizvi N A, Lesokhin A M, et al. Nivolumab plus ipilimumab in advanced melanoma. N Engl J Med. 2013; 369:122-133.
Yao S, Chen L. PD-1 as an immune modulatory receptor. Cancer J (United States). 2014; 20:262-264.
Zamarin D, Postow M A. Immune checkpoint modulation: Rational design of combination strategies. Pharmacol Ther. 2015 Jan. 10 pii: S0163-7258(15)00004-2. doi: 10.1016/j.pharmthera.2015.01.003. [Epub ahead of print].
Zhang X, Schwartz J-C D, Guo X, Bhatia S, Cao E, Lorenz M, et al. Structural and functional analysis of the costimulatory receptor programmed death-1. Immunity. 2004; 20: 337-347.

Abbreviations

| | |
|---|---|
| ACT | Adoptive cell transfer |
| ADA | Antidrug antibody |
| ADCC | Antibody-dependent cellular cytotoxicity |
| AE | Adverse event(s) |
| AESI | Adverse events of special interest |
| ALT | Alanine aminotransferase |
| ANC | Absolute neutrophil count |
| AST | Aspartate aminotransferase |
| AUC(0-t) | Area under the plasma concentration-time curve from time |
| | 0 to the time of the last quantifiable concentration) |
| AUC(0-τ) | Area under the concentration-time curve over the dosing |
| | interval |
| BAL | Bronchoalveolar lavage |
| β-hCG | Beta-human chorionic gonadotropin |
| BUN | Blood urea nitrogen |
| CKD-EPI | Chronic Kidney Disease Epidemiology Collaboration |
| CL | Systemic clearance of parent drug |
| CrCl | Calculated creatinine clearance |
| Cmax | Maximum observed concentration |
| Cmin | Minimum observed concentration |
| CNS | Central nervous system |
| CONSORT | Consolidated Standards of Reporting Trials |
| CPK | Creatine phosphokinase |
| CPMS | Clinical Pharmacology Modeling and Simulation |
| CR | Complete response |
| CRM | Continual reassessment method |
| CRP | C-reactive protein |
| CRS | Cytokine release syndrome |
| CSR | Clinical Study Report |
| CT | Computed tomography |
| CTLA-4 | Cytotoxic T-lymphocyte-associated antigen 4 |
| CV | Cardiovascular |
| DCR | Disease Control Rate |
| DFS | Disease-free survival |
| DFSFU | Disease-free survival follow-up |
| DILI | Drug-induced liver injury |
| dL | Deciliter |
| DLT | Dose-limiting toxicity |
| DNA | Deoxyribonucleic acid |
| DRE | Disease-related event |
| ECG | Electrocardiogram(s) |
| ECOG | Eastern Cooperative Oncology Group |
| EOI | End of infusion |
| EOPI | End of pembrolizumab infusion |
| eCRF | Electronic case report form |
| FACTS | Fixed and Adaptive Clinical Trial Simulator |
| FcγR | Antibody receptor crystalizable fragments gamma |
| FDA | Food and Drug Administration |
| FDG-PET | Fluorodeoxyglucose-positron-emission tomography |
| FRP | Females of reproductive potential |
| FSH | Follicle stimulating hormone |
| FTIH | First time in human |
| GCP | Good Clinical Practice |
| G-CSF | Granulocyte colony-stimulating factor |
| GITR | Glucocorticoid-induced TNFR family related gene |
| GM-CSF | Granulocyte-macrophage colony-stimulating factor |
| GSK | GlaxoSmithKline |
| HED | Human equivalent dose |
| HNSTD | Highest non-severely toxic dose |
| HPLC | High-performance liquid chromatography |
| h | Hour(s) |
| HRT | Hormone replacement therapy |
| IB | Investigator's Brochure |
| ICH | International Council on Harmonization of Technical |
| | Requirements for Registration of Pharmaceuticals for |
| | Human Use |
| IEC | Independent Ethics Committee |
| IgG | Immunoglobulin G |
| IgM | Immunoglobulin M |
| IHC | Immunohistochemistry |
| IL-10 | Interleukin 10 |
| INR | International normalized ratio |
| IP | Intraperitoneal |
| irAE | Immune-related adverse event(s) |
| IRB | Institutional Review Board(s) |
| irRECIST | Immune-related RECIST |
| ITIM | Immunoreceptor tyrosine-based inhibition motif |
| ITSM | Immunoreceptor tyrosine-based switch motif |
| IV | Intravenous |
| Kd | Equilibrium dissociation constant |
| kg | Kilogram(s) |
| L | Liter |
| LDH | Lactate dehydrogenase |
| LFT | Liver function Tests |
| μg | Microgram |
| mAb | Monoclonal antibody |
| MABEL | Minimum anticipated biological effect level |
| MAD | Maximum administered dose |
| MCH | Mean corpuscular hemoglobin |
| MCV | Mean corpuscular volume |
| MedDRA | Medical Dictionary for Regulatory Activities |
| mg | Milligram(s) |
| min | Minute(s) |
| mL | Milliliter(s) |
| mmHg | Millimeters of mercury |
| MRI | Magnetic resonance imaging |
| MSDS | Material Safety Data Sheet |
| MSI CRC | Colorectal carcinoma displaying microsatellite |
| | instability |
| MTD | Maximum tolerated dose |
| NCI-CTCAE | National Cancer Institute - Common Terminology |
| | Criteria for Adverse Events |
| NK | Natural killer |
| NOAEL | No observed adverse effect level |
| NSCLC | Non-small cell lung cancer |
| nTregs | Natural Tregs |
| NYHA | New York Heart Association |
| ORR | Objective response rate |
| OS | Overall survival |
| PBMC | Peripheral blood mononuclear cell |
| PD-1 | Programmed death receptor-1 |
| PD | Progressive disease |
| PD-L1 | Programmed death ligand 1 |
| PD-L2 | Programmed death ligand 2 |
| PFS | Progression-free survival |
| PGx | Pharmacogenetics |
| PI | Principal investigator |
| PK | Pharmacokinetic(s) |
| PR | Partial response |
| PS | Performance status |
| Q2W | Every 2 weeks |
| Q3W | Every 3 weeks |
| QTc | Corrected QT interval duration |
| QTcF | QT duration corrected for heart rate by Fridericia's |
| | formula |
| RAP | Reporting and Analysis Plan |
| RANKL | Receptor activator of nuclear factor-kappaB ligand |
| RBC | Red blood cells |
| RCC | Renal cell carcinoma |
| RECIST | Response Evaluation Criteria in Solid Tumors |
| RNA | Ribonucleic acid |
| RP2D | Recommended Phase 2 dose |
| SAE | Serious adverse event(s) |
| SCCHN | Squamous cell carcinoma of the head and neck |
| sCRS | Severe cytokine release syndrome |
| SD | Stable disease |
| SFU | Survival follow-up |
| SLD | Sum of the longest diameters |
| SRM | Study Reference Manual |
| STS | Soft tissue sarcoma |
| TCR | T-cell receptor |
| TDV | Treatment discontinuation visit |
| TILs | Tumor infiltrating lymphocytes |
| TNBC | Triple-negative breast cancer |
| TNF | Tumor necrosis factor |
| TNFR | Tumor necrosis factor receptor |
| Tregs | Regulatory T cells |
| Tr1 | Type 1 regulatory |
| TSH | Thyroid stimulating hormone |
| ULN | Upper limit of normal |
| WBC | White blood cells |
| WNL | Within normal limits |

Trademark Information

Trademarks of the GlaxoSmithKline group of companies

NONE

Trademarks not owned by the GlaxoSmithKline
group of companies

KEYTRUDA ®
OPDIVO ®
YERVOY ®

Guidelines for Assessment of Disease, Disease Progression and Response Criteria—Adapted from RECIST Version 1.1

Assessment Guidelines

Please note the following:
The same diagnostic method, including use of contrast when applicable, must be used throughout the study to evaluate a lesion. Contrast agents must be used in accordance with the Image Acquisition Guidelines.
All measurements should be taken and recorded in millimeters (mm), using a ruler or calipers.
Ultrasound is not a suitable modality of disease assessment. If new lesions are identified by ultrasound, confirmation by CT or MRI is required.
Fluorodeoxyglucose (FDG)-PET is generally not suitable for ongoing assessments of disease. However FDG-PET can be useful in confirming new sites of disease where a positive FDG-PET scans correlates with the new site of disease present on CT/MRI or when a baseline FDG-PET was previously negative for the site of the new lesion. FDG-PET may also be used in lieu of a standard bone scan providing coverage allows interrogation of all likely sites of bone disease and FDG-PET is performed at all assessments.
If PET/CT is performed then the CT component can only be used for standard response assessments if performed to diagnostic quality, which includes the required anatomical coverage and prescribed use of contrast. The method of assessment should be noted as CT on the eCRF.

Clinical Examination:

Clinically detected lesions will only be considered measurable when they are superficial (e.g., skin nodules). In the case of skin lesions, documentation by color photography, including a ruler/calipers to measure the size of the lesion, is required.

CT and MRI: Contrast Enhanced CT with 5 mm Contiguous Slices is Recommended.

Minimum size of a measurable baseline lesion should be twice the slice thickness, with a minimum lesion size of 10 mm when the slice thickness is 5 mm. MRI is acceptable, but when used, the technical specification of the scanning sequences should be optimized for the evaluation of the type and site of disease and lesions must be measured in the same anatomic plane by use of the same imaging examinations. Whenever possible, the same scanner should be used.

X-ray:

In general, X-ray should not be used for target lesion measurements owing to poor lesion definition. Lesions on chest X-ray may be considered measurable if they are clearly defined and surrounded by aerated lung; however chest CT is preferred over chest X-ray.

Brain Scan:

If brain scans are required, then contrast enhanced MRI is preferable to contrast enhanced CT.

Guidelines for Evaluation of Disease

Measurable and Non-Measurable Definitions

Measurable Lesion:

A non-nodal lesion that can be accurately measured in at least one dimension (longest dimension) of
≥10 mm with MRI or CT when the scan slice thickness is no greater than 5 mm. If the slice thickness is greater than 5 mm, the minimum size of a measurable lesion must be at least double the slice thickness (e.g., if the slice thickness is 10 mm, a measurable lesion must be ≥20 mm).
≥10 mm caliper/ruler measurement by clinical exam or medical photography.
≥20 mm by chest X-ray.
Additionally lymph nodes can be considered pathologically enlarged and measurable if

≥15 mm in the short axis when assessed by CT or MRI (slice thickness recommended to be no more than 5 mm). At baseline and follow-up, only the short axis will be measured.

Non-Measurable Lesion:

All other lesions including lesions too small to be considered measurable (longest diameter <10 mm or pathological lymph nodes with ≥10 mm and <15 mm short axis) as well as truly non-measurable lesions, which include: leptomeningeal disease, ascites, pleural or pericardial effusions, inflammatory breast disease, lymphangitic involvement of the skin or lung, abdominal masses/abdominal organomegaly identified by physical exam that is not measurable by reproducible imaging techniques

Measurable Disease:

The presence of at least one measurable lesion. Palpable lesions that are not measurable by radiologic or photographic evaluations may not be utilized as the only measurable lesion.

Non-Measurable Only Disease:

The presence of only non-measurable lesions. Note: non-measurable only disease is not allowed per protocol.

Immune-Related RECIST Response Criteria

TABLE 22
Evaluation of target lesions

| | |
|---|---|
| New, | Incorporated into tumor burden |
| measurable^{a} | |
| lesions | |
| New, | Do not define progression (but preclude mCR) |
| nonmeasurable | |
| lesions | |
| irCR | Disappearance of all lesions in two consecutive |
| | observations not less than 4 weeks apart. Any |
| | pathological lymph nodes (whether target or non- |
| | target) must have reduction in short axis to < |
| | 10 mm. |
| irPR | ≥30% decrease in tumor burden compared with |
| | baseline in two observations at least 4 weeks |
| | apart |
| irSD | 30% decrease in tumor burden compared with |
| | baseline cannot be established nor 20% increase |
| | compared with nadir |
| irPD^{b} | At least 20% increase in tumor burden compared |
| | with nadir (at any single time point) in two |
| | consecutive observations at least 4 weeks apart. |
| | In addition to the relative increase of 20%, the |
| | sum must also demonstrate an absolute increase |
| | of at least 5 mm. |

^{a}Measureable according to RECIST v1.1.
^{b}Treatment decisions will be based upon the immune-related RECIST criteria.

Antitumor Response Based on Total Measurable Tumor Burden

For the Modified RECIST criteria based on RECIST v1.1 and Immune-Related RECIST Criteria [Wolchok, 2009], the initial index and measurable new lesions are taken into account. At the baseline tumor assessment, the sum of the longest diameters (SLD) in the plane of measurement of all index lesions (maximum of five lesions total (and a maximum of two lesions per organ) representative of all involved) is calculated. Note: If lymph nodes are included in the SLD, only the short axis of the lymph node(s) is added into the sum. The short axis is the longest perpendicular diameter to the longest diameter of a lymph node or nodal mass. At each subsequent tumor assessment, the SLD of the baseline index lesions and of new, measurable lesions (≥10 mm; up to 5 new lesions per organ: 5 new cutaneous lesions and 10 visceral lesions) are added together to provide the total tumor burden:

Tumor Burden=SLD_{index lesions}+SLD_{new, measurable lesions}

Time-Point Response Assessment Using the Immune-Related RECIST Criteria

Percentage changes in tumor burden per assessment time point describe the size and growth kinetics of both conventional and new, measurable lesions as they appear. At each tumor assessment, the response in index and new, measurable lesions is defined based on the change in tumor burden (after ruling out irPD). Decreases in tumor burden must be assessed relative to baseline measurements (i.e., the SLD of all index lesions at screening).

Evaluation of Non-Target Lesions

Definitions for assessment of response for non-target lesions are as follows:
Complete Response (CR): The disappearance of all non-target lesions. All lymph nodes identified as a site of disease at baseline must be non-pathological (e.g. <10 mm short axis).
Non-CR/Non-PD: The persistence of 1 or more non-target lesion(s) or lymph nodes identified as a site of disease at baseline ≥10 mm short axis.
Progressive Disease (PD): Unequivocal progression of existing non-target lesions.
Not Applicable (NA): No non-target lesions at baseline.
Not Evaluable (NE): Cannot be classified by one of the four preceding definitions.

Note:
In the presence of measurable disease, progression on the basis of solely non-target disease requires substantial worsening such that even in the presence of SD or PR in target disease, the overall tumor burden has increased sufficiently to merit discontinuation of therapy.
Sites of non-target lesions, which are not assessed at a particular time point based on the assessment schedule, should be excluded from the response determination (e.g. non-target response does not have to be “Not Evaluable”).

New Lesions

New malignancies denoting disease progression must be unequivocal. Lesions identified in follow-up in an anatomical location not scanned at baseline are considered new lesions.

Any equivocal new lesions should continue to be followed. Treatment can continue at the discretion of the investigator until the next scheduled assessment. If at the next assessment the new lesion is considered to be unequivocal, progression should be documented.

Evaluation of Overall Response

Table 23 presents the overall response at an individual time point for all possible combinations of tumor responses in target and non-target lesions with or without the appearance of new lesions for subjects with measurable disease at baseline.

TABLE 23
Evaluation of Overall Response for Subjects
with Measurable Disease at Baseline

| Target | | | |
|---|---|---|---|
| Lesions | Non-Target Lesions | New Lesions | Overall Response |
| CR | CR or NA | No | CR |
| CR | Non-CR/Non-PD or NE | No | PR |
| PR | Non-PD or NA or NE | No | PR |
| SD | Non-PD or NA or NE | No | SD |
| NE | Non-PD or NA or NE | No | NE |
| PD | Any | Yes or No | PD |
| Any | PD | Yes or No | PD |
| Any | Any | Yes | PD |

CR = Complete response,
PR = Partial response,
SD = Stable disease,
PD = Progressive disease,
NA = Not applicable, and
NE = Not Evaluable

Evaluation of Best Overall Response

The best overall response is the best response recorded from the start of the treatment until disease progression/recurrence and will be determined programmatically by GSK based on the investigators assessment of response at each time point.
To be assigned a status of SD, follow-up disease assessment must have met the SD criteria at least once after the first dose at a minimum interval of days.
If the minimum time for SD is not met, best response will depend on the subsequent assessments. For example if an assessment of PD follows the assessment of SD and SD does not meet the minimum time requirement the best response will be PD. Alternatively, subjects lost to follow-up after an SD assessment not meeting the minimum time criteria will be considered not evaluable.

Confirmation Criteria:

To be assigned a status of PR or CR, a confirmatory disease assessment should be performed no less than 4 weeks (28 days) after the criteria for response are first met.

ECOG Performance Status^{a}

| Grade | Descriptions |
|---|---|
| 0 | Normal activity. Fully active, able to carry on all |
| | pre-disease performance without restriction. |
| 1 | Symptoms, but ambulatory. Restricted in physically |
| | strenuous activity, but ambulatory and able to carry |
| | out work of a light or sedentary nature (e.g., light |
| | housework, office work). |
| 2 | In bed < 50% of the time. Ambulatory and capable of |
| | all self-care, but unable to carry out any work |
| | activities. Up and about more than 50% of waking |
| | hours. |
| 3 | In bed > 50% of the time. Capable of only limited |
| | self-care, confined to bed or chair more than 50% |
| | of waking hours. |
| 4 | 100% bedridden. Completely disabled. Cannot carry |
| | on any self-care. Totally confined to bed or chair. |
| 5 | Dead. |

^{a}Oken, 1982.

### Appendix 7: Genetic Research

Genetic Research Objectives and Analyses

The objectives of the genetic research are to investigate the relationship between genetic variants and:
Response to medicine, including ANTIBODY 106-222 or pembrolizumab or any concomitant medicines;
NSCLC, SCCHN, RCC, melanoma, bladder, STS, TNBC or MSI CRC, susceptibility, severity, and progression and related conditions

Genetic data may be generated while the study is underway or following completion of the study. Genetic evaluations may include focused candidate gene approaches and/or examination of a large number of genetic variants throughout the genome (whole genome analyses). Genetic analyses will utilize data collected in the study and will be limited to understanding the objectives highlighted above. Analyses may be performed using data from multiple clinical studies to investigate these research objectives.

Appropriate descriptive and/or statistical analysis methods will be used. A detailed description of any planned analyses will be documented in a Reporting and Analysis Plan (RAP) prior to initiation of the analysis. Planned analyses and results of genetic investigations will be reported either as part of the clinical RAP and study report, or in a separate genetics RAP and report, as appropriate.

Study Population

Any subject who is enrolled in the study can participate in genetic research. Any subject who has received an allogeneic bone marrow transplant must be excluded from the genetic research.

Study Assessments and Procedures

A key component of successful genetic research is the collection of samples during clinical studies. Collection of samples, even when no a priori hypothesis has been identified, may enable future genetic analyses to be conducted to help understand variability in disease and medicine response.
A 6 mL blood sample will be taken for deoxyribonucleic acid (DNA) extraction. A blood sample is collected at the baseline visit, after the subject has met all eligibility requirements and provided informed consent for genetic research. Instructions for collection and shipping of the genetic sample are described in the laboratory manual. The DNA from the blood sample may undergo quality control analyses to confirm the integrity of the sample. If there are concerns regarding the quality of the sample, then the sample may be destroyed. The blood sample is taken on a single occasion unless a duplicate sample is required due to an inability to utilize the original sample.

The genetic sample is labeled (or “coded”) with the same study specific number used to label other samples and data in the study. This number can be traced or linked back to the subject by the investigator or site staff. Coded samples do not carry personal identifiers (such as name or social security number).

Samples will be stored securely and may be kept for up to 15 years after the last subject completes the study, or GSK may destroy the samples sooner. GSK or those working with GSK (for example, other researchers) will only use samples collected from the study for the purpose stated in this protocol and in the informed consent form. Samples may be used as part of the development of a companion diagnostic to support the GSK medicinal product.

Subjects can request their sample to be destroyed at any time.

Informed Consent

Subjects who do not wish to participate in the genetic research may still participate in the study. Genetic informed consent must be obtained prior to any blood sample for genetic research being taken.

Subject Withdrawal from Study

If a subject who has consented to participate in genetic research withdraws from the clinical study for any reason other than being lost to follow-up, the subject will be given a choice of one of the following options concerning the genetic sample, if already collected:
Continue to participate in the genetic research in which case the genetic DNA sample is retained
Discontinue participation in the genetic research and destroy the genetic DNA sample

If a subject withdraws consent for genetic research or requests sample destruction for any reason, the investigator must complete the appropriate documentation to request sample destruction within the timeframe specified by GSK and maintain the documentation in the site study records.

Genotype data may be generated during the study or after completion of the study and may be analyzed during the study or stored for future analysis.
If a subject withdraws consent for genetic research and genotype data has not been analyzed, it will not be analyzed or used for future research.
Genetic data that has been analyzed at the time of withdrawn consent will continue to be stored and used, as appropriate.

Screen and Baseline Failures

If a sample for genetic research has been collected and it is determined that the subject does not meet the entry criteria for participation in the study, then the investigator should instruct the subject that their genetic sample will be destroyed. No forms are required to complete this process as it will be completed as part of the consent and sample reconciliation process. In this instance a sample destruction form will not be available to include in the site files.

Provision of Study Results and Confidentiality of Subject's Genetic Data

GSK may summarize the genetic research results in the clinical study report, or separately and may publish the results in scientific journals.

GSK may share genetic research data with other scientists to further scientific understanding in alignment with the informed consent. GSK does not inform the subject, family members, insurers, or employers of individual genotyping results that are not known to be relevant to the subject's medical care at the time of the study, unless required by law. This is due to the fact that the information generated from genetic studies is generally preliminary in nature, and therefore the significance and scientific validity of the results are undetermined. Further, data generated in a research laboratory may not meet regulatory requirements for inclusion in clinical care.

### Example 2

The MC38 colon adenocarcinoma tumor model, syngeneic to the C57/BL6 strain, was used to provide evidence for improved anti-tumor activity using the combination of an anti-OX40 agonist antibody with an anti-PD-1 antagonist antibody. This experiment compared the anti-tumor response of MC38 tumor-bearing mice to treatment with one of three regimens: monotherapy with a mouse anti-mouse PD-1 monoclonal antibody (anti-PD-1), monotherapy with a rat anti-mouse OX40 antibody, clone OX-86, (anti-OX40) and combination therapy with these two agents administered concurrently. In this study, anti-PD-1 was administered at 5 mg/kg, intraperitoneally (IP), every 5 days for each of 4 cycles. Anti-OX40 was administered at 10 mg/kg, IP, every 5 days for each of 4 cycles. The isotype control arm entailed a combination of a mouse monoclonal antibody specific for adenoviral hexon 25 of the isotype IgG1, administered at 5 mg/kg, IP, every 5 days for each of 4 cycles, and a rat monoclonal antibody specific for human IL-4 of the isotype IgG1, administered at 10 mg/kg, IP, every 5 days for each of 4 cycles. Treatment was initiated when the mean tumor volume reached 115 mm³ (Day 0).

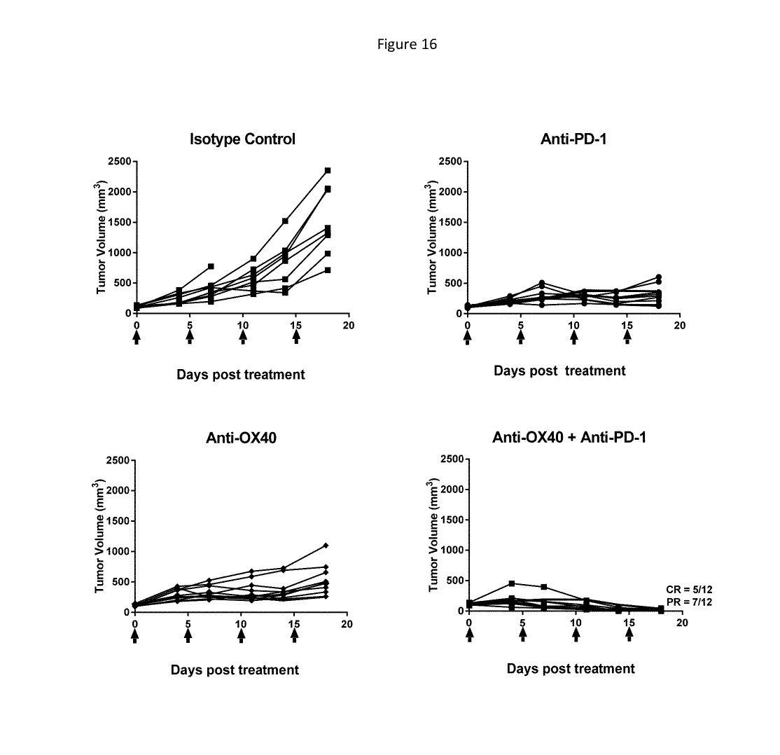

As demonstrated by results shown in FIG. 16, the mean anti-tumor response of combination therapy with the PD-1 antagonist and anti-OX40 agonist was greater than the anti-tumor response observed with either anti-OX40 single agent treatment (p=0.00005 two-sided p-values Gehan-Breslow nonparametric test) or anti-PD-1 (p=0.00005 two-sided p-values Gehan-Breslow nonparametric test) single agent treatment at Day 18. As shown in FIG. 16, the combination of these two agents demonstrated 5 out of 12 complete regressions such that no measurable tumor remained and 7 out of 12 partial regressions wherein tumor volume on Day 18 was less than the starting tumor volume on Day 0. Complete and partial regressions were not observed in the single agent treatment groups. The statistical significance of the responses to the different treatments was determined using a Fishers Exact Pair-Wise Test, and the results are shown in Table 24. Treatment tolerability was assessed by monitoring animal body weight. There was no significant body weight loss associated with administration of single agents or combination therapy indicating that treatments were well tolerated. Three animal deaths (two in the isotype control group, one in the combination group) appeared to be due to fighting-associated wounds and were not treatment related.

TABLE 24
Two-tailed Fisher's exact test: pairwise
comparison of tumor volumes at day 18

| | Tumor Volume (Day 18) | | | |
|---|---|---|---|---|
| Treatment Pairs | Non-Zero | Zero | Total | P Value |
| rat Isotype | 12 | 0 | 12 | >0.9999 |
| 10 mg/kg anti- | 12 | 0 | 12 | |
| OX40 | | | | |
| Total | 24 | 0 | 24 | |
| murine Isotype | 12 | 0 | 12 | >0.9999 |
| 5 mg/kg anti-mPD- | 12 | 0 | 12 | |
| 1 | | | | |
| Total | 24 | 0 | 24 | |
| 5 mg/kg anti-mPD- | 12 | 0 | 12 | >0.9999 |
| 1 | | | | |
| 10 mg/kg anti- | 12 | 0 | 12 | |
| OX40 | | | | |
| Total | 24 | 0 | 24 | |
| 5 mg/kg anti-mPD- | 12 | 0 | 12 | 0.0373 |
| 1 | | | | |
| 5 mg/kg anti-mPD- | 7 | 5 | 12 | |
| 1 + 10 mg/kg anti- | | | | |
| OX40 | | | | |
| Total | 19 | 5 | 24 | |
| 10 mg/kg anti- | 12 | 0 | 12 | 0.0373 |
| OX40 | | | | |
| 5 mg/kg anti-mPD- | 7 | 5 | 12 | |
| 1 + 10 mg/kg anti- | | | | |
| OX40 | | | | |
| Total | 19 | 5 | 24 | |

*Two-tailed Fisher's exact test: pairwise comparison of tumor volumes at Day 18

1. A method of treating cancer in a human in need thereof comprising administering to the human:
a therapeutically effective amount of a monoclonal antibody that binds to human OX40 comprising: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9; and
a therapeutically effective amount of a monoclonal antibody that binds to human PD-1 comprising: (a) a heavy chain variable region CDR1 comprising the amino acid sequence of SEQ ID NO:54; (b) a heavy chain variable region CDR2 comprising the amino acid sequence of SEQ ID NO:55; (c) a heavy chain variable region CDR3 comprising the amino acid sequence of SEQ ID NO:56; (d) a light chain variable region CDR1 comprising the amino acid sequence of SEQ ID NO:57; (e) a light chain variable region CDR2 comprising the amino acid sequence of SEQ ID NO:58; and (f) a light chain variable region CDR3 comprising the amino acid sequence of SEQ ID NO:59.

2. The method of claim 1, wherein the monoclonal antibody that binds to human OX40 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:5 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:11.

3. The method of claim 1, wherein the monoclonal antibody that binds to human PD-1 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:52 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:53.

4. The method of claim 1, wherein the monoclonal antibody that binds to human OX40 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:5 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:11, and the monoclonal antibody that binds to human PD-1 comprises a VH region comprising an amino acid sequence with at least 90%, 91%, 92%, 93%, 94%, 95%, 96%, 97%, 98%, 99% or 100% sequence identity to the amino acid sequence as set forth in SEQ ID NO:52 and a VL region comprising an amino acid sequence with at least 90%, 91%, 92%, 93%, 94%, 95%, 96%, 97%, 98%, 99% or 100% sequence identity to the amino acid sequence as set forth in SEQ ID NO:53.

5. The method of claim 1, wherein the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49.

6. The method of claim 1, wherein the monoclonal antibody that binds to human PD-1 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:50 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:51.

7. The method of claim 1, wherein the monoclonal antibody that binds to human OX40 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO:48 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:49, and the monoclonal antibody that binds to human PD-1 comprises a heavy chain comprising an amino acid sequence with at least 90%, 91%, 92%, 93%, 94%, 95%, 96%, 97%, 98%, 99% or 100% sequence identity to the amino acid sequence as set forth in SEQ ID NO: 50 and a light chain comprising an amino acid sequence with at least 90%, 91%, 92%, 93%, 94%, 95%, 96%, 97%, 98%, 99% or 100% sequence identity to the amino acid sequence as set forth in SEQ ID NO:51.

8. The method of claim 1, wherein the monoclonal antibody that binds to human OX40 comprises a heavy chain comprising the amino acid sequence set forth in SEQ ID NO:48 and a light chain comprising the amino acid sequence set forth in SEQ ID NO:49, and the monoclonal antibody that binds to human PD-1 comprises a heavy chain comprising the amino acid sequence set forth in SEQ ID NO: 50 and a light chain comprising the amino acid sequence set forth in SEQ ID NO:51.

9. The method of claim 1, wherein the cancer is a solid tumor.

10. The method of claim 1, wherein the cancer is selected from the group consisting of: melanoma, lung cancer, kidney cancer, breast cancer, head and neck cancer, colon cancer, ovarian cancer, pancreatic cancer, liver cancer, prostate cancer, bladder cancer, and gastric cancer.

11. The method of claim 1, wherein the monoclonal antibody that binds to OX40 and the monoclonal antibody that binds to human PD-1 are administered at the same time.

12. The method of claim 1, wherein the monoclonal antibody that binds to human OX40 and the monoclonal antibody that binds to human PD-1 are administered sequentially, in any order.

13. The method of claim 1, wherein the monoclonal antibody that binds to OX40 and/or the monoclonal antibody that binds to human PD-1 are administered intravenously.

14. The method of claim 1, wherein the monoclonal antibody that binds to OX40 and/or the monoclonal antibody that binds to human PD-1 are administered intratumorally.

15. The method of claim 1, wherein the monoclonal antibody that binds to OX40 is administered at a dose of about 0.1 mg/kg to about 10 mg/kg.

16. The method of claim 1, wherein the monoclonal antibody that binds to OX40 is administered at a frequency selected from the group consisting of: once daily, once weekly, once every two weeks (Q2W), and once every three weeks (Q3W).

17. The method of claim 1, wherein the monoclonal antibody that binds to human PD-1 is administered at a dose of about 200 mg.

18. The method of claim 1, wherein the monoclonal antibody that binds to human PD-1 is administered Q3W.

19. A method of reducing tumor size in a human having cancer comprising administering a therapeutically effective amount of a monoclonal antibody that binds to human OX40 that comprises a heavy chain comprising the amino acid sequence set forth in SEQ ID NO:48 and a light chain comprising the amino acid sequence set forth in SEQ ID NO:49, and a therapeutically effective amount of a monoclonal antibody that binds to human PD-1 that comprises a heavy chain comprising the amino acid sequence set forth in SEQ ID NO: 50 and a light chain comprising the amino acid sequence set forth in SEQ ID NO:51. to said human.

20. (canceled)

21. The method of claim 1, wherein the monoclonal antibody that binds to human PD-1 is intravenously administered to the human starting at least 1 hour and no more than 2 hours following the end of intravenous administration of the monoclonal antibody that binds to human OX40.

22. A pharmaceutical composition or kit comprising
a therapeutically effective amount of a monoclonal antibody that binds to human OX40 comprising: (a) a heavy chain variable region CDR1 comprising the amino acid sequence of SEQ ID NO:1; (b) a heavy chain variable region CDR2 comprising the amino acid sequence of SEQ ID NO:2; (c) a heavy chain variable region CDR3 comprising the amino acid sequence of SEQ ID NO:3; (d) a light chain variable region CDR1 comprising the amino acid sequence of SEQ ID NO:7; (e) a light chain variable region CDR2 comprising the amino acid sequence of SEQ ID NO:8; and (f) a light chain variable region CDR3 comprising the amino acid sequence of SEQ ID NO:9; and
a therapeutically effective amount of a monoclonal antibody that binds to human PD-1 comprising: (a) a heavy chain variable region CDR1 comprising the amino acid sequence of SEQ ID NO:54; (b) a heavy chain variable region CDR2 comprising the amino acid sequence of SEQ ID NO:55; (c) a heavy chain variable region CDR3 comprising the amino acid sequence of SEQ ID NO:56; (d) a light chain variable region CDR1 comprising the amino acid sequence of SEQ ID NO:57; (e) a light chain variable region CDR2 comprising the amino acid sequence of SEQ ID NO:58; and (f) a light chain variable region CDR3 comprising the amino acid sequence of SEQ ID NO:59.

23.-31. (canceled)


SEQUENCE LISTING (115 sequences)

SEQ ID NO: 1 - protein, 5 aa, Mus sp.
DYSMH

SEQ ID NO: 2 - protein, 17 aa, Mus sp.
WINTETGEPTYADDFKG

SEQ ID NO: 3 - protein, 13 aa, Mus sp.
PYYDYVSYYAMDY

SEQ ID NO: 4 - protein, 122 aa, Mus sp.
QIQLVQSGPELKKPGETVKISCKASGYTFTDYSMHWVKQAPGKGLKWMGWINTETGEPTYADDFKGRFAFSLETSASTAYLQINNLKNEDTATYFCANPYYDYVSYYAMDYWGHGTSVTVSS

SEQ ID NO: 5 - protein, 122 aa, Artificial Sequence
  source: /note=“Description of Artificial Sequence: Synthetic polypeptide”
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 6 - DNA, 458 bp, Artificial Sequence
  source: /note=“Description of Artificial Sequence: Synthetic polynucleotide”
actagtaccaccatggcttgggtgtggaccttgctattcctgatggcagctgcccaaagtatccaagcacaggttcagttggtgcagtctggatctgagctgaagaagcctggagcctcagtcaaggtttcctgcaaggcttctggttataccttcacagactattcaatgcactgggtgcgacaggctccaggacaaggtttaaagtggatgggctggataaacactgagactggtgagccaacatatgcagatgacttcaagggacggtttgtcttctctttggacacctctgtcagcactgcctatttgcagatcagcagcctcaaagctgaggacacggctgtgtattactgtgctaatccctactatgattacgtctcttactatgctatggactactggggtcagggaaccacggtcaccgtctcctcaggtaagaatggcctctcaagctt

SEQ ID NO: 7 - protein, 11 aa, Mus sp.
KASQDVSTAVA

SEQ ID NO: 8 - protein, 7 aa, Mus sp.
SASYLYT

SEQ ID NO: 9 - protein, 9 aa, Mus sp.
QQHYSTPRT

SEQ ID NO: 10 - protein, 107 aa, Mus sp.
DIVMTQSHKFMSTSVRDRVSITCKASQDVSTAVAWYQQKPGQSPKLLIYSASYLYTGVPDRFTGSGSGTDFTFTISSVQAEDLAVYYCQQHYSTPRTFGGGTKLEIK

SEQ ID NO: 11 - protein, 107 aa, Artificial Sequence
  source: /note=“Description of Artificial Sequence: Synthetic polypeptide”
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 12 - DNA, 416 bp, Artificial Sequence
  source: /note=“Description of Artificial Sequence: Synthetic polynucleotide”
gctagcaccaccatggagtcacagattcaggtctttgtattcgtgtttctctggttgtctggtgttgacggagacattcagatgacccagtctccatcctccctgtccgcatcagtgggagacagggtcaccatcacctgcaaggccagtcaggatgtgagtactgctgtagcctggtatcaacagaaaccaggaaaagcccctaaactactgatttactcggcatcctacctctacactggagtcccttcacgcttcagtggcagtggatctgggacggatttcactttcaccatcagcagtctgcagcctgaagacattgcaacatattactgtcagcaacattatagtactcctcggacgttcggtcagggcaccaagctggaaatcaaacgtaagtagaatccaaagaattc

SEQ ID NO: 13 - protein, 5 aa, Mus sp.
SHDMS

SEQ ID NO: 14 - protein, 17 aa, Mus sp.
AINSDGGSTYYPDTMER

SEQ ID NO: 15 - protein, 11 aa, Mus sp.
HYDDYYAWFAY

SEQ ID NO: 16 - protein, 120 aa, Mus sp.
EVQLVESGGGLVQPGESLKLSCESNEYEFPSHDMSWVRKTPEKRLELVAAINSDGGSTYYPDTMERRFIISRDNTKKTLYLQMSSLRSEDTALYYCARHYDDYYAWFAYWGQGTLVTVSA

SEQ ID NO: 17 - protein, 120 aa, Artificial Sequence
  source: /note=“Description of Artificial Sequence: Synthetic polypeptide”
EVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 18 - DNA, 451 bp, Artificial Sequence
  source: /note=“Description of Artificial Sequence: Synthetic polynucleotide”
actagtaccaccatggacttcgggctcagcttggttttccttgtccttattttaaaaagtgtacagtgtgaggtgcagctggtggagtctgggggaggcttagtgcagcctggagggtccctgagactctcctgtgcagcctctgaatacgagttcccttcccatgacatgtcttgggtccgccaggctccggggaaggggctggagttggtcgcagccattaatagtgatggtggtagcacctactatccagacaccatggagagacgattcaccatctccagagacaatgccaagaactcactgtacctgcaaatgaacagtctgagggccgaggacacagccgtgtattactgtgcaagacactatgatgattactacgcctggtttgcttactggggccaagggactatggtcactgtctcttcaggtgagtcctaacttcaagctt

SEQ ID NO: 19 - protein, 15 aa, Mus sp.
RASKSVSTSGYSYMH

SEQ ID NO: 20 - protein, 7 aa, Mus sp.
LASNLES

SEQ ID NO: 21 - protein, 9 aa, Mus sp.
QHSRELPLT

SEQ ID NO: 22 - protein, 111 aa, Mus sp.
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELK

SEQ ID NO: 23 - protein, 111 aa, Artificial Sequence
  source: /note=“Description of Artificial Sequence: Synthetic polypeptide”
EIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

SEQ ID NO: 24 - DNA, 428 bp, Artificial Sequence
  source: /note=“Description of Artificial Sequence: Synthetic polynucleotide”
gctagcaccaccatggagacagacacactcctgttatgggtactgctgctctgggttccaggttccactggtgaaattgtgctgacacagtctcctgctaccttatctttgtctccaggggaaagggccaccctctcatgcagggccagcaaaagtgtcagtacatctggctatagttatatgcactggtaccaacagaaaccaggacaggctcccagactcctcatctatcttgcatccaacctagaatctggggtccctgccaggttcagtggcagtgggtctgggacagacttcaccctcaccatcagcagcctagagcctgaggattttgcagtttattactgtcagcacagtagggagcttccgctcacgttcggcggagggaccaaggtcgagatcaaacgtaagtacacttttctgaattc

SEQ ID NO: 25 - protein, 5 aa, Mus sp.
DAWMD

SEQ ID NO: 26 - protein, 19 aa, Mus sp.
EIRSKANNHATYYAESVNG

SEQ ID NO: 27 - protein, 8 aa, Mus sp.
GEVFYFDY

SEQ ID NO: 28 - DNA, 414 bp, Mus sp.
atgtacttgggactgaactatgtattcatagtttttctcttaaatggtgtccagagtgaagtgaagcttgaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctca

SEQ ID NO: 29 - protein, 138 aa, Mus sp.
MYLGLNYVFIVFLLNGVQSEVKLEESGGGLVQPGGSMKLSCAASGFTFSDAWMDWVRQSPEKGLEWVAEIRSKANNHATYYAESVNGRFTISRDDSKSSVYLQMNSLRAEDTGIYYCTWGEVFYFDYWGQGTTLTVSS

SEQ ID NO: 30 - DNA, 448 bp, Artificial Sequence
  source: /note=“Description of Artificial Sequence: Synthetic polynucleotide”
actagtaccaccatgtacttgggactgaactatgtattcatagtttttctcttaaatggtgtccagagtgaagtgaagctggaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctcaggtgagtccttaaaacaagctt

SEQ ID NO: 31 - protein, 138 aa, Artificial Sequence; identical to SEQ ID NO: 29
  source: /note=“Description of Artificial Sequence: Synthetic polypeptide”

SEQ ID NO: 32 - protein, 11 aa, Mus sp.
KSSQDINKYIA

SEQ ID NO: 33 - protein, 7 aa, Mus sp.
YTSTLQP

SEQ ID NO: 34 - protein, 8 aa, Mus sp.
LQYDNLLT

SEQ ID NO: 35 - DNA, 378 bp, Mus sp.
atgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaa

SEQ ID NO: 36 - protein, 126 aa, Mus sp.
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELK

SEQ ID NO: 37 - DNA, 413 bp, Artificial Sequence
  source: /note=“Description of Artificial Sequence: Synthetic polynucleotide”
gctagcaccaccatgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaacgtaagtacacttttctgaattc

SEQ ID NO: 38 - protein, 126 aa, Artificial Sequence; identical to SEQ ID NO: 36
  source: /note=“Description of Artificial Sequence: Synthetic polypeptide”

SEQ ID NO: 39 - DNA, 20 bp, Artificial Sequence
  source: /note=“Description of Artificial Sequence: Synthetic primer”
cgctgttttgacctccatag

SEQ ID NO: 40 - DNA, 20 bp, Artificial Sequence
  source: /note=“Description of Artificial Sequence: Synthetic primer”
tgaaagatgagctggaggac

SEQ ID NO: 41 - DNA, 20 bp, Artificial Sequence
  source: /note=“Description of Artificial Sequence: Synthetic primer”
ctttcttgtccaccttggtg

SEQ ID NO: 42 - DNA, 19 bp, Artificial Sequence
  source: /note=“Description of Artificial Sequence: Synthetic primer”
gctgtcctacagtcctcag

SEQ ID NO: 43 - DNA, 18 bp, Artificial Sequence
  source: /note=“Description of Artificial Sequence: Synthetic primer”
acgtgccaagcatcctcg

SEQ ID NO: 44 - DNA, 1407 bp, Artificial Sequence
  source: /note=“Description of Artificial Sequence: Synthetic polynucleotide”
atgtacttgggactgaactatgtattcatagtttttctcttaaatggtgtccagagtgaagtgaagctggaggagtctggaggaggcttggtgcaacctggaggatccatgaaactctcttgtgctgcctctggattcacttttagtgacgcctggatggactgggtccgccagtctccagagaaggggcttgagtgggttgctgaaattagaagcaaagctaataatcatgcaacatactatgctgagtctgtgaatgggaggttcaccatctcaagagatgattccaaaagtagtgtctacctgcaaatgaacagcttaagagctgaagacactggcatttattactgtacgtggggggaagtgttctactttgactactggggccaaggcaccactctcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 45 - protein, 469 aa, Artificial Sequence [sequence omitted]

SEQ ID NO: 46 - DNA, 702 bp, Artificial Sequence
  source: /note=“Description of Artificial Sequence: Synthetic polynucleotide”
atgagaccgtctattcagttcctggggctcttgttgttctggcttcatggtgctcagtgtgacatccagatgacacagtctccatcctcactgtctgcatctctgggaggcaaagtcaccatcacttgcaagtcaagccaagacattaacaagtatatagcttggtaccaacacaagcctggaaaaggtcctaggctgctcatacattacacatctacattacagccaggcatcccatcaaggttcagtggaagtgggtctgggagagattattccttcagcatcagcaacctggagcctgaagatattgcaacttattattgtctacagtatgataatcttctcacgttcggtgctgggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 47 - protein, 233 aa, Artificial Sequence
  source: /note=“Description of Artificial Sequence: Synthetic polypeptide”
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 452 aa, Artificial Sequence
  source: /note=“Description of Artificial Sequence: Synthetic polypeptide”
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 214 aa, Artificial Sequence
  source: /note=“Description of Artificial Sequence: Synthetic polypeptide”
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 446 aa, Artificial Sequence
  source: /note=“Description of Artificial Sequence: Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 218 aa, Artificial Sequence
  source: /note=“Description of Artificial Sequence: Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 120 aa, Artificial Sequence [sequence omitted]

SEQ ID NO: 53 - protein, 111 aa, Artificial Sequence [sequence omitted]

SEQ ID NO: 54 - protein, 5 aa, Artificial Sequence
  source: /note=“Description of Artificial Sequence: Synthetic peptide”
NYYMY

SEQ ID NO: 55 - protein, 17 aa, Artificial Sequence
  source: /note=“Description of Artificial Sequence: Synthetic peptide”
GINPSNGGTNFNEKFKN

SEQ ID NO: 56 - protein, 11 aa, Artificial Sequence
  source: /note=“Description of Artificial Sequence: Synthetic peptide”
RDYRFDMGFDY

SEQ ID NO: 57 - protein, 15 aa, Artificial Sequence
  source: /note=“Description of Artificial Sequence: Synthetic peptide”
RASKGVSTSGYSYLH

SEQ ID NO: 58 - protein, 7 aa, Artificial Sequence
  source: /note=“Description of Artificial Sequence: Synthetic peptide”
LASYLES

SEQ ID NO: 59 - protein, 9 aa, Artificial Sequence
  source: /note=“Description of Artificial Sequence: Synthetic peptide”
QHSRDLPLT

SEQ ID NO: 60 - DNA, 435 bp, Artificial Sequence
  source: /note=“Description of Artificial Sequence: Synthetic hPD-1.08A heavy chain variable region sequence”
atgrgatggagctgtatcatkctctttttggtagcaacagctacaggtgtccactcccaggtccaactgcagcagcctggggctgaactggtgaagcctggggcttcagtgaagttgtcctgcaaggcctctggctacaccttcaccagttattatctgtactggatgaaacagaggcctggacaaggccttgagtggattgggggggttaatcctagtaatggtggtactaacttcagtgagaagttcaagagcaaggccacactgactgtagacaaatcctccagcacagcctacatgcaactcagcagcctgacatctgaggactctgcggtctattactgtacaagaagggattctaactacgacgggggctttgactactggggccaaggcactactctcacagtctcctcagccaaaacgacaccccca

SEQ ID NO: 61 - DNA, 453 bp, Artificial Sequence
  source: /note=“Description of Artificial Sequence: Synthetic hPD-1.08A light chain variable region sequence”
atggagacagacacactcctgctatgggtactgctgctctgggttccaggttccactggtgacattgtgctgacacagtctcctacttccttagctgtatctctggggcagagggccaccatctcatgcagggccagcaaaagtgtcagtacatctggctttagttatttgcactggtaccaacagaaaccaggacagccacccaaactcctcatctttcttgcatccaacctagagtctggggtccctgccaggttcagtggcagtgggtctgggacagacttcaccctcaacatccatcctgtggaggaggaggacgctgcaacctattattgtcagcacagttgggagcttccgctcacgttcggtgctgggaccaagctggagctgaaacgggctgatgctgcaccaactgtatccatcttcccaccatccagtaagcttgggaagggc

SEQ ID NO: 62 - DNA, 464 bp, Artificial Sequence
  source: /note=“Description of Artificial Sequence: Synthetic hPD-1.09A heavy chain variable region sequence”
atgraatgcagctgggttatyctctttttggtagcaacagctacaggtgtccactcccaggtccaactgcagcagcctggggctgaactggtgaagcctgggacttcagtgaagttgtcctgcaaggcttctggctacaccttcaccaactactatatgtactgggtgaagcagaggcctggacaaggccttgagtggattggggggattaatcctagcaatggtggtactaacttcaatgagaagttcaagaacaaggccacactgactgtagacagttcctccagcacaacctacatgcaactcagcagcctgacatctgaggactctgcggtctattactgtacaagaagggattataggttcgacatgggctttgactactggggccaaggcaccactctcacagtctcctcagccaaaacgacacccccatccgtytatcccbtggcccctggaagctt

SEQ ID NO: 63 - DNA, 438 bp, Artificial Sequence
  source: /note=“Description of Artificial Sequence: Synthetic hPD-1.09A light chain variable region sequence”
atggagwcagacacactsctgytatgggtactgctgctctgggttccaggttccactggcgacattgtgctgacacagtctcctgcttccttagctgtatctctgggacagagggccgccatctcatgcagggccagcaaaggtgtcagtacatctggctatagttatttgcactggtaccaacagaaaccaggacagtcacccaaactcctcatctatcttgcatcctacctagaatctggggtccctgccaggttcagtggcagtgggtctgggacagacttcaccctcaacatccatcctgtggaggaggaggatgctgcaacctattactgtcagcacagtagggaccttccgctcacgttcggtactgggaccaagctggagctgaaacgggctgatgctgcaccaactgtatccatcttcccaccatccagt

SEQ ID NO: 64 - protein, 122 aa, Artificial Sequence
  source: /note=“Description of Artificial Sequence: Synthetic hPD-1.08A heavy chain variable region sequence”
QVQLQQPGAELVKPGASVKLSCKASGYTFTSYYLYWMKQRPGQGLEWIGGVNPSNGGTNFSEKFKSKATLTVDKSSSTAYMQLSSLTSEDSAVYYCTRRDSNYDGGFDYWGQGTTLTVSSAK

SEQ ID NO: 65 - protein, 111 aa, Artificial Sequence
  source: /note=“Description of Artificial Sequence: Synthetic hPD-1.08A light chain variable region sequence”
DIVLTQSPTSLAVSLGQRATISCRASKSVSTSGFSYLHWYQQKPGQPPKLLIFLASNLESGVPARFSGSGSGTDFTLNIHPVEEEDAATYYCQHSWELPLTFGAGTKLELK

SEQ ID NO: 66 - protein, 122 aa, Artificial Sequence
  source: /note=“Description of Artificial Sequence: Synthetic hPD-1.09A heavy chain variable region sequence”
QVQLQQPGAELVKPGTSVKLSCKASGYTFTNYYMYWVKQRPGQGLEWIGGINPSNGGTNFNEKFKNKATLTVDSSSSTTYMQLSSLTSEDSAVYYCTRRDYRFDMGFDYWGQGTTLTVSSAK

SEQ ID NO: 67 - protein, 111 aa, Artificial Sequence
  source: /note=“Description of Artificial Sequence: Synthetic hPD-1.09A light chain variable region sequence”
DIVLTQSPASLAVSLGQRAAISCRASKGVSTSGYSYLHWYQQKPGQSPKLLIYLASYLESGVPARFSGSGSGTDFTLNIHPVEEEDAATYYCQHSRDLPLTFGTGTKLELK

SEQ ID NO: 68 - protein, 15 aa, Artificial Sequence
  source: /note=“Description of Artificial Sequence: Synthetic hPD-1.08A light chain CDR1 sequence”
RASKSVSTSGFSYLH

SEQ ID NO: 69 - protein, 7 aa, Artificial Sequence
  source: /note=“Description of Artificial Sequence: Synthetic hPD-1.08A light chain CDR2 sequence”
LASNLES

SEQ ID NO: 70 - protein, 9 aa, Artificial Sequence
  source: /note=“Description of Artificial Sequence: Synthetic hPD-1-08A light chain CDR3 sequence”
QHSWELPLT

SEQ ID NO: 71 - protein, 5 aa, Artificial Sequence
  source: /note=“Description of Artificial Sequence: Synthetic hPD-1.08A heavy chain CDR1 sequence”
SYYLY

SEQ ID NO: 72 - protein, 17 aa, Artificial Sequence
  source: /note=“Description of Artificial Sequence: Synthetic hPD-1.08A heavy chain CDR2 sequence”
GVNPSNGGTNFSEKFKS

SEQ ID NO: 73 - protein, 11 aa, Artificial Sequence
  source: /note=“Description of Artificial Sequence: Synthetic hPD-1.08A heavy chain CDR3 sequence”
RDSNYDGGFDY

SEQ ID NO: 74 - protein, 15 aa, Artificial Sequence
  source: /note=“Description of Artificial Sequence: Synthetic hPD-1.09A light chain CDR1 sequence”
RASKGVSTSGYSYLH

SEQ ID NO: 75 - protein, 7 aa, Artificial Sequence
  source: /note=“Description of Artificial Sequence: Synthetic hPD-1.09A light chain CDR2 sequence”
LASYLES

SEQ ID NO: 76 - protein, 9 aa, Artificial Sequence
  source: /note=“Description of Artificial Sequence: Synthetic hPD-1.09A light chain CDR3 sequence”
QHSRDLPLT

SEQ ID NO: 77 - protein, 5 aa, Artificial Sequence
  source: /note=“Description of Artificial Sequence: Synthetic hPD-1.09A heavy chain CDR1 sequence”
NYYMY

SEQ ID NO: 78 - protein, 17 aa, Artificial Sequence
  source: /note=“Description of Artificial Sequence: Synthetic hPD-1.09A heavy chain CDR2 sequence”
GINPSNGGTNFNEKFKN

SEQ ID NO: 79 - protein, 11 aa, Artificial Sequence
  source: /note=“Description of Artificial Sequence: Synthetic hPD-1.09A heavy chain CDR3 sequence”
RDYRFDMGFDY

SEQ ID NO: 80 - DNA, 417 bp, Artificial Sequence
  source: /note=“Description of Artificial Sequence: Synthetic 109A-H heavy chain variable region sequence”
  sig_peptide (1)..(57)
atggactggacctggagcatccttttcttggtggcagcaccaacaggagcccactcccaagtgcagctggtgcagtctggagttgaagtgaagaagcccggggcctcagtgaaggtctcctgcaaggcttctggctacacctttaccaactactatatgtactgggtgcgacaggcccctggacaagggcttgagtggatgggagggattaatcctagcaatggtggtactaacttcaatgagaagttcaagaacagagtcaccttgaccacagactcatccacgaccacagcctacatggaactgaagagcctgcaatttgacgacacggccgtttattactgtgcgagaagggattataggttcgacatgggctttgactactggggccaagggaccacggtcaccgtctcgagc

SEQ ID NO: 81 - DNA, 417 bp, Artificial Sequence
  source: /note=“Description of Artificial Sequence: Synthetic Codon optimized 109A-H heavy chain variable region sequence”
  sig_peptide (1)..(57)
atggactggacctggtctatcctgttcctggtggccgctcctaccggcgctcactcccaggtgcagctggtgcagtccggcgtggaggtgaagaagcctggcgcctccgtcaaggtgtcctgcaaggcctccggctacaccttcaccaactactacatgtactgggtgcggcaggctcccggccagggactggagtggatgggcggcatcaacccttccaacggcggcaccaacttcaacgagaagttcaagaaccgggtgaccctgaccaccgactcctccaccaccaccgcctacatggagctgaagtccctgcagttcgacgacaccgccgtgtactactgcgccaggcgggactaccggttcgacatgggcttcgactactggggccagggcaccaccgtgaccgtgtcctcc

SEQ ID NO: 82 - DNA, 1398 bp, Artificial Sequence
  source: /note=“Description of Artificial Sequence: Synthetic Codon optimized 409A-H heavy chain full length sequence”
  sig_peptide (1)..(57)
atggccgtgctgggcctgctgttctgcctggtgaccttcccttcctgcgtgctgtcccaggtgcagctggtgcagtccggcgtggaggtgaagaagcctggcgcctccgtcaaggtgtcctgtaaggcctccggctacaccttcaccaactactacatgtactgggtgcggcaggccccaggccagggactggagtggatgggcggcatcaacccttccaacggcggcaccaacttcaacgagaagttcaagaaccgggtgaccctgaccaccgactcctccaccacaaccgcctacatggaactgaagtccctgcagttcgacgacaccgccgtgtactactgcgccaggcgggactaccggttcgacatgggcttcgactactggggccagggcaccaccgtgaccgtgtcctccgctagcaccaagggcccttccgtgttccctctggccccttgctcccggtccacctccgagtccaccgccgctctgggctgtctggtgaaggactacttccctgagcctgtgaccgtgagctggaactctggcgccctgacctccggcgtgcacaccttccctgccgtgctgcagtcctccggcctgtactccctgtcctccgtggtgaccgtgccttcctcctccctgggcaccaagacctacacctgcaacgtggaccacaagccttccaacaccaaggtggacaagcgggtggagtccaagtacggccctccttgccctccctgccctgcccctgagttcctgggcggaccctccgtgttcctgttccctcctaagcctaaggacaccctgatgatctcccggacccctgaggtgacctgcgtggtggtggacgtgtcccaggaagatcctgaggtccagttcaattggtacgtggatggcgtggaggtgcacaacgccaagaccaagcctcgggaggaacagttcaactccacctaccgggtggtgtctgtgctgaccgtgctgcaccaggactggctgaacggcaaggaatacaagtgcaaggtcagcaacaagggcctgccctcctccatcgagaaaaccatctccaaggccaagggccagcctcgcgagcctcaggtgtacaccctgcctcctagccaggaagagatgaccaagaatcaggtgtccctgacatgcctggtgaagggcttctacccttccgatatcgccgtggagtgggagagcaacggccagccagagaacaactacaagaccacccctcctgtgctggactccgacggctccttcttcctgtactccaggctgaccgtggacaagtcccggtggcaggaaggcaacgtcttttcctgctccgtgatgcacgaggccctgcacaaccactacacccagaagtccctgtccctgtctctgggcaag

SEQ ID NO: 83 - DNA, 393 bp, Artificial Sequence
  source: /note=“Description of Artificial Sequence: Synthetic K09A-L-11 light chain variable region sequence”
  sig_peptide (1)..(60)
atggaagccccagctcagcttctcttcctcctgctactctggctcccagataccaccggagaaattgtgttgacacagtctccagccaccctgtctttgtctccaggggaaagagccaccctctcctgcagggccagcaaaggtgtcagtacatctggctatagttatttgcactggtatcaacagaaacctggccaggctcccaggctcctcatctatcttgcatcctacctagaatctggcgtcccagccaggttcagtggtagtgggtctgggacagacttcactctcaccatcagcagcctagagcctgaagattttgcagtttattactgtcagcacagcagggaccttccgctcacgttcggcggagggaccaaagtggagatcaaa

SEQ ID NO: 84 - DNA, 393 bp, Artificial Sequence
  source: /note=“Description of Artificial Sequence: Synthetic K09A-L-16 light chain variable region sequence”
  sig_peptide (1)..(60)
atggaaaccccagcgcagcttctcttcctcctgctactctggctcccagataccaccggagaaattgtgctgactcagtctccactctccctgcccgtcacccctggagagccggcctccatctcctgcagggccagcaaaggtgtcagtacatctggctatagttatttgcattggtacctccagaagccagggcagtctccacagctcctgatctatcttgcatcctacctagaatctggggtccctgacaggttcagtggcagtggatcaggcacagattttacactgaaaatcagcagagtggaggctgaggatgttggggtttattactgccagcatagtagggaccttccgctcacgtttggccaggggaccaagctggagatcaaa

SEQ ID NO: 85 - DNA, 393 bp, Artificial Sequence
  source: /note=“Description of Artificial Sequence: Synthetic K09A-L-17 light chain variable region sequence”
  sig_peptide (1)..(60)
atgaggctccctgctcagctcctggggctgctaatgctctgggtctctggatccagtggggatattgtgatgacccagactccactctccctgcccgtcacccctggagagccggcctccatctcctgcagggccagcaaaggtgtcagtacatctggctatagttatttgcattggtatctgcagaagccagggcagtctccacagctcctgatctatcttgcatcctacctagaatctggagtcccagacaggttcagtggcagtgggtcaggcactgctttcacactgaaaatcagcagggtggaggctgaggatgttggactttattactgccagcatagtagggaccttccgctcacgtttggccaggggaccaagctggagatcaaa

SEQ ID NO: 86 - DNA, 390 bp, Artificial Sequence
  source: /note=“Description of Artificial Sequence: Synthetic Codon optimized K09A-L-11 light chain variable region sequence”
  sig_peptide (1)..(57)
atggcccctgtgcagctgctgggcctgctggtgctgttcctgcctgccatgcggtgcgagatcgtgctgacccagtcccctgccaccctgtccctgagccctggcgagcgggctaccctgagctgcagagcctccaagggcgtgtccacctccggctactcctacctgcactggtatcagcagaagccaggccaggcccctcggctgctgatctacctggcctcctacctggagtccggcgtgcctgcccggttctccggctccggaagcggcaccgacttcaccctgaccatctcctccctggagcctgaggacttcgccgtgtactactgccagcactcccgggacctgcctctgacctttggcggcggaacaaaggtggagatcaag

SEQ ID NO: 87 - DNA, 390 bp, Artificial Sequence
  source: /note=“Description of Artificial Sequence: Synthetic Codon optimized K09A-L-16 light chain variable region sequence”
  sig_peptide (1)..(57)
atggcccctgtgcagctgctgggcctgctggtgctgttcctgcctgccatgcggtgcgagatcgtgctgacccagtcccctctgtccctgcctgtgacccctggcgagcctgcctccatctcctgccgggcctccaagggcgtgtccacctccggctactcctacctgcactggtatctgcagaagcctggccagtccccccagctgctgatctacctggcctcctacctggagtccggcgtgcctgaccggttctccggctccggcagcggcaccgacttcaccctgaagatctcccgggtggaggccgaggacgtgggcgtgtactactgccagcactcccgggacctgcctctgaccttcggccagggcaccaagctggagatcaag

SEQ ID NO: 88 - DNA, 390 bp, Artificial Sequence
  source: /note=“Description of Artificial Sequence: Synthetic Codon optimized K09A-L-17 light chain variable region sequence”
  sig_peptide (1)..(57)
atggcccctgtgcagctgctgggcctgctggtgctgttcctgcctgccatgcggtgcgacatcgtgatgacccagacccctctgtccctgcctgtgacccctggcgagcctgcctccatctcctgccgggcctccaagggcgtgtccacctccggctactcctacctgcactggtatctgcagaagcctggccagtcccctcagctgctgatctacctggcctcctacctggagtccggcgtgcctgaccggttctccggctccggaagcggcaccgcttttaccctgaagatctccagagtggaggccgaggacgtgggcctgtactactgccagcactcccgggacctgcctctgaccttcggccagggcaccaagctggagatcaag

SEQ ID NO: 89 - protein, 139 aa, Artificial Sequence
  source: /note=“Description of Artificial Sequence: Synthetic 109A-H heavy chain variable region sequence”
  MISC_FEATURE (1)..(19): /note=“Signal peptide”
MDWTWSILFLVAAPTGAHSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 90 - protein, 466 aa, Artificial Sequence
  source: /note=“Description of Artificial Sequence: Synthetic 409A-H heavy chain full length sequence”
  MISC_FEATURE (1)..(19): /note=“Signal peptide”
MAVLGLLFCLVTFPSCVLSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 91 - protein, 130 aa, Artificial Sequence
  source: /note=“Description of Artificial Sequence: Synthetic K09A-L-11 light chain variable region sequence”
  MISC_FEATURE (1)..(19): /note=“Signal peptide”
MAPVQLLGLLVLFLPAMRCEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 92 - protein, 130 aa, Artificial Sequence
  source: /note=“Description of Artificial Sequence: Synthetic K09A-L-16 light chain variable region sequence”
  MISC_FEATURE (1)..(19): /note=“Signal peptide”
MAPVQLLGLLVLFLPAMRCEIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 93 - protein, 130 aa, Artificial Sequence
  source: /note=“Description of Artificial Sequence: Synthetic K09A-L-17 light chain variable region sequence”
  MISC_FEATURE (1)..(19): /note=“Signal peptide”
MAPVQLLGLLVLFLPAMRCDIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 94 - protein, 469 aa, Artificial Sequence
  source: /note=“Description of Artificial Sequence: Synthetic 109A-H heavy chain full length sequence”
  MISC_FEATURE (1)..(19): /note=“Signal peptide”
MAVLGLLFCLVTFPSCVLSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 237 aa, Artificial Sequence
  source: /note=“Description of Artificial Sequence: Synthetic K09A-L-11 light chain full length sequence”
  MISC_FEATURE (1)..(19): /note=“Signal peptide”
MAPVQLLGLLVLFLPAMRCEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 237 aa, Artificial Sequence
  source: /note=“Description of Artificial Sequence: Synthetic K09A-L-16 light chain full length sequence”
  MISC_FEATURE (1)..(19): /note=“Signal peptide”
MAPVQLLGLLVLFLPAMRCEIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 237 aa, Artificial Sequence
  source: /note=“Description of Artificial Sequence: Synthetic K09A-L-17 light chain full length sequence”
  MISC_FEATURE (1)..(19): /note=“Signal peptide”
MAPVQLLGLLVLFLPAMRCDIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 440 aa, Artificial Sequence
  source: /note=“Description of Artificial Sequence: Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 214 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 113 aa, Artificial Sequence [sequence omitted]

SEQ ID NO: 101 - protein, 107 aa, Artificial Sequence [sequence omitted]

SEQ ID NO: 102 - protein, 5 aa, Artificial Sequence
  source: /note=“Description of Artificial Sequence: Synthetic peptide”
NSGMH

SEQ ID NO: 103 - protein, 17 aa, Artificial Sequence
  source: /note=“Description of Artificial Sequence: Synthetic peptide”
VIWYDGSKRYYADSVKG

SEQ ID NO: 104 - protein, 4 aa, Artificial Sequence
  source: /note=“Description of Artificial Sequence: Synthetic peptide”
NDDY

SEQ ID NO: 105 - protein, 11 aa, Artificial Sequence
  source: /note=“Description of Artificial Sequence: Synthetic peptide”
RASQSVSSYLA

SEQ ID NO: 106 - protein, 7 aa, Artificial Sequence
  source: /note=“Description of Artificial Sequence: Synthetic peptide”
DASNRAT

SEQ ID NO: 107 - protein, 9 aa, Artificial Sequence
  source: /note=“Description of Artificial Sequence: Synthetic peptide”
QQSSNWPRT

SEQ ID NO: 108 - protein, 131 aa, Homo sapiens
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARDSRGYSYYDFWSGYFYYYYMDVWGKGTTVTVSS

SEQ ID NO: 109 - protein, 113 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPPWASYTFGQGTKLEIKRT

SEQ ID NO: 110 - protein, 141 aa, Artificial Sequence
  source: /note=“Description of Artificial Sequence: Synthetic polypeptide”
MAWVWTLLFLMAAAQSIQAQVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 111 - protein, 127 aa, Artificial Sequence
  source: /note=“Description of Artificial Sequence: Synthetic polypeptide”
MESQIQVFVFVFLWLSGVDGDIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 112 - protein, 136 aa, Homo sapiens
GLSWVFLVAILEGVQCEVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARGLTGATDAFDIWGQGTMVTVSS

SEQ ID NO: 113 - protein, 128 aa, Homo sapiens
MEAPAQLLFLLLLWLPDTTGEIVLTQSPATLSLSPGERATLSCRASQSVSSYLAWYQQKPGQAPRLLIYDASNKATGVPARFSGSGSGTDFTLTISSLEPEDFAVYYCQQSSKWPLTFGGGTKVEIKG

SEQ ID NO: 114 - protein, 139 aa, Artificial Sequence
  source: /note=“Description of Artificial Sequence: Synthetic polypeptide”
MDFGLSLVFLVLILKSVQCEVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 115 - protein, 131 aa, Artificial Sequence
  source: /note=“Description of Artificial Sequence: Synthetic polypeptide”
METDTLLLWVLLLWVPGSTGEIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK